SEQUENCE LISTING (53 sequences)

SEQ ID NO: 1 - DNA, 9000 bp, Homo sapiens
  source 1..9000: mol_type = genomic DNA
acgcctctcacctcagctcttccctgcaggctctgtggatagcaactggatcgtgggtgccacgctggagaagaagctcccacccctgcccctgacactggcccttggggccttcctgaatcaccgcaagaacaagtttcagtgtggctttggcctcaccatcggctgagccctcctggcccccgccttccacgcccttccgattccacctccacctccacctccccctgccacagaggggagacctgagcccccctcccttccctccccccttgggggtcgggggggacattggaaaggagggaccccgccaccccagcagctgaggaggggattctggaactgaatggcgcttcgggattctgagtagcaggggcagcatgcccagtgggcctggggtcccgggagggattccggaattgaggggcacgcaggattctgagcaccaggggcagaggcggccagacaacctcagggaggagtgtcctggcgtccccatcctccaaagggcctgggcccgccccgagggggcagcgagaggagcttccccatccccggtcagtccaccctgccccgtccactttcccatctcctcggtataaatcatgtttataagttatggaagaaccgggacattttacagaaaaaaaacaaaaaacaacaaaaaatatacgtgggaaaaaaaacgatgggaggcctccgttttctcaagtgtgtctggcctgttttgagcatttcatccggagtctggccgccctgaccttcccccagccgcctgcagggggcgccagagggccggagcacggaaagcagcggatccttgatgctgccttaagtccggctcagaggggcgcagcgtggcctggggtcgctatcttcccatccggaacatctgccctgctgggggacactacgggccttcccttgcctgagggtagggtctcaaggtcacttgcccccagcttgacctggccggagtggctatagaggactttgtccctgcagactgcagcagcagagatgacactgtctctgagtgcagagatgggggcagggagctgggagagggttcaagctactggaacagcttcagaacaactagggtactaggaactgctgtgtcagggagaaggggctcaaggactcgcaggcctgggaggaggggcctaggccagccatgggagttgggtcacctgtgtctgaggacttggtgctgtctggattttgccaacctagggctggggtcagctgatgcccaccacgactcccgagcctccaggaactgaaaccctgtctgcccccagggtctggggaaggaggctgctgagtagaaccaaccccaggttaccaaccccacctcagccaccccttgccagccaaagcaaacaggcccggcccggcactgggggttccttctcgaaccaggagttcagcctcccctgacccgcagaatcttctgatcccacccgctccaggagccaggaatgagtcccagtctctcccagttctcactgtgtggttttgccattcgtcttgctgctgaaccacgggtttctcctctgaaacatctgggatttataacagggcttaggaaagtgacagcgtctgagcgttcactgtggcctgtccattgctagccctaacataggaccgctgtgtgccagggctgtcctccatgctcaatacacgttagcttgtcaccaaacatacccgtgccgctgctttcccagtctgatgagcaaaggaacttgatgctcagagaggacaagtcatttgcccaaggtcacacagctggcaactggcagagccaggattcacgccctggcaatttgactccagaatcctaaccttaacccagaagcacggcttcaagcccctggaaaccacaatacctgtggcagccagggggaggtgctggaatctcatttcacatgtggggagggggctcccctgtgctcaaggtcacaaccaaagaggaagctgtgattaaaacccaggtcccatttgcaaagcctcgacttttagcaggtgcatcatactgttcccacccctcccatcccacttctgtccagccgcctagccccactttcttttttttctttttttgagacagtctccctcttgctgaggctggagtgcagtggcgagatctcggctcactgtaacctccgcctcccgggttcaagcgattctcctgcctcagcctcccaagtagctaggattacaggcgcccgccaccacgcctggctaacttttgtatttttagtagagatggggtttcaccatgttggccaggctggtctcaaactcctgaccttaagtgattcgcccactgtggcctcccaaagtgctgggattacaggcgtgagctaccgcccccagcccctcccatcccacttctgtccagccccctagccctactttctttctgggatccaggagtccagatccccagccccctctccagattacattcatccaggcacaggaaaggacagggtcaggaaaggaggactctgggcggcagcctccacattccccttccacgcttggcccccagaatggaggagggtgtctgtattactgggcgaggtgtcctcccttcctggggactgtggggggtggtcaaaagacctctatgccccacctccttcctccctctgccctgctgtgcctggggcagggggagaacagcccacctcgtgactgggggctggcccagcccgccctatccctgggggagggggcgggacagggggagccctataattggacaagtctgggatccttgagtcctactcagccccagcggaggtgaaggacgtccttccccaggagccggtgagaagcgcagtcgggggcacggggatgagctcaggggcctctagaaagagctgggaccctgggaacccctggcctccaggtagtctcaggagagctactcggggtcgggcttggggagaggaggagcgggggtgaggcaagcagcaggggactggacctgggaagggctgggcagcagagacgacccgacccgctagaaggtggggtggggagagcagctggactgggatgtaagccatagcaggactccacgagttgtcactatcatttatcgagcacctactgggtgtccccagtgtcctcagatctccataactggggagccaggggcagcgacacggtagctagccgtcgattggagaactttaaaatgaggactgaattagctcataaatggaacacggcgcttaactgtgaggttggagcttagaatgtgaagggagaatgaggaatgcgagactgggactgagatggaaccggcggtggggagggggtggggggatggaatttgaaccccgggagaggaagatggaattttctatggaggccgacctggggatggggagataagagaagaccaggagggagttaaatagggaatgggttgggggcggcttggtaaatgtgctgggattaggctgttgcagataatgcaacaaggcttggaaggctaacctggggtgaggccgggttggggccgggctgggggtgggaggagtcctcactggcggttgattgacagtttctccttccccagactggccaatcacaggcaggaagatgaaggttctgtgggctgcgttgctggtcacattcctggcaggtatgggggcggggcttgctcggttccccccgctcctccccctctcatcctcacctcaacctcctggccccattcaggcagaccctgggccccctcttctgaggcttctgtgctgcttcctggctctgaacagcgatttgacgctctctgggcctcggtttcccccatccttgagataggagttagaagttgttttgttgttgttgtttgttgttgttgttttgtttttttgagatgaagtctcgctctgtcgcccaggctggagtgcagtggcgggatctcggctcactgcaagctccgcctcccaggtccacgccattctcctgcctcagcctcccaagtagctgggactacaggcacatgccaccacacccgactaacttttttgtattttcagtagagacggggtttcaccatgttggccaggctggtctggaactcctgacctcaggtgatctgcccgtttcgatctcccaaagtgctgggattacaggcgtgagccaccgcacctggctgggagttagaggtttctaatgcattgcaggcagatagtgaataccagacacggggcagctgtgatctttattctccatcacccccacacagccctgcctggggcacacaaggacactcaatacatgcttttccgctgggcgcggtggctcacccctgtaatcccagcactttgggaggccaaggtgggaggatcacttgagcccaggagttcaacaccagcctgggcaacatagtgagaccctgtctctactaaaaatacaaaaattagccaggcatggtgccacacacctgtgctctcagctactcaggaggctgaggcaggaggatcgcttgagcccagaaggtcaaggttgcagtgaaccatgttcaggccgctgcactccagcctgggtgacagagcaagaccctgtttataaatacataatgctttccaagtgattaaaccgactcccccctcaccctgcccaccatggctccaaagaagcatttgtggagcaccttctgtgtgcccctaggtactagatgcctggacggggtcagaaggaccctgacccaccttgaacttgttccacacaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgaggtgagtgtccccatcctggcccttgaccctcctggtgggcggctatacctccccaggtccaggtttcattctgcccctgtcgctaagtcttggggggcctgggtctctgctggttctagcttcctcttcccatttctgactcctggctttagctctctggaattctctctctcagctttgtctctctctcttcccttctgactcagtctctcacactcgtcctggctctgtctctgtccttccctagctcttttatatagagacagagagatggggtctcactgtgttgcccaggctggtcttgaacttctgggctcaagcgatcctcccgcctcggcctcccaaagtgctgggattagaggcatgagccaccttgcccggcctcctagctccttcttcgtctctgcctctgccctctgcatctgctctctgcatctgtctctgtctccttctctcggcctctgccccgttccttctctccctcttgggtctctctggctcatccccatctcgcccgccccatcccagcccttctccccgcctcccactgtgcgacaccctcccgccctctcggccgcagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgcatctgctggcctccccctgtgatttcctctaagccccagcctcagtttctctttctgcccacatactggccacacaattctcagccccctcctctccatctgtgtctgtgtgtatctttctctctgcccttttttttttttttagacggagtctggctctgtcacccaggctagagtgcagtggcacgatcttggctcactgcaacctctgcctcttgggttcaagcgattctgctgcctcagtagctgggattacaggctcacaccaccacacccggctaatttttgtatttttagtagagacgagctttcaccatgttggccaggcaggtctcaaactcctgaccaagtgatccacccgccggcctcccaaagtgctgagattacaggcctgagccaccatgcccggcctctgcccctctttcttttttagggggcagggaaaggtctcaccctgtcacccgccatcacagctcactgcagcctccacctcctggactcaagtgataagtgatcctcccgcctcagcctttccagtagctgagactacaggcgcataccactaggattaatttgggggggggggtggtgtgtgtggagatggggtctggctttgttggccaggctgatgtggaattcctgggctcaagcgatactcccaccttggcctcctgagtagctgagactactggctagcaccaccacacccagctttttattattatttgtagagacaaggtctcaatatgttgcccaggctagtctcaaacccctgggctcaagagatcctccgccatcggcctcccaaagtgctgggattccaggcatggggctccgagcccggcctgcccaacttaataatacttgttcctcagagttgcaactccaaatgacctgagattggtgcctttattctaagctattttcattttttttctgctgtcattattctcccccttctctcctccagtcttatctgatatctgcctccttcccacccaccctgcaccccatcccacccctctgtctctccctgttctcctcaggagactctggcttcctgttttcctccacttctatcttttatctctccctcctacggtttcttttctttctccccggcctgcttgtttctcccccaacccccttcatctggatttcttcttctgccattcagtttggtttgagctctctgcttctccggttccctctgagctagctgtcccttcacccactgtgaactgggtttccctgcccaaccctcattctctttctttctttctttttttttttttttttttttttttttttttgagacagagtcttgctctgttgcccagcctggagtgcagtggtgcaatcttggttcactgcaacctccacttcccagattcaagcaattctcctgcctcagcctccagagtagctgggattacaggcgtgtcccaccacacccgactaatttttgtatttttggtagagacaaggcttcggcattgttggccaggcaggtctcgaactcctgacctcaagtaatctgcctgcctcaccctcccaaagtgctgggattacaggcatgagccacctcacccggaccatccctcattctccatcctttcctccagttgtgatgtctacccctcatgtttcccaacaagcctactgggtgctgaatccaggctgggaagagaagggagcggctcttctgtcggagtctgcaccaggcccatgctgagacgagagctggcgctcagagaggggaagcttggatggaagcccaggagccgccggcactctcttctcctcccaccccctcagttctcagagacggggaggagggttcccaccaacgggggacaggctgagacttgagcttgtatctcctgggccagctgcaacatctgcttgtccctctgcccatcttggctcctgcacaccctgaacttggtgctttccctggcactgctctgatcacccacgtggaggcagcacccctcccctggagatgactcaccagggctgagtgaggaggggaagggtcagtgtgctcacaggcagggggcctggtctgctgggcctgctgctgattcaccgtatgtccaggagctggagcagcaccctctcagagcctcagtttccccagatgtcaatgagagaatcagcatcagccacatctcccacctgaagaatcgaaccttgagttctcacctttaagaatttttttttttttttgagacagtgccttgctcagtcgaccacgctggagtgcagtagcacgatcatcaatcactgcagcctccaactcaagcaaccctcctgcctcaacctcctgagtagctggggccacaggcacccaccaccatgcctggctaattctttcttttttgagatggagtctcactctgtcgcccaggctggagtgcagtggcaccatcttggctcaccgcaacctctgccccccgggttcaagcaattctcctgcctcagcctcctgagtagctagaattacaggcgcacaccaccacaccaggctaatttttgtatttttagtagaaacgaggtttcaccacggtggtcaggttggtctcaaactcctgacatcaggtga

SEQ ID NO: 2 - RNA, 1316 nt, Homo sapiens
  source 1..1316: mol_type = mRNA
gggacagggggagccctataattggacaagtctgggatccttgagtcctactcagccccagcggaggtgaaggacgtccttccccaggagccggtgagaagcgcagtcgggggcacggggatgagctcaggggcctctagaaagagctgggaccctgggaacccctggcctccagactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgcatcaaaaaaaaaaaaaaaaaaa

SEQ ID NO: 3 - RNA, 774 nt, Homo sapiens
  source 1..774: mol_type = mRNA
tactcagccccagcggaggtgaaggacgtccttccccaggagccgactggccaatcacaggcaggaagatgaaggttctgtgggctgcgttgctggtcacattcctggcaggatgccagccaaggtggagcaagcggtggagacagagccggagcccgagctgcgccacagaccgagtggcagagcggccagcgctgggaactggcactgggtcgcttttgggattacctgcgctgggtgcagacactgtctgagcagtgcagaggagctgctcaatcccaggtcacccaggaactgagggcgctgatggacgagaccatgaaggagttgaaggcctacaaatcggaactggaggaacaactgaccccggtggcggaggagacgcgggcacggctgtccaaggagctgcaggcgncgcaggcccggctgggcgcggacatggaggacgtgtgcggccgcctggtgcagtaccgcggccgaggtgcaggccatgctcggccagagcaccgaggagctgcgggtgcgcctcgcctcccacctgcgcaactgcgtaacggctcctccgcgatgccgatgacctgcagaagcgcctggcagtgtaccaggccggggcccgcgaagcgccgancgcggcctcagcgccatccgcgagcgcctggggcccctggtggaanagggccgcgtgcgggcccgccactgttgggctcccttggccggccaaccgctanaggagtggaccctaagtttaataaagattccccaagttttcacgcc

SEQ ID NO: 4 - RNA, 884 nt, Homo sapiens
  source 1..884: mol_type = mRNA
agcggacttagccccagcggaggtgaaggacgtccttccccaggagccggtgagaagcgcagtcgggggcacggggatgagctcaggggcctctagaaagagctgggaccctgggaagccctggcctccagactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtacgaggccggggcccggagggcgccgagcgcggcctcagcgccatccgcgagcgcctggggccctggtggaacagggccgggtgcggccggcactgtgggctccctgagccgggccagccgctacaggagcgggccaggcctggggagaaccggttgaacacgcggatggagagaaatgggcagcgggaccg

SEQ ID NO: 5 - RNA, 704 nt, Homo sapiens
  source 1..704: mol_type = mRNA
agaattcggcacgaggcaggtcacncaggaactgagggcgctgcgggtgcgcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgcaaaaaaaaaaaaaaaaaa

SEQ ID NO: 6 - RNA, 1223 nt, Homo sapiens
  source 1..1223: mol_type = mRNA
gggatccttgagtcctactcagccccagcggaggtgaaggacgtccttccccaggagccgactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgcaaaaaaaaaaaaaaaaaaaaaaaaaaaaaaaaaaaaaaaaaaaa

SEQ ID NO: 7 - DNA, 20 bp, synthetic construct
  source 1..20: mol_type = other DNA
tgggtcgcttttgggattac

SEQ ID NO: 8 - DNA, 18 bp, synthetic construct
  source 1..18: mol_type = other DNA
ccatcagcgccctcagtt

SEQ ID NO: 9 - DNA, 23 bp, synthetic construct
  source 1..23: mol_type = other DNA
ctgctcagctcccaggtcaccca

SEQ ID NO: 10 - DNA, 19 bp, synthetic construct
  source 1..19: mol_type = other DNA
gaaggtgaaggtcggagtc

SEQ ID NO: 11 - DNA, 20 bp, synthetic construct
  source 1..20: mol_type = other DNA
gaagatggtgatgggatttc

SEQ ID NO: 12 - DNA, 20 bp, synthetic construct
  source 1..20: mol_type = other DNA
caagcttcccgttctcagcc

SEQ ID NO: 13 - DNA, 20 bp, synthetic construct
  source 1..20: mol_type = other DNA
tgctggacccaacacaaatg

SEQ ID NO: 14 - DNA, 20 bp, synthetic construct
  source 1..20: mol_type = other DNA
tgccatccaaccactcagtc

SEQ ID NO: 15 - DNA, 26 bp, synthetic construct
  source 1..26: mol_type = other DNA
ttcccagtttttcatctgcactgcca

SEQ ID NO: 16 - DNA, 15 bp, synthetic construct
  source 1..15: mol_type = other DNA
tcgccgcttgctgca

SEQ ID NO: 17 - DNA, 17 bp, synthetic construct
  source 1..17: mol_type = other DNA
atcggccgtgatgtcga

SEQ ID NO: 18 - DNA, 23 bp, synthetic construct
  source 1..23: mol_type = other DNA
ccatggtcaaccccaccgtgttc

SEQ ID NO: 19 - DNA, 20 bp, synthetic construct
  source 1..20: mol_type = other DNA
cgcgtggtgaaacacttcta

SEQ ID NO: 20 - DNA, 22 bp, synthetic construct
  source 1..22: mol_type = other DNA
gatcggcacagatctccttatc

SEQ ID NO: 21 - DNA, 23 bp, synthetic construct
  source 1..23: mol_type = other DNA
tggcgtggtgttgctaaccttca

SEQ ID NO: 22 - DNA, 20 bp, synthetic construct
  source 1..20: mol_type = other DNA
acttggtgaatctttattaa

SEQ ID NO: 23 - DNA, 20 bp, synthetic construct
  source 1..20: mol_type = other DNA
cttggtgaatctttattaaa

SEQ ID NO: 24 - DNA, 20 bp, synthetic construct
  source 1..20: mol_type = other DNA
ggtgaatctttattaaacta

SEQ ID NO: 25 - DNA, 20 bp, synthetic construct
  source 1..20: mol_type = other DNA
tggtgaatctttattaaact

SEQ ID NO: 26 - DNA, 20 bp, synthetic construct
  source 1..20: mol_type = other DNA
gtgaatctttattaaactag

SEQ ID NO: 27 - DNA, 20 bp, synthetic construct
  source 1..20: mol_type = other DNA
ttggtgaatctttattaaac

SEQ ID NO: 28 - DNA, 20 bp, synthetic construct
  source 1..20: mol_type = other DNA
ccgtgttccatttatgagct

SEQ ID NO: 29 - DNA, 20 bp, synthetic construct
  source 1..20: mol_type = other DNA
gtcctcattttaaagttctc

SEQ ID NO: 30 - DNA, 20 bp, synthetic construct
  source 1..20: mol_type = other DNA
ctggtcttctcttatctccc

SEQ ID NO: 31 - DNA, 20 bp, synthetic construct
  source 1..20: mol_type = other DNA
gttcaaattccatcccccca

SEQ ID NO: 32 - DNA, 20 bp, synthetic construct
  source 1..20: mol_type = other DNA
ctgcaatgcattagaaacct

SEQ ID NO: 33 - DNA, 20 bp, synthetic construct
  source 1..20: mol_type = other DNA
gctaattcagtcctcatttt

SEQ ID NO: 34 - DNA, 20 bp, synthetic construct
  source 1..20: mol_type = other DNA
aagttctccaatcgacggct

SEQ ID NO: 35 - DNA, 20 bp, synthetic construct
  source 1..20: mol_type = other DNA
tcctcattttaaagttctcc

SEQ ID NO: 36 - DNA, 20 bp, synthetic construct
  source 1..20: mol_type = other DNA
cttcacattctaagctccaa

SEQ ID NO: 37 - DNA, 20 bp, synthetic construct
  source 1..20: mol_type = other DNA
ccgtgtcgctgcccctggct

SEQ ID NO: 38 - DNA, 18 bp, synthetic construct
  source 1..18: mol_type = other DNA
ttggtgaatctttattaa

SEQ ID NO: 39 - DNA, 19 bp, synthetic construct
  source 1..19: mol_type = other DNA
cttggtgaatctttattaa

SEQ ID NO: 40 - DNA, 21 bp, synthetic construct
  source 1..21: mol_type = other DNA
cttggtgaatctttattaaac

SEQ ID NO: 41 - DNA, 20 bp, synthetic construct
  source 1..20: mol_type = other DNA
tcccatttcaggagacctgg

SEQ ID NO: 42 - DNA, 20 bp, synthetic construct
  source 1..20: mol_type = other DNA
gctgattagagagaggtccc

SEQ ID NO: 43 - DNA, 16 bp, synthetic construct
  source 1..16: mol_type = other DNA
ttcaaggtgaactgtt

SEQ ID NO: 44 - DNA, 16 bp, synthetic construct
  source 1..16: mol_type = other DNA
tatttctgctccaggt

SEQ ID NO: 45 - DNA, 20 bp, synthetic construct
  source 1..20: mol_type = other DNA
  modified_base 1: mod_base = OTHER; note = 2'-O-methoxyethyl adenosine 3'-phosphorothioate
  modified_base 2: mod_base = OTHER; note = 2'-O-methoxyethyl 5-methylcytidine 3'-phosphodiester
  modified_base 3: mod_base = OTHER; note = 2'-O-methoxyethyl thymidine 3'-phosphodiester
  modified_base 4: mod_base = OTHER; note = 2'-O-methoxyethyl thymidine 3'-phosphorothioate
  modified_base 5: mod_base = OTHER; note = 2'-O-methoxyethyl guanosine 3'-phosphorothioate
  modified_base 6: mod_base = OTHER; note = constrained ethyl guanosine 3'-phosphorothioate
  modified_base 7: mod_base = OTHER; note = 2'-deoxythymidine 3'-phosphorothioate
  modified_base 8: mod_base = OTHER; note = 2'-deoxyguanosine 3'-mesyl phosphoramidate
  modified_base 9..10: mod_base = OTHER; note = 2'-deoxyadenosine 3'-mesyl phosphoramidate
  modified_base 11: mod_base = OTHER; note = 2'-deoxythymidine 3'-phosphorothioate
  modified_base 12: mod_base = OTHER; note = 5-methyl-2'-deoxycytidine 3'-phosphorothioate
  modified_base 13..15: mod_base = OTHER; note = 2'-deoxythymidine 3'-phosphorothioate
  modified_base 16: mod_base = OTHER; note = 2'-deoxyadenosine 3'-mesyl phosphoramidate
  modified_base 17..18: mod_base = OTHER; note = constrained ethyl thymidine 3'-phosphorothioate
  modified_base 19: mod_base = OTHER; note = 2'-O-methoxyethyl adenosine 3'-phosphorothioate
  modified_base 20: mod_base = OTHER; note = 2'-O-methoxyethyl adenosine
acttggtgaatctttattaa

SEQ ID NO: 46 - DNA, 20 bp, synthetic construct
  source 1..20: mol_type = other DNA
  modified_base 1: mod_base = OTHER; note = constrained ethyl 5-methylcytidine 3'-phosphorothioate
  modified_base 2..3: mod_base = OTHER; note = constrained ethyl thymidine 3'-phosphorothioate
  modified_base 4..5: mod_base = OTHER; note = 2'-deoxyguanosine 3'-mesyl phosphoramidate
  modified_base 6: mod_base = OTHER; note = 2'-deoxythymidine 3'-mesyl phosphoramidate
  modified_base 7: mod_base = OTHER; note = 2'-deoxyguanosine 3'-mesyl phosphoramidate
  modified_base 8..9: mod_base = OTHER; note = 2'-deoxyadenosine 3'-mesyl phosphoramidate
  modified_base 10: mod_base = OTHER; note = 2'-deoxythymidine 3'-phosphorothioate
  modified_base 11: mod_base = OTHER; note = 5-methyl-2'-deoxycytidine 3'-phosphorothioate
  modified_base 12..14: mod_base = OTHER; note = 2'-deoxythymidine 3'-phosphorothioate
  modified_base 15: mod_base = OTHER; note = 2'-deoxyadenosine 3'-phosphorothioate
  modified_base 16: mod_base = OTHER; note = 2'-deoxythymidine 3'-phosphorothioate
  modified_base 17: mod_base = OTHER; note = constrained ethyl thymidine 3'-phosphorothioate
  modified_base 18: mod_base = OTHER; note = constrained ethyl adenosine 3'-phosphorothioate
  modified_base 19: mod_base = OTHER; note = 2'-deoxyadenosine 3'-mesyl phosphoramidate
  modified_base 20: mod_base = OTHER; note = constrained ethyl adenosine
cttggtgaatctttattaaa

SEQ ID NO: 47 - DNA, 20 bp, synthetic construct
  source 1..20: mol_type = other DNA
  modified_base 1: mod_base = OTHER; note = constrained ethyl adenosine 3'-phosphorothioate
  modified_base 2: mod_base = OTHER; note = 2'-O-methoxyethyl 5-methylcytidine 3'-phosphodiester
  modified_base 3: mod_base = OTHER; note = 2'-O-methoxyethyl thymidine 3'-phosphodiester
  modified_base 4: mod_base = OTHER; note = 2'-O-methoxyethyl thymidine 3'-phosphorothioate
  modified_base 5: mod_base = OTHER; note = 2'-O-methoxyethyl guanosine 3'-phosphorothioate
  modified_base 6: mod_base = OTHER; note = constrained ethyl guanosine 3'-phosphorothioate
  modified_base 7: mod_base = OTHER; note = 2'-deoxythymidine 3'-phosphorothioate
  modified_base 8: mod_base = OTHER; note = 2'-deoxyguanosine 3'-mesyl phosphoramidate
  modified_base 9..10: mod_base = OTHER; note = 2'-deoxyadenosine 3'-mesyl phosphoramidate
  modified_base 11: mod_base = OTHER; note = 2'-deoxythymidine 3'-phosphorothioate
  modified_base 12: mod_base = OTHER; note = 5-methyl-2'-deoxycytidine 3'-phosphorothioate
  modified_base 13..15: mod_base = OTHER; note = 2'-deoxythymidine 3'-phosphorothioate
  modified_base 16: mod_base = OTHER; note = 2'-deoxyadenosine 3'-mesyl phosphoramidate
  modified_base 17..18: mod_base = OTHER; note = constrained ethyl thymidine 3'-phosphorothioate
  modified_base 19: mod_base = OTHER; note = 2'-O-methoxyethyl adenosine 3'-phosphorothioate
  modified_base 20: mod_base = OTHER; note = 2'-O-methoxyethyl adenosine
acttggtgaatctttattaa

SEQ ID NO: 48 - DNA, 20 bp, synthetic construct
  source 1..20: mol_type = other DNA
  modified_base 1: mod_base = OTHER; note = 2'-O-methoxyethyl thymidine 3'-mesyl phosphoramidate
  modified_base 2: mod_base = OTHER; note = 2'-O-methoxyethyl thymidine 3'-phosphodiester
  modified_base 3: mod_base = OTHER; note = 2'-O-methoxyethyl guanosine 3'-phosphodiester
  modified_base 4: mod_base = OTHER; note = constrained ethyl guanosine 3'-phosphorothioate
  modified_base 5: mod_base = OTHER; note = 2'-deoxythymidine 3'-phosphorothioate
  modified_base 6: mod_base = OTHER; note = 2'-deoxyguanosine 3'-mesyl phosphoramidate
  modified_base 7..8: mod_base = OTHER; note = 2'-deoxyadenosine 3'-mesyl phosphoramidate
  modified_base 9: mod_base = OTHER; note = 2'-deoxythymidine 3'-phosphorothioate
  modified_base 10: mod_base = OTHER; note = 5-methyl-2'-deoxycytidine 3'-phosphorothioate
  modified_base 11..13: mod_base = OTHER; note = 2'-deoxythymidine 3'-phosphorothioate
  modified_base 14: mod_base = OTHER; note = 2'-deoxyadenosine 3'-mesyl phosphoramidate
  modified_base 15..16: mod_base = OTHER; note = constrained ethyl thymidine 3'-phosphorothioate
  modified_base 17..19: mod_base = OTHER; note = 2'-O-methoxyethyl adenosine 3'-phosphorothioate
  modified_base 20: mod_base = OTHER; note = 2'-O-methoxyethyl 5-methylcytidine
ttggtgaatctttattaaac

SEQ ID NO: 49 - DNA, 20 bp, synthetic construct
  source 1..20: mol_type = other DNA
  modified_base 1: mod_base = OTHER; note = 2'-O-methoxyethyl-3'-phosphorothioate modified nucleotide
  modified_base 2: mod_base = OTHER; note = 2'-O-methoxyethyl 5-methylcytidine 3'-phosphodiester
  modified_base 3: mod_base = OTHER; note = 2'-O-methoxyethyl thymidine 3'-phosphodiester
  modified_base 4: mod_base = OTHER; note = 2'-O-methoxyethyl thymidine 3'-phosphorothioate
  modified_base 5: mod_base = OTHER; note = 2'-O-methoxyethyl guanosine 3'-phosphorothioate
  modified_base 6: mod_base = OTHER; note = constrained ethyl guanosine 3'-phosphorothioate
  modified_base 7: mod_base = OTHER; note = 2'-deoxythymidine 3'-phosphorothioate
  modified_base 8: mod_base = OTHER; note = 2'-deoxyguanosine 3'-mesyl phosphoramidate
  modified_base 9..10: mod_base = OTHER; note = 2'-deoxyadenosine 3'-mesyl phosphoramidate
  modified_base 11: mod_base = OTHER; note = 2'-deoxythymidine 3'-phosphorothioate
  modified_base 12: mod_base = OTHER; note = 5-methyl-2'-deoxycytidine 3'-phosphorothioate
  modified_base 13..15: mod_base = OTHER; note = 2'-deoxythymidine 3'-phosphorothioate
  modified_base 16: mod_base = OTHER; note = 2'-deoxyadenosine 3'-mesyl phosphoramidate
  modified_base 17..18: mod_base = OTHER; note = constrained ethyl thymidine 3'-phosphorothioate
  modified_base 19: mod_base = OTHER; note = 2'-O-methoxyethyl-3'-phosphorothioate modified nucleotide
  modified_base 20: mod_base = OTHER; note = 2'-O-methoxyethyl modified nucleoside
ncttggtgaatctttattnn

SEQ ID NO: 50 - DNA, 20 bp, synthetic construct
  source 1..20: mol_type = other DNA
  modified_base 1: mod_base = OTHER; note = constrained ethyl 3'-phosphorothioate nucleotide
  modified_base 2..3: mod_base = OTHER; note = constrained ethyl thymidine 3'-phosphorothioate
  modified_base 4..5: mod_base = OTHER; note = 2'-deoxyguanosine 3'-mesyl phosphoramidate
  modified_base 6: mod_base = OTHER; note = 2'-deoxythymidine 3'-mesyl phosphoramidate
  modified_base 7: mod_base = OTHER; note = 2'-deoxyguanosine 3'-mesyl phosphoramidate
  modified_base 8..9: mod_base = OTHER; note = 2'-deoxyadenosine 3'-mesyl phosphoramidate
  modified_base 10: mod_base = OTHER; note = 2'-deoxythymidine 3'-phosphorothioate
  modified_base 11: mod_base = OTHER; note = 5-methyl-2'-deoxycytidine 3'-phosphorothioate
  modified_base 12..14: mod_base = OTHER; note = 2'-deoxythymidine 3'-phosphorothioate
  modified_base 15: mod_base = OTHER; note = 2'-deoxyadenosine 3'-phosphorothioate
  modified_base 16: mod_base = OTHER; note = 2'-deoxythymidine 3'-phosphorothioate
  modified_base 17: mod_base = OTHER; note = constrained ethyl thymidine 3'-phosphorothioate
  modified_base 18: mod_base = OTHER; note = constrained ethyl adenosine 3'-phosphorothioate
  modified_base 19: mod_base = OTHER; note = 2'-deoxy- 3'-mesyl phosphoramidate modified nucleotide
  modified_base 20: mod_base = OTHER; note = constrained ethyl modified nucleoside
nttggtgaatctttattnnn

SEQ ID NO: 51 - DNA, 20 bp, synthetic construct
  source 1..20: mol_type = other DNA
  modified_base 1: mod_base = OTHER; note = constrained ethyl 3'-phosphorothioate nucleotide
  modified_base 2: mod_base = OTHER; note = 2'-O-methoxyethyl 5-methylcytidine 3'-phosphodiester
  modified_base 3: mod_base = OTHER; note = 2'-O-methoxyethyl thymidine 3'-phosphodiester
  modified_base 4: mod_base = OTHER; note = 2'-O-methoxyethyl thymidine 3'-phosphorothioate
  modified_base 5: mod_base = OTHER; note = 2'-O-methoxyethyl guanosine 3'-phosphorothioate
  modified_base 6: mod_base = OTHER; note = constrained ethyl guanosine 3'-phosphorothioate
  modified_base 7: mod_base = OTHER; note = 2'-deoxythymidine 3'-phosphorothioate
  modified_base 8: mod_base = OTHER; note = 2'-deoxyguanosine 3'-mesyl phosphoramidate
  modified_base 9..10: mod_base = OTHER; note = 2'-deoxyadenosine 3'-mesyl phosphoramidate
  modified_base 11: mod_base = OTHER; note = 2'-deoxythymidine 3'-phosphorothioate
  modified_base 12: mod_base = OTHER; note = 5-methyl-2'-deoxycytidine 3'-phosphorothioate
  modified_base 13..15: mod_base = OTHER; note = 2'-deoxythymidine 3'-phosphorothioate
  modified_base 16: mod_base = OTHER; note = 2'-deoxyadenosine 3'-mesyl phosphoramidate
  modified_base 17..18: mod_base = OTHER; note = constrained ethyl thymidine 3'-phosphorothioate
  modified_base 19: mod_base = OTHER; note = 2'-O-methoxyethyl-3'-phosphorothioate modified nucleotide
  modified_base 20: mod_base = OTHER; note = 2'-O-methoxyethyl modified nucleoside
ncttggtgaatctttattnn

SEQ ID NO: 52 - DNA, 17 bp, synthetic construct
  source 1..17: mol_type = other DNA
  modified_base 1: mod_base = OTHER; note = 2'-O-methoxyethyl 5-methylcytidine 3'-phosphodiester, linked at the 5' oxygen to R1 where R1 is 1, 2, or 3 linked nucleosides, an abasic nucleoside, a terminal group, or an H
  modified_base 2: mod_base = OTHER; note = 2'-O-methoxyethyl thymidine 3'-phosphodiester
  modified_base 3: mod_base = OTHER; note = 2'-O-methoxyethyl thymidine 3'-phosphorothioate
  modified_base 4: mod_base = OTHER; note = 2'-O-methoxyethyl guanosine 3'-phosphorothioate
  modified_base 5: mod_base = OTHER; note = constrained ethyl guanosine 3'-phosphorothioate
  modified_base 6: mod_base = OTHER; note = 2'-deoxythymidine 3'-phosphorothioate
  modified_base 7: mod_base = OTHER; note = 2'-deoxyguanosine 3'-mesyl phosphoramidate
  modified_base 8..9: mod_base = OTHER; note = 2'-deoxyadenosine 3'-mesyl phosphoramidate
  modified_base 10: mod_base = OTHER; note = 2'-deoxythymidine 3'-phosphorothioate
  modified_base 11: mod_base = OTHER; note = 5-methyl-2'-deoxycytidine 3'-phosphorothioate
  modified_base 12..14: mod_base = OTHER; note = 2'-deoxythymidine 3'-phosphorothioate
  modified_base 15: mod_base = OTHER; note = 2'-deoxyadenosine 3'-mesyl phosphoramidate
  modified_base 16: mod_base = OTHER; note = constrained ethyl thymidine 3'-phosphorothioate
  modified_base 17: mod_base = OTHER; note = constrained ethyl thymidine, linked at the 3' oxygen to R2, where R2 is 1, 2, or 3 linked nucleosides, an abasic nucleoside, a terminal group, or an H
cttggtgaatctttatt

SEQ ID NO: 53 - DNA, 16 bp, synthetic construct
  source 1..16: mol_type = other DNA
  modified_base 1: mod_base = OTHER; note = constrained ethyl thymidine 3'-phosphorothioate, linked at the 5' oxygen to R3 where R is 1, 2, or 3 linked nucleosides, an abasic nucleoside, a terminal group, or an H
  modified_base 2: mod_base = OTHER; note = constrained ethyl thymidine 3'-phosphorothioate
  modified_base 3..4: mod_base = OTHER; note = 2'-deoxyguanosine 3'-mesyl phosphoramidate
  modified_base 5: mod_base = OTHER; note = 2'-deoxythymidine 3'-mesyl phosphoramidate
  modified_base 6: mod_base = OTHER; note = 2'-deoxyguanosine 3'-mesyl phosphoramidate
  modified_base 7..8: mod_base = OTHER; note = 2'-deoxyadenosine 3'-mesyl phosphoramidate
  modified_base 9: mod_base = OTHER; note = 2'-deoxythymidine 3'-phosphorothioate
  modified_base 10: mod_base = OTHER; note = 5-methyl-2'-deoxycytidine 3'-phosphorothioate
  modified_base 11..13: mod_base = OTHER; note = 2'-deoxythymidine 3'-phosphorothioate
  modified_base 14: mod_base = OTHER; note = 2'-deoxyadenosine 3'-phosphorothioate
  modified_base 15: mod_base = OTHER; note = 2'-deoxythymidine 3'-phosphorothioate
  modified_base 16: mod_base = OTHER; note = constrained ethyl thymidine, linked at the 3' oxygen to R4, where R4 is 1, 2, or 3 linked nucleosides, an abasic nucleoside, a terminal group, or an H
ttggtgaatctttatt

(19) United States
(12) Patent Application Publication
(10) Pub. No.: US 2025/0353873 A1
(43) Pub. Date: Nov. 20, 2025

(54) COMPOUNDS AND METHODS FOR MODULATING APOE EXPRESSION

(71) Applicant: Ionis Pharmaceuticals, Inc., Carlsbad, CA (US)

(72) Inventor: Eric E. Swayze, Encinitas, CA (US)

(73) Assignee: Ionis Pharmaceuticals, Inc., Carlsbad, CA (US)

(21) Appl. No.: 19/210,608

(22) Filed: May 16, 2025

Related U.S. Application Data
(60) Provisional application No. 63/648,915, filed on May 17, 2024.

(51) Int. Cl. C07H 21/04; A61K 31/7125

(52) U.S. Cl. CPC C07H 21/04; A61K 31/7125

(57) ABSTRACT

Provided are oligomeric agents, pharmaceutical compositions, and methods for reducing the amount or activity of APOE RNA in a cell or subject, and in certain instances reducing the amount of ApoE protein in a cell or subject. Such oligomeric agents, pharmaceutical compositions, and methods are useful to ameliorate at least one symptom of a neurodegenerative disease. Such symptoms include cognitive impairment, progressive memory loss, a decline in language skills, behavioral abnormality, dementia, difficulty performing daily activities, aphasia, agnosia, apraxia, loss of motor function, amyloid plaque, neurofibrillary tangle, and/or neuroinflammation.

### SEQUENCE LISTING

The present application is being filed along with a Sequence Listing in electronic format. The Sequence Listing is provided as a file entitled BIOL0446SEQ.xml, created on Apr. 21, 2025, which is 136 KB in size. The information in the electronic format of the sequence listing is incorporated herein by reference in its entirety.

### FIELD

Provided are oligomeric agents, pharmaceutical compositions, and methods for reducing the amount or activity of APOE RNA in a cell or subject, and in certain instances reducing the amount of apolipoprotein E (ApoE) in a cell or subject. Certain such oligomeric agents, pharmaceutical compositions, and methods are useful to ameliorate at least one symptom of a neurodegenerative disease, e.g., a neurodegenerative disease associated with APOE. Such symptoms include cognitive impairment, progressive memory loss, a decline in language skills, behavioral abnormality, dementia, difficulty performing daily activities, aphasia, agnosia, apraxia, and loss of motor function. In certain embodiments, oligomeric agents, pharmaceutical compositions, and methods are useful to ameliorate amyloid plaques, neurofibrillary tangles, and/or neuroinflammation that are associated with a neurodegenerative disease associated with APOE. Such neurodegenerative diseases include Alzheimer's Disease.

### BACKGROUND

Alzheimer's Disease (AD) is the most common cause of age-associated dementia, affecting an estimated 5.7 million Americans a year (Alzheimer's Association. 2018 Alzheimer's Disease Facts and Figures. Alzheimer 's Dement. 2018; 14(3):367-429). Symptoms of AD include cognitive impairment, a decline in memory and language skills, behavioral and psychological symptoms such as apathy and lack of motivation, gait disturbances and seizures, and dementia. Hallmarks of AD include the presence of amyloid plaques and neurofibrillary tangles in the brains of patients. Amyloid plaques are toxic aggregates composed mainly of peptides that are encoded by the amyloid precursor protein gene, APP. Neurofibrillary tangles are hyperphosphorylated, insoluble aggregates of tau proteins.

Apolipoprotein E (ApoE) is a fat-binding protein that is associated with lipoprotein particles. ApoE is produced mainly by the liver. ApoE is also produced in the brain by astrocytes, where it plays a role in transporting cholesterol to neurons via APOE receptors. There are three main APOE alleles that encode three different ApoE protein isoforms: APOE-ε2, APOE-ε3, and APOE-ε4. These alleles encode ApoE protein variants having different combinations of amino acids at positions 112 and 158 of the ApoE protein: APOE-ε2 (Cys112, Cys158), APOE-ε3 (Cys112, Arg 158), and APOE-ε4 (Arg 112, Arg 158). These amino acid differences result in variable ApoE structure and function.

APOE has been linked to pathological hallmarks of AD (e.g., amyloid plaques and neurofibrillary tangles), and to pathways including synaptic plasticity, lipid transport, glucose metabolism, mitochondrial function, and vascular integrity. Polymorphisms in the APOE promoter result in increased APOE promoter activity and ApoE protein levels, and an increased risk of developing AD. The APOE4 allele, encoding isoform ε4, is the strongest genetic risk factor for late-onset Alzheimer's disease. Patients homozygous for the APOE4 allele account for about 16% of AD population.

Currently there is a lack of acceptable options for treating neurodegenerative diseases such as AD. It is therefore an objective herein to provide oligomeric agents, methods, and pharmaceutical compositions for the treatment of such diseases.

### SUMMARY

Provided herein are oligomeric agents, pharmaceutical compositions, and methods for reducing the amount or activity of APOE RNA, and in certain embodiments reducing the amount of ApoE protein in a cell or subject. In certain embodiments, the subject has a neurodegenerative disease. In certain embodiments, the subject has Alzheimer's Disease (AD). In certain embodiments, oligomeric agents useful for reducing expression of APOE RNA comprise modified oligonucleotides. In certain embodiments, oligomeric agents useful for reducing expression of APOE RNA comprise oligomeric duplexes and/or antisense oligonucleotides. In certain embodiments, oligomeric agents useful for reducing the amount of ApoE protein comprise modified oligonucleotides. In certain embodiments, oligomeric agents useful for reducing the amount of ApoE protein comprise oligomeric duplexes and/or antisense oligonucleotides.

Also provided are methods useful for ameliorating at least one symptom of a neurodegenerative disease. In certain embodiments, the neurodegenerative disease is Alzheimer's Disease. In certain embodiments, the symptom includes cognitive impairment, progressive memory loss, a decline in language skills, behavioral abnormality, dementia, difficulty performing daily activities, aphasia, agnosia, apraxia, and/or loss of motor function. In certain embodiments, amelioration of one or more of these symptoms result in decreasing the rate of cognitive impairment or progressive memory loss, reducing the rate of decline in language skills, reducing the rate of progression of behavioral abnormality, reducing the rate of progression of dementia, improving the performance in daily activities, reducing the rate of progression of aphasia, agnosia, and/or apraxia, and decreasing the rate of decline in motor function.

### DETAILED DESCRIPTION

It is to be understood that both the foregoing general description and the following detailed description are explanatory only and are not restrictive. Herein, the use of the singular includes the plural unless specifically stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

### Definitions

The following definitions are provided, along with additional definitions throughout the specification, for a complete understanding of the instant disclosure. Unless specific definitions are provided herein, nomenclature used in connection with, and procedures and techniques of, analytical chemistry, synthetic organic chemistry, and medicinal and pharmaceutical chemistry described herein are those well-known and commonly used in the art. Unless otherwise indicated, certain terms have the following meanings:

As used herein, a substituent at the “2′-position” means that the substituent is directly attached to the carbon at the 2′-position of a furanosyl sugar moiety.

As used herein, “2′-deoxynucleoside” means a nucleoside comprising a 2′-deoxyfuranosyl sugar moiety. Unless otherwise indicated, a 2′-deoxynucleoside is a 2′-β-D-deoxynucleoside which comprises a 2′-β-D-deoxyribosyl sugar moiety, and which is in the β-D ribosyl configuration as found in naturally occurring deoxyribonucleic acid (DNA). A 2′-deoxynucleoside or a nucleoside comprising an unmodified 2′-deoxyribosyl sugar moiety may be abasic, comprise a modified nucleobase, or may comprise an RNA nucleobase (uracil).

As used herein, “2′-deoxy sugar moiety” means a 2′-H(H) deoxyfuranosyl sugar moiety. Unless otherwise indicated, a 2′-deoxy sugar moiety is a 2′-β-D-deoxyribosyl sugar moiety, which has the β-D ribosyl stereochemical configuration as found in naturally occurring deoxyribonucleic acid (DNA).

As used herein, “2′-MOE” means a 2′-OCH₂CH₂OCH₃ group at the 2′-position of a furanosyl sugar moiety. A “2′-MOE sugar moiety” means a sugar moiety with a 2′-OCH₂CH₂OCH₃ group at the 2′-position of a furanosyl sugar moiety. Unless otherwise indicated, a 2′-MOE sugar moiety is in the β-D-ribosyl stereochemical configuration. “MOE” means O-methoxyethyl.

As used herein, “2′-MOE nucleoside” or “2′-OCH₂CH₂OCH₃ nucleoside” means a nucleoside comprising a 2′-MOE sugar moiety (or 2′-OCH₂CH₂OCH₃ furanosyl sugar moiety).

As used herein, “2′-OMe” means a 2′-OCH₃ group at the 2′-position of a furanosyl sugar moiety. A “2′-OMe sugar moiety” means a sugar moiety with a 2′-OCH₃ group at the 2′-position of a furanosyl sugar moiety. Unless otherwise indicated, a 2′-OMe sugar moiety is in the β-D-ribosyl stereochemical configuration.

As used herein, “2′-OMe nucleoside” means a nucleoside comprising a 2′-OMe sugar moiety.

As used herein, “2′-F” means a 2′-fluoro group at the 2′-position of a furanosyl sugar moiety. A “2′-F sugar moiety” means a sugar moiety with a 2′-F group at the 2′-position of a furanosyl sugar moiety. Unless otherwise indicated, a 2′-F sugar moiety is in the β-D-ribosyl configuration.

As used herein, “2′-F nucleoside” means a nucleoside comprising a 2′-F sugar moiety.

As used herein “2′-NMA” means a 2′-OCH₂C(═O)—N(H)CH₃ group at the 2′-position of a furanosyl sugar moiety. A “2′-NMA sugar moiety” means a sugar moiety with a 2′-OCH₂C(═O)—N(H)CH₃ group at the 2′-position of a furanosyl sugar moiety.

As used herein, “2′-NMA nucleoside” means a nucleoside comprising a 2′-NMA sugar moiety.

As used herein, “2′-substituted nucleoside” means a modified nucleoside comprising a 2′-substituted furanosyl sugar moiety.

As used herein, “2′-substituted sugar moiety” means a modified furanosyl sugar moiety wherein the 2′-position is attached to at least one substituent other than H or OH. A 2′-substituted sugar moiety includes a bicyclic sugar moiety wherein the second ring is joined to the furanosyl ring at the 2′-position. 2′-substituted sugar moieties include, but are not limited to, 2′-OMe sugar moieties, 2′-MOE sugar moieties, 2′-F sugar moieties, 2′-NMA sugar moieties, cEt sugar moieties, and LNA sugar moieties.

As used herein, “5-methylcytosine” means a cytosine modified with a methyl group attached to the 5 position. A 5-methylcytosine is a modified nucleobase.

As used herein, “abasic nucleoside” means a modified nucleoside in which the sugar moiety is not attached to a nucleobase (i.e., the nucleobase is absent).

As used herein, “acyclic sugar surrogate nucleoside” means a nucleoside having Formula II, Formula III, or Formula IIIa:

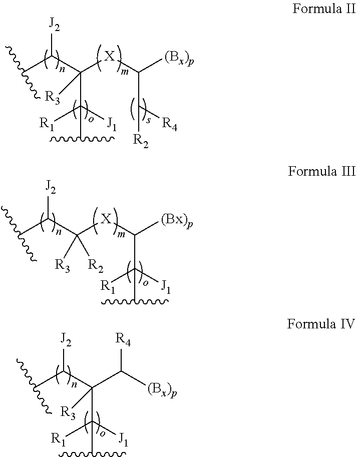

wherein
X is O, S, C(R₅R₆), N(E₁), NC(═O)-(E₁);
each J₁ and J₂ are independently H or C₁-C₆ alkyl;
n is 0, 1 or 2;
m is 0, 1, or 2;
p is 0 or 1;
o is 0 or 1;
s is 0 or 1;
R₁ is H, OH, halogen, C₁-C₆ alkyl, C₁-C₆ alkoxy, C₂-C₆ alkenyl, C₂-C₆ alkynyl, or (CH₂)_{q}R₈;
R₂, R₃, and R₄ are each independently H, OH, halogen, C₁-C₆ alkyl, C₁-C₆ alkoxy, C₂-C₆ alkenyl, C₂-C₆ alkynyl, S—CH₃, N(CH₃)(CH₃), OCH₂CH₂OCH₃, O-alkylamino, or (CH₂)_{q}R;
E₁ is H, C₁-C₆ alkyl or substituted C₁-C₆ alkyl;
R₅ and R₆ are independently H, OH, C₁-C₆ alkyl, or N(R₇); wherein if R₅ is OH, then R₆ is not OH;
R₇ is H, C₁-C₆ alkyl, or C(═O)R₉, wherein R₉ is C₁-C₆ alkyl;
R₈ is OH, halogen, methoxy, ethoxy, azido, C₂-C₆ alkenyl, or C₂-C₆ alkynyl, and q is 1, 2, or 3; and Bx is a nucleobase.

As used herein, “acyclic sugar surrogate” means the sugar moiety of an acyclic sugar surrogate nucleoside.

As used herein, “administration” or “administering” means providing a pharmaceutical agent or composition to a subject.

As used herein, “ameliorate” with reference to a symptom of a disease, means improvement in, or lessening of, or preclusion of, at least one symptom of a disease. As used herein, “disease” includes disorders, conditions, and injuries. Amelioration may be reduction in severity or frequency of a symptom or the delayed onset, prevention of occurrence of, or slowing of progression in the severity or frequency of, a symptom. In certain embodiments, the symptom is cognitive impairment, progressive memory loss, a decline in language skills, behavioral abnormality, dementia, difficulty performing daily activities, aphasia, agnosia, apraxia, loss of motor function, amyloid plaque, neurofibrillary tangle, and/or neuroinflammation. Progression, frequency, or severity indicators may be determined by subjective or objective measures known in the art and/or described herein.

As used herein, “amyloid plaque” means an aggregate of peptides that are encoded by a human amyloid precursor protein gene, APP.

As used herein, “antisense activity” means any detectable and/or measurable change attributable (whether directly and/or indirectly) to the hybridization of an antisense oligonucleotide to a target nucleic acid. For example, compounds have antisense activity when they alter the amount or activity of a target nucleic acid by 25% or more in an in vitro assay; or, for example compounds have antisense activity when they alter the amount or activity of a target nucleic acid by 25% or more in an in vivo assay. Antisense activity may be assessed in a standard assay. Herein, antisense activity is a decrease in the amount or expression of a target nucleic acid or protein encoded by such target nucleic acid compared to target nucleic acid levels or target protein levels in the absence of the antisense oligonucleotide.

As used herein, “antisense agent” means an oligomeric agent comprising an antisense oligonucleotide.

As used herein, “antisense oligonucleotide” means an oligonucleotide having at least one region (a “targeting region”) that is complementary to a target nucleic acid (e.g., a target region). An antisense oligonucleotide may be paired with a second oligonucleotide (herein, a “sense oligonucleotide”) that is complementary to the antisense oligonucleotide (for example, forming an “oligomeric duplex”), may be an unpaired antisense oligonucleotide (herein, a single-stranded antisense oligonucleotide), or may be a “hairpin oligonucleotide” that has at least one region that is self-complementary.

As used herein, “behavioral abnormality” means a behavior exhibited by a subject that is atypical or out of the ordinary for the subject. In certain embodiments, the behavior abnormality is a dysfunctional or deviant behavior (e.g., causes distress for others).

As used herein, “bicyclic nucleoside” or “BNA” means a nucleoside comprising a bicyclic sugar moiety.

As used herein, “bicyclic sugar” or “bicyclic sugar moiety” means a modified sugar moiety comprising a furanosyl sugar moiety and a second ring, wherein the second ring is formed via a bridge connecting two non-geminal atoms in the ring of the furanosyl sugar moiety, thereby forming a bicyclic structure. Examples of bicyclic sugar moieties include locked nucleic acid (LNA) sugar moieties and constrained ethyl (cEt) sugar moieties as defined herein.

As used herein, “blunt” or “blunt ended” in reference to an oligomeric duplex mean that there are no terminal unpaired nucleotides (i.e., no overhanging nucleotides). One or both ends of an oligomeric duplex may be blunt.

As used herein, “cell-targeting moiety” means a conjugate moiety or portion of a conjugate moiety that has affinity for a particular cell type or particular cell types. For example, a cell-targeting moiety may have affinity for a cell surface moiety, such as a cell surface receptor on a particular cell type.

As used herein, “cerebrospinal fluid” or “CSF” means the fluid filling the space around the brain and spinal cord. “Artificial cerebrospinal fluid” or “aCSF” means a prepared or manufactured fluid that has certain properties (e.g., osmolarity, pH, and/or electrolytes) similar to cerebrospinal fluid and is biocompatible with CSF.

As used herein, “cleavable moiety” means a group of atoms comprising at least one bond that is cleaved under physiological conditions, e.g., in a cell and/or a subject. For example, a cleavable moiety cleaved inside a cell or sub-cellular compartment, such as an endosome or lysosome. A cleavable moiety may be cleaved by endogenous enzymes, such as nucleases.

As used herein, “complementary nucleobase(s)” or “complementary” in reference to nucleobase(s) means nucleobases that form hydrogen bonds with one another. Complementary nucleobase pairs include, but are not limited to, adenine (A) and thymine (T), adenine (A) and uracil (U), cytosine (C) and guanine (G), 5-methylcytosine (^{m}C) and guanine (G). Certain modified nucleobases that are complementary to unmodified nucleobases or to other modified nucleobases are known in the art. For example, hypoxanthine, the nucleobase of the nucleoside inosine (I), can pair with adenine, cytosine, thymine, or uracil. Herein, hypoxanthine (I) is considered a complementary nucleobase to thymine (T), adenine (A), uracil (U), and cytosine (C).

As used herein, “complementary nucleobase sequence(s)” or “complementary” in reference to nucleobase sequence(s) refers to two nucleobase sequences in which some, a majority, or all of the nucleobases in the two nucleobase sequences are complementary nucleobases when the sequences are aligned. A “nucleobase sequence” means the order of nucleobases in a strand of linked nucleosides or a region thereof (e.g., an oligonucleotide or region thereof, or a target nucleic acid or region thereof) independent of any sugar or internucleoside linkage modification or presence of an intervening spacer. Complementary nucleobase sequences may be nucleobase sequences of two separate strands of linked nucleosides or regions thereof (e.g., an oligonucleotide and a region of a target nucleic acid, or an antisense oligonucleotide and its paired sense oligonucleotide) or complementary nucleobase sequences may be nucleobase sequences of two regions of a single strand of linked nucleosides (e.g., self-complementary regions of a hairpin oligonucleotide). As used herein, when a first strand of linked nucleosides (e.g., an oligonucleotide) or region thereof is described as being complementary to a second strand of linked nucleosides or region thereof (e.g., a target nucleic acid or another oligonucleotide), it means that the nucleobase sequence of the first strand of linked nucleosides or region thereof is complementary to the nucleobase sequence of the second strand of linked nucleosides or region thereof when aligned. Not every pair of nucleobases in the aligned nucleobase sequences needs to be complementary for the two sequences to be “complementary.” Rather, some mismatches are tolerated. Where nucleobase sequence complementarity is expressed as a percent, such percent represents the percentage of nucleobases within one nucleobase sequence that are complementary to nucleobases within an equal length second nucleobase sequence when the nucleobase sequences are aligned. Unless otherwise specified, “complementary” is assumed to be at least 70%. Complementary nucleobase sequences may be 75%, 80%, 85%, 90%, 95%, or 100% complementary. For example, if a nucleobase sequence of an oligonucleotide consisting of 20 nucleosides is 80% complementary to another nucleobase sequence, then 16 of the nucleobase pairs are complementary nucleobases, and there are 4 mismatches when the sequences are aligned. If a nucleobase sequence of an oligonucleotide consisting of 20 nucleosides is at least 80% complementary to another nucleobase sequence, then 16, 17, 18, 19, or 20 of the nucleobase pairs are complementary nucleobases, and there are 0-4 mismatches when the sequences are aligned. As used herein, “fully complementary” or “100% complementary” means that each nucleobase pair of the two nucleobase sequences is complementary when the equal length sequences are aligned.

As used herein, “complementary region” in reference to a strand of linked nucleosides (e.g., an oligonucleotide or a target nucleic acid) is a region of the strand of linked nucleosides in which the nucleobase sequence of the region is complementary with the nucleobase sequence of an equal-length region of a separate strand of linked nucleosides (e.g., an oligonucleotide and a target nucleic acid, or an antisense oligonucleotide and a sense oligonucleotide), or the nucleobase sequence of an equal-length region within the strand of linked nucleosides (e.g., in a “hairpin oligonucleotide”). A complementary region of a strand of linked nucleosides may be a portion of a strand of linked nucleosides or may include the entire strand of linked nucleosides. A complementary region may include a mismatch, but the nucleobases of the terminal nucleosides of a complementary region are complementary to the nucleobases of the terminal nucleosides of the equal-length region of the separate strand of linked nucleosides or to the nucleobases of the terminal nucleosides of the equal-length region within the strand of linked nucleosides. A “targeting region” of an oligonucleotide, is a complementary region in which the nucleobase sequence of the region is complementary to the nucleobase sequence of a target region of a target nucleic acid. A targeting region of a strand of linked nucleosides may be a portion of a strand of linked nucleosides or may include the entire strand of linked nucleosides. A “duplexing region” is a complementary region of an oligonucleotide (e.g., an antisense or sense oligonucleotide) having a nucleobase sequence that is complementary to the nucleobase sequence of a second oligonucleotide or region thereof. A duplexing region may be a portion of a strand of linked nucleosides or may include the entire strand of linked nucleosides.

As used herein, “conjugate group” means a group of atoms that is directly or indirectly attached to an oligonucleotide. A conjugate group comprises a conjugate moiety and a conjugate linker that attaches the conjugate moiety to the oligonucleotide.

As used herein, “conjugate linker” means a single bond or a group of atoms comprising at least one bond that connects a conjugate moiety to an oligonucleotide.

As used herein, “conjugate moiety” means a group of atoms that when covalently bound to a molecule (e.g., an oligonucleotide) modifies one or more properties of such molecule compared to the same molecule lacking the conjugate moiety, wherein such properties include, but are not limited to pharmacodynamics, pharmacokinetics, stability, binding, absorption, tissue distribution, cellular distribution, cellular uptake, charge, and clearance.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “constrained ethyl” or “cEt” or “cEt sugar moiety” means a β-D ribosyl bicyclic sugar moiety wherein the second ring of the bicyclic sugar is formed via a bridge connecting the 4′-carbon and the 2′-carbon of the β-D ribosyl sugar moiety, wherein the bridge has the formula 4′-CH(CH₃)—O-2′, and wherein the methyl group of the bridge is in the S configuration.

As used herein, “cEt nucleoside” means a nucleoside comprising a cEt sugar moiety.

As used herein a “cyclic sugar surrogate nucleoside” is a nucleoside having Formula I.

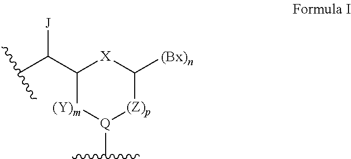

wherein
J is H, C₁-C₆ alkyl, or C₂-C₆ alkenyl;
X is O, S, C(R₁R₂), N(R₃), or X₁-X₂, wherein X₁-X₂ is C(R₁)═C(R₂), C(R₁R₂)—C(R₁R₂), O—C(R₁R₂), C(R₁R₂)—O, S—C(R₁R₂), C(R₁R₂)—S, N(R₃)—C(R₁R₂), or C(R₁R₂)—N(R₃);
Y is C(R₁R₂) or Y₁—Y₂, wherein Y₁—Y₂ is C(R₁)═C(R₂) or C(R₁R₂)—C(R₁R₂);
Z is C(G₁G₂) or Z₁—Z₂, wherein Z₁-Z₂ is C(G₁)═C(R₁), C(R₁)═C(G₁); C(G₁G₂)-C(R₁R₂), C(R₁R₂)—C(G₁G₂); or Z₁-Z₂—Z₃, wherein Z₁-Z₂—Z₃ is C(G₁G₂)-C(R₁R₂)—C(R₁R₂) or C(R₁R₂)—C(R₁R₂)—C(G₁G₂); Q is CH or N;
each R₁ and R₂ is independently H, OH, C₁-C₆ alkyl, or N(R₄); wherein if R₁ is OH, then R₂ is not OH;
each R₃ and R₄ is independently H, C₁-C₆ alkyl, or C(═O)R₅, wherein R₅ is C₁-C₆ alkyl;
each G₁ and G₂ is independently H, OH, halogen or O—[C(R₆)(R₇)]_{q}—[(C═O)ₛ—X^{G}]ⱼ-R₈; wherein if G₁ is OH, then G₂ is not OH;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is 0 or 1;
m is 0 or 1;
p is 0 or 1;
q is from 1 to 6;
s is 0 or 1;
j is 0 or 1;
Bx is a nucleobase; and
provided that if X is O, Z is C(G₁G₂), and Q is CH, then m is 1.

As used herein, “cyclic sugar surrogate” means the sugar moiety of a cyclic sugar surrogate nucleoside.

As used herein, “daily activities” mean one or more activities selected from dressing, eating, walking, bathing, cooking, shopping, cleaning, and exercising.

As used herein, “dementia” means any combination of symptoms selected from memory loss, difficulty communicating, disorientation, difficulty reasoning, difficulty planning, discoordination, compromised motor function, confusion, disorientation, depression, anxiety, paranoia, agitation, and hallucination.

As used herein, “deoxy region” means a region of 5-13 contiguous nucleotides, wherein at least 70% of the nucleosides are DNA nucleosides. Each nucleoside of a deoxy region is selected from a 2′-deoxynucleoside and 2′-substituted nucleoside. A deoxy region supports RNase H activity.

As used herein, “DNA nucleoside” means a nucleoside comprising an unmodified DNA sugar moiety. A DNA nucleoside may comprise a modified or unmodified nucleobase. A DNA nucleoside may comprise an uracil nucleobase or a modified nucleobase, or may be an abasic nucleoside.

As used herein, “DNA sugar moiety” means an unmodified DNA sugar moiety.

As used herein, “diluent” means an ingredient in a composition that lacks pharmacological activity, but is pharmaceutically necessary or desirable. For example, the diluent in an injected composition can be a liquid, e.g., aCSF, PBS, or saline solution.

As used herein, “double-stranded” refers to hybridized or bound complementary regions, including those between two separate strands of linked nucleosides (e.g., an antisense oligonucleotide and a sense oligonucleotide) and those within a single strand of linked nucleosides (e.g., a hairpin oligonucleotide). Hybridized complementary regions of two separate strands of linked nucleosides form a “duplex” of the separate strands. Hybridized complementary regions of a single strand of linked nucleosides (i.e., a first region of the strand of linked nucleosides and a second region of the strand of linked nucleosides) form a “hairpin.”

As used herein, “duplex” means a structure formed by two separate strands of linked nucleosides (e.g., two separate oligonucleotides) or regions thereof, at least a portion of which are complementary to and hybridize to each other. For clarity, herein a “hairpin oligonucleotide” is one strand of linked nucleosides that comprises a region that is double stranded and is not a duplex.

As used herein, a “furanosyl sugar moiety” is a group of atoms that comprises a furanose ring, and is numbered according to the structure below, with optional additional substituents at any of the 1′, 2′, 3′, 4′, and 5′ positions,

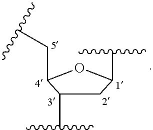 [SMILES: *CC1OC(*)CC1*]

As used herein, “hybridize” or “hybridization” means the act or process of two complementary regions of linked nucleosides (e.g., oligonucleotides, nucleic acids) annealing together to form a double-stranded region. While not limited to a particular mechanism, the most common mechanism of hybridization involves hydrogen bonding, which may be Watson-Crick, Hoogsteen, or reversed Hoogsteen hydrogen bonding, between complementary nucleobases.

As used herein, “internucleoside linkage” means the covalent linkage between immediately adjacent (e.g., contiguous) nucleosides in an oligonucleotide. As used herein, “unmodified internucleoside linkage” means a phosphodiester internucleoside linkage. As used herein, “modified internucleoside linkage” means any internucleoside linkage other than a phosphodiester internucleoside linkage. A “phosphorothioate internucleoside linkage” is a modified internucleoside linkage in which one of the non-bridging oxygen atoms of a phosphodiester internucleoside linkage is replaced with a sulfur atom. A “mesyl phosphoramidate internucleoside linkage” is a modified internucleoside linkage in which one of the non-bridging oxygen atoms of a phosphodiester internucleoside linkage is replaced with NS(═O)₂CH₃. Unless otherwise indicated, and in the context of linked nucleosides each comprising a furanosyl sugar moiety, an internucleoside linkage joins the 3′-carbon of one furanosyl sugar moiety to the 5′-carbon of the other furanosyl sugar moiety.

As used herein, “inverted nucleoside” means a nucleotide having a 3′ to 3′ and/or 5′ to 5′ internucleoside linkage.

As used herein, “inverted sugar moiety” means the sugar moiety of an inverted nucleoside or an abasic sugar moiety having a 3′ to 3′ and/or 5′ to 5′ internucleoside linkage.

As used herein, “linked nucleosides” are nucleosides that are connected in a contiguous sequence (i.e., nucleosides immediately adjacent to one another, no additional nucleosides are present between those that are linked).

As used herein, a “mismatch” between two aligned nucleic acid sequences (e.g., nucleic acid sequence of separate strands of linked nucleosides or separate regions of a strand of linked nucleosides) means that the two nucleobases at a specified position of the aligned nucleobase sequences are not complementary nucleobases.

As used herein, “modified nucleoside” means a compound or subunit comprising a sugar moiety and optionally a nucleobase, wherein the sugar moiety is modified and/or the nucleobase is modified or is absent (i.e., abasic nucleoside).

As used herein, “modified sugar moiety” means a sugar moiety other than a β-D-ribosyl sugar moiety in RNA or a β-D-deoxyribosyl sugar moiety in DNA. A modified sugar moiety is selected from a modified furanosyl sugar moiety, a cyclic sugar surrogate, an acyclic sugar surrogate, or a sugar mimic.

As used herein, a “modified nucleobase” means a nucleobase other than unmodified A, T, C, U, or G capable of pairing with at least one unmodified nucleobase. A “5-methylcytosine” is a modified nucleobase. Inosine (I) is a nucleoside comprising the modified nucleobase hypoxanthine.

As used herein, “motif” means a pattern of independently unmodified and/or independently modified sugar moieties, nucleobases, and/or internucleoside linkages in an oligonucleotide.

As used herein, “neurofibrillary tangles” mean hyperphosphorylated insoluble aggregates of tau protein. Tau protein is encoded by a human MAPT gene.

As used herein, “neuroinflammation” means inflammation of the peripheral nervous system or the central nervous system. In certain embodiments, the amount of a cytokine in the cerebrospinal fluid of a subject with neuroinflammation is significantly greater than the amount of the cytokine in the cerebrospinal fluid of a subject that does not have neuroinflammation. In certain embodiments, a subject with neuroinflammation has Alzheimer's Disease. In certain embodiments, a subject that does not have neuroinflammation does not have Alzheimer's Disease.

As used herein, “non-bicyclic modified sugar moiety” means a modified furanosyl sugar moiety comprising a modification, such as a substituent, that does not form a bridge between two atoms of the sugar to form a second ring.

As used herein, “nucleobase” means an unmodified nucleobase or a modified nucleobase.

As used herein, “nucleobase sequence” means the order of contiguous nucleobases in a strand of linked nucleosides independent of any sugar or internucleoside linkage modification. As used herein, “the nucleobase sequence of” a reference SEQ ID NO refers only to the nucleobase sequence provided in such SEQ ID NO and therefore, unless otherwise indicated, includes compounds wherein each sugar moiety and each internucleoside linkage, independently, is modified or unmodified, irrespective of the presence or absence of modifications indicated in the referenced SEQ ID NO.

As used herein, “nucleoside” means an “unmodified nucleoside” or a “modified nucleoside”.

As used herein, a “nucleoside mimic” means a compound or subunit comprising a sugar mimic and a nucleobase.

As used herein, “nucleoside overhang” or “overhang” refers to unpaired nucleosides at either or both ends of an oligomeric duplex or at an end of a hairpin structure. The nucleosides of an overhang are not part of the “duplexing region” of either of the two strands of linked nucleosides.

As used herein, “oligomeric agent” means a compound or complex comprising or consisting of at least one modified oligonucleotide and optionally one or more additional associated features selected from: (a) one or more additional modified or unmodified oligonucleotides, each of which may be hybridized to or covalently linked to the at least one modified oligonucleotide and/or to each other; (b) one or more conjugate groups, which may be covalently attached directly or indirectly to any oligonucleotide of such oligomeric agent; and (c) one or more terminal groups. Herein, where two oligonucleotides are described as being covalently attached to one another, such attachment is other than through a direct internucleoside linkage. Thus, a single, unbranched oligonucleotide comprising only direct internucleoside linkages cannot be described as two separate covalently linked oligonucleotides.

As used herein, “oligonucleotide” means a strand of linked nucleosides, wherein each nucleoside and/or each internucleoside linkage of the strand of linked nucleosides may be independently modified or unmodified. Unless otherwise indicated, oligonucleotides consist of 12-80 linked nucleosides. Unless otherwise indicated, no more than 10% of the nucleosides of an oligonucleotide are abasic nucleosides. As used herein, “modified oligonucleotide” means an oligonucleotide, wherein at least one nucleoside and/or at least one internucleoside linkage is modified. As used herein, “unmodified oligonucleotide” means an oligonucleotide consisting of unmodified nucleosides linked by phosphodiester internucleoside linkages. An oligonucleotide may be paired with a second oligonucleotide that is complementary to the oligonucleotide to form an oligomeric duplex, or it may be unpaired.

As used herein, “pharmaceutically acceptable diluent” means an ingredient in a pharmaceutical composition suitable for use in administering to a subject. Typically, a “diluent” lacks pharmacological activity but is desirable in preparing a pharmaceutical composition.

As used herein “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein “pharmaceutical composition” means a mixture of substances suitable for administration to a subject. For example, a pharmaceutical composition may comprise an oligomeric agent and a sterile aqueous solution. A pharmaceutical composition may show activity in certain cell lines.

As used herein “prodrug” means a therapeutic agent in a first form outside the body that is converted to a second form within a subject or cells thereof. Typically, conversion of a prodrug within the subject is facilitated by the action of an enzyme (e.g., endogenous or viral enzyme) or chemical present in cells or tissues and/or by physiologic conditions. The first form of the prodrug may be less active than the second form.

As used herein, “progressive memory loss” means occurrences of forgetfulness (e.g., inability to remember events, names, and facts) that increase in frequency over time.

As used herein, “reducing or inhibiting the amount or activity” refers to a reduction or blockade of the transcriptional expression or activity relative to the transcriptional expression or activity in an untreated or control sample and does not necessarily indicate a total elimination of transcriptional expression or activity.

As used herein, “RNA nucleoside” means a nucleoside comprising an unmodified RNA sugar moiety. An RNA nucleoside may comprise a modified or unmodified nucleobase. An RNA nucleoside may comprise a thymine nucleobase or a modified nucleobase, or may be an abasic nucleoside.

As used herein, “RNA sugar moiety” means an unmodified RNA sugar moiety.

As used herein, “RNAi agent” means an antisense agent that acts, at least in part, through RISC or Ago2 to modulate a target nucleic acid and/or a protein encoded by a target nucleic acid. RNAi agents include, but are not limited to double-stranded siRNA, single-stranded RNAi (ssRNAi), and microRNA, including microRNA mimics. RNAi agents may comprise conjugate groups and/or terminal groups. In certain embodiments, an RNAi agent modulates the amount and/or activity of a target nucleic acid. The term RNAi agent excludes antisense agents that act through RNase H.

As used herein, “RNase H agent” means an antisense agent that acts, at least in part, through RNase H to modulate a target nucleic acid and/or protein encoded by a target nucleic acid. RNase H agents may be single-stranded or RNase H agents may be double-stranded. RNase H agents may comprise conjugate groups and/or terminal groups. RNase H agents may modulate the amount and/or activity of a target nucleic acid. The term RNase H agent excludes antisense agents that act through RISC/Ago2.

As used herein, “single-stranded” in reference to a strand of linked nucleosides (e.g., an oligonucleotide) means that the strand of linked nucleosides is not part of a duplex or part of a double-stranded region. Single-stranded nucleic acids (e.g., single-stranded oligonucleotides) are capable of hybridizing to complementary nucleic acids to form duplexes, at which point they are no longer single-stranded.

As used herein, “stabilized phosphate group” means a 5′-phosphate analog that is metabolically more stable than a 5′-phosphate as naturally occurs on DNA or RNA.

As used herein, “standard in vitro assay” means the assay described in Example 4, 5, or 6, and reasonable variations thereof.

As used herein, “standard in vivo assay” means the assay described in Example 7, 8, 9, 10, 11, 12, 13, 14, 15, 16, or 17, and reasonable variations thereof.

As used herein, “stereorandom” or “stereorandom chiral center” in the context of a population of molecules of identical molecular formula means a chiral center that is not intentionally controlled during synthesis, or enriched following synthesis, for a particular absolute stereochemical configuration at that chiral center. It is understood that a stereorandom chiral center may not be racemic because one absolute configuration predominates following synthesis, e.g., due to steric and electronic interactions of reagents with the reactant molecule. The stereorandom chiral center may be at the phosphorous atom of a stereorandom phosphorothioate or stereorandom mesyl phosphoramidate internucleoside linkage.

As used herein, a “strand” or “strand of linked nucleosides” means linked nucleosides and/or nucleoside surrogates connected via internucleoside linkages. A strand of linked nucleosides has a nucleobase sequence.

As used herein, “subject” means a human or non-human animal. In certain embodiments, the subject is a human.

As used herein, “sugar moiety” means an unmodified sugar moiety or a modified sugar moiety.

As used herein, “sugar mimic” means a group of atoms forming the portion of a nucleoside corresponding to the β-D-ribosyl sugar in RNA other than a modified furanosyl sugar moiety, a cyclic sugar surrogate, or an acyclic sugar surrogate.

As used herein, “sugar surrogate nucleoside” means a cyclic sugar surrogate nucleoside or an acyclic sugar surrogate nucleoside.

As used herein, “symptom” of a disease means any manifestation, indication, sign, or evidence of a disease. Symptoms include subjective and objective indicia of a disease and may be perceived, experienced, detected, observed, measured, and/or quantified. A symptom may be apparent only upon invasive diagnostic testing, including, but not limited to, post-mortem tests. A symptom may be an absence of a feature, such as failing to reach expected developmental milestones. Symptoms may include cognitive impairment, progressive memory loss, a decline in language skills, behavioral abnormality, dementia, difficulty performing daily activities, aphasia, agnosia, apraxia, loss of motor function, amyloid plaque, neurofibrillary tangle, and/or neuroinflammation.

As used herein, “target nucleic acid” means an APOE nucleic acid that an antisense oligonucleotide is designed to affect. As used herein, “target RNA” means an APOE RNA transcript and includes pre-mRNA and/or mRNA unless otherwise specified.

As used herein, “target region” refers to a portion of a target nucleic acid that is complementary to the targeting region of an antisense oligonucleotide.

As used herein, “terminal group” means a chemical group or group of atoms that is covalently linked to a terminus of an oligonucleotide.

As used herein, “treating” or “treatment,” with respect to a disease, means administering a compound or agent to a subject having or at risk for developing such disease. Treating a disease may result in amelioration of at least one symptom of such disease. Treatment may reduce, improve, and/or prevent one or more symptom(s) such that a symptom of the disease is diminished, is no longer apparent, or is never apparent.

As used herein, “therapeutically effective amount” means an amount of a pharmaceutical agent or composition that provides a therapeutic benefit to a subject. For example, a therapeutically effective amount ameliorates at least one symptom of a disease.

As used herein, “unmodified nucleobase” means unmodified adenine (A), unmodified thymine (T), unmodified cytosine (C), unmodified uracil (U), or unmodified guanine (G).

As used herein, an “unmodified nucleoside” means a compound or subunit comprising an unmodified sugar moiety and an unmodified nucleobase.

As used herein, “unmodified sugar moiety” means a 2′-OH(H) β-D-ribosyl sugar moiety, as found in RNA (an “unmodified RNA sugar moiety”), or a 2′-H(H) β-D-deoxyribosyl sugar moiety, as found in DNA (an “unmodified DNA sugar moiety”). Unmodified sugar moieties are furanosyl or deoxyfuranosyl sugar moieties in the β-D-ribosyl stereochemical configuration, and have one hydrogen at each of the 1′, 3′, and 4′ positions, an oxygen at the 3′ position, two hydrogens at the 5′ position, and two hydrogens (DNA) or a hydrogen and an OH (RNA) at the 2′ position.

### CERTAIN EMBODIMENTS

The present disclosure provides the following non-limiting numbered embodiments:

Embodiment 1. An oligomeric agent comprising a modified oligonucleotide consisting of 12 to 50 linked nucleosides wherein the nucleobase sequence of the modified oligonucleotide is at least 80% complementary to an equal length portion of an APOE nucleic acid, and wherein at least one internucleoside linkage of the modified oligonucleotide is a mesyl phosphoramidate internucleoside linkage.

Embodiment 2. An oligomeric agent comprising a modified oligonucleotide consisting of 12 to 50 linked nucleosides and having a nucleobase sequence comprising at least 12, at least 13, at least 14, at least 15, at least 16, at least 17, at least 18, at least 19, at least 20, or 21 contiguous nucleobases of any of the nucleobase sequences of SEQ ID NOs: 22-40, wherein at least one internucleoside linkage of the modified oligonucleotide is a mesyl phosphoramidate internucleoside linkage.

Embodiment 3. The oligomeric agent of embodiment 1 or embodiment 2, wherein the modified oligonucleotide has a nucleobase sequence that is at least 85%, at least 90%, at least 95%, or 100% complementary to the nucleobase sequence of any one of SEQ ID NOs: 1-5 when measured across the entire nucleobase sequence of the modified oligonucleotide.

Embodiment 4. The oligomeric agent of any of embodiments 1-3, wherein the modified oligonucleotide consists of 12 to 20, 12 to 21, 12 to 25, 12 to 30, 12 to 50, 13 to 20, 13 to 21, 13 to 25, 13 to 30, 13 to 50, 14 to 20, 14 to 21, 14 to 25, 14 to 30, 14 to 50, 15 to 20, 15 to 21, 15 to 25, 15 to 30, 15 to 50, 16 to 18, 16 to 20, 16 to 21, 16 to 25, 16 to 30, 16 to 50, 17 to 20, 17 to 21, 17 to 25, 17 to 30, 17 to 50, 18 to 20, 18 to 21, 18 to 25, 18 to 30, 18 to 50, 19 to 20, 19 to 21, 19 to 25, 19 to 30, 19 to 50, 20 to 25, 20 to 30, 20 to 50, 21 to 25, 21 to 30, or 21 to 50 linked nucleosides.

Embodiment 5. The oligomeric agent of any of embodiments 1-4, wherein the modified oligonucleotide has a nucleobase sequence comprising at least 15, at least 16, at least 17, at least 18, at least 19, or 20 contiguous nucleobases of any of the nucleobase sequences of SEQ ID NOs: 22-37.

Embodiment 6. The oligomeric agent of any of embodiments 1-4, wherein the modified oligonucleotide has a nucleobase sequence comprising at least 15, at least 16, at least 17, at least 18, at least 19, or 20 contiguous nucleobases of the nucleobase sequence of SEQ ID NO: 23.

Embodiment 7. The oligomeric agent of any of embodiments 1-6, wherein at least one nucleoside of the modified oligonucleotide is a modified nucleoside.

Embodiment 8. The oligomeric agent of embodiment 7, wherein the modified nucleoside comprises a modified sugar moiety.

Embodiment 9. The oligomeric agent of embodiment 8, wherein the modified sugar moiety comprises a bicyclic sugar moiety.

Embodiment 10. The oligomeric agent of embodiment 9, wherein the bicyclic sugar moiety comprises a 2′-4′ bridge selected from —O—CH₂—; and —O—CH(CH₃)—.

Embodiment 11. The oligomeric agent of embodiment 7, wherein the modified nucleoside comprises a non-bicyclic modified sugar moiety.

Embodiment 12. The oligomeric agent of embodiment 11, wherein the non-bicyclic modified sugar moiety is a 2′-MOE sugar moiety, a 2′-OMe sugar moiety, or a 2′-β-D-deoxyxylosyl sugar moiety.

Embodiment 13. The oligomeric agent of embodiment 7, wherein the modified nucleoside comprises a sugar surrogate.

Embodiment 14. The oligomeric agent of embodiment 13, wherein the sugar surrogate is any of morpholino, modified morpholino, glycol nucleic acid (GNA), six-membered tetrahydropyran (THP), and F-hexitol nucleic acid (FHNA).

Embodiment 15. The oligomeric agent of any of embodiments 1-14, wherein the modified oligonucleotide is a gapmer.

Embodiment 16. The oligomeric agent of any of embodiments 1-15, wherein the modified oligonucleotide comprises:
a 5′-region consisting of 1-6 linked 5′-region nucleosides;
a central region consisting of 6-10 linked central region nucleosides; and
a 3′-region consisting of 1-6 linked 3′-region nucleosides;
   wherein each of the 5′-region nucleosides and each of the 3′-region nucleosides comprises a modified sugar moiety and each of the central region nucleosides comprises a 2′-β-D-deoxyribosyl sugar moiety.

Embodiment 17. The oligomeric agent of any of embodiments 1-16, wherein the modified oligonucleotide comprises:
a 5′-region consisting of 6 linked 5′-region nucleosides;
a central region consisting of 10 linked central region nucleosides; and
a 3′-region consisting of 4 linked 3′-region nucleosides;
   wherein each of the 5′-region nucleosides and each of the 3′-region nucleosides comprises a modified sugar moiety and each of the central region nucleosides comprises a 2′-β-D-deoxyribosyl sugar moiety.

Embodiment 18. The oligomeric agent of any of embodiments 1-16, wherein the modified oligonucleotide comprises:
a 5′-region consisting of 4 linked 5′-region nucleosides;
a central region consisting of 10 linked central region nucleosides; and
a 3′-region consisting of 6 linked 3′-region nucleosides;
   wherein each of the 5′-region nucleosides and each of the 3′-region nucleosides comprises a modified sugar moiety and each of the central region nucleosides comprises a 2′-β-D-deoxyribosyl sugar moiety.

Embodiment 19. The oligomeric agent of any of embodiments 16-18, wherein the modified sugar moiety is a 2′-MOE sugar moiety or a cEt sugar moiety.

Embodiment 20. The oligomeric agent of any of embodiments 16-19, wherein the modified oligonucleotide comprises:
a 5′-region consisting of 6 linked 5′-region nucleosides;
a central region consisting of 10 linked central region nucleosides; and
a 3′-region consisting of 4 linked 3′-region nucleosides;
   wherein each of the 5′-region nucleosides and each of the 3′-region nucleosides comprises a modified sugar moiety selected from the group consisting of a 2′-MOE sugar moiety and a cEt sugar moiety, and each of the central region nucleosides comprises a 2′-β-D-deoxyribosyl sugar moiety.

Embodiment 21. The oligomeric agent of any of embodiments 16-19, wherein the modified oligonucleotide comprises:
a 5′-region consisting of 4 linked 5′-region nucleosides;
a central region consisting of 10 linked central region nucleosides; and
a 3′-region consisting of 6 linked 3′-region nucleosides;
   wherein each of the 5′-region nucleosides and each of the 3′-region nucleosides comprises a modified sugar moiety selected from the group consisting of a 2′-MOE sugar moiety and a cEt sugar moiety, and each of the central region nucleosides comprises a 2′-β-D-deoxyribosyl sugar moiety.

Embodiment 22. An oligomeric agent comprising a modified oligonucleotide consisting of 12 to 50 linked nucleosides, wherein the modified oligonucleotide comprises:
a 5′-region consisting of 1-6 linked 5′-region nucleosides;
a central region consisting of 10-13 linked central region nucleosides; and
a 3′-region consisting of 1-5 linked 3′-region nucleosides;
   wherein:
each nucleoside in the 5′-region nucleosides comprises either a modified sugar moiety or a 2′-β-D-deoxyribosyl sugar moiety;
each nucleoside in the 3′-region nucleosides comprises either a modified sugar moiety or a 2′-β-D-deoxyribosyl sugar moiety;
at least one nucleoside in either the 5′-region or the 3′-region is a 2′-β-D-deoxyribosyl sugar moiety; and
each of the central region nucleosides comprises a 2′-β-D-deoxyribosyl sugar moiety; and
   wherein the nucleobase sequence of the modified oligonucleotide is at least 80% complementary to an equal length portion of an APOE nucleic acid.

Embodiment 23. The oligomeric agent of embodiment 22, wherein the modified sugar moiety comprises a bicyclic sugar moiety.

Embodiment 24. The oligomeric agent of embodiment 23, wherein the bicyclic sugar moiety is a cEt sugar moiety.

Embodiment 25. The oligomeric agent of embodiment 22, wherein the modified sugar moiety comprises a non-bicyclic modified sugar moiety.

Embodiment 26. The oligomeric agent of embodiment 25, wherein the non-bicyclic modified sugar moiety is a 2′-MOE sugar moiety, a 2′-OMe sugar moiety, or a 2′-β-D-deoxyxylosyl sugar moiety.

Embodiment 27. The oligomeric agent of embodiment 22, wherein the modified sugar moiety comprises a sugar surrogate.

Embodiment 28. The oligomeric agent of embodiment 27, wherein the sugar surrogate is any of morpholino, modified morpholino, glycol nucleic acid (GNA), six-membered tetrahydropyran (THP), and F-hexitol nucleic acid (FHNA).

Embodiment 29. The oligomeric agent of any of embodiments 22-28, wherein the modified oligonucleotide comprises:
a 5′-region consisting of 3-6 linked 5′-region nucleosides;
a central region consisting of 10-13 linked central region nucleosides; and
a 3′-region consisting of 3-5 linked 3′-region nucleosides;
   wherein:
each nucleoside in the 5′-region nucleosides comprises either a cEt sugar moiety or a 2′-β-D-deoxyribosyl sugar moiety;
each nucleoside in the 3′-region nucleosides comprises either a cEt sugar moiety or a 2′-β-D-deoxyribosyl sugar moiety;
at least one nucleoside in either the 5′-region or the 3′-region is a 2′-β-D-deoxyribosyl sugar moiety; and
each of the central region nucleosides comprises a 2′-β-D-deoxyribosyl sugar moiety and
wherein the nucleobase sequence of the modified oligonucleotide is at least 80% complementary to an equal length portion of an APOE nucleic acid.

Embodiment 30. The oligomeric agent of any of embodiments 22-29, wherein the modified oligonucleotide comprises:
a 5′-region consisting of 3-6 linked 5′-region nucleosides;
a central region consisting of 10-13 linked central region nucleosides; and
a 3′-region consisting of 4 linked 3′-region nucleosides;
   wherein:
each nucleoside in the 5′-region nucleosides comprises either a cEt sugar moiety or a 2′-β-D-deoxyribosyl sugar moiety;
each nucleoside in the 3′-region nucleosides comprises either a cEt sugar moiety or a 2′-β-D-deoxyribosyl sugar moiety;
at least one nucleoside in either the 5′-region or the 3′-region is a 2′-β-D-deoxyribosyl sugar moiety; and
each of the central region nucleosides comprises a 2′-β-D-deoxyribosyl sugar moiety and
wherein the nucleobase sequence of the modified oligonucleotide is at least 80% complementary to an equal length portion of an APOE nucleic acid.

Embodiment 31. The oligomeric agent of any of embodiments 22-29, wherein the modified oligonucleotide comprises:
a 5′-region consisting of 5-6 linked 5′-region nucleosides;
a central region consisting of 11-13 linked central region nucleosides; and
a 3′-region consisting of 3 linked 3′-region nucleosides;
   wherein:
each nucleoside in the 5′-region nucleosides comprises either a cEt sugar moiety or a 2′-β-D-deoxyribosyl sugar moiety;
each nucleoside in the 3′-region nucleosides comprises either a cEt sugar moiety or a 2′-β-D-deoxyribosyl sugar moiety;
at least one nucleoside in either the 5′-region or the 3′-region is a 2′-β-D-deoxyribosyl sugar moiety; and
each of the central region nucleosides comprises a 2′-β-D-deoxyribosyl sugar moiety and
wherein the nucleobase sequence of the modified oligonucleotide is at least 80% complementary to an equal length portion of an APOE nucleic acid.

Embodiment 32. The oligomeric agent of any of embodiments 22-30, wherein the modified oligonucleotide comprises:
a 5′-region consisting of 3 linked 5′-region nucleosides;
a central region consisting of 13 linked central region nucleosides; and
a 3′-region consisting of 4 linked 3′-region nucleosides;
   wherein:
each nucleoside in the 5′-region nucleosides comprises either a cEt sugar moiety or a 2′-β-D-deoxyribosyl sugar moiety;
each nucleoside in the 3′-region nucleosides comprises either a cEt sugar moiety or a 2′-β-D-deoxyribosyl sugar moiety;
at least one nucleoside in either the 5′-region or the 3′-region is a 2′-β-D-deoxyribosyl sugar moiety; and
each of the central region nucleosides comprises a 2′-β-D-deoxyribosyl sugar moiety and
wherein the nucleobase sequence of the modified oligonucleotide is at least 80% complementary to an equal length portion of an APOE nucleic acid.

Embodiment 33. The oligomeric agent of any of embodiments 22-32, wherein the nucleobase sequence of the modified oligonucleotide is at least 85%, at least 90%, at least 95%, or 100% complementary to an equal length portion of an APOE nucleic acid.

Embodiment 34. The oligomeric agent of any of embodiments 22-33, wherein the nucleobase sequence of the modified oligonucleotide is at least 85%, at least 90%, at least 95%, or 100% complementary to the nucleobase sequence of any one of SEQ ID NOs: 1-5 when measured across the entire nucleobase sequence of the modified oligonucleotide.

Embodiment 35. The oligomeric agent of any of embodiments 1-34, wherein the modified oligonucleotide comprises at least one phosphodiester internucleoside linkage.

Embodiment 36. The oligomeric agent of any of embodiments 1-35, wherein the modified oligonucleotide comprises at least one phosphorothioate internucleoside linkage.

Embodiment 37. The oligomeric agent of embodiment 35 or embodiment 36, wherein the modified oligonucleotide comprises at least one mesyl phosphoramidate internucleoside linkage and each remaining internucleoside linkage is independently selected from a phosphodiester internucleoside linkage, a phosphorothioate internucleoside linkage, and a mesyl phosphoramidate internucleoside linkage.

Embodiment 38. The oligomeric agent of embodiment 35 or embodiment 36, wherein the modified oligonucleotide comprises at least one mesyl phosphoramidate internucleoside linkage and each remaining internucleoside linkage is independently selected from a phosphorothioate internucleoside linkage and a mesyl phosphoramidate internucleoside linkage.

Embodiment 39. The oligomeric agent of any of embodiments 35-38, wherein at least 4, at least 5, at least 6, at least 7, at least 8, at least 9, at least 10, at least 11, at least 12, at least 13, at least 14, at least 15, at least 16, at least 17, at least 18, or 19 internucleoside linkages of the modified oligonucleotide are phosphorothioate internucleoside linkages.

Embodiment 40. The oligomeric agent of any of embodiments 35-39, wherein at least 1, at least 2, at least 3, at least 4, at least 5, at least 6, at least 7, or at least 8 internucleoside linkages of the modified oligonucleotide are mesyl phosphoramidate internucleoside linkages.

Embodiment 41. The oligomeric agent of any of embodiments 35-40, wherein the modified oligonucleotide has an internucleoside linkage motif selected from soossssssssssooss, sosssszzzssssszoss, ssoooszzsssssszsooss, ssoooszzzssssszsooss, ssoosszzzssssszsooss, zsoooszzsssssszsoosz, zsossszzzssssszsoosz, soooosszzsssssszoss, sooosssssssssssooss, sooossszzsssssszoss, sooossszzsssssszsss, soooszzzsssssssssss, soooszzzsssszssooss, soooszzzsssszzsooss, soosssszzzssssszsss, soossszzsssssssssss, soossszzzssssszssss, soosszzzssssszsooss, soosszzzssssszsssss, sosssszzzssssszssss, sossszzzzssssszssss, soszsszzzssssszssss, ssoooszzssssssssoos, ssssoszzzssssszssss, sssssozzzssssszssss, ssszzszzzssssszsszz, ssszzzssssszzsssssz, ssszzzssssszzzssssz, ssszzzssszzsssssssz, ssszzzsszzssssssssz, ssszzzszzssssszsssz, ssszzzzzssssssssssz, ssszzzzzzsssssssssz, ssszzzzzzsssssszssz, ssszzzzzzssssszsssz, ssszzzzzzssssszsszs, ssszzzzzzssssszszss, ssszzzzzzzssssssssz, sszszszzzssssszsszz, sszszzzzzssssszsssz, sszszzzzzssssszsszs, sszszzzzzssssszszss, sszzszzzzssssszsssz, sszzszzzzssssszsszs, sszzszzzzssssszszss, sszzzszzzssssszsssz, sszzzszzzssssszsszs, sszzzszzzssssszszss, szsszszzzssssszsszz, szszsszzzssssszszsz, szszszzzzssssszsssz, szszszzzzssssszsszs, szszszzzzssssszszss, szszzszzzssssszsssz, szszzszzzssssszsszs, szszzszzzssssszszss, szzssszzzssssszsszz, szzsszzzzssssszsssz, szzsszzzzssssszsszs, szzsszzzzssssszszss, szzszsszzssssszsssz, szzszszzssssssssssz, szzszszzzsssssszssz, szzszszzzssssszsssz, szzszszzzssssszsszs, szzszszzzssssszszss, szzszszzzzsssssssz, szzzsszzzssssszsssz, szzzsszzzssssszsszs, szzzsszzzssssszszss, zoosszzzssssszsssss, zossszzzssssszsssss, or zossszzzssssszssssz, wherein each “s” represents a phosphorothioate internucleoside linkage, each “o” represents a phosphodiester internucleoside linkage, and each “z” represents a mesyl phosphoramidate internucleoside linkage.

Embodiment 42. The oligomeric agent of any of embodiments 35-40, wherein the modified oligonucleotide has an internucleoside linkage motif selected from soooosszzsssssszoss, sooosssssssssssooss, sooossszzsssssszoss, sooossszzsssssszsss, soooszzzsssssssssss, soooszzzsssszssooss, soooszzzsssszzsooss, soosssszzzssssszsss, soossszzsssssssssss, soossszzzssssszssss, soosszzzssssszsooss, soosszzzssssszsssss, sosssszzzssssszssss, sossszzzzssssszssss, soszsszzzssssszssss, ssoooszzssssssssoos, ssssoszzzssssszssss, sssssozzzssssszssss, ssszzszzzssssszsszz, ssszzzssssszzsssssz, ssszzzssssszzzssssz, ssszzzssszzsssssssz, ssszzzsszzssssssssz, ssszzzszzssssszsssz, ssszzzzzssssssssssz, ssszzzzzzsssssssssz, ssszzzzzzsssssszssz, ssszzzzzzssssszsssz, ssszzzzzzssssszsszs, ssszzzzzzssssszszss, ssszzzzzzzssssssssz, sszszszzzssssszsszz, sszszzzzzssssszsssz, sszszzzzzssssszsszs, sszszzzzzssssszszss, sszzszzzzssssszsssz, sszzszzzzssssszsszs, sszzszzzzssssszszss, sszzzszzzssssszsssz, sszzzszzzssssszsszs, sszzzszzzssssszszss, szsszszzzssssszsszz, szszsszzzssssszszsz, szszszzzzssssszsssz, szszszzzzssssszsszs, szszszzzzssssszszss, szszzszzzssssszsssz, szszzszzzssssszsszs, szszzszzzssssszszss, szzssszzzssssszsszz, szzsszzzzssssszsssz, szzsszzzzssssszsszs, szzsszzzzssssszszss, szzszsszzssssszsssz, szzszszzssssssssssz, szzszszzzsssssszssz, szzszszzzssssszsssz, szzszszzzssssszsszs, szzszszzzssssszszss, szzszszzzzssssssssz, szzzsszzzssssszsssz, szzzsszzzssssszsszs, szzzsszzzssssszszss, zoosszzzssssszsssss, zossszzzssssszsssss, or zossszzzssssszssssz, wherein each “s” represents a phosphorothioate internucleoside linkage, each “o” represents a phosphodiester internucleoside linkage, and each “z” represents a mesyl phosphoramidate internucleoside linkage.

Embodiment 43. The oligomeric agent of any of embodiments 1-42, wherein at least one nucleoside of the modified oligonucleotide comprises a modified nucleobase.

Embodiment 44. The oligomeric agent of embodiment 43, wherein the modified nucleobase is a 5-methylcytosine.

Embodiment 45. The oligomeric agent of embodiment 44, wherein each cytosine is a 5-methylcytosine.

Embodiment 46. The oligomeric agent of any of embodiments 1-45, wherein each nucleoside of the modified oligonucleotide is unmodified adenine, unmodified guanine, unmodified thymine, unmodified cytosine, or 5-methylcytosine.

Embodiment 47. The oligomeric agent of any of embodiments 1-46, wherein the modified oligonucleotide consists of 12-30, 12-22, 12-21, 12-20, 14-18, 14-20, 14-21, 15-17, 15-21, 15-25, 16-20, 16-21, 18-22, 18-21, or 18-20 linked nucleosides.

Embodiment 48. The oligomeric agent of any of embodiments 1-47, wherein the modified oligonucleotide consists of 18 linked nucleosides.

Embodiment 49. The oligomeric agent of any of embodiments 1-47, wherein the modified oligonucleotide consists of 19 linked nucleosides.

Embodiment 50. The oligomeric agent of any of embodiments 1-47, wherein the modified oligonucleotide consists of 20 linked nucleosides.

Embodiment 51. The oligomeric agent of any of embodiments 1-47, wherein the modified oligonucleotide consists of 21 linked nucleosides.

Embodiment 52. The oligomeric agent of any of embodiments 1-51, wherein the oligomeric agent consists of the modified oligonucleotide.

Embodiment 53. The oligomeric agent of any of embodiments 1-51, wherein the oligomeric agent comprises a conjugate group.

Embodiment 54. The oligomeric agent of embodiment 53, wherein the conjugate group comprises a conjugate moiety and a conjugate linker.

Embodiment 55. The oligomeric agent of embodiment 54, wherein the conjugate linker is a phosphodiester linker.

Embodiment 56. The oligomeric agent of embodiment 54, wherein the conjugate linker consists of a single bond.

Embodiment 57. The oligomeric agent of any of embodiments 54-56, wherein the conjugate linker is cleavable.

Embodiment 58. The oligomeric agent of any of embodiments 54, 55, or 57, wherein the conjugate linker is attached to one or more nucleosides that are attached to the remainder of the modified oligonucleotide through at least one phosphodiester bond.

Embodiment 59. The oligomeric agent of any of embodiments 53-58, wherein the conjugate group is attached to the modified oligonucleotide at the 5′-end of the modified oligonucleotide.

Embodiment 60. The oligomeric agent of any of embodiments 53-58, wherein the conjugate group is attached to the modified oligonucleotide at the 3′-end of the modified oligonucleotide.

Embodiment 61. The oligomeric agent of any of embodiments 1-60, wherein the modified oligonucleotide comprises a terminal group.

Embodiment 62. The oligomeric agent of embodiment 61, wherein the terminal group is an abasic sugar moiety.

Embodiment 63. The oligomeric agent of any of embodiments 1-62, wherein the oligomeric agent is an antisense agent.

Embodiment 64. The oligomeric agent of any of embodiments 1-63, wherein the oligomeric agent is a single-stranded antisense agent.

Embodiment 65. The oligomeric agent of any of embodiments 1-64, wherein the oligomeric agent is an RNase H agent.

Embodiment 66. The oligomeric agent of any of embodiments 1-51 or 53-63, wherein the oligomeric agent is an RNAi agent.

Embodiment 67. The oligomeric agent of any of embodiments 1-51, 53-63, or 66, comprising a second modified oligonucleotide consisting of 12 to 50 linked nucleosides, and wherein the nucleobase sequence of the second modified oligonucleotide comprises a complementary region of at least 8 nucleobases that is at least 90% complementary to an equal length portion of the modified oligonucleotide.

Embodiment 68. The oligomeric agent of embodiment 67, wherein the modified oligonucleotide comprises a 5′-stabilized phosphate group.

Embodiment 69. The oligomeric agent of embodiment 68, wherein the stabilized phosphate group comprises a cyclopropyl phosphonate or a vinyl phosphonate.

Embodiment 70. The oligomeric agent of any of embodiments 67-69, wherein at least one nucleoside of the second modified oligonucleotide comprises a modified sugar moiety.

Embodiment 71. The oligomeric agent of embodiment 70, wherein the modified sugar moiety of the second modified oligonucleotide comprises a bicyclic sugar moiety.

Embodiment 72. The oligomeric agent of embodiment 71, wherein the bicyclic sugar moiety of the second modified oligonucleotide comprises a 2′-4′ bridge selected from —O—CH₂—; and —O—CH(CH₃)—.

Embodiment 73. The oligomeric agent of embodiment 70, wherein the modified sugar moiety of the second modified oligonucleotide comprises a non-bicyclic modified sugar moiety.

Embodiment 74. The oligomeric agent of embodiment 73, wherein the non-bicyclic modified sugar moiety of the second modified oligonucleotide is a 2′-MOE sugar moiety, a 2′-F modified sugar moiety, or 2′-OMe modified sugar moiety.

Embodiment 75. The oligomeric agent of any of embodiments 67-74, wherein at least one internucleoside linkage of the second modified oligonucleotide is a modified internucleoside linkage.

Embodiment 76. The oligomeric agent of embodiment 75, wherein at least one modified internucleoside linkage of the second modified oligonucleotide is a phosphorothioate internucleoside linkage.

Embodiment 77. The oligomeric agent of any of embodiments 75-76, wherein at least one internucleoside linkage of the second modified oligonucleotide is a phosphodiester internucleoside linkage.

Embodiment 78. The oligomeric agent of any of embodiments 75-77, wherein at least one internucleoside linkage of the second modified oligonucleotide is a mesyl phosphoramidate internucleoside linkage.

Embodiment 79. The oligomeric agent of any of embodiments 75-78, wherein each internucleoside linkage of the second modified oligonucleotide is independently selected from a phosphodiester internucleoside linkage, a phosphorothioate internucleoside linkage, or a mesyl phosphoramidate internucleoside linkage.

Embodiment 80. The oligomeric agent of any of embodiments 67-79, wherein the second modified oligonucleotide comprises at least one modified nucleobase.

Embodiment 81. The oligomeric agent of embodiment 80, wherein the at least one modified nucleobase of the second modified oligonucleotide is 5-methylcytosine.

Embodiment 82. The oligomeric agent of any of embodiments 67-81, wherein the second modified oligonucleotide comprises a conjugate group.

Embodiment 83. The oligomeric agent of embodiment 82, wherein the conjugate group comprises a conjugate moiety and a conjugate linker.

Embodiment 84. The oligomeric agent of embodiment 83, wherein the conjugate linker consists of a single bond.

Embodiment 85. The oligomeric agent of embodiment 83 or embodiment 84, wherein the conjugate linker is cleavable.

Embodiment 86. The oligomeric agent of embodiment 83 or embodiment 85, wherein the conjugate linker is attached to one or more nucleosides that are attached to the remainder of the modified oligonucleotide through at least one phosphodiester bond.

Embodiment 87. The oligomeric agent of any of embodiments 83-86, wherein the conjugate linker is a phosphodiester linker.

Embodiment 88. The oligomeric agent of any of embodiments 82-87, wherein the conjugate group is attached to the 5′-end of the second modified oligonucleotide.

Embodiment 89. The oligomeric agent of any of embodiments 82-87, wherein the conjugate group is attached to the 3′-end of the second modified oligonucleotide.

Embodiment 90. The oligomeric agent of any of embodiments 82-87, wherein the conjugate group is attached via the 2′ position of a ribosyl sugar moiety at an internal position of the second modified oligonucleotide.

Embodiment 91. The oligomeric agent of any of embodiments 82-90, wherein the conjugate group comprises a C22 alkyl, C20 alkyl, C16 alkyl, C10 alkyl, C21 alkyl, C19 alkyl, C18 alkyl, C17 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C17 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl.

Embodiment 92. The oligomeric agent of any of embodiments 82-91, wherein the conjugate group comprises a cell-targeting moiety.

Embodiment 93. The oligomeric agent of any of embodiments 67-92, wherein the second modified oligonucleotide comprises a terminal group.

Embodiment 94. The oligomeric agent of embodiment 93, wherein the terminal group is an abasic sugar moiety.

Embodiment 95. An oligomeric agent comprising a modified oligonucleotide according to the following chemical notation: Aₑₛ^{m}CₑₒTₑₒTₑₛGₑₛGₖₛT_{ds}G_{dz}A_{dz}A_{dz}T_{ds}^{m}C_{ds}T_{ds}T_{ds}T_{ds}A_{dz}TₖₛTₖₛAₑₛAₑ (SEQ ID NO: 45), wherein:
A=an adenine nucleobase,
^{m}C=a 5-methylcytosine nucleobase,
G=a guanine nucleobase,
T=a thymine nucleobase,
e=a 2′-MOE sugar moiety,
k=a cEt sugar moiety,
d=a 2′-β-D-deoxyribosyl sugar moiety,
s=a phosphorothioate internucleoside linkage,
o=a phosphodiester internucleoside linkage, and
z=a mesyl phosphoramidate internucleoside linkage; and the oligomeric agent does not include a conjugate group or a terminal group.

Embodiment 96. An oligomeric agent comprising a modified oligonucleotide according to the following chemical notation: ^{m}CₖₛTₖₛTₖₛG_{dz}G_{dz}T_{dz}G_{dz}A_{dz}A_{dz}T_{ds}^{m}C_{ds}T_{ds}T_{ds}T_{ds}A_{ds}T_{ds}TₖₛAₖₛA_{dz}Aₖ (SEQ ID NO: 46), wherein:
A=an adenine nucleobase,
^{m}C=a 5-methylcytosine nucleobase,
G=a guanine nucleobase,
T=a thymine nucleobase,
k=a cEt sugar moiety,
d=a 2′-β-D-deoxyribosyl sugar moiety,
s=a phosphorothioate internucleoside linkage, and
z=a mesyl phosphoramidate internucleoside linkage; and the oligomeric agent does not include a conjugate group or a terminal group.

Embodiment 97. An oligomeric agent comprising a modified oligonucleotide according to the following chemical notation: Aₖₛ^{m}CₑₒTₑₒTₑₛGₑₛGₖₛT_{ds}G_{dz}A_{dz}A_{dz}T_{ds}^{m}C_{ds}T_{ds}T_{ds}T_{ds}A_{dz}TₖₛTₖₛAₑₛAₑ (SEQ ID NO: 47), wherein:
A=an adenine nucleobase,
^{m}C=a 5-methylcytosine nucleobase,
G=a guanine nucleobase,
T=a thymine nucleobase,
e=a 2′-MOE sugar moiety,
k=a cEt sugar moiety,
d=a 2′-β-D-deoxyribosyl sugar moiety,
s=a phosphorothioate internucleoside linkage,
o=a phosphodiester internucleoside linkage, and
z=a mesyl phosphoramidate internucleoside linkage; and the oligomeric agent does not include a conjugate group or a terminal group.

Embodiment 98. An oligomeric agent comprising a modified oligonucleotide according to the following chemical notation: T_{ez}TₑₒGₑₒGₖₛT_{ds}G_{dz}A_{dz}A_{dz}T_{ds}^{m}C_{ds}T_{ds}T_{ds}T_{ds}A_{dz}TₖₛTₖₛAₑₛAₑₛAₑₛ^{m}Cₑ (SEQ ID NO: 48), wherein:
A=an adenine nucleobase,
^{m}C=a 5-methylcytosine nucleobase,
G=a guanine nucleobase,
T=a thymine nucleobase,
e=a 2′-MOE sugar moiety,
k=a cEt sugar moiety,
d=a 2′-β-D-deoxyribosyl sugar moiety,
s=a phosphorothioate internucleoside linkage,
o=a phosphodiester internucleoside linkage, and
z=a mesyl phosphoramidate internucleoside linkage; and the oligomeric agent does not include a conjugate group or a terminal group.

Embodiment 99. The oligomeric agent of any of embodiments 95-98, wherein the modified oligonucleotide is a pharmaceutically acceptable salt.

Embodiment 100. The oligomeric agent of embodiment 99, wherein the pharmaceutically acceptable salt comprises one or more cations selected from sodium, potassium, calcium, and magnesium.

Embodiment 101. An oligomeric agent according to the following chemical notation: N¹ₑₛ^{m}CₑₒTₑₒTₑₛGₑₛGₖₛT_{ds}G_{dz}A_{dz}A_{dz}T_{ds}^{m}C_{ds}T_{ds}T_{ds}T_{ds}A_{dz}TₖₛTₖₛN³ₑₛN²ₑ (SEQ ID NO: 49), wherein:
A=an adenine nucleobase,
^{m}C=a 5-methylcytosine nucleobase,
G=a guanine nucleobase,
T=a thymine nucleobase,
N¹=an adenine nucleobase, a modified adenine, a hypoxanthine, an abasic sugar moiety, a terminal group, or is absent, wherein when N¹ is absent its sugar and internucleoside linkage are also absent,
N²=an adenine nucleobase, a modified adenine, a hypoxanthine, an abasic sugar moiety, a terminal group, or is absent, wherein when N² is absent its sugar and internucleoside linkage are also absent,
N³=an adenine nucleobase, a modified adenine, a hypoxanthine, an abasic sugar moiety, a terminal group, or is absent, wherein when N³ is absent its sugar and internucleoside linkage are also absent,
e=a 2′-MOE sugar moiety,
k=a cEt sugar moiety,
d=a 2′-β-D-deoxyribosyl sugar moiety,
s=a phosphorothioate internucleoside linkage,
o=a phosphodiester internucleoside linkage, and
z=a mesyl phosphoramidate internucleoside linkage; and wherein the oligomeric agent optionally comprises a conjugate group.

Embodiment 102. An oligomeric agent according to the following chemical notation: N⁵ₖₛTₖₛTₖₛG_{dz}G_{dz}T_{dz}G_{dz}A_{dz}A_{dz}T_{ds}^{m}C_{ds}T_{ds}T_{ds}T_{ds}A_{ds}T_{ds}TₖₛN⁴ₖₛN³_{dz}N²ₖ (SEQ ID NO: 50), wherein:
A=an adenine nucleobase,
^{m}C=a 5-methylcytosine nucleobase,
G=a guanine nucleobase,
T=a thymine nucleobase,
N²=an adenine nucleobase, a modified adenine, a hypoxanthine, an abasic sugar moiety, a terminal group, or is absent, wherein when N² is absent its sugar and internucleoside linkage are also absent,
N³=an adenine nucleobase, a modified adenine, a hypoxanthine, an abasic sugar moiety, a terminal group, or is absent, wherein when N³ is absent its sugar and internucleoside linkage are also absent,
N⁴=an adenine nucleobase, a modified adenine, a hypoxanthine, an abasic sugar moiety, a terminal group, or is absent, wherein when N⁴ is absent its sugar and internucleoside linkage are also absent,
N⁵=a cytosine nucleobase, a modified cytosine, an abasic sugar moiety, a terminal group, or is absent, wherein when N⁵ is absent its sugar is also absent,
k=a cEt sugar moiety,
d=a 2′-β-D-deoxyribosyl sugar moiety,
s=a phosphorothioate internucleoside linkage, and
z=a mesyl phosphoramidate internucleoside linkage; and
   wherein the oligomeric agent optionally comprises a conjugate group.

Embodiment 103. An oligomeric agent according to the following chemical notation: N¹ₖₛ^{m}CₑₒTₑₒTₑₛGₑₛGₖₛT_{ds}G_{dz}A_{dz}A_{dz}T_{ds}^{m}C_{ds}T_{ds}T_{ds}T_{ds}A_{dz}TₖₛTₖₛN³ₑₛN²ₑ (SEQ ID NO: 51), wherein:
A=an adenine nucleobase,
^{m}C=a 5-methylcytosine nucleobase,
G=a guanine nucleobase,
T=a thymine nucleobase,
N¹=an adenine nucleobase, a modified adenine, a hypoxanthine, an abasic sugar moiety, a terminal group, or is absent, wherein when N¹ is absent its sugar and internucleoside linkage are also absent,
N²=an adenine nucleobase, a modified adenine, a hypoxanthine, an abasic sugar moiety, a terminal group, or is absent, wherein when N² is absent its sugar and internucleoside linkage are also absent,
N³=an adenine nucleobase, a modified adenine, a hypoxanthine, an abasic sugar moiety, a terminal group, or is absent, wherein when N³ is absent its sugar and internucleoside linkage are also absent,
e=a 2′-MOE sugar moiety,
k=a cEt sugar moiety,
d=a 2′-β-D-deoxyribosyl sugar moiety,
s=a phosphorothioate internucleoside linkage,
o=a phosphodiester internucleoside linkage, and
z=a mesyl phosphoramidate internucleoside linkage; and
   wherein the oligomeric agent optionally comprises a conjugate group.

Embodiment 104. The oligomeric agent of embodiment 101 or embodiment 103, wherein N¹ is an adenine nucleobase.

Embodiment 105. The oligomeric agent of embodiment 101 or embodiment 103, wherein N¹ is an unmodified adenine.

Embodiment 106. The oligomeric agent of embodiment 101 or embodiment 103, wherein N¹ is a modified adenine.

Embodiment 107. The oligomeric agent of embodiment 101 or embodiment 103, wherein N¹ is a hypoxanthine.

Embodiment 108. The oligomeric agent of embodiment 101 or embodiment 103, wherein N¹ is an abasic sugar moiety.

Embodiment 109. The oligomeric agent of embodiment 101 or embodiment 103, wherein N¹ is a terminal group.

Embodiment 110. The oligomeric agent of embodiment 101 or embodiment 103, wherein N¹ is absent.

Embodiment 111. The oligomeric agent of any of embodiments 101-110, wherein N² is an adenine nucleobase.

Embodiment 112. The oligomeric agent of any of embodiments 101-110, wherein N² is an unmodified adenine.

Embodiment 113. The oligomeric agent of any of embodiments 101-110, wherein N² is a modified adenine.

Embodiment 114. The oligomeric agent of any of embodiments 101-110, wherein N² is a hypoxanthine.

Embodiment 115. The oligomeric agent of any of embodiments 101-110, wherein N² is an abasic sugar moiety.

Embodiment 116. The oligomeric agent of any of embodiments 101-110, wherein N² is a terminal group.

Embodiment 117. The oligomeric agent of any of embodiments 101-110, wherein N² is absent.

Embodiment 118. The oligomeric agent of any of embodiments 101-117, wherein N³ is an adenine nucleobase.

Embodiment 119. The oligomeric agent of any of embodiments 101-117, wherein N³ is an unmodified adenine.

Embodiment 120. The oligomeric agent of any of embodiments 101-117, wherein N³ is a modified adenine.

Embodiment 121. The oligomeric agent of any of embodiments 101-117, wherein N³ is a hypoxanthine.

Embodiment 122. The oligomeric agent of any of embodiments 101-117, wherein N³ is an abasic sugar moiety.

Embodiment 123. The oligomeric agent of any of embodiments 101-117, wherein N³ is a terminal group.

Embodiment 124. The oligomeric agent of any of embodiments 101-117, wherein N³ is absent.

Embodiment 125. The oligomeric agent of any of embodiments 102 or 111-124, wherein N⁴ is an adenine nucleobase.

Embodiment 126. The oligomeric agent of any of embodiments 102 or 111-124, wherein N⁴ is an unmodified adenine.

Embodiment 127. The oligomeric agent of any of embodiments 102 or 111-124, wherein N⁴ is a modified adenine.

Embodiment 128. The oligomeric agent of any of embodiments 102 or 111-124, wherein N⁴ is a hypoxanthine.

Embodiment 129. The oligomeric agent of any of embodiments 102 or 111-124, wherein N⁴ is an abasic sugar moiety.

Embodiment 130. The oligomeric agent of any of embodiments 102 or 111-124, wherein N⁴ is a terminal group.

Embodiment 131. The oligomeric agent of any of embodiments 102 or 111-124, wherein N⁴ is absent.

Embodiment 132. The oligomeric agent of any of embodiments 102 or 111-131, wherein N⁵ is a modified cytosine.

Embodiment 133. The oligomeric agent of any of embodiments 102 or 111-131, wherein N⁵ is 5-methylcytosine.

Embodiment 134. The oligomeric agent of any of embodiments 102 or 111-131, wherein N⁵ is an unmodified cytosine.

Embodiment 135. The oligomeric agent of any of embodiments 102 or 111-131, wherein N⁵ is an abasic sugar moiety.

Embodiment 136. The oligomeric agent of any of embodiments 102 or 111-131, wherein N⁵ is a terminal group.

Embodiment 137. The oligomeric agent of any of embodiments 102 or 111-131, wherein N⁵ is absent.

Embodiment 138. The oligomeric agent of any of embodiments 101 or 103-117, wherein N¹ is an unmodified adenine, a modified adenine, a hypoxanthine, an abasic sugar moiety, a terminal group, or is absent, and N² is an unmodified adenine, a modified adenine, a hypoxanthine, an abasic sugar moiety, a terminal group, or is absent.

Embodiment 139. The oligomeric agent of any of embodiments 101, 103-117, or 138, wherein N¹ is an adenine nucleobase and N² is an unmodified adenine, a modified adenine, a hypoxanthine, an abasic sugar moiety, a terminal group, or is absent.

Embodiment 140. The oligomeric agent of any of embodiments 101, 103-117, or 138, wherein N¹ is an adenine nucleobase and N² is an unmodified adenine.

Embodiment 141. The oligomeric agent of any of embodiments 101, 103-117, or 138, wherein N¹ is an adenine nucleobase and N² is a modified adenine.

Embodiment 142. The oligomeric agent of any of embodiments 101, 103-117, or 138, wherein N¹ is an adenine nucleobase and N² is a hypoxanthine.

Embodiment 143. The oligomeric agent of any of embodiments 101, 103-117, or 138, wherein N¹ is an adenine nucleobase and N² is an abasic sugar moiety.

Embodiment 144. The oligomeric agent of any of embodiments 101, 103-117, or 138, wherein N¹ is an adenine nucleobase and N² is a terminal group.

Embodiment 145. The oligomeric agent of any of embodiments 101, 103-117, or 138, wherein N¹ is an adenine nucleobase and N² is absent.

Embodiment 146. The oligomeric agent of any of embodiments 101, 103-117, or 138, wherein N¹ is a modified adenine and N² is an unmodified adenine.

Embodiment 147. The oligomeric agent of any of embodiments 101, 103-117, or 138, wherein N¹ is a modified adenine and N² is a modified adenine.

Embodiment 148. The oligomeric agent of any of embodiments 101, 103-117, or 138, wherein N¹ is a modified adenine and N² is a hypoxanthine.

Embodiment 149. The oligomeric agent of any of embodiments 101, 103-117, or 138, wherein N¹ is a modified adenine and N² is an abasic sugar moiety.

Embodiment 150. The oligomeric agent of any of embodiments 101, 103-117, or 138, wherein N¹ is a modified adenine and N² is a terminal group.

Embodiment 151. The oligomeric agent of any of embodiments 101, 103-117, or 138, wherein N¹ is a modified adenine and N² is absent.

Embodiment 152. The oligomeric agent of any of embodiments 101, 103-117, or 138, wherein N¹ is a hypoxanthine and N² is an unmodified adenine.

Embodiment 153. The oligomeric agent of any of embodiments 101, 103-117, or 138, wherein N¹ is a hypoxanthine and N² is a modified adenine.

Embodiment 154. The oligomeric agent of any of embodiments 101, 103-117, or 138, wherein N¹ is a hypoxanthine and N² is a hypoxanthine.

Embodiment 155. The oligomeric agent of any of embodiments 101, 103-117, or 138, wherein N¹ is a hypoxanthine and N² is an abasic sugar moiety.

Embodiment 156. The oligomeric agent of any of embodiments 101, 103-117, or 138, wherein N¹ is a hypoxanthine and N² is a terminal group.

Embodiment 157. The oligomeric agent of any of embodiments 101, 103-117, or 138, wherein N¹ is a hypoxanthine and N² is absent.

Embodiment 158. The oligomeric agent of any of embodiments 101, 103-117, or 138, wherein N¹ is an abasic sugar moiety, a terminal group, or is absent, and N² is an unmodified adenine.

Embodiment 159. The oligomeric agent of any of embodiments 101, 103-117, or 138, wherein N¹ is an abasic sugar moiety, a terminal group, or is absent, and N² is a modified adenine.

Embodiment 160. The oligomeric agent of any of embodiments 101, 103-117, or 138, wherein N¹ is an abasic sugar moiety, a terminal group, or is absent, and N² is a hypoxanthine.

Embodiment 161. The oligomeric agent of any of embodiments 101, 103-117, or 138, wherein N¹ is an abasic sugar moiety, a terminal group, or is absent, and N² is an abasic sugar moiety.

Embodiment 162. The oligomeric agent of any of embodiments 101, 103-117, or 138, wherein N¹ is an abasic sugar moiety, a terminal group, or is absent, and N² is a terminal group.

Embodiment 163. The oligomeric agent of any of embodiments 101, 103-117, or 138, wherein N¹ is an abasic sugar moiety, a terminal group, or is absent, and N² is absent.

Embodiment 164. The oligomeric agent of any of embodiments 101, 103-110, or 117-124, wherein: a) N¹ is an unmodified adenine, a modified adenine, a hypoxanthine, an abasic sugar moiety, a terminal group, or is absent;
b) N² is absent; and
c) N³ is an unmodified adenine, a modified adenine, a hypoxanthine, an abasic sugar moiety, a terminal group, or is absent.

Embodiment 165. The oligomeric agent of any of embodiments 101, 103-110, 117-124, or 164, wherein N¹ is an adenine nucleobase, N² is absent, and N³ is an unmodified adenine.

Embodiment 166. The oligomeric agent of any of embodiments 101, 103-110, 117-124, or 164, wherein N¹ is an adenine nucleobase, N² is absent, and N³ is a modified adenine.

Embodiment 167. The oligomeric agent of any of embodiments 101, 103-110, 117-124, or 164, wherein N¹ is an adenine nucleobase, N² is absent, and N³ is a hypoxanthine.

Embodiment 168. The oligomeric agent of any of embodiments 101, 103-110, 117-124, or 164, wherein N¹ is an adenine nucleobase, N² is absent, and N³ is an abasic sugar moiety.

Embodiment 169. The oligomeric agent of any of embodiments 101, 103-110, 117-124, or 164, wherein N¹ is an adenine nucleobase, N² is absent, and N³ is a terminal group.

Embodiment 170. The oligomeric agent of any of embodiments 101, 103-110, 117-124, or 164, wherein N¹ is an adenine nucleobase, N² is absent, and N³ is absent.

Embodiment 171. The oligomeric agent of any of embodiments 101, 103-110, 117-124, or 164, wherein N¹ is a modified adenine, N² is absent, and N³ is an unmodified adenine.

Embodiment 172. The oligomeric agent of any of embodiments 101, 103-110, 117-124, or 164, wherein N¹ is a modified adenine, N² is absent, and N³ is a modified adenine.

Embodiment 173. The oligomeric agent of any of embodiments 101, 103-110, 117-124, or 164, wherein N¹ is a modified adenine, N² is absent, and N³ is a hypoxanthine.

Embodiment 174. The oligomeric agent of any of embodiments 101, 103-110, 117-124, or 164, wherein N¹ is a modified adenine, N² is absent, and N³ is an abasic sugar moiety.

Embodiment 175. The oligomeric agent of any of embodiments 101, 103-110, 117-124, or 164, wherein N¹ is a modified adenine, N² is absent, and N³ is a terminal group.

Embodiment 176. The oligomeric agent of any of embodiments 101, 103-110, 117-124, or 164, wherein N¹ is a modified adenine, N² is absent, and N³ is absent.

Embodiment 177. The oligomeric agent of any of embodiments 101, 103-110, 117-124, or 164, wherein N¹ is a hypoxanthine, N² is absent, and N³ is an unmodified adenine.

Embodiment 178. The oligomeric agent of any of embodiments 101, 103-110, 117-124, or 164, wherein N¹ is a hypoxanthine, N² is absent, and N³ is a modified adenine.

Embodiment 179. The oligomeric agent of any of embodiments 101, 103-110, 117-124, or 164, wherein N¹ is a hypoxanthine, N² is absent, and N³ is a hypoxanthine.

Embodiment 180. The oligomeric agent of any of embodiments 101, 103-110, 117-124, or 164, wherein N¹ is a hypoxanthine, N² is absent, and N³ is an abasic sugar moiety.

Embodiment 181. The oligomeric agent of any of embodiments 101, 103-110, 117-124, or 164, wherein N¹ is a hypoxanthine, N² is absent, and N³ is a terminal group.

Embodiment 182. The oligomeric agent of any of embodiments 101, 103-110, 117-124, or 164, wherein N¹ is a hypoxanthine, N² is absent, and N³ is absent.

Embodiment 183. The oligomeric agent of any of embodiments 101, 103-110, 117-124, or 164, wherein N¹ is an abasic sugar moiety, a terminal group, or is absent, N² is absent, and N³ is an unmodified adenine.

Embodiment 184. The oligomeric agent of any of embodiments 101, 103-110, 117-124, or 164, wherein N¹ is an abasic sugar moiety, a terminal group, or is absent, N² is absent, and N³ is a modified adenine.

Embodiment 185. The oligomeric agent of any of embodiments 101, 103-110, 117-124, or 164, wherein N¹ is an abasic sugar moiety, a terminal group, or is absent, N² is absent, and N³ is a hypoxanthine.

Embodiment 186. The oligomeric agent of any of embodiments 101, 103-110, 117-124, or 164, wherein N¹ is an abasic sugar moiety, a terminal group, or is absent, N² is absent, and N³ is an abasic sugar moiety.

Embodiment 187. The oligomeric agent of any of embodiments 101, 103-110, 117-124, or 164, wherein N¹ is an abasic sugar moiety, a terminal group, or is absent, N² is absent, and N³ is a terminal group.

Embodiment 188. The oligomeric agent of any of embodiments 101, 103-110, 117-124, or 164, wherein N¹ is an abasic sugar moiety, a terminal group, or is absent, N² is absent, and N³ is absent.

Embodiment 189. The oligomeric agent of any of embodiments 102, 111-117, or 132-137, wherein N⁵ is a cytosine nucleobase, a modified cytosine, an abasic sugar moiety, a terminal group, or is absent, and N² is an unmodified adenine, a modified adenine, a hypoxanthine, an abasic sugar moiety, a terminal group, or is absent.

Embodiment 190. The oligomeric agent of any of embodiments 102, 111-117, 132-137, or 189, wherein N⁵ is a cytosine nucleobase and N² is an unmodified adenine.

Embodiment 191. The oligomeric agent of any of embodiments 102, 111-117, 132-137, or 189, wherein N⁵ is a cytosine nucleobase and N² is a modified adenine.

Embodiment 192. The oligomeric agent of any of embodiments 102, 111-117, 132-137, or 189, wherein N⁵ is a cytosine nucleobase and N² is a hypoxanthine.

Embodiment 193. The oligomeric agent of any of embodiments 102, 111-117, 132-137, or 189, wherein N⁵ is a cytosine nucleobase and N² is an abasic sugar moiety.

Embodiment 194. The oligomeric agent of any of embodiments 102, 111-117, 132-137, or 189, wherein N⁵ is a cytosine nucleobase and N² is a terminal group.

Embodiment 195. The oligomeric agent of any of embodiments 102, 111-117, 132-137, or 189, wherein N⁵ is a cytosine nucleobase and N² is absent.

Embodiment 196. The oligomeric agent of any of embodiments 102, 111-117, 132-137, or 189, wherein N⁵ is a modified cytosine and N² is an unmodified adenine.

Embodiment 197. The oligomeric agent of any of embodiments 102, 111-117, 132-137, or 189, wherein N⁵ is a modified cytosine and N² is a modified adenine.

Embodiment 198. The oligomeric agent of any of embodiments 102, 111-117, 132-137, or 189, wherein N⁵ is a modified cytosine and N² is a hypoxanthine.

Embodiment 199. The oligomeric agent of any of embodiments 102, 111-117, 132-137, or 189, wherein N⁵ is a modified cytosine and N² is an abasic sugar moiety.

Embodiment 200. The oligomeric agent of any of embodiments 102, 111-117, 132-137, or 189, wherein N⁵ is a modified cytosine and N² is a terminal group.

Embodiment 201. The oligomeric agent of any of embodiments 102, 111-117, 132-137, or 189, wherein N⁵ is a modified cytosine and N² is absent.

Embodiment 202. The oligomeric agent of any of embodiments 102, 111-117, 132-137, or 189, wherein N⁵ is an abasic sugar moiety, a terminal group, or is absent, and N² is an unmodified adenine.

Embodiment 203. The oligomeric agent of any of embodiments 102, 111-117, 132-137, or 189, wherein N⁵ is an abasic sugar moiety, a terminal group, or is absent, and N² is a modified adenine.

Embodiment 204. The oligomeric agent of any of embodiments 102, 111-117, 132-137, or 189, wherein N⁵ is an abasic sugar moiety, a terminal group, or is absent, and N² is a hypoxanthine.

Embodiment 205. The oligomeric agent of any of embodiments 102, 111-117, 132-137, or 189, wherein N⁵ is an abasic sugar moiety, a terminal group, or is absent, and N² is an abasic sugar moiety.

Embodiment 206. The oligomeric agent of any of embodiments 102, 111-117, 132-137, or 189, wherein N⁵ is an abasic sugar moiety, a terminal group, or is absent, and N² is a terminal group.

Embodiment 207. The oligomeric agent of any of embodiments 102, 111-117, 132-137, or 189, wherein N⁵ is an abasic sugar moiety, a terminal group, or is absent, and N² is absent.

Embodiment 208. The oligomeric agent of any of embodiments 102, 117-124, or 132-137, wherein:
a) N⁵ is a cytosine nucleobase, a modified cytosine, an abasic sugar moiety, a terminal group, or is absent;
b) N² is absent; and
c) N³ is an unmodified adenine, a modified adenine, a hypoxanthine, an abasic sugar moiety, a terminal group, or is absent.

Embodiment 209. The oligomeric agent of any of embodiments 102, 117-124, 132-137, or 208, wherein N⁵ is a cytosine nucleobase, N² is absent, and N³ is an unmodified adenine.

Embodiment 210. The oligomeric agent of any of embodiments 102, 117-124, 132-137, or 208, wherein N⁵ is a cytosine nucleobase, N² is absent, and N³ is a modified adenine.

Embodiment 211. The oligomeric agent of any of embodiments 102, 117-124, 132-137, or 208, wherein N⁵ is a cytosine nucleobase, N² is absent, and N³ is a hypoxanthine.

Embodiment 212. The oligomeric agent of any of embodiments 102, 117-124, 132-137, or 208, wherein N⁵ is a cytosine nucleobase, N² is absent, and N³ is an abasic sugar moiety.

Embodiment 213. The oligomeric agent of any of embodiments 102, 117-124, 132-137, or 208, wherein N⁵ is a cytosine nucleobase, N² is absent, and N³ is a terminal group.

Embodiment 214. The oligomeric agent of any of embodiments 102, 117-124, 132-137, or 208, wherein N⁵ is a cytosine nucleobase, N² is absent, and N³ is absent.

Embodiment 215. The oligomeric agent of any of embodiments 102, 117-124, 132-137, or 208, wherein N⁵ is a modified cytosine, N² is absent, and N³ is an unmodified adenine.

Embodiment 216. The oligomeric agent of any of embodiments 102, 117-124, 132-137, or 208, wherein N⁵ is a modified cytosine, N² is absent, and N³ is a modified adenine.

Embodiment 217. The oligomeric agent of any of embodiments 102, 117-124, 132-137, or 208, wherein N⁵ is a modified cytosine, N² is absent, and N³ is a hypoxanthine.

Embodiment 218. The oligomeric agent of any of embodiments 102, 117-124, 132-137, or 208, wherein N⁵ is a modified cytosine, N² is absent, and N³ is an abasic sugar moiety.

Embodiment 219. The oligomeric agent of any of embodiments 102, 117-124, 132-137, or 208, wherein N⁵ is a modified cytosine, N² is absent, and N³ is a terminal group.

Embodiment 220. The oligomeric agent of any of embodiments 102, 117-124, 132-137, or 208, wherein N⁵ is a modified cytosine, N² is absent, and N³ is absent.

Embodiment 221. The oligomeric agent of any of embodiments 102, 117-124, 132-137, or 208, wherein N⁵ is an abasic sugar moiety, a terminal group, or is absent, N² is absent, and N³ is an unmodified adenine.

Embodiment 22. The oligomeric agent of any of embodiments 102, 117-124, 132-137, or 208, wherein N⁵ is an abasic sugar moiety, a terminal group, or is absent, N² is absent, and N³ is a modified adenine.

Embodiment 223. The oligomeric agent of any of embodiments 102, 117-124, 132-137, or 208, wherein N⁵ is an abasic sugar moiety, a terminal group, or is absent, N² is absent, and N³ is a hypoxanthine.

Embodiment 224. The oligomeric agent of any of embodiments 102, 117-124, 132-137, or 208, wherein N⁵ is an abasic sugar moiety, a terminal group, or is absent, N² is absent, and N³ is an abasic sugar moiety.

Embodiment 225. The oligomeric agent of any of embodiments 102, 117-124, 132-137, or 208, wherein N⁵ is an abasic sugar moiety, a terminal group, or is absent, N² is absent, and N³ is a terminal group.

Embodiment 226. The oligomeric agent of any of embodiments 102, 117-124, 132-137, or 208, wherein N⁵ is an abasic sugar moiety, a terminal group, or is absent, N² is absent, and N³ is absent.

Embodiment 227. The oligomeric agent of any of embodiments 102, 117, or 124-137, wherein:
a) N⁵ is a cytosine nucleobase, a modified cytosine, an abasic sugar moiety, a terminal group, or is absent;
b) N² is absent;
c) N³ is absent; and
d) N⁴ is an unmodified adenine, a modified adenine, a hypoxanthine, an abasic sugar moiety, a terminal group, or is absent.

Embodiment 228. The oligomeric agent of any of embodiments 102, 117, 124-137, or 227, wherein N⁵ is a cytosine nucleobase, N² is absent, N³ is absent, and N⁴ is an unmodified adenine.

Embodiment 229. The oligomeric agent of any of embodiments 102, 117, 124-137, or 227, wherein N⁵ is a cytosine nucleobase, N² is absent, N³ is absent, and N⁴ is a modified adenine.

Embodiment 230. The oligomeric agent of any of embodiments 102, 117, 124-137, or 227, wherein N⁵ is a cytosine nucleobase, N² is absent, N³ is absent, and N⁴ is a hypoxanthine.

Embodiment 231. The oligomeric agent of any of embodiments 102, 117, 124-137, or 227, wherein N⁵ is a cytosine nucleobase, N² is absent, N³ is absent, and N⁴ is an abasic sugar moiety.

Embodiment 232. The oligomeric agent of any of embodiments 102, 117, 124-137, or 227, wherein N⁵ is a cytosine nucleobase, N² is absent, N³ is absent, and N⁴ is a terminal group.

Embodiment 233. The oligomeric agent of any of embodiments 102, 117, 124-137, or 227, wherein N⁵ is a cytosine nucleobase, N² is absent, N³ is absent, and N⁴ is absent.

Embodiment 234. The oligomeric agent of any of embodiments 102, 117, 124-137, or 227, wherein N⁵ is a modified cytosine, N² is absent, N³ is absent, and N⁴ is an unmodified adenine.

Embodiment 235. The oligomeric agent of any of embodiments 102, 117, 124-137, or 227, wherein N⁵ is a modified cytosine, N² is absent, N³ is absent, and N⁴ is a modified adenine.

Embodiment 236. The oligomeric agent of any of embodiments 102, 117, 124-137, or 227, wherein N⁵ is a modified cytosine, N² is absent, N³ is absent, and N⁴ is a hypoxanthine.

Embodiment 237. The oligomeric agent of any of embodiments 102, 117, 124-137, or 227, wherein N⁵ is a modified cytosine, N² is absent, N³ is absent, and N⁴ is an abasic sugar moiety.

Embodiment 238. The oligomeric agent of any of embodiments 102, 117, 124-137, or 227, wherein N⁵ is a modified cytosine, N² is absent, N³ is absent, and N⁴ is a terminal group.

Embodiment 239. The oligomeric agent of any of embodiments 102, 117, 124-137, or 227, wherein N⁵ is a modified cytosine, N² is absent, N³ is absent, and N⁴ is absent.

Embodiment 240. The oligomeric agent of any of embodiments 102, 117, 124-137, or 227, wherein N⁵ is an abasic sugar moiety, a terminal group, or is absent, N² is absent, N³ is absent, and N⁴ is an unmodified adenine.

Embodiment 241. The oligomeric agent of any of embodiments 102, 117, 124-137, or 227, wherein N⁵ is an abasic sugar moiety, a terminal group, or is absent, N² is absent, N³ is absent, and N⁴ is a modified adenine.

Embodiment 242. The oligomeric agent of any of embodiments 102, 117, 124-137, or 227, wherein N⁵ is an abasic sugar moiety, a terminal group, or is absent, N² is absent, N³ is absent, and N⁴ is a hypoxanthine.

Embodiment 243. The oligomeric agent of any of embodiments 102, 117, 124-137, or 227, wherein N⁵ is an abasic sugar moiety, a terminal group, or is absent, N² is absent, N³ is absent, and N⁴ is an abasic sugar moiety.

Embodiment 244. The oligomeric agent of any of embodiments 102, 117, 124-137, or 227, wherein N⁵ is an abasic sugar moiety, a terminal group, or is absent, N² is absent, N³ is absent, and N⁴ is a terminal group.

Embodiment 245. The oligomeric agent of any of embodiments 102, 117, 124-137, or 227, wherein N⁵ is an abasic sugar moiety, a terminal group, or is absent, N² is absent, N³ is absent, and N⁴ is absent.

Embodiment 246. An oligomeric agent comprising a modified oligonucleotide according to the following chemical structure:

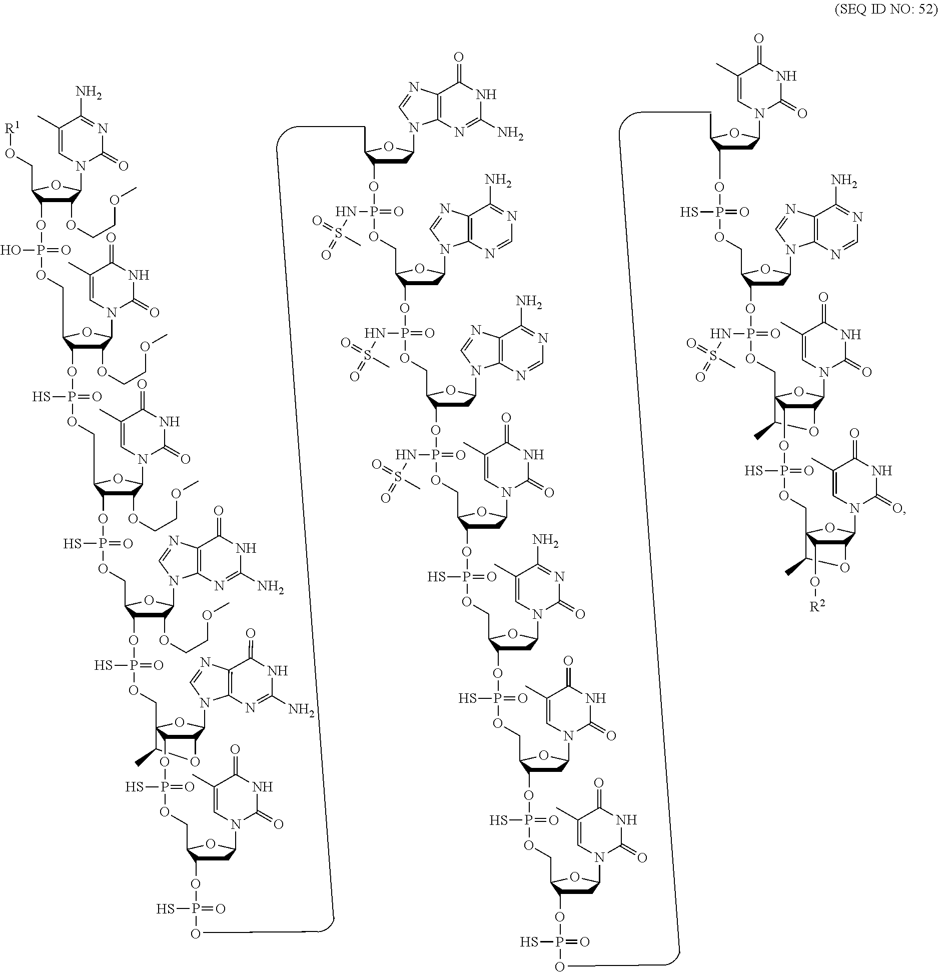

or a pharmaceutically acceptable salt thereof;
wherein R¹ and R² are each independently 1, 2, or 3 linked nucleosides, an abasic nucleoside, a terminal group, or an H; and
wherein the oligomeric agent optionally comprises a conjugate group.

Embodiment 247. An oligomeric agent comprising a modified oligonucleotide according to the following chemical structure:

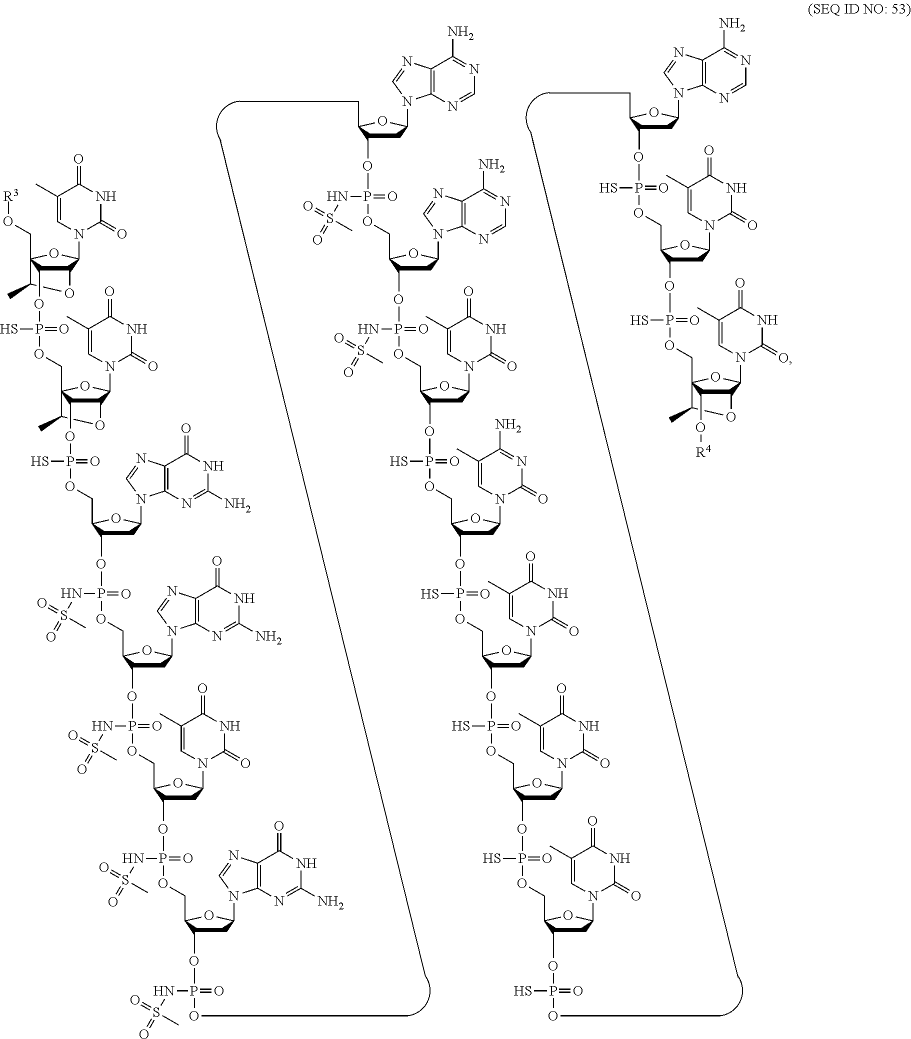

or a pharmaceutically acceptable salt thereof;
wherein R₃ and R⁴ are each independently 1, 2, or 3 linked nucleosides, an abasic nucleoside, a terminal group, or an H; and
wherein the oligomeric agent optionally comprises a conjugate group.

Embodiment 248. The oligomeric agent of embodiment 246, wherein R¹ is one nucleoside.

Embodiment 249. The oligomeric agent of embodiment 246, wherein R¹ is 2 linked nucleosides.

Embodiment 250. The oligomeric agent of embodiment 246, wherein R¹ is 3 linked nucleosides.

Embodiment 251. The oligomeric agent of any of embodiments 246 or 248-250, wherein R² is one nucleoside.

Embodiment 252. The oligomeric agent of any of embodiments 246 or 248-250, wherein R² is 2 linked nucleosides.

Embodiment 253. The oligomeric agent of any of embodiments 246 or 248-250, wherein R² is 3 linked nucleosides.

Embodiment 254. The oligomeric agent of any of embodiments 246, 248, or 252, wherein R¹ is one nucleoside and R² is 2 linked nucleosides.

Embodiment 255. The oligomeric agent of any of embodiments 246, 248, or 253, wherein R¹ is one nucleoside and R² is 3 linked nucleosides.

Embodiment 256. The oligomeric agent of any of embodiments 246, 248, or 252-255, wherein R¹ is an adenosine, a modified adenosine, an unmodified adenosine, or an inosine; and R² is 2 or 3 linked nucleosides.

Embodiment 257. The oligomeric agent of any of embodiments 246, 248, or 252-256, wherein R¹ is an adenosine and R² comprises an adenosine, a modified adenosine, an unmodified adenosine, or an inosine.

Embodiment 258. The oligomeric agent of any of embodiments 246, 248, or 252-256, wherein R¹ is an adenosine and R² comprises two or more adenosines, two or more modified adenosines, two or more unmodified adenosines, or two or more inosines.

Embodiment 259. The oligomeric agent of any of embodiments 246, 248, or 252-256, wherein R¹ is an adenosine and R² is an abasic nucleoside, a terminal group, or an H.

Embodiment 260. The oligomeric agent of any of embodiments 246, 248, or 252-256, wherein R¹ is a modified adenosine and R² comprises an adenosine, a modified adenosine, an unmodified adenosine, or an inosine.

Embodiment 261. The oligomeric agent of any of embodiments 246, 248, or 252-256, wherein R¹ is a modified adenosine and R² comprises two or more adenosines, two or more modified adenosines, two or more unmodified adenosines, or two or more inosines.

Embodiment 262. The oligomeric agent of any of embodiments 246, 248, or 252-256, wherein R¹ is a modified adenosine and R² is an abasic nucleoside, a terminal group, or an H.

Embodiment 263. The oligomeric agent of any of embodiments 246, 248, or 252-256, wherein R¹ is an unmodified adenosine and R² comprises an adenosine, a modified adenosine, an unmodified adenosine, or an inosine.

Embodiment 264. The oligomeric agent of any of embodiments 246, 248, or 252-256, wherein R¹ is an unmodified adenosine and R² comprises two or more adenosines, two or more modified adenosines, two or more unmodified adenosines, or two or more inosines.

Embodiment 265. The oligomeric agent of any of embodiments 246, 248, or 252-256, wherein R¹ is an unmodified adenosine and R² is an abasic nucleoside, a terminal group, or an H.

Embodiment 266. The oligomeric agent of any of embodiments 246, 248, or 252-256, wherein R¹ is an inosine and R comprises an adenosine, a modified adenosine, an unmodified adenosine, or an inosine.

Embodiment 267. The oligomeric agent of any of embodiments 246, 248, or 252-256, wherein R¹ is an inosine and R comprises two or more adenosines, two or more modified adenosines, two or more unmodified adenosines, or two or more inosines.

Embodiment 268. The oligomeric agent of any of embodiments 246, 248, or 252-256, wherein R¹ is an inosine and R² is an abasic nucleoside, a terminal group, or an H.

Embodiment 269. The oligomeric agent of any of embodiments 246, 248, or 252-255, wherein R¹ is an abasic nucleoside, a terminal group, or an H; and R² comprises an adenosine, a modified adenosine, an unmodified adenosine, or an inosine.

Embodiment 270. The oligomeric agent of any of embodiments 246, 248, or 252-255, wherein R¹ is an abasic nucleoside, a terminal group, or an H; and R² comprises two or more adenosines, two or more modified adenosines, two or more unmodified adenosines, or two or more inosines.

Embodiment 271. The oligomeric agent of any of embodiments 246, 248, or 252-255, wherein R¹ is an abasic nucleoside, a terminal group, or an H; and R² is an abasic nucleoside, a terminal group, or an H.

Embodiment 272. The oligomeric agent of any of embodiments 246 or 249-253, wherein R¹ is 2 or 3 linked nucleosides, optionally comprising an adenosine, a modified adenosine, an unmodified adenosine, or an inosine; and R² comprises an adenosine, a modified adenosine, an unmodified adenosine, or an inosine.

Embodiment 273. The oligomeric agent of any of embodiments 246 or 249-253, wherein R¹ is 2 or 3 linked nucleosides, optionally comprising an adenosine, a modified adenosine, an unmodified adenosine, or an inosine; and R² is an abasic nucleoside, a terminal group, or an H.

Embodiment 274. The oligomeric agent of embodiment 247, wherein R³ is one nucleoside.

Embodiment 275. The oligomeric agent of embodiment 247, wherein R³ is 2 linked nucleosides.

Embodiment 276. The oligomeric agent of embodiment 247, wherein R³ is 3 linked nucleosides.

Embodiment 277. The oligomeric agent of any of embodiments 247 or 274-276, wherein R⁴ is one nucleoside.

Embodiment 278. The oligomeric agent of any of embodiments 247 or 274-276, wherein R⁴ is 2 linked nucleosides.

Embodiment 279. The oligomeric agent of any of embodiments 247 or 274-276, wherein R⁴ is 3 linked nucleosides.

Embodiment 280. The oligomeric agent of any of embodiments 247, 274, or 278-279, wherein R³ is one nucleoside and R⁴ is 2 or 3 linked nucleosides.

Embodiment 281. The oligomeric agent of any of embodiments 247, 274, or 278-280, wherein R³ is a cytidine, a modified cytidine, or an unmodified cytidine, and R⁴ comprises an adenosine, a modified adenosine, an unmodified adenosine, or an inosine.

Embodiment 282. The oligomeric agent of any of embodiments 247, 274, or 278-281, wherein R³ is a cytidine and R⁴ comprises an adenosine, a modified adenosine, an unmodified adenosine, or an inosine.

Embodiment 283. The oligomeric agent of any of embodiments 247, 274, or 278-281, wherein R³ is a cytidine and R⁴ comprises two or more adenosines, two or more modified adenosines, two or more unmodified adenosines, or two or more inosines.

Embodiment 284. The oligomeric agent of any of embodiments 247, 274, or 278-281, wherein R³ is a cytidine and R⁴ is an abasic nucleoside, a terminal group, or an H.

Embodiment 285. The oligomeric agent of any of embodiments 247, 274, or 278-281, wherein R³ is a modified cytidine and R⁴ comprises an adenosine, a modified adenosine, an unmodified adenosine, or an inosine.

Embodiment 286. The oligomeric agent of any of embodiments 247, 274, or 278-281, wherein R³ is a modified cytidine and R⁴ comprises two or more adenosines, two or more modified adenosines, two or more unmodified adenosines, or two or more inosines.

Embodiment 287. The oligomeric agent of any of embodiments 247, 274, or 278-281, wherein R³ is a modified cytidine and R⁴ is an abasic nucleoside, a terminal group, or an H.

Embodiment 288. The oligomeric agent of any of embodiments 247, 274, or 278-281, wherein R³ is a 5-methylcytidine and R⁴ comprises an adenosine, a modified adenosine, an unmodified adenosine, or an inosine.

Embodiment 289. The oligomeric agent of any of embodiments 247, 274, or 278-281, wherein R³ is a 5-methylcytidine and R⁴ comprises two or more adenosines, two or more modified adenosines, two or more unmodified adenosines, or two or more inosines.

Embodiment 290. The oligomeric agent of any of embodiments 247, 274, or 278-281, wherein R³ is a 5-methylcytidine and R⁴ is an abasic nucleoside, a terminal group, or an H.

Embodiment 291. The oligomeric agent of any of embodiments 247, 274, or 278-281, wherein R³ is an unmodified cytidine and R⁴ comprises an adenosine, a modified adenosine, an unmodified adenosine, or an inosine.

Embodiment 292. The oligomeric agent of any of embodiments 247, 274, or 278-281, wherein R³ is an unmodified cytidine and R⁴ comprises two or more adenosines, two or more modified adenosines, two or more unmodified adenosines, or two or more inosines.

Embodiment 293. The oligomeric agent of any of embodiments 247, 274, or 278-281, wherein R³ is an unmodified cytidine and R⁴ is an abasic nucleoside, a terminal group, or an H.

Embodiment 294. The oligomeric agent of any of embodiments 247 or 275-279, wherein R³ is 2 or 3 linked nucleosides, optionally comprises a cytidine, a modified cytidine, or an unmodified cytidine, and R⁴ comprises an adenosine, a modified adenosine, an unmodified adenosine, or an inosine.

Embodiment 295. The oligomeric agent of any of embodiments 247 or 275-279, wherein R³ is 2 or 3 linked nucleosides, optionally comprises a cytidine, a modified cytidine, or an unmodified cytidine, and R⁴ is an abasic nucleoside, a terminal group, or an H.

Embodiment 296. The oligomeric agent of any of embodiments 246-295, wherein if R¹, R², R³, and R⁴ are each independently 1, 2, or 3 linked nucleosides or are each independently an abasic nucleoside, R¹, R², R³, and R⁴ also include an internucleoside linkage selected from a phosphodiester internucleoside linkage, a phosphorothioate internucleoside linkage, and a mesyl phosphoramidate internucleoside linkage.

Embodiment 297. The oligomeric agent of any of embodiments 246-296, wherein the modified oligonucleotide is a pharmaceutically acceptable salt comprising one or more cations selected from sodium, potassium, calcium, and magnesium.

Embodiment 298. A modified oligonucleotide according to the following chemical structure:

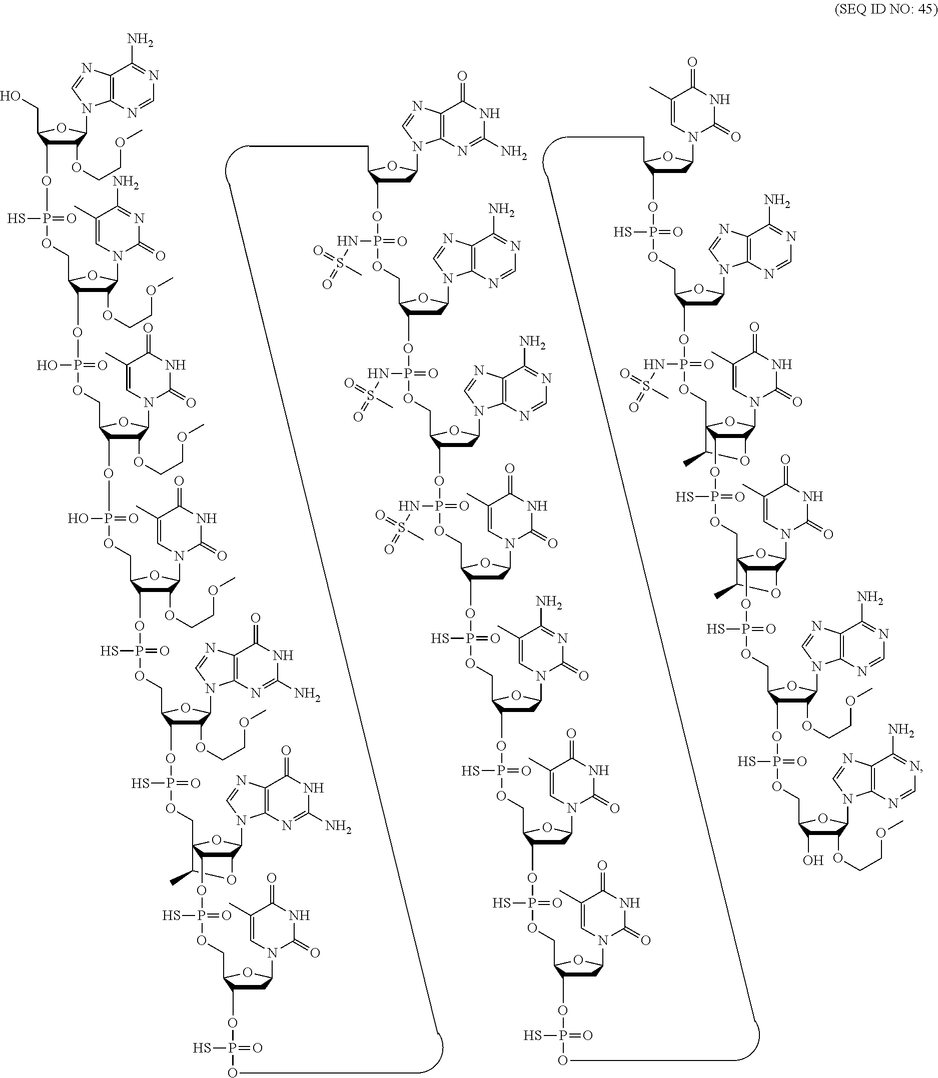

or a pharmaceutically acceptable salt thereof.

Embodiment 299. A modified oligonucleotide according to the following chemical structure:

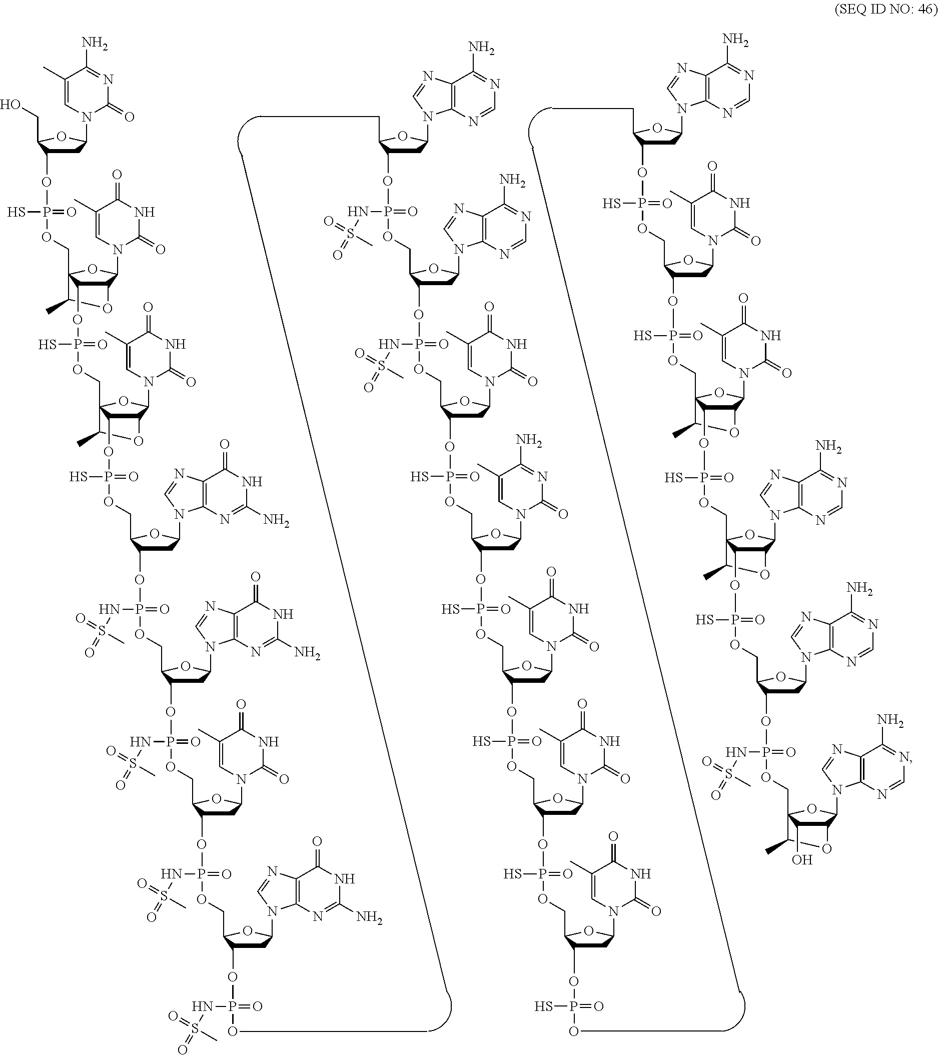

or a pharmaceutically acceptable salt thereof.

Embodiment 300. A modified oligonucleotide according to the following chemical structure:

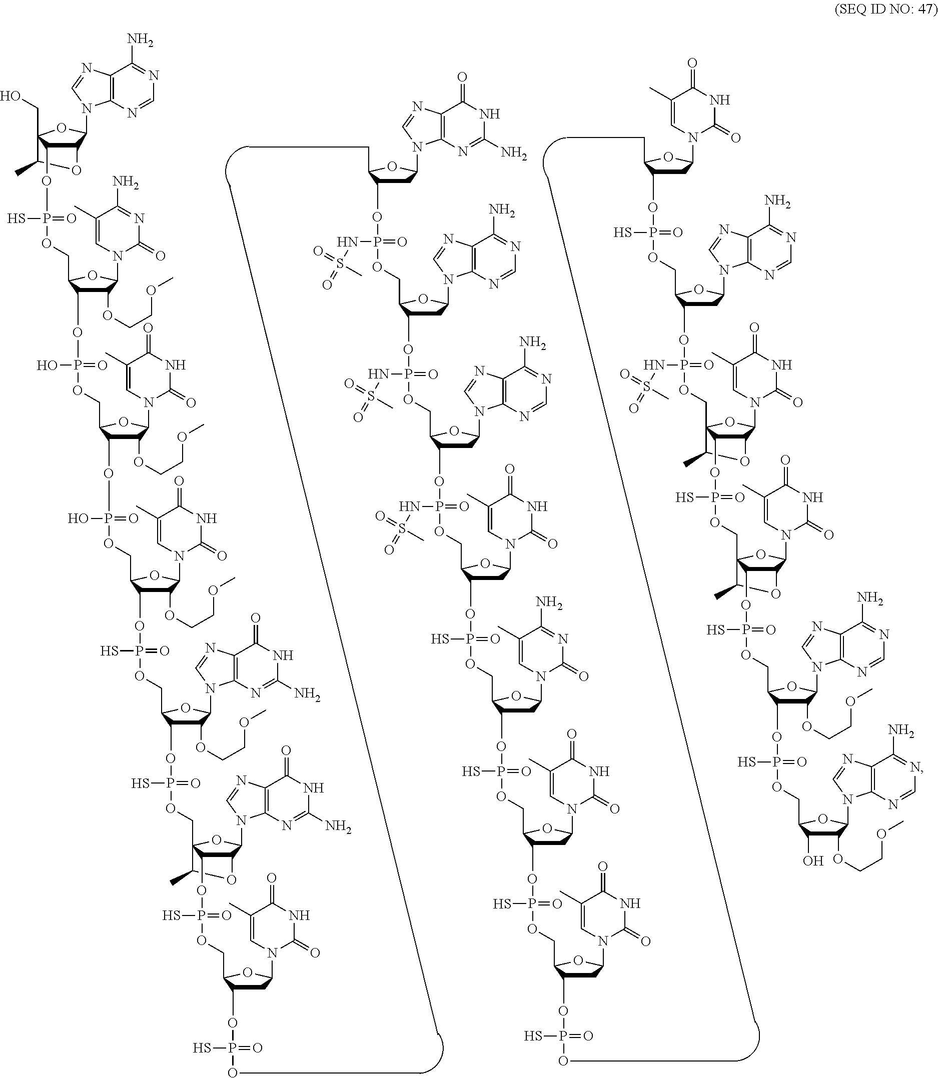

or a pharmaceutically acceptable salt thereof.

Embodiment 301. A modified oligonucleotide according to the following chemical structure:

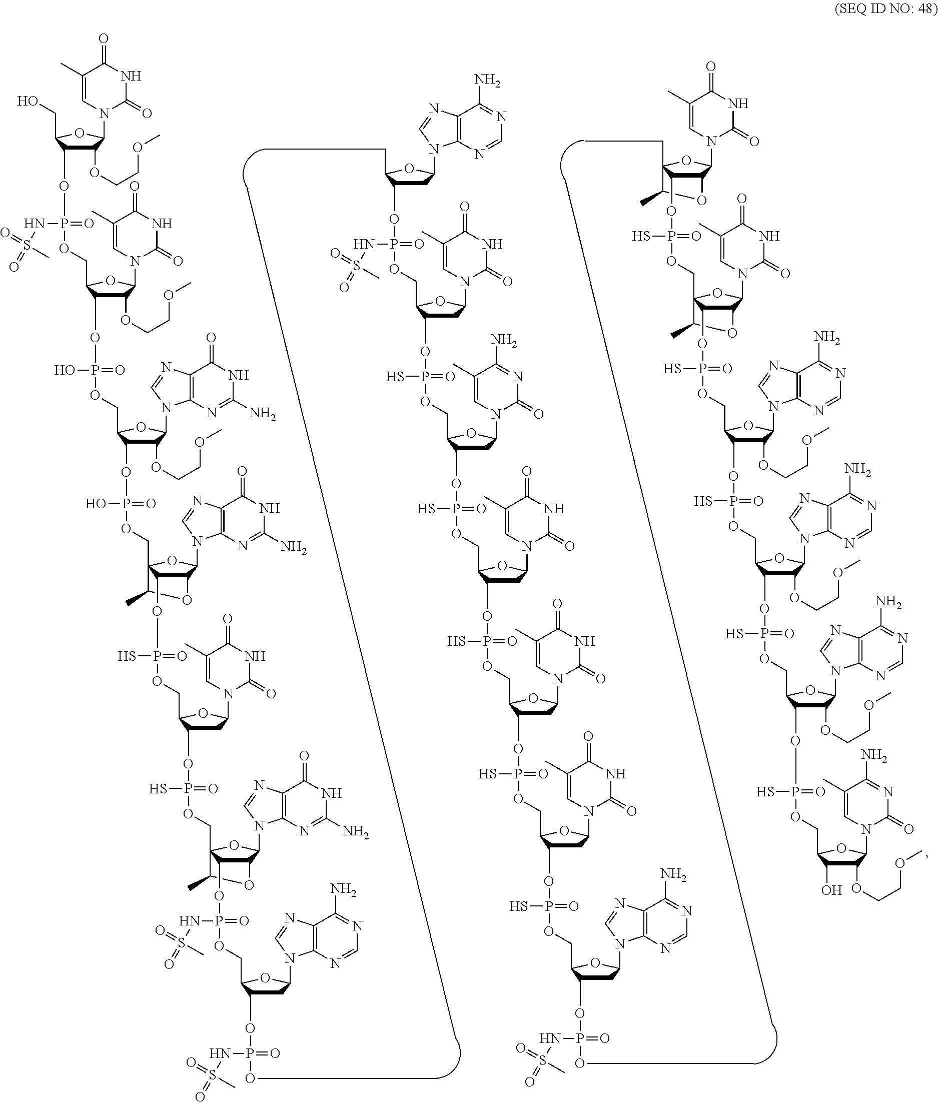

or a pharmaceutically acceptable salt thereof.

Embodiment 302. The modified oligonucleotide of any one of embodiments 298-301, wherein the modified oligonucleotide is a pharmaceutically acceptable salt comprising one or more cations selected from sodium, potassium, calcium, and magnesium.

Embodiment 303. A modified oligonucleotide according to the following chemical structure:

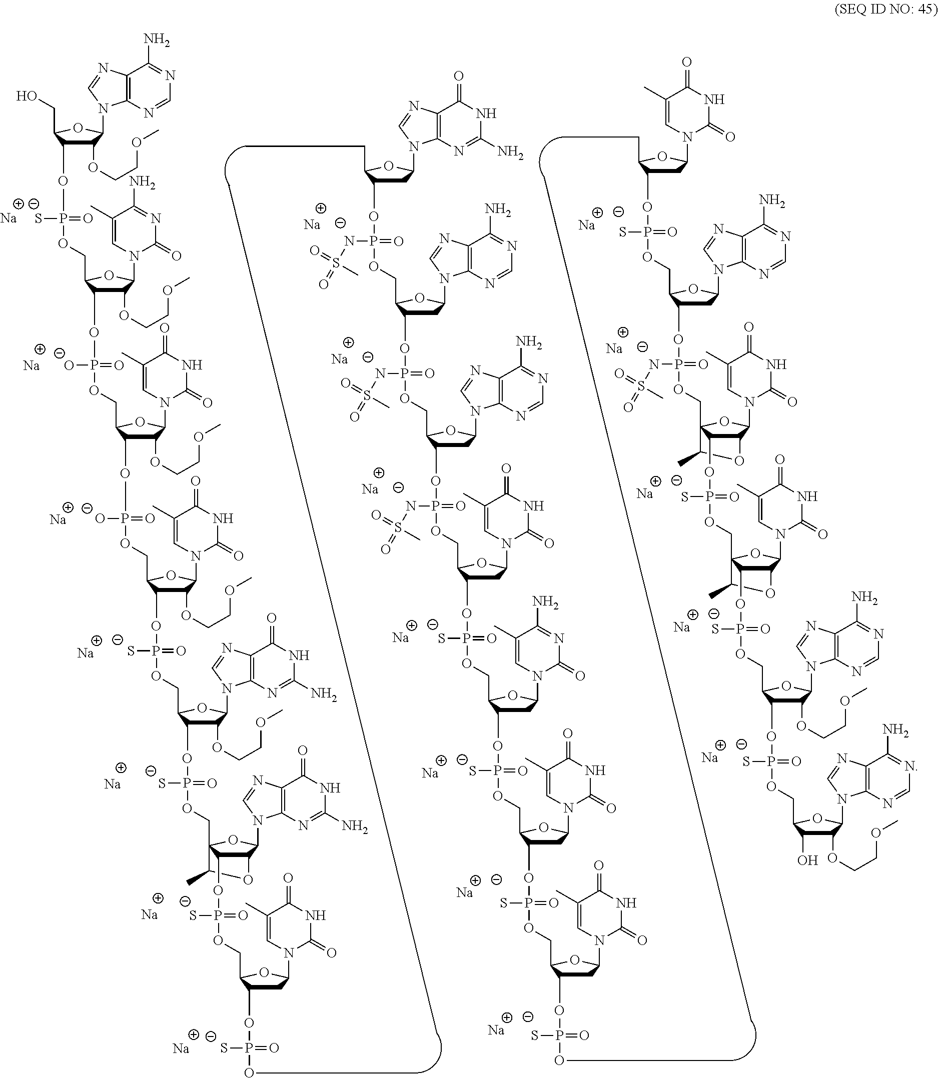

Embodiment 304. A modified oligonucleotide according to the following chemical structure:

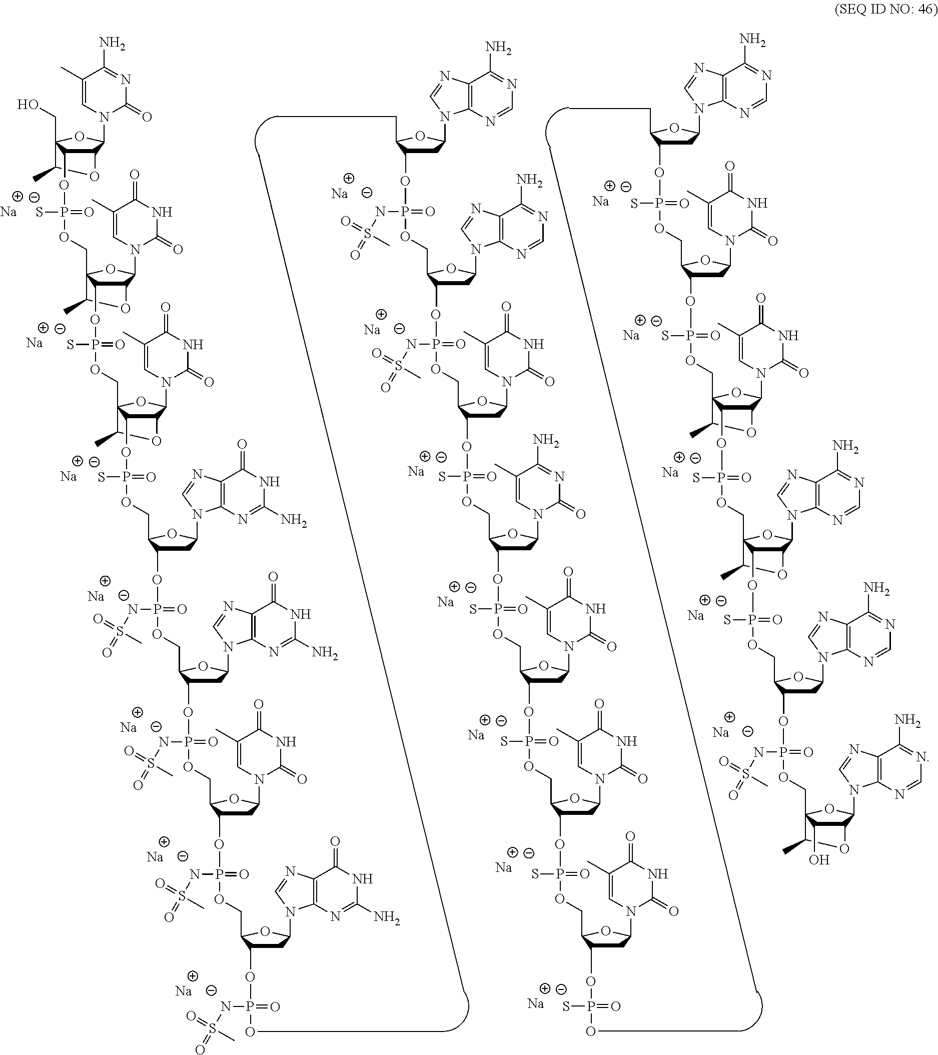

Embodiment 305. A modified oligonucleotide according to the following chemical structure:

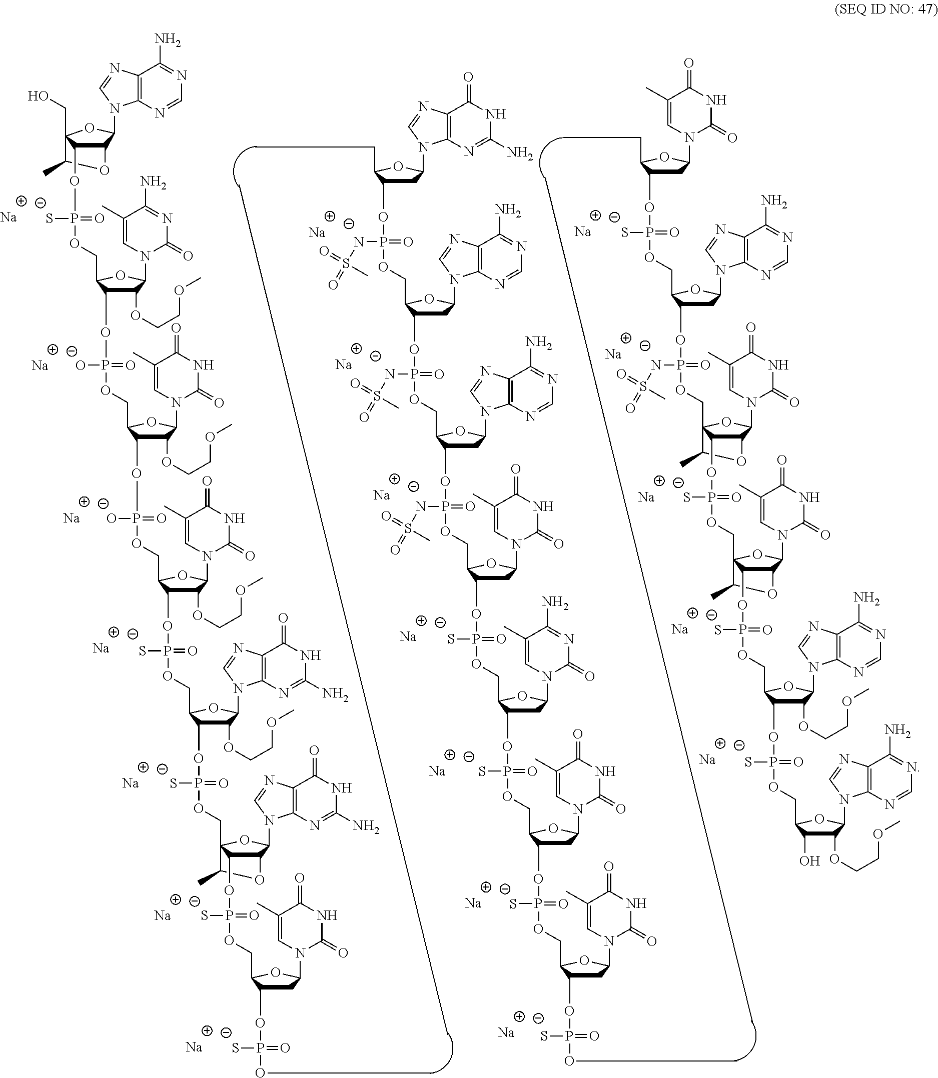

Embodiment 306. A modified oligonucleotide according to the following chemical structure:

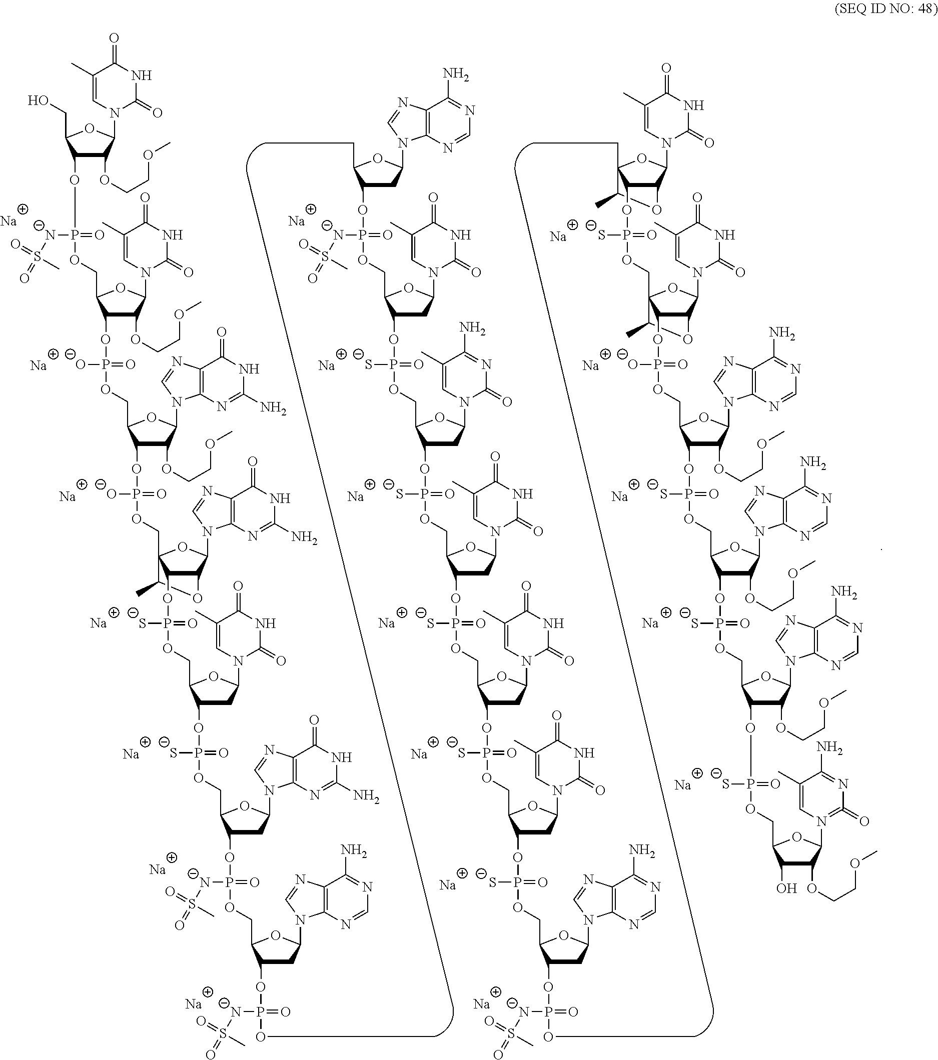

Embodiment 307. A population of oligomeric agents of any of embodiments 1-297 or a population of modified oligonucleotides of any of embodiments 298-306, wherein the population is chirally enriched for modified oligonucleotides comprising at least one particular phosphorothioate internucleoside linkage having a particular stereochemical configuration.

Embodiment 308. The population of embodiment 307, wherein the population is chirally enriched for modified oligonucleotides comprising at least one particular phosphorothioate internucleoside linkage having the (Sp) configuration.

Embodiment 309. The population of embodiment 307, wherein the population is chirally enriched for modified oligonucleotides comprising at least one particular phosphorothioate internucleoside linkage having the (Rp) configuration.

Embodiment 310. The population of embodiment 307, wherein the population is chirally enriched for modified oligonucleotides having a particular, independently selected stereochemical configuration at each phosphorothioate internucleoside linkage.

Embodiment 311. The population of embodiment 307, wherein the population is chirally enriched for modified oligonucleotides having the (Sp) configuration at each phosphorothioate internucleoside linkage or for modified oligonucleotides having the (Rp) configuration at each phosphorothioate internucleoside linkage.

Embodiment 312. The population of embodiment 307, wherein the population is chirally enriched for modified oligonucleotides having the (Rp) configuration at one particular phosphorothioate internucleoside linkage and the (Sp) configuration at each of the remaining phosphorothioate internucleoside linkages.

Embodiment 313. The population of embodiment 307, wherein the population is chirally enriched for modified oligonucleotides having at least 3 contiguous phosphorothioate internucleoside linkages in the Sp, Sp, and Rp configurations, in the 5′ to 3′ direction.

Embodiment 314. A population of oligomeric agents of any of embodiments 1-297 or a population of modified oligonucleotides of any of embodiments 298-306, wherein all of the phosphorothioate internucleoside linkages of the modified oligonucleotide are stereorandom.

Embodiment 315. A population of oligomeric agents of any of embodiments 1-297 or a population of modified oligonucleotides of any of embodiments 298-306, wherein all of the mesyl phosphoramidate internucleoside linkages of the modified oligonucleotide are stereorandom.

Embodiment 316. A pharmaceutical composition comprising an oligomeric agent of any of embodiments 1-297, a modified oligonucleotide of any of embodiments 298-306, or a population of any of embodiments 307-315, and a pharmaceutically acceptable diluent.

Embodiment 317. The pharmaceutical composition of embodiment 316, wherein the pharmaceutically acceptable diluent is artificial cerebrospinal fluid (aCSF) or phosphate-buffered saline (PBS).

Embodiment 318. The pharmaceutical composition of embodiment 317, wherein the pharmaceutical composition consists essentially of the oligomeric agent of any of embodiments 1-297, the modified oligonucleotide of any of embodiments 298-306, or the population of any of embodiments 307-315, and aCSF.

Embodiment 319. The pharmaceutical composition of embodiment 317, wherein the pharmaceutical composition consists essentially of the oligomeric agent of any of embodiments 1-297, the modified oligonucleotide of any of embodiments 298-306, or the population of any of embodiments 307-315, and PBS.

Embodiment 320. A method comprising administering to a subject an oligomeric agent of any of embodiments 1-297, a modified oligonucleotide of any of embodiments 298-306, a population of any of embodiments 307-315, or a pharmaceutical composition of any of embodiments 316-319.

Embodiment 321. The method of embodiment 320, wherein the subject has or is at risk of developing Alzheimer's disease.

Embodiment 322. A method of treating a neurodegenerative disease associated with APOE comprising administering to a subject having or at risk of developing a neurodegenerative disease associated with APOE a therapeutically effective amount of an oligomeric agent of any of embodiments 1-297, a modified oligonucleotide of any of embodiments 298-306, a population of any of embodiments 307-315, or a pharmaceutical composition of any of embodiments 316-319.

Embodiment 323. The method of embodiment 322, wherein the neurodegenerative disease associated with APOE is Alzheimer's disease.

Embodiment 324. The method of embodiment 322, wherein at least one symptom of the neurodegenerative disease associated with APOE is ameliorated.

Embodiment 325. The method of embodiment 324, wherein the symptom is cognitive impairment, progressive memory loss, a decline in language skills, behavioral abnormality, dementia, difficulty performing daily activities, aphasia, agnosia, apraxia, loss of motor function, amyloid plaque, neurofibrillary tangle, and/or neuroinflammation.

Embodiment 326. The method of any of embodiments 322-325, wherein administering the oligomeric agent of any of embodiments 1-297, the modified oligonucleotides of any of embodiments 298-306, the population of any of embodiments 307-315, or the pharmaceutical composition of any of embodiments 316-319 reduces the rate of cognitive impairment or progressive memory loss, reduces the rate of decline in language skills, reduces the rate of progression of behavioral abnormality, reduces the rate of progression of dementia, improves the performance in daily activities, reduces the rate of progression of aphasia, agnosia, and/or apraxia, and/or decreases the rate of decline in motor function in the subject.

Embodiment 327. The method of any of embodiments 322-326, wherein administering the oligomeric agent of any of embodiments 1-297, the modified oligonucleotides of any of embodiments 298-306, the population of any of embodiments 307-315, or the pharmaceutical composition of any of embodiments 316-319 reduces amyloid plaques, neurofibrillary tangles, or neuroinflammation in the brain of the subject.

Embodiment 328. The method of any of embodiments 320-327, wherein the oligomeric agent of any of embodiments 1-297, the modified oligonucleotides of any of embodiments 298-306, the population of any of embodiments 307-315, or the pharmaceutical composition of any of embodiments 316-319 is administered to the central nervous system or systemically.

Embodiment 329. The method of any of embodiments 320-328, wherein the oligomeric agent of any of embodiments 1-297, the modified oligonucleotides of any of embodiments 298-306, the population of any of embodiments 307-315, or the pharmaceutical composition of any of embodiments 316-319 is administered intrathecally.

Embodiment 330. The method of any of embodiments 320-329, wherein the subject is a human.

Embodiment 331. A method of reducing the amount of APOE RNA in a cell comprising contacting the cell with an oligomeric agent of any of embodiments 1-297, a modified oligonucleotide of any of embodiments 298-306, a population of any of embodiments 307-315, or a pharmaceutical composition of any of embodiments 316-319.

Embodiment 332. The method of embodiment 331, wherein the cell is a brain cell.

Embodiment 333. The method of embodiment 331 or embodiment 332, wherein the cell is a glial cell or a neuron.

Embodiment 334. The method of any of embodiments 331-333, wherein the cell is an astrocyte or a microglial cell.

Embodiment 335. The method of any of embodiments 331-334, wherein the cell is a human cell.

Embodiment 336. Use of an oligomeric agent of any of embodiments 1-297, a modified oligonucleotide of any of embodiments 298-306, a population of any of embodiments 307-315, or a pharmaceutical composition of any of embodiments 316-319 for treating a neurodegenerative disease associated with APOE.

Embodiment 337. Use of an oligomeric agent of any of embodiments 1-297, a modified oligonucleotide of any of embodiments 298-306, a population of any of embodiments 307-315, or a pharmaceutical composition of any of embodiments 316-319 in the manufacture of a medicament for treating a neurogenerative disease.

Embodiment 338. Use of an oligomeric agent of any of embodiments 1-297, a modified oligonucleotide of any of embodiments 298-306, a population of any of embodiments 307-315, or a pharmaceutical composition of any of embodiments 316-319 in the manufacture of a medicament for treating a neurogenerative disease associated with APOE.

Embodiment 339. The use of any of embodiments 336-338, wherein the neurodegenerative disease associated with APOE is Alzheimer's disease.

Embodiment 340. An oligomeric agent of any of embodiments 1-297, a modified oligonucleotide of any of embodiments 298-306, or a population of any of embodiments 307-315, for use as a therapeutically active substance.

Embodiment 341. An oligomeric agent of any of embodiments 1-297, a modified oligonucleotide of any of embodiments 298-306, a population of any of embodiments 307-315, or a pharmaceutical composition of any of embodiments 316-319, for use in the treatment of a neurodegenerative disease.

Embodiment 342. An oligomeric agent of any of embodiments 1-297, a modified oligonucleotide of any of embodiments 298-306, a population of any of embodiments 307-315, or a pharmaceutical composition of any of embodiments 316-319, for use in the treatment of a neurodegenerative disease associated with APOE.

Embodiment 343. An oligomeric agent of any of embodiments 1-297, a modified oligonucleotide of any of embodiments 298-306, a population of any of embodiments 307-315, or a pharmaceutical composition of any of embodiments 316-319, for use in the treatment of Alzheimer's disease.

### I. Oligomeric Agents

Provided herein are oligomeric agents comprising or consisting of at least one modified oligonucleotide and optionally one or more additional associated features selected from: (a) one or more additional modified or unmodified oligonucleotides, each of which may be hybridized to or covalently linked to the at least one modified oligonucleotide and/or to each other; (b) one or more conjugate groups, which may be covalently attached to any oligonucleotide of such oligomeric agent; and (c) one or more terminal groups. In some embodiments, provided herein are oligomeric agents comprising or consisting of a modified antisense oligonucleotide complementary to APOE RNA. In certain such embodiments, an oligomeric agent consists of a modified antisense oligonucleotide complementary to APOE RNA and a conjugate group attached to the modified antisense oligonucleotide. In some embodiments, provided herein are oligomeric agents comprising an oligomeric duplex comprising or consisting of an antisense oligonucleotide complementary to APOE RNA, and a sense oligonucleotide complementary to the antisense oligonucleotide, wherein one or both of the antisense and sense oligonucleotides is/are modified. In certain such embodiments, an oligomeric agent consists of an antisense oligonucleotide complementary to APOE RNA, a sense oligonucleotide complementary to the antisense oligonucleotide, and a conjugate group and/or terminal group attached to one or both of the antisense and sense oligonucleotides. Modified antisense and/or sense oligonucleotides comprise at least one modified nucleoside (comprising a modified sugar moiety and/or a modified nucleobase and/or lacking a nucleobase) and/or at least one modified internucleoside linkage. Examples of certain modified nucleosides and modified internucleoside linkages suitable for use in modified antisense and/or sense oligonucleotides are described herein.

### A. Modified Nucleosides

Modified nucleosides comprise a modified sugar moiety, a modified nucleobase, or a combination thereof. In certain embodiments, modified nucleosides comprising the following modified sugar moieties and/or the following modified nucleobases may be incorporated into oligonucleotides, e.g., into modified antisense and/or sense oligonucleotides described herein.

### 1. Modified Sugar Moieties

Modified sugar moieties include modified furanosyl sugar moieties, cyclic sugar surrogates, acyclic sugar surrogates, and sugar mimics. In certain embodiments, modified sugar moieties are non-bicyclic modified furanosyl sugar moieties. In certain embodiments, modified sugar moieties are bicyclic or tricyclic furanosyl sugar moieties. In certain embodiments, modified sugar moieties are sugar surrogates. Sugar surrogates may comprise one or more substitutions corresponding to those of other types of modified sugar moieties.

In certain embodiments, modified sugar moieties are non-bicyclic modified furanosyl sugar moieties comprising one or more substituent groups including, but not limited to, substituents at the 2′, 3′, 4′, and/or 5′ positions, as numbered below:

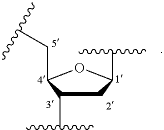

In certain embodiments, the modified furanosyl sugar moiety is a ribosyl sugar moiety that is not an unmodified sugar moiety (i.e., an unmodified RNA or unmodified DNA moiety). In certain embodiments, the modified furanosyl sugar moiety is a xylosyl, lyxosyl, or arabinosyl sugar moiety.

In certain embodiments, non-bicyclic modified sugar moieties are 2′-substituted sugar moieties and comprise a substituent group at the 2′-position. Examples of substituent groups suitable for the 2′-position of modified sugar moieties include but are not limited to: F, OCH₃ (“OMe” or “O-methyl”), and O(CH₂)₂OCH₃ (“MOE” or “O-methoxyethyl” or OCH₂CH₂OCH₃). In certain embodiments, 2′-substituent groups are selected from: halo, allyl, amino, azido, SH, CN, OCN, CF₃, OCF₃, C₁-C₁₀ alkoxy, substituted C₁-C₁₀ alkoxy, C₁-C₁₀ alkyl, substituted C₁-C₁₀ alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, O(CH₂)₂ON(CH₃)₂ (“DMAOE”), or O(CH₂)₂O(CH₂)₂N(CH₃)₂ (“DMAEOE”). Synthetic methods for some of these 2′-substituent groups may be found, e.g., in Cook et al., U.S. Pat. No. 6,531,584; Cook et al., U.S. Pat. No. 5,859,221; and Cook et al., U.S. Pat. No. 6,005,087. Certain embodiments of these 2′-substituent groups may be further substituted with one or more substituent groups independently selected from: halo, cyano, OR^{a2}, NO₂, NH₂, NHR^{a2}, N(R^{a2})₂, C₁-C₆ alkyl, C₁-C₆ haloalkyl, C₂-C₆ alkenyl, C₂-C₆ alkynyl, C₃-C₁₀ cycloalkyl, C₆-C₁₀ aryl, heteroaryl, heterocyclyl, C₁-C₆ alkylene-NH₂, C₁-C₆alkylene-NHR^{a2}, C₁-C₆alkylene-N(R^{a2})₂, C(O)R^{a3}, C(O)OR^{a3}, C(O)NHR^{a3}, C(O)N(C₁-C₄ alkyl)R^{a3}, SR^{a3} S(O)₂R^{a3} S(O)R^{a3}, NHC(O)R^{a3}, N(C₁-C₄ alkyl)C(O)R^{a3}, NHS(O)R^{a3}, N(C₁-C₄alkyl)S(O)R^{a3}, NHS(O)₂R^{a3}, and N(C₁-C₄ alkyl)S(O)₂R^{a3}; where each R² is independently selected from C₂-C₆ alkyl, C₂-C₆ alkenyl, C₂-C₆ alkynyl, C₃-C₁₀ cycloalkyl, C₆-C₁₀ aryl, heteroaryl, and heterocyclyl; and each R³ is independently hydrogen, OH, C₁-C₆ alkyl, C₁-C₆ haloalkyl, C₃-C₁₀ cycloalkyl, C₆-C₁₀ aryl, heteroaryl, or heterocyclyl. In certain embodiments, a sugar moiety comprises two of the above substituents at the 2′-position. In certain embodiments, a sugar moiety comprises a 2′-fluoro and a second 2′-substituent.

In certain embodiments, a 2′-substituted sugar moiety comprises a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl. In certain embodiments, a 2′-substituted sugar moiety comprises a non-bridging 2′-substituent group selected from: F, OCF₃, OCH₃, OCH₂CH₂OCH₃, O(CH₂)₂SCH₃, O(CH₂)₂ON(CH₃)₂ (“DMAOE”), O(CH₂)₂O(CH₂)₂N(CH₃)₂ (“DMAEOE”), and OCH₂C(═O)—N(H)CH₃ (“NMA”). In certain embodiments one or more non-bridging substituent of non-bicyclic modified sugar moieties is branched.

In certain embodiments, a 2′-substituted sugar moiety comprises a 2′-substituent group selected from: F, OCH₃, and O(CH₂)₂OCH₃.

In certain embodiments, modified furanosyl sugar moieties and nucleosides incorporating such modified furanosyl sugar moieties are further defined by stereochemical configuration. For example, a 2′-deoxyfuranosyl sugar moiety (i.e., 2′-(H)H furanosyl sugar moiety) may be in seven isomeric configurations other than the naturally occurring β-D-deoxyribosyl configuration. Such modified sugar moieties are described in, e.g., WO 2020/072991. A 2′-modified sugar moiety has an additional stereocenter at the 2′-position relative to a 2′-deoxyfuranosyl sugar moiety; therefore, such sugar moieties have a total of sixteen possible stereochemical configurations. Modified furanosyl sugar moieties described herein are in the β-D-ribosyl stereochemical configuration unless otherwise specified.

In certain embodiments, non-bicyclic modified sugar moieties comprise a substituent group at the 4′-position. Examples of substituent groups suitable for the 4′-position of modified sugar moieties include, but are not limited to, alkoxy (e.g., methoxy), alkyl, and those described in Manoharan et al., WO 2015/106128.

In certain embodiments, non-bicyclic modified sugar moieties comprise a substituent group at the 3′-position. Examples of substituent groups suitable for the 3′-position of modified sugar moieties include, but are not limited to, alkoxy (e.g., methoxy), alkyl (e.g., methyl, ethyl).

In certain embodiments, non-bicyclic modified sugar moieties comprise a substituent group at the 5′-position. Examples of substituent groups suitable for the 5′-position of modified sugar moieties include, but are not limited to, vinyl, alkoxy (e.g., methoxy), alkynyl, allyl, and alkyl (e.g., methyl (R or S), ethyl (R or S)).

In certain embodiments, non-bicyclic modified sugar moieties comprise more than one non-bridging sugar substituent, for example, 2′-F-5′-methyl sugar moieties, such as described in Migawa et al., US 2010/0190837, or alternative 2′- and 5′-modified sugar moieties as described in Rajeev et al., US 2013/0203836.

Certain modified sugar moieties are bicyclic sugar moieties and comprise a substituent that bridges two atoms of the furanosyl ring to form a second ring. In certain embodiments, the bicyclic sugar moiety comprises a bridge between the 4′ and the 2′ furanose ring atoms. Examples of such 4′ to 2′ bridging sugar substituents include, but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof, 4′-C(CH₃)(CH₃)—O-2′ and analogs thereof, 4′-CH₂—N(OCH₃)-2′ and analogs thereof, 4′-CH₂—O—N(CH₃)-2′, 4′-CH₂—C(H)(CH₃)-2′, 4′-CH₂—C(═CH₂)-2′ and analogs thereof, 4′-C(R^{a}R_{b})—N(R)—O-2′, 4′-C(RₐR_{b})—O—N(R)-2′, 4′-CH₂—O—N(R)-2′, and 4′-CH₂—N(R)—O-2′, wherein each R, R^{a}, and R^{b} is, independently, H, a protecting group, or C₁-C₁₂ alkyl. Representative U.S. patents that teach the preparation of such bicyclic sugar moieties include, but are not limited to: Imanishi et al., U.S. Pat. No. 7,427,672; Swayze et al., U.S. Pat. No. 7,741,457; Swayze et al., U.S. Pat. No. 8,022,193; Seth et al., U.S. Pat. No. 8,278,283; Prakash et al., U.S. Pat. No. 8,278,425; and Seth et al., U.S. Pat. No. 8,278,426.

In certain embodiments, such 4′ to 2′ bridges independently comprise from 1 to 4 linked groups independently selected from: —[C(Rₐ)(R_{b})]ₙ—, —[C(Rₐ)(R_{b})]n-O—, —C(Rₐ)═C(R_{b}), —C(Rₐ)═N—, —C(═NRₐ)—, —C(═O)—, —C(═S)—, —O—, —Si(Rₐ)₂—, —S(═O)ₓ—, and —N(Rₐ)—;
wherein:
x is 0, 1, or 2;
n is 1, 2, 3, or 4;
each Rₐ and R_{b} is, independently, halo, cyano, OR^{a2}, NO₂, NH₂, NHR^{a2}, N(R^{a2})₂, C₁-C₆ alkyl, C₁-C₆ haloalkyl, C₂-C₆ alkenyl, C₂-C₆ alkynyl, C₃-C₁₀ cycloalkyl, C₆₀ aryl, heteroaryl, heterocyclyl, C₁-C₆ alkylene-NH₂, C₁-C₆alkylene-NHR^{a2}, C₁-C₆alkylene-N(R^{a2})₂, C(O)R^{a3}, C(O)OR^{a3}, C(O)NHR^{a3}, C(O)N(C₁-C₄ alkyl)R^{a3}, SR^{a3}, S(O)₂R^{a3}, S(O)R^{a3}, NHC(O)R^{a3}, N(C₁-C₄alkyl)C(O)R^{a3}, NHS(O)R^{a3}, N(C₁-C₄alkyl)S(O)R^{a3} NHS(O)₂R^{a3}, and N(C₁-C₄alkyl)S(O)₂R^{a3}; each R^{a2} is independently selected from C₁-C₆ alkyl, C₂-C₆ alkenyl, C₂-C₆ alkynyl, C₃-C₁₀ cycloalkyl, C₆-C₁₀ aryl, heteroaryl, and heterocyclyl; each R₃ is independently hydrogen, OH, C₁-C₆ alkyl, C₁-C₆ haloalkyl, C₃-C₁₀ cycloalkyl, C₆-C₁₀ aryl, heteroaryl, or heterocyclyl.

In certain embodiments, the bicyclic sugar moiety comprises a bridge between the 5′ and the 3′ furanose ring atoms. Examples of such 5′ to 3′ bridging sugar substituents include, but are not limited to, 5′-(CH₂)₂-3′ (bcDNA), 5′-(CH₂)₃-3′ (bc^{4,3}DNA), 5′-C(F)═CH—CH₂-3′, and 5′-CH₂—CHQ-3′, wherein Q is an attachment to an internucleoside linkage.

Additional bicyclic sugar moieties are known in the art, see, for example: Wan, et al., J. Medicinal Chemistry, 2016, 59, 9645-9667; Wengel et al., U.S. Pat. No. 8,080,644; Ramasamy et al., U.S. Pat. No. 6,525,191; Seth et al., U.S. Pat. No. 7,547,684; and Seth et al., U.S. Pat. No. 7,666,854.

In certain embodiments, bicyclic sugar moieties and nucleosides incorporating such bicyclic sugar moieties are further defined by stereochemical configuration. For example, an LNA nucleoside (described herein) may be in the α-L configuration or in the β-D configuration.

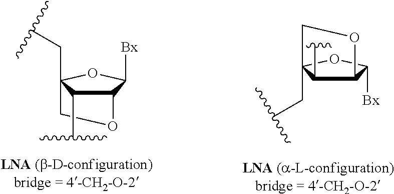

α-L-methyleneoxy (4′-CH₂—O-2′) or α-L-LNA bicyclic nucleosides have been incorporated into oligonucleotides that showed antisense activity (Frieden et al. Nucleic Acids Res. 2003, 21, 6365-6372). The addition of locked nucleic acids to siRNAs has been shown, in certain studies, to increase siRNA stability in serum, and to reduce off-target effects (Elmén, J. et al. Nucleic Acids Res. 2005, 33(1), 439-447; Mook, O. R. et al. Mol. Canc. Ther. 2007, 6(3), 833-843; Grunweller, A. et al. Nucleic Acids Res. 2003, 31(12), 3185-3193). Herein, general descriptions of bicyclic nucleosides include both stereochemical configurations. When the positions of specific bicyclic nucleosides (e.g., LNA or cEt) are identified in exemplified embodiments herein, they are in the β-D stereochemical configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates, selected from cyclic sugar surrogates and acyclic sugar surrogates.

A cyclic sugar surrogate is represented by Formula Ia:

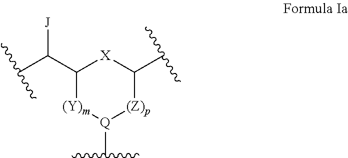

wherein:
J is H, C₁-C₆ alkyl, or C₂-C₆ alkenyl;
X is O, S, C(R₁R₂), N(R₃), or X₁-X₂, wherein X₁-X₂ is C(R₁)═C(R₂), C(R₁R₂)—C(R₁R₂), O—C(R₁R₂), C(R₁R₂)—O, S—C(R₁R₂), C(R₁R₂)—S, N(R₃)—C(R₁R₂), or C(R₁R₂)—N(R₃);
Y is C(R₁R₂) or Y₁—Y₂, wherein Y₁—Y₂ is C(R₁)═C(R₂), or C(R₁R₂)—C(R₁R₂);
Z is C(G₁G₂) or Z₁—Z₂, wherein Z₁—Z₂ is C(G₁)═C(R₁), C(R₁)═C(G₁), C(G₁G₂)-C(R₁R₂), C(R₁R₂)—C(G₁G₂), C(G₁G₂)-C(R₁R₂)—C(R₁R₂), or C(R₁R₂)—C(R₁R₂)—C(G₁G₂);
Q is CH or N;
each R₁ and R₂ is independently H, OH, C₁-C₆ alkyl, or N(R₁); wherein if R₁ is OH, then R₂ is not OH;
each R₃ and R₄ is independently H, C₁-C₆ alkyl, or C(═O)R₅, wherein R₅ is C₁-C₆ alkyl;
each G₁ and G₂ is independently H, OH, halogen or O—[C(R₆)(R₇)]_{q}—[(C═O)ₛ—X^{G}]ⱼ—R₈; wherein if G₁ is OH, then G₂ is not OH;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
m is 0 or 1;
p is 0 or 1;
q is from 1 to 6;
s is 0 or 1;
j is 0 or 1; and
with the proviso that if X is O, Z is C(G₁G₂), and Q is CH, then m is 1.

In certain such embodiments, the oxygen atom of the sugar moiety is replaced, e.g., with a sulfur, carbon or nitrogen atom (X is S, C(R₁R₂), or N(R₃)). In certain such embodiments, such modified sugar moieties also comprise bridging and/or non-bridging substituents as described herein. For example, certain cyclic sugar surrogates comprise a 4′-sulfur atom and a substitution at the 2′-position and/or the 5′ position.

In certain embodiments, cyclic sugar surrogates comprise rings having other than 5 atoms. For example, in certain embodiments, a cyclic sugar surrogate comprises a six-membered tetrahydropyran (“THP”), where X in Formula Ia is O—C(R₁R₂), p is 1, Q is CH, Z is C(G₁G₂), and m is 0. Such tetrahydropyrans may be further modified or substituted. Nucleosides comprising such modified tetrahydropyrans include but are not limited to hexitol nucleic acid (“HNA”), altritol nucleic acid (G₁=OH; G₂=H; “ANA”), and fluoro HNA (FHNA):

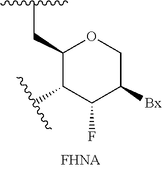

(G₁=F; G₂=H; “FHNA”, see e.g., Egli, M. et al. J Am. Chem. Soc. 2011, 133(41), 16642-16649; Swayze et al., U.S. Pat. No. 8,088,904; and Swayze et al., U.S. Pat. No. 8,440,803); FHNA can also be referred to as a F-THP or 3′-fluoro tetrahydropyran or 3′-FHNA).

In certain embodiments, cyclic sugar surrogates comprise rings having more than 5 atoms and more than one heteroatom. For example, nucleosides comprising morpholino sugar moieties and their use in oligonucleotides have been reported. As used here, the term “morpholino” means a sugar surrogate having Formula Ia, above, wherein X is O, Y and Z are each CH₂, and Q is N.

In certain embodiments, a morpholino is modified, for example by adding or altering various substituent groups from the above morpholino structure. Such sugar surrogates are referred to herein as “modified morpholinos.”

In certain embodiments, sugar surrogates are acyclic sugar surrogates and have Formula IIa, IIIa, or IVa:

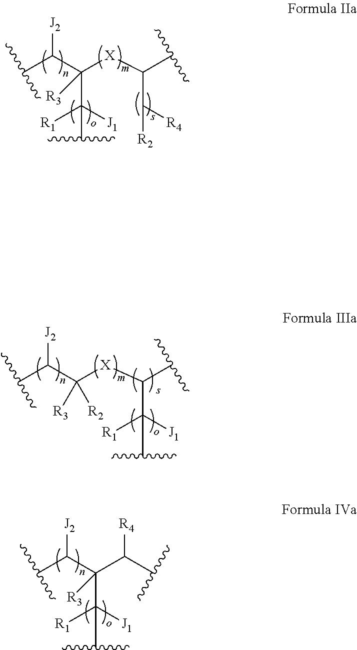

wherein:
X is O, S, C(R₅R₆), N(E₁), NC(═O)-(E₁);
each J₁ and J₂ are independently H or C₁-C₆ alkyl;
n is 0, 1 or 2;
m is 0, 1, or 2;
p is 0 or 1;
o is 0 or 1;
s is 0 or 1;
R₁ is H, OH, halogen, C₁-C₆ alkyl, C₁-C₆ alkoxy, C₂-C₆ alkenyl, C₂-C₆ alkynyl, or (CH₂)_{q}R₈;
R₂, R₃, and R₄ are each independently H, OH, halogen, C₁-C₆ alkyl, C₁-C₆ alkoxy, C₂-C₆ alkenyl, C₂-C₆ alkynyl, S—CH₃, N(CH₃)(CH₃), OCH₂CH₂OCH₃, O-alkylamino, or (CH₂)_{q}R₈;
E₁ is H, C₁-C₆ alkyl or substituted C₁-C₆ alkyl;
R₅ and R₆ are independently H, OH, C₁-C₆ alkyl, or N(R₇); wherein if R₅ is OH, then R₆ is not OH;
R₇ is H, C₁-C₆ alkyl, or C(═O)R₉, wherein R₉ is C₁-C₆ alkyl;
R₈ is OH, halogen, methoxy, ethoxy, azido, C₂-C₆ alkenyl, or C₂-C₆ alkynyl, and q is 1, 2, or 3. In certain embodiments, acyclic sugar surrogates are the “unlocked” sugar structure of UNA (“unlocked nucleic acid”) nucleosides. Representative U.S. publications that teach the preparation of UNA include, but are not limited to, U.S. Patent Publication No. 2011/0313020.

In certain embodiments, acyclic sugar surrogates are the glycerol as found in GNA (“glycol nucleic acid”) nucleosides, having Formula IIa wherein n is 1, m and o are 0, s is 1, and J₂, R₂, and R₃ are each H, or the butyl as found in acyclic butyl nucleic acid, having Formula IIa wherein n is 2, m and o are 0, s is 1, and J₂, R₂, and R₃ are each H. In certain embodiments, acyclic sugar surrogates are also known as “C₃ spacers” and have Formula IIa wherein n and o are 1; m and s are 0, and J₁, J₂, R₁, and R₃ are each H. In certain embodiments, GNA is (S)-GNA.

Further acyclic sugar surrogates include those described in Manoharan et al., U.S. Pat. No. 10,913,767; US patent publication US 2021/0238595; and PCT publication WO 2023/109940.

In certain embodiments, modified oligonucleotides include one or more sugar mimic, in which a group of atoms other than a “furanosyl sugar moiety” or a “sugar surrogate” form the portion of a nucleoside corresponding to the β-D-ribosyl sugar in RNA. In certain embodiments, a sugar mimic is a portion of the backbone of a peptide nucleic acid, while the remainder of the backbone of the peptide nucleic acid is an internucleoside linkage. Representative U.S. patents that teach the preparation of PNA compounds include, but are not limited to, U.S. Pat. Nos. 5,539,082; 5,714,331; and 5,719,262.

In certain embodiments, the modified oligonucleotide comprises a modification disclosed in U.S. Pat. Nos. 10,233,448 or 11,504,391.

### 2. Modified Nucleobases

In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside in which the sugar moiety is not attached to a nucleobase (e.g., the nucleoside does not comprise a nucleobase), referred to as an abasic nucleoside. In certain embodiments, modified oligonucleotides do not comprise abasic nucleosides. In certain embodiments, modified oligonucleotides comprise one or more inosine nucleosides (i.e., nucleosides comprising a hypoxanthine nucleobase). A universal base is a modified nucleobase that can pair with any one of the five unmodified nucleobases.

Unless otherwise indicated, modified adenine has structure (I):

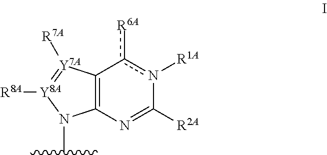

wherein: R^{1A} is absent or H; R^{2A} is H, C₁-C₆ alkyl, substituted C₁-C₆ alkyl, C₁-C₆ thioalkyl, or substituted C₁-C₆ thioalkyl, C₁-C₆ alkyloxy, or substituted C₁-C₆ alkyloxy; R^{6A} is H, N(R^{a})(R^{b}), oxo, acetyl, formyl, or O-phenyl; Y^{7A} is N and R^{7A} is absent or is C₁-C₆ alkyl; or Y^{7A} is C and R_{A} is H, C₁-C₆ alkyl, or N(R^{a})(R^{b}); Y^{8A} is N and R^{8A} is absent, or Y^{8A} is C and R^{8A} is H, a halogen, OH, C₁-C₆ alkyl, or substituted C₁-C₆ alkyl; R^{a} and R^{b} are each independently H, C₁-C₆ alkyl, substituted C₁-C₆ alkyl, C₁-C₆ alkenyl, substituted C₁-C₆ alkenyl, acetyl, or formyl, or together form a 5-7-membered heterocycle; excluding where Y^{7A} is N and R^{7A} is absent; Y^{8A} is C, R^{8A} is H, R^{1A} is absent, R^{2A} is H, and R^{6A} is NH₂ (unmodified adenine).

Unless otherwise indicated, modified guanine has structure (II):

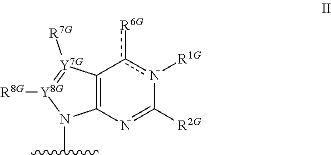

wherein: R_{2G} is N(R^{a})(R^{b}); R^{6G} is oxo and R^{1G} is H, or R^{6G} is O—C₁-C₆ alkyl or S—C₁-C₆ alkyl and R^{1G} is absent; Y^{7G} is N and R^{7G} is absent or is C₁-C₆ alkyl; or Y^{7G} is C and R^{7G} is H, C₁-C₆ alkyl, or N(R^{a})(R^{b}); Y^{8G} is N and R^{8G} is absent, or Y^{8G} is C and R^{8G} is H, a halogen, OH, C₁-C₆ alkyl, or substituted C₁-C₆ alkyl; R^{a} and R^{b} are independently H, C₁-C₆ alkyl, substituted C₁-C₆ alkyl, C₁-C₆ alkenyl, substituted C₁-C₆ alkenyl, acetyl, or formyl, or together form a 5-7-membered heterocycle; excluding where Y^{7G} is N and R^{7G} is absent; Y^{8G} is C, R^{8G} is H, R^{2G} is NH₂, R^{6G} is ═O, and R^{1G} is H (unmodified guanine).

Unless otherwise indicated, modified thymine or modified uracil has structure (III):

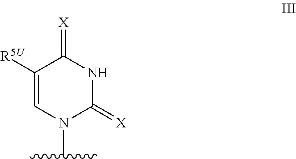

wherein: each X is independently O or S and R^{5U} is H, OH, halogen, O—C₁-C₂₀ alkyl, O—C₁-C₁₂ substituted alkyl, C₁-C₁₂ alkyl, substituted C₁-C₁₂ alkyl, C₁-C₁₂ alkenyl, substituted C₁-C₁₂ alkenyl, C₁-C₁₂ alkynyl, or substituted C₁-C₁₂ alkynyl; wherein if each X is O, R^{5U} is not H or CH₃ (unmodified uracil and unmodified thymine, respectively).

Unless otherwise indicated, modified cytosine has structure (IV):

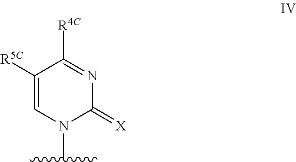

wherein: X is O or S; R^{4C} is N(R^{a})(R^{b}); R^{5C} is H, OH, halogen, O—C₁-C₁₂ alkyl, O—C₁-C₁₂ substituted alkyl, C₁-C₁₂ alkyl, substituted C₁-C₁₂ alkyl, C₁-C₁₂ alkenyl, or substituted C₁-C₁₂ alkenyl; R^{a} and R^{b} are independently H, C₁-C₆ alkyl, substituted C₁-C₆ alkyl, C₁-C₆ alkenyl, substituted C₁-C₆ alkenyl, C₁-C₁₂ alkynyl, substituted C₁-C₁₂ alkynyl, acetyl, or formyl, or together form a 5-7-membered heterocycle; excluding where X is O, R^{4C} is NH₂, and R^{5C} is H (unmodified cytosine).

Hypoxanthine has structure (V):

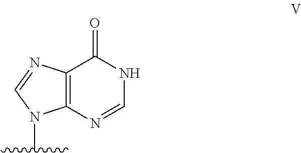 [SMILES: *n1cnc2c(=O)[nH]cnc21]

Hypoxanthine is considered a modified adenine, where Y^{7A} is N and R^{7A} is absent; Y^{8A} is C, R^{8A} is H, R^{1A} is H, R^{2A} is H, and R^{6A} is oxo.

In certain embodiments, modified nucleobases of a modified oligonucleotide are selected from: 5-substituted pyrimidines, 6-azapyrimidines, alkyl or alkynyl substituted pyrimidines, alkyl substituted purines, and N-2, N-6, and O-6 substituted purines. In certain embodiments, modified nucleobases are selected from: 5-methylcytosine, hypoxanthine, 1-methylpseudouridine, 2-aminopropyladenine, 5-hydroxymethyl cytosine, xanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo (particularly 5-bromo), 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one, and 9-(2-aminoethoxy)-1,3-diazaphenoxazine-2-one (G-clamp). Modified nucleobases may also include those in which the purine or pyrimidine base is replaced with other heterocycles, for example, 7-deaza-adenine, 7-deazaguanosine, 2-aminopyridine, and 2-pyridone. Further nucleobases include those disclosed in Englisch, U. et al., Angew. Chem. Int. Ed. 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S.T., Ed., CRC Press, 2008, 163-166 and 442-443.

Preparation of certain of the above noted modified nucleobases, as well as other modified nucleobases, are known in the art and can be readily identified in publications, including, without limitation, Rogers et al., U.S. Pat. No. 5,134,066; Benner et al., U.S. Pat. No. 5,432,272; Matteucci et al., U.S. Pat. No. 5,502,177; Froehler et al., U.S. Pat. No. 5,594,121; and Cook et al., U.S. Pat. No. 5,681,941.

In certain embodiments, at least one nucleobase of a modified oligonucleotide is a modified nucleobase selected from modified adenine (A) having a structure represented by structure I, modified guanine (G) having a structure represented by structure II, modified thymine (T) or modified uracil (U) having a structure represented by structure III, and modified cytosine (C) having a structure represented by structure IV.

In certain embodiments, each nucleobase of a modified oligonucleotide is selected from unmodified A, unmodified G, unmodified C, unmodified T, unmodified U, and 5-methylcytosine (^{m}C). 5-methylcytosine is a modified nucleobase having structure IV, where X is O, R^{4C} is NH₂, and R^{5C} is CH₃.

In certain embodiments, each nucleobase of a modified oligonucleotide is selected from unmodified A, unmodified G, unmodified C, unmodified T, unmodified U, 5-methylcytosine (^{m}C), and hypoxanthine. Hypoxanthine is a modified nucleobase having structure V and is also a modified A represented by structure I, where Y^{7A} is N and R^{7A} is absent; Y^{8A} is C, R^{8A} is H, R^{1A} is H, R^{2A} is H, and R^{6A} is oxo.

In certain embodiments, there are no modified nucleobases in a modified oligonucleotide and each nucleobase of a modified oligonucleotide is selected from unmodified A, unmodified G, unmodified C, unmodified T, and unmodified U.

### 3. Modified Internucleoside Linkages

In certain embodiments, oligomeric agents provided herein comprise or consist of a modified oligonucleotide comprising at least one modified internucleoside linkage. The naturally occurring internucleoside linkage of RNA and DNA is a 3′ to 5′ phosphodiester linkage. Herein, all internucleoside linkages between furanosyl sugar moieties are 3′ to 5′ internucleoside linkages unless otherwise indicated. In certain embodiments, nucleosides of modified oligonucleotides are linked together using one or more modified internucleoside linkages. The two main classes of internucleoside linkages are defined by the presence or absence of a phosphorus atom. Representative phosphorus-containing internucleoside linkages include but are not limited to phosphates, which contain a phosphodiester bond (“P═O”) (also referred to as unmodified linkages), phosphotriesters, methylphosphonates, phosphoramidates, phosphorothioates (“P═S”), and phosphorodithioates (“HS—P═S”). Representative non-phosphorus containing internucleoside linkages include but are not limited to methylenemethylimino (—CH₂—N(CH₃)—O—CH₂—), thiodiester, thionocarbamate (—O—C(═O)(NH)—S—), siloxane (—O—SiH₂—O—), and N,N′-dimethylhydrazine (—CH₂—N(CH₃)—N(CH₃)—). Modified internucleoside linkages, compared to naturally occurring phosphodiester linkages, may be used to alter, typically increase, nuclease resistance of the oligonucleotide.

In certain embodiments, a modified internucleoside linkage is any of those described in WO 2021/030778. In certain embodiments, a modified internucleoside linkage has the formula:

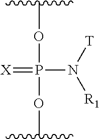

wherein independently for each internucleoside linkage of the modified oligonucleotide:
X is selected from O and S;
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl, substituted C₁-C₆ alkynyl, and a conjugate group;
R₃ is selected from an aryl, a substituted aryl, CH₃, N(CH₃)₂, OCH₃, and a conjugate group;
R₄ is selected from OCH₃, OH, C₁-C₆ alkyl, substituted C₁-C₆ alkyl, and a conjugate group; and
R₅ is selected from OCH₃, OH, C₁-C₆ alkyl, and substituted C₁-C₆ alkyl.

In certain embodiments, a modified oligonucleotide comprises a mesyl phosphoramidate linkage having a formula:

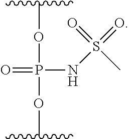 [SMILES: *OP(=O)(NS(C)(=O)=O)O*]

Certain internucleoside linkages having reduced charge (referred to as “neutral internucleoside linkages”) have been described. Such neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl (MOP) (see U.S. Pat. No. 9,926,556), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

In certain embodiments, a modified oligonucleotide comprises an internucleoside linkage comprising a triazole, alkyne, or cyclic guanidine moiety. In certain embodiments, a modified oligonucleotide comprises an internucleoside linkage having a formula:

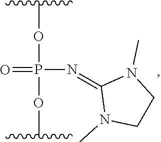 [SMILES: *OP(=O)(N=C1N(C)CCN1C)O*]

which may be stereorandom or may be enriched for the (Rp) or (Sp) configuration.

In certain embodiments, internucleoside linkages are not 3′-to-5′ internucleoside linkages.

In certain embodiments, modified oligonucleotides comprise one or more inverted nucleoside, where a sugar moiety is linked 3′ to 3′ and/or 5′ to 5′, as shown below:

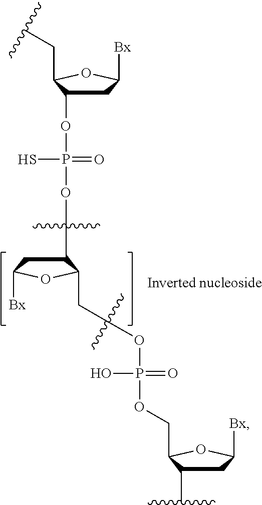

wherein each Bx independently represents any nucleobase.

In certain embodiments, an inverted nucleoside is terminal (i.e., the last nucleoside on one end of an oligonucleotide) and so only one internucleoside linkage depicted above will be present. In certain embodiments, additional features (e.g., a conjugate group) are attached to the inverted nucleoside. Such terminal inverted nucleosides may be attached to either or both ends of an oligonucleotide.

In certain embodiments, inverted nucleosides lack a nucleobase (are abasic nucleosides). In certain such embodiments, additional features (e.g., a conjugate group) are attached to the inverted abasic nucleoside. A terminal inverted nucleoside may be attached to either or both ends of an oligonucleotide.

In certain embodiments, nucleosides are linked 2′ to 5′ rather than the 3′ to 5′ linkage. Such a linkage is illustrated below.

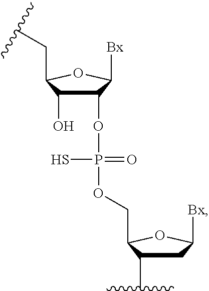

wherein each Bx represents any nucleobase.

In certain embodiments, a bicyclic sugar moiety may be linked via an atom on the non-furanosyl ring. In certain such embodiments, a bicyclic sugar moiety is linked 7′ to 5′, as shown below:

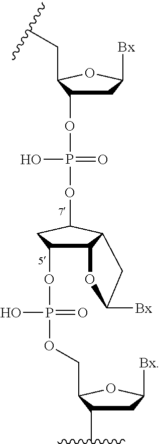

In certain embodiments, internucleoside linkages have at least one chiral center. In such embodiments, a chiral atom can be prepared as a racemic mixture, or as separate enantiomers. Representative internucleoside linkages having a chiral center include but are not limited to alkylphosphonates, mesyl phosphoramidates, and phosphorothioates.

The mesyl phosphoramidate internucleoside linkage comprises a chiral center. In certain embodiments, modified oligonucleotides comprising (Rp) and/or (Sp) mesyl phosphoramidates comprise one or more of the following formulas, respectively, wherein “Bx” indicates a nucleobase:

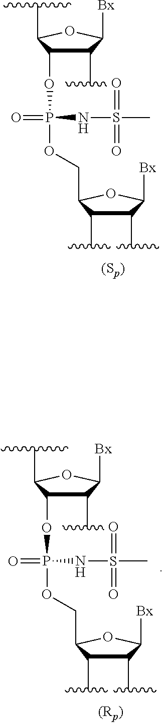

The phosphorothioate internucleoside linkage comprises a chiral center. In certain embodiments, modified oligonucleotides comprising (Rp) and/or (Sp) phosphorothioates comprise one or more of the following formulas, respectively, wherein “Bx” indicates a nucleobase:

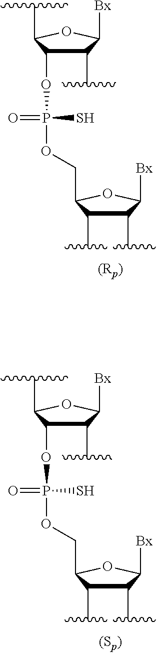

Modified oligonucleotides comprising internucleoside linkages having a chiral center may be prepared as populations of modified oligonucleotides comprising stereorandom internucleoside linkages, or as populations of modified oligonucleotides comprising internucleoside linkages containing chiral centers in particular stereochemical configurations. In certain embodiments, populations of modified oligonucleotides comprise one or more phosphorothioate internucleoside linkages wherein all of the phosphorothioate internucleoside linkages are stereorandom. In certain embodiments, populations of modified oligonucleotides comprise one or more mesyl phosphoramidate internucleoside linkages wherein all of the mesyl phosphoramidate internucleoside linkages are stereorandom. Such modified oligonucleotides can be generated using synthetic methods that result in random selection of the stereochemical configuration of each phosphorothioate and/or mesyl phosphoramidate linkage. Nonetheless, each individual phosphorothioate and/or mesyl phosphoramidate of each individual oligonucleotide molecule has a defined stereoconfiguration. In certain embodiments, populations of modified oligonucleotides are enriched for modified oligonucleotides comprising one or more particular phosphorothioate and/or mesyl phosphoramidate internucleoside linkages in a particular, independently selected stereochemical configuration (e.g., Rp or Sp). In certain embodiments, the particular phosphorothioate and/or mesyl phosphoramidate linkage is present in the selected configuration in at least 65%, 70%, 80, 90%, or 99% of the molecules in the population. Such chirally enriched populations of modified oligonucleotides can be generated using synthetic methods known in the art, e.g., methods described in Oka, N., et al. J. Am. Chem. Soc. 2003, 125, 8307-8317; Wan, W. B., et al. Nucleic Acids Res. 2014, 42, 13456. and WO 2017/015555.

As used herein, “chirally enriched” in reference to a population means a plurality of molecules of identical molecular formula, wherein one or more particular chiral centers are not stereorandom as defined herein. Chirally enriched stereocenters are intentionally controlled during synthesis, or enriched following synthesis, for a particular absolute stereochemical configuration at that center. Populations of molecules having multiple chiral centers within each molecule may contain one or more stereorandom chiral centers. In certain embodiments, the molecules are modified oligonucleotides. In certain embodiments, the chiral center is at the phosphorous atom of a phosphorothioate internucleoside linkage. In certain embodiments, the chiral center is at the phosphorous atom of a mesyl phosphoramidate internucleoside linkage. In certain embodiments, a population of modified oligonucleotides is enriched for modified oligonucleotides having at least one indicated phosphorothioate and/or mesyl phosphoramidate in the (Sp) configuration. In certain embodiments, a population of modified oligonucleotides is enriched for modified oligonucleotides having at least one phosphorothioate and/or mesyl phosphoramidate in the (Rp) configuration. Unless otherwise indicated, chiral internucleoside linkages of modified oligonucleotides described herein may be stereorandom or chirally enriched.

### B. Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In certain such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

### 1. Sugar Motifs

In certain embodiments, oligonucleotides comprise one or more type of modified sugar and/or unmodified sugar moiety arranged along the oligonucleotide or region thereof in a defined pattern or sugar motif. In certain instances, such sugar motifs include, but are not limited to, any of the sugar modifications discussed herein. In certain embodiments, the sugar moiety of at least one nucleoside of an antisense oligonucleotide is a modified sugar moiety.

In certain embodiments, modified oligonucleotides comprise or consist of a region having a fully modified sugar motif. In such embodiments, each nucleoside of the fully modified region of the modified oligonucleotide comprises a modified sugar moiety. In certain embodiments, each nucleoside of the entire modified oligonucleotide comprises a modified sugar moiety and the oligonucleotide is referred to as a fully modified oligonucleotide. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, each nucleoside of a uniformly modified oligonucleotide is a 2′-substituted nucleoside comprising the same 2′-substituent. In certain embodiments, every other nucleoside of a fully modified oligonucleotide comprises the same 2′-substitutent, resulting in alternating 2′-substituents.

In certain embodiments, a modified oligonucleotide comprises a deoxy region. In certain embodiments, each nucleoside of the deoxy region is a deoxynucleoside. In certain embodiments, each nucleoside of the deoxy region is a 2′-β-D-deoxynucleoside. In certain embodiments, the deoxy region consists of 5-13, 7-13, or 10-13 linked nucleosides. In certain embodiments, the deoxy region consists of 6, 7, 8, 9, 10, 11, 12, 13, or 6-13 linked nucleosides. In certain embodiments, at least one nucleoside within the deoxy region comprises a modified sugar moiety. In certain embodiments, exactly one nucleoside within the deoxy region comprises a modified sugar moiety. In certain embodiments, two or three nucleosides within the deoxy region comprise a modified sugar moiety. In certain embodiments, the nucleosides within the deoxy region does not comprise a modified sugar moiety.

In certain embodiments, the deoxy region is flanked on the 5′-side by a 5′-region consisting of linked 5′-region nucleosides and on the 3′-side by a 3′-region consisting of linked 3′-region nucleosides, wherein the 3′-most nucleoside of the 5′-region comprises a modified sugar moiety and the 5′-most nucleoside of the 3′-region comprises a modified sugar moiety. The three regions (the 5′-region, the deoxy region, and the 3′-region) form a contiguous sequence of nucleosides. In certain embodiments, the sugar moiety of the 3′-most nucleoside of the 5′-region and the sugar moiety of the 5′-most nucleoside of the 3′-region each differ from the sugar moiety of the respective adjacent nucleoside of the deoxy region, thus defining the boundary between the 5′-region, the deoxy region, and the 3′-region. In certain embodiments, each nucleoside of the 5′-region and each nucleoside of the 3′-region comprises a modified sugar moiety. In certain embodiments, at least two nucleosides of the 5′-region and at least two nucleosides of the 3′-region comprise a modified sugar moiety. In certain embodiments, at least three nucleosides of the 5′-region and at least three nucleosides of the 3′-region comprise a modified sugar moiety. In certain embodiments, at least four nucleosides of each nucleoside of the 5′-region and each nucleoside of the 3′-region comprises a modified sugar moiety. In certain embodiments, each of the nucleosides within the 5′-region comprise the same modified sugar moiety. In certain embodiments, the nucleosides within the 5′-region comprise two or more different modified sugar moieties. In certain embodiments, each of the nucleosides within the 3′-region comprise the same modified sugar moiety. In certain embodiments, the nucleosides within the 3′-region comprise two or more different modified sugar moieties.

In certain embodiments, the 5′-region and the 3′-region of a modified oligonucleotide each consist of 1-8 nucleosides. In certain embodiments, the 5′-region consists of 1-7 nucleosides. In certain embodiments, the 5′-region consists of 1-6, 2-6, 3-6, or 3-5 nucleosides. In certain embodiments, the 5′-region consists of 1, 2, 3, 4, 5, 6, 7, or 8 nucleosides. In certain embodiments, the 3′-region consists of 1-7 nucleosides. In certain embodiments, the 3′-region consist of 1-6, 2-6, 3-6, or 3-5 nucleosides. In certain embodiments, the 3′-region consists of 1, 2, 3, 4, 5, 6, 7, or 8 nucleosides.

In certain embodiments, the deoxy region consists of 8, 9, 10, 11, 12, or 13 nucleosides, with each nucleoside comprising a 2′-β-D-deoxyribosyl sugar moiety. In certain embodiments, such modified oligonucleotides are referred to as “gapmers”. Herein, the lengths (number of nucleosides) of the 5′-region, the deoxy region, and the 3′-region of an oligonucleotide may be provided using the notation [#of nucleosides in the 5′-region]−[#of nucleosides in the deoxy region]−[#of nucleosides in the 3′-region]. Thus, a 3-10-3 gapmer consists of 3 linked nucleosides in each wing and 10 linked nucleosides in the gap. Where such nomenclature is followed by a specific modification, that modification is the modification in each sugar moiety of the 5′-region and the 3′-region and the gap nucleosides comprise 2′-β-D-deoxyribosyl sugar moieties. Thus, a 5-10-5 MOE gapmer consists of 5 linked 2′-MOE nucleosides in the 5′-region (or “wing”), 10 linked 2′-β-D-deoxynucleosides in the deoxy region (or “gap”), and 5 linked 2′-MOE nucleosides in the 3′-region (or “wing”). A 5-8-5 gapmer consists of 5 linked nucleosides comprising a modified sugar moiety in the 5′-region, 8 linked 2′-β-D-deoxynucleosides in the deoxy region, and 5 linked nucleosides comprising a modified sugar moiety in the 3′-region. A 5-8-5 mixed gapmer has at least two differently modified sugar moieties in the 5′- and/or the 3′-regions.

In certain embodiments, modified oligonucleotides disclosed herein are modified by two or more sugar modifications. In certain embodiments, modified oligonucleotides are 3-10-3 gapmers, wherein each nucleoside within the 5′- and the 3′-regions comprises a modified sugar moiety selected from a 2′-MOE sugar moiety and a 2′-cEt sugar moiety, and the gap nucleosides comprise 2′-β-D-deoxyribosyl sugar moieties. In certain embodiments, modified oligonucleotides are 3-10-4 gapmers, wherein each nucleoside within the 5′- and the 3′-regions comprises a modified sugar moiety selected from a 2′-MOE sugar moiety and a 2′-cEt sugar moiety, and the gap nucleosides comprise 2′-β-D-deoxyribosyl sugar moieties. In certain embodiments, modified oligonucleotides are 3-10-5 gapmers, wherein each nucleoside within the 5′- and the 3′-regions comprises a modified sugar moiety selected from a 2′-MOE sugar moiety and a 2′-cEt sugar moiety, and the gap nucleosides comprise 2′-β-D-deoxyribosyl sugar moieties. In certain embodiments, modified oligonucleotides are 4-9-4 gapmers, wherein each nucleoside within the 5′- and the 3′-regions comprises a modified sugar moiety selected from a 2′-MOE sugar moiety and a 2′-cEt sugar moiety, and the gap nucleosides comprise 2′-β-D-deoxyribosyl sugar moieties. In certain embodiments, modified oligonucleotides are 4-10-6 gapmers, wherein each nucleoside within the 5′- and the 3′-regions comprises a modified sugar moiety selected from a 2′-MOE sugar moiety and a 2′-cEt sugar moiety, and the gap nucleosides comprise 2′-β-D-deoxyribosyl sugar moieties. In certain embodiments, modified oligonucleotides are 5-10-4 gapmers, wherein each nucleoside within the 5′- and the 3′-regions comprises a modified sugar moiety selected from a 2′-MOE sugar moiety and a 2′-cEt sugar moiety, and the gap nucleosides comprise 2′-β-D-deoxyribosyl sugar moieties. In certain embodiments, modified oligonucleotides are 5-10-5 gapmers, wherein each nucleoside within the 5′- and the 3′-regions comprises a modified sugar moiety selected from a 2′-MOE sugar moiety and a 2′-cEt sugar moiety, and the gap nucleosides comprise 2′-β-D-deoxyribosyl sugar moieties. In certain embodiments, modified oligonucleotides are 6-10-4 gapmers, wherein each nucleoside within the 5′- and the 3′-regions comprises a modified sugar moiety selected from a 2′-MOE sugar moiety and a 2′-cEt sugar moiety, and the gap nucleosides comprise 2′-β-D-deoxyribosyl sugar moieties.

In certain embodiments, modified oligonucleotides disclosed herein are gapmers in which the 5′- and the 3′-regions each independently comprises modified and unmodified nucleosides. In certain embodiments, modified oligonucleotides are 3-13-4 gapmers, wherein each nucleoside within the 5′- and the 3′-regions comprises a modified sugar moiety (e.g., a 2′-MOE sugar moiety or a 2′-cEt sugar moiety) or a 2′-β-D-deoxyribosyl sugar moiety, and the gap nucleosides comprise 2′-β-D-deoxyribosyl sugar moieties. In certain embodiments, modified oligonucleotides are 5-11-4 gapmers, wherein each nucleoside within the 5′- and the 3′-regions comprises a modified sugar moiety (e.g., a 2′-MOE sugar moiety or a 2′-cEt sugar moiety) or a 2′-β-D-deoxyribosyl sugar moiety, and the gap nucleosides comprise 2′-β-D-deoxyribosyl sugar moieties. In certain embodiments, modified oligonucleotides are 4-12-4 gapmers, wherein each nucleoside within the 5′- and the 3′-regions comprises a modified sugar moiety (e.g., a 2′-MOE sugar moiety or a 2′-cEt sugar moiety) or a 2′-β-D-deoxyribosyl sugar moiety, and the gap nucleosides comprise 2′-β-D-deoxyribosyl sugar moieties. In certain embodiments, modified oligonucleotides are 6-10-4 gapmers, wherein each nucleoside within the 5′- and the 3′-regions comprises a modified sugar moiety (e.g., a 2′-MOE sugar moiety or a 2′-cEt sugar moiety) or a 2′-β-D-deoxyribosyl sugar moiety, and the gap nucleosides comprise 2′-β-D-deoxyribosyl sugar moieties. In certain embodiments, modified oligonucleotides are 5-12-3 gapmers, wherein each nucleoside within the 5′- and the 3′-regions comprises a modified sugar moiety (e.g., a 2′-MOE sugar moiety or a 2′-cEt sugar moiety) or a 2′-β-D-deoxyribosyl sugar moiety, and the gap nucleosides comprise 2′-β-D-deoxyribosyl sugar moieties. In certain embodiments, modified oligonucleotides are 6-11-3 gapmers, wherein each nucleoside within the 5′- and the 3′-regions comprises a modified sugar moiety (e.g., a 2′-MOE sugar moiety or a 2′-cEt sugar moiety) or a 2′-β-D-deoxyribosyl sugar moiety, and the gap nucleosides comprise 2′-β-D-deoxyribosyl sugar moieties. In certain embodiments, modified oligonucleotides are 5-10-5 gapmers, wherein each nucleoside within the 5′- and the 3′-regions comprises a modified sugar moiety (e.g., a 2′-MOE sugar moiety or a 2′-cEt sugar moiety) or a 2′-β-D-deoxyribosyl sugar moiety, and the gap nucleosides comprise 2′-β-D-deoxyribosyl sugar moieties.

In certain embodiments, modified oligonucleotides have a sugar motif selected from 5′-eeeeeeddddddddddeeee-3′, 5′-eeeeeddddddddddeeeee-3′, 5′-eeeeeddddddddeekke-3′, 5′-eeeeeddddddddekkee-3′, 5′-eeeekddddddddeeeee-3′, 5′-eeeekddddddddkeeee-3′, 5′-eeeekddddddddkeeke-3′, 5′-eeeekddddddddkekee-3′, 5′-eeeekddddddddkkeee-3′, 5′-eeekkddddddddeeeee-3′, 5′-eeekkddddddddkeeee-3′, 5′-ekeeeddddddddeeeee-3′, 5′-kkeeeddddddddeeeee-3′, 5′-eeeekddddddddddkeek-3′, 5′-eeeekddddddddddkkee-3′, 5′-eeeekddddddddddkkek-3′, 5′-eeeeeddddddddddkeeee-3′, 5′-eeeeeddddddddddkkeee-3′, 5′-eeeeeeddddddddddkeee-3′, 5′-eeeeeeddddddddddkkee-3′, 5′-eeeeekddddddddddeeee-3′, 5′-eeeeekddddddddddkkee-3′, 5′-eeeekddddddddddeeeee-3′, 5′-eeeekddddddddddkkeee-3′, 5′-eeeekkddddddddddeeee-3′, 5′-eeeekkddddddddddkeee-3′, 5′-eeekddddddddddkkeeee-3′, 5′-eeekkddddddddddeeeee-3′, 5′-eeekkddddddddddkeeee-3′, 5′-keeeekddddddddddkkee-3′, 5′-keeeekddddddddddkkek-3′, 5′-keeeekddddddddddkkkk-3′, 5′-eeekkeddddddddddeeee-3′, 5′-eeekkdddddddddddeeee-3′, 5′-eeeeeeddddddddddkkeee-3′, 5′-eeeeekddddddddddeeeee-3′, 5′-eeeekkddddddddddeeeee-3′, 5′-kkkdddddddddddddkkdk-3′, 5′-kdkdkdddddddddddkdkk-3′, 5′-kdkddkddddddddddkdkk-3′, 5′-kdkdkddddddddddddkkk-3′, 5′-kkdkddddddddddddkkdk-3′, 5′-kkddkdddddddddddkkdk-3′, 5′-kkdddkddddddddddkkdk-3′, 5′-kdkdkdddddddddddkkdk-3′, 5′-kdkddkddddddddddkkdk-3′, 5′-kddkkdddddddddddkkdk-3′, 5′-kddkdkddddddddddkkdk-3′, 5′-kdddkkddddddddddkkdk-3′, 5′-kkkdddddddddddddkdkk-3′, 5′-kkdkddddddddddddkdkk-3′, 5′-kkddkdddddddddddkdkk-3′, 5′-kkdddkddddddddddkdkk-3′, 5′-kddkkdddddddddddkdkk-3′, 5′-kdddkkddddddddddkdkk-3′, 5′-kddkdkddddddddddkdkk-3′, 5′-kkkddddddddddddddkkk-3′, 5′-kkdkdddddddddddddkkk-3′, 5′-kkddkddddddddddddkkk-3′, 5′-kkdddkdddddddddddkkk-3′, 5′-kdkddkdddddddddddkkk-3′, 5′-kddkkddddddddddddkkk-3′, 5′-kdddkkdddddddddddkkk-3′, 5′-kddkdkdddddddddddkkk-3′, 5′-kkdkdkddddddddddkddk-3′, 5′-kdkkdkddddddddddkddk-3′, 5′-kddkkkddddddddddkddk-3′, 5′-kdkdkddddddddddkdkdk-3′, or 5′-kkkddkddddddddddkddk-3′, wherein each “d” represents a 2′-β-D-deoxyribosyl sugar moiety, each “e” represents a 2′-MOE sugar moiety, and each “k” represents a cEt sugar moiety.

In certain embodiments, modified oligonucleotides have a sugar motif selected from 5′-eeeeeeeeeeeeeeeeee-3′, 5′-eeeeeeddddddddddeeee-3′, 5′-eeeeeddddddddeekke-3′, 5′-eeeeeddddddddekkee-3′, 5′-eeeekddddddddeeeee-3′, 5′-eeeekddddddddkeeee-3′, 5′-eeeekddddddddkeeke-3′, 5′-eeeekddddddddkekee-3′, 5′-eeeekddddddddkkeee-3′, 5′-eeekkddddddddeeeee-3′, 5′-eeekkddddddddkeeee-3′, 5′-ekeeeddddddddeeeee-3′, 5′-kkeeeddddddddeeeee-3′, 5′-eeeekddddddddddkeek-3′, 5′-eeeekddddddddddkkee-3′, 5′-eeeekddddddddddkkek-3′, 5′-eeeeeddddddddddkeeee-3′, 5′-eeeeeddddddddddkkeee-3′, 5′-eeeeeeddddddddddkeee-3′, 5′-eeeeeeddddddddddkeek-3′, 5′-eeeeeeddddddddddkkee-3′, 5′-eeeeeeddddddddddkkek-3′, 5′-eeeeekddddddddddeeee-3′, 5′-eeeeekddddddddddkeek-3′, 5′-eeeeekddddddddddkkee-3′, 5′-eeeeekddddddddddkkek-3′, 5′-eeeekddddddddddeeeee-3′, 5′-eeeekddddddddddkkeee-3′, 5′-eeeekkddddddddddeeee-3′, 5′-eeeekkddddddddddkeee-3′, 5′-eeekddddddddddkkeeee-3′, 5′-eeekkdddddddddddeeee-3′, 5′-eeekkddddddddddeeeee-3′, 5′-eeekkddddddddddkeeee-3′, 5′-eeekkeddddddddddeeee-3′, 5′-keeeekddddddddddkeee-3′, 5′-keeeekddddddddddkeek-3′, 5′-keeeekddddddddddkkee-3′, 5′-keeeekddddddddddkkek-3′, 5′-keeeekddddddddddkkkk-3′, 5′-eeeeeeddddddddddkeee-3′, 5′-eeeeeeddddddddddkkee-3′, 5′-eeeeekddddddddddeeee-3′, 5′-eeeekkddddddddddeeee-3′, 5′-eeeeeeddddddddddkkeee-3′, 5′-eeeeekddddddddddeeeee-3′, or 5′-eeeekkddddddddddeeeee-3′, wherein each “d” represents a 2′-β-D-deoxyribosyl sugar moiety, each “e” represents a 2′-MOE sugar moiety, and each “k” represents a cEt sugar moiety. In certain embodiments, modified oligonucleotides have a sugar motif of 5′-keeeekddddddddddkkee-3′, wherein each “d” represents a 2′-β-D-deoxyribosyl sugar moiety, each “e” represents a 2′-MOE sugar moiety, and each “k” represents a cEt sugar moiety. In certain embodiments, modified oligonucleotides have a sugar motif of 5′-eeeeekddddddddddkkee-3′, wherein each “d” represents a 2′-β-D-deoxyribosyl sugar moiety, each “e” represents a 2′-MOE sugar moiety, and each “k” represents a cEt sugar moiety. In certain embodiments, modified oligonucleotides have a sugar motif of 5′-eeekddddddddddkkeeee-3′, wherein each “d” represents a 2′-β-D-deoxyribosyl sugar moiety, each “e” represents a 2′-MOE sugar moiety, and each “k” represents a cEt sugar moiety.

In certain embodiments, modified oligonucleotides have a sugar motif selected from 5′-kkkdddddddddddddkkdk-3′, 5′-kdkdkdddddddddddkdkk-3′, 5′-kdkddkddddddddddkdkk-3′, 5′-kdkdkddddddddddddkkk-3′, 5′-kkdkddddddddddddkkdk-3′, 5′-kkddkdddddddddddkkdk-3′, 5′-kkdddkddddddddddkkdk-3′, 5′-kdkdkdddddddddddkkdk-3′, 5′-kdkddkddddddddddkkdk-3′, 5′-kddkkdddddddddddkkdk-3′, 5′-kddkdkddddddddddkkdk-3′, 5′-kdddkkddddddddddkkdk-3′, 5′-kkkdddddddddddddkdkk-3′, 5′-kkdkddddddddddddkdkk-3′, 5′-kkddkdddddddddddkdkk-3′, 5′-kkdddkddddddddddkdkk-3′, 5′-kddkkdddddddddddkdkk-3′, 5′-kdddkkddddddddddkdkk-3′, 5′-kddkdkddddddddddkdkk-3′, 5′-kkkddddddddddddddkkk-3′, 5′-kkdkdddddddddddddkkk-3′, 5′-kkddkddddddddddddkkk-3′, 5′-kkdddkdddddddddddkkk-3′, 5′-kdkddkdddddddddddkkk-3′, 5′-kddkkddddddddddddkkk-3′, 5′-kdddkkdddddddddddkkk-3′, 5′-kddkdkdddddddddddkkk-3′, 5′-kkdkdkddddddddddkddk-3′, 5′-kdkkdkddddddddddkddk-3′, 5′-kddkkkddddddddddkddk-3′, 5′-kdkdkddddddddddkdkdk-3′, 5′-kkkddkddddddddddkddk-3′, 5′-kkkdddddddddddddkkdk-3′, or 5′-kddkdkddddddddddkkdk-3′, wherein each “d” represents a 2′-β-D-deoxyribosyl sugar moiety and each “k” represents a cEt sugar moiety. In certain embodiments, modified oligonucleotides have a sugar motif of 5′-kkkdddddddddddddkkdk-3′, wherein each “d” represents a 2′-β-D-deoxyribosyl sugar moiety and each “k” represents a cEt sugar moiety.

### 2. Nucleobase Motifs

In certain embodiments, oligonucleotides comprise modified and/or unmodified nucleobases arranged along the oligonucleotide or region thereof in a defined pattern or motif. In certain embodiments, at least one nucleobase is modified. In certain embodiments, none of the nucleobases are modified. In certain embodiments, at least one purine and/or at least pyrimidine is modified. In certain embodiments, at least one adenine is modified. In certain embodiments, at least one guanine is modified. In certain embodiments, at least one thymine is modified. In certain embodiments, at least one uracil is modified. In certain embodiments, at least one cytosine is modified. In certain embodiments, at least one of the cytosine nucleobases in a modified oligonucleotide is 5-methylcytosine. In certain embodiments, all of the cytosine nucleobases are 5-methylcytosines and all of the other nucleobases of the modified oligonucleotide are unmodified nucleobases. In certain embodiments, one or two of the cytosine nucleobases are 5-methylcytosines and all of the other nucleobases of the modified oligonucleotide are unmodified nucleobases. In certain embodiments, each nucleobase is selected from 5-methylcytosine, unmodified cytosine, unmodified thymine, unmodified uracil, unmodified adenine, unmodified guanine, and hypoxanthine. In certain embodiments, each nucleobase is selected from 5-methylcytosine, unmodified cytosine, unmodified thymine, unmodified adenine, and unmodified guanine. In certain embodiments, each nucleobase is selected from unmodified cytosine, unmodified thymine, unmodified uracil, unmodified adenine, and unmodified guanine. In certain embodiments, each nucleobase is selected from unmodified cytosine, unmodified thymine, unmodified adenine, and unmodified guanine.

### 3. Internucleoside Linkage Motifs

In certain embodiments, oligonucleotides comprise modified and/or unmodified internucleoside linkages arranged along the oligonucleotide or region thereof in a defined pattern or motif. In certain embodiments, each internucleoside linkage of a modified oligonucleotide is a phosphorothioate internucleoside linkage. In certain embodiments, each internucleoside linkage of a modified oligonucleotide is independently selected from a phosphorothioate internucleoside linkage, a mesyl phosphoramidate internucleoside linkage, and a phosphodiester internucleoside linkage. In certain embodiments, each internucleoside linkage of a modified oligonucleotide is independently selected from a phosphorothioate internucleoside linkage and a phosphodiester internucleoside linkage. In certain embodiments, each internucleoside linkage of a modified oligonucleotide is independently selected from a mesyl phosphoramidate internucleoside linkage and a phosphorothioate internucleoside linkage. In certain embodiments, each phosphorothioate internucleoside linkage is independently selected from a stereorandom phosphorothioate, a (Sp) phosphorothioate, and a (Rp) phosphorothioate. In certain embodiments, each mesyl phosphoramidate internucleoside linkage is independently selected from a stereorandom mesyl phosphoramidate, a (Sp) mesyl phosphoramidate, and a (Rp) mesyl phosphoramidate.

In certain embodiments, the sugar motif of a modified oligonucleotide is a gapmer and the internucleoside linkages within the gap are all modified. In certain embodiments, some or all of the internucleoside linkages in the wings are unmodified phosphodiester internucleoside linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester linkage is not a terminal internucleoside linkage, and the remaining internucleoside linkages are phosphorothioate internucleoside linkages. In certain such embodiments, all of the phosphorothioate linkages are stereorandom. In certain embodiments, all of the phosphorothioate linkages in the wings are (Sp) phosphorothioates, and the gap comprises at least one Sp or Rp motif. In certain embodiments, populations of modified oligonucleotides are enriched for modified oligonucleotides comprising such internucleoside linkage motifs.

In certain embodiments, modified oligonucleotides have an internucleoside linkage motif comprising one or more mesyl phosphoramidate linking groups. In certain embodiments, a modified oligonucleotide described herein has an internucleoside linkage motif comprising at least 1, at least 2, at least 3, at least 4, at least 5, at least 6, at least 7, at least 8, at least 9, or at least 10 mesyl phosphoramidate linking groups. In certain embodiments, a modified oligonucleotide described herein has an internucleoside linkage motif comprising 1, 2, 3, 4, 5, 6, 7, 8, 9, or 10 mesyl phosphoramidate linking groups. In certain embodiments, a modified oligonucleotide described herein has an internucleoside linkage motif comprising 1, 2, 3, 4, 5, 6, 7, or 8 mesyl phosphoramidate linking groups. In certain embodiments, a modified oligonucleotide described herein has an internucleoside linkage motif comprising 4 mesyl phosphoramidate linking groups. In certain embodiments, a modified oligonucleotide described herein has an internucleoside linkage motif comprising 3 mesyl phosphoramidate linking groups. In certain embodiments, a modified oligonucleotide described herein has an internucleoside linkage motif comprising 5 mesyl phosphoramidate linking groups. In certain embodiments, a modified oligonucleotide described herein has an internucleoside linkage motif comprising 6 mesyl phosphoramidate linking groups. In certain embodiments, a modified oligonucleotide described herein has an internucleoside linkage motif comprising 7 mesyl phosphoramidate linking groups. In certain embodiments, a modified oligonucleotide described herein has an internucleoside linkage motif comprising 8 mesyl phosphoramidate linking groups. In certain embodiments, one or more phosphorothioate internucleoside linkages or one or more phosphodiester internucleoside linkages of the internucleoside linkage motifs herein is substituted with a mesyl phosphoramidate linking group.

In certain embodiments, modified oligonucleotides have an internucleoside linkage motif of 5′-soosssszzzssssszsss-3′, 5′-sossoszzzssssssssss-3′, 5′-sooosszzssssssssoss-3′, 5′-ssoooszzssssssssoss-3′ 5′-sssooozzssssssssoss-3′, 5′-sooosszzsssssssssss-3′, 5′-ssoooszzsssssssssss-3′, 5′-sssooozzsssssssssss-3′, 5′-sooooossssssssssoss-3′, 5′-sssssozzzssssszssss-3′, 5′-ssssoszzzssssszssss-3′, 5′-sosssszzzssssszssss-3′, 5′-soossszzzssssssssss-3′, 5′-sososszzzssssssssss-3′, 5′-sosssszzzsssssssoss-3′, 5′-sooossssssssssssoss-3′, 5′-sooooossssszsszzoss-3′, 5′-sooooozzzsssssssoss-3′, 5′-soooooszzzssssssoss-3′, 5′-sosssszzszssszssoss-3′, 5′-ssossszzszssszssoss-3′, 5′-sssosszzszssszssoss-3′, 5′-ssssoszzszssszssoss-3′, 5′-sssssozzszssszssoss-3′, 5′-ssssoozzszssszsssss-3′, 5′-ssssszzzszsszzsssss-3′ 5′-ssssszzzsssszzsssss-3′, 5′-sssssszzzssssszsoss-3′, 5′-ssossszzzssssszssss-3′, 5′-sssssszzzsssszzssss-3′ 5′-sssssozzzsssszzsoss-3′, 5′-ssssssszzzssssszsss-3′, 5′-sssssoszzzssssszoss-3′, 5′-ssssooszzzssssszsss-3′, 5′-sssoosszzzssssszsss-3′, 5′-ssoossszzzssssszsss-3′, 5′-s[pn][pn][pn]oossssssssssoss-3′, 5′-s[pn][pn][pn]oosssssszzssoss-3′, 5′-s[pn][pn][pn]oossssssssss[pn]ss-3′, 5′-s[pn][pn][pn]oosssssszzss[pn]ss-3′, 5′-s[pn][pn][pn]oossssssszzsoss-3′, 5′-s[pn][pn][pn]oossssssszzs[pn]ss-3′, 5′-sssosszzzssssszssss-3′, 5′-sssssszzzssssszssss-3′, 5′-sooossszzsssssszsss-3′, 5′-sooossszzsssssszoss-3′, 5′-soooosszzsssssszoss-3′, 5′-soooszzssssssssooss-3′, 5′-soooszzzsssssssooss-3′, 5′-soooszzzzssssssooss-3′, 5′-sooosszzsssssssooss-3′, 5′-sooosszzzssssssooss-3′, 5′-sooossssssszsszooss-3′, 5′-soooszssssszsszooss-3′ 5′-soooszzssssssszooss-3′, 5′-soooszzzsssssszooss-3′, 5′-soooszsssszzszsooss-3′, 5′-ssooszsssszzszsooss-3′, 5′-ssssszsssszzszsssss-3′, 5′-ssssszsssszzszzssss-3′, 5′-zsssszsssszzszssszz-3′, 5′-zsssszsssszzszssssz-3′, 5′-zsssszsssszzssssssz-3′, 5′-sssosszzzssssssooss-3′, 5′-sssosszzzssssssosss-3′, 5′-ssossszzzssssssosss-3′, 5′-sssosszzzssssssssss-3′, 5′-sssoszzzzssssssosss-3′, 5′-sssoszzzzssssssssss-3′ 5′-ssssozzzzssssssssss-3′, 5′-ssssszzzzssssssssss-3′, 5′-sssoszzzzssssszosss-3′, 5′-ssssszzzzssssszosss-3′, 5′-ssssszzzzssssszssss-3′, 5′-soosszzzsssssssosss-3′, 5′-ssosszzzsssssssosss-3′, 5′-sossszzzsssssssosss-3′, 5′-ssssszzzsssssssosss-3′, 5′-sososzzzsssssssosss-3′, 5′-ssosszzzssssszsosss-3′, 5′-ssssszzzssssszsosss-3′, 5′-sssoszzzssssszsosss-3′, 5′-ssssszzzssssszsssss-3′, 5′-ssssszzzsssszzsosss-3′, 5′-sssoszzzsssszzsssss-3′, 5′-ssssozzzsssszzsssss-3′, 5′-soossssszzsssssosss-3′, 5′-soossssszzsssssssss-3′, 5′-sssssssszzsssssooss-3′, 5′-ssosssszzzszsssssss-3′, 5′-sssossszzzszsssssss-3′, 5′-ssssosszzzszsssssss-3′, 5′-ssssssszzzszsssosss-3′, 5′-ssssssszzzzzsssosss-3′, 5′-ssssosszzzzzsssssss-3′, 5′-sssossszzzzzsssssss-3′, 5′-ssosssszzzzzsssssss-3′, 5′-ssssssszzzzzsssssss-3′, 5′-soossssssssssooss-3′, 5′-sosssszzzssssszoss-3′, 5′-sooosssssssssssooss-3′, 5′-soooszzzsssszssooss-3′, 5′-soooszzzsssszzsooss-3′, 5′-ssoooszzssssssssoos-3′, 5′-soosszzzssssszsssss-3′, 5′-zoosszzzssssszsssss-3′, 5′-soosszzzssssszsooss-3′ 5′-soossszzzssssszssss-3′, 5′-sossszzzzssssszssss-3′, 5′-soooszzzsssssssssss-3′, 5′-soszsszzzssssszssss-3′, 5′-soossszzsssssssssss-3′, 5′-zossszzzssssszsssss-3′, 5′-zossszzzssssszssssz-3′, 5′-ssoosszzzssssszsooss-3′, 5′-ssoooszzsssssszsooss-3′, 5′-zsoooszzsssssszsoosz-3′, 5′-ssoooszzzssssszsooss-3′, 5′-zsossszzzssssszsoosz-3′, 5′-ssszzzzzzssssszsssz-3′, 5′-szszszzzzssssszsszs-3′, 5′-szszzszzzssssszsszs-3′, 5′-szszszzzzssssszszss-3′, 5′-sszszzzzzssssszsssz-3′, 5′-sszzszzzzssssszsssz-3′, 5′-sszzzszzzssssszsssz-3′, 5′-szszszzzzssssszsssz-3′, 5′-szszzszzzssssszsssz-3′ 5′-szzsszzzzssssszsssz-3′, 5′-szzszszzzssssszsssz-3′, 5′-szzzsszzzssssszsssz-3′, 5′-ssszzzzzzssssszsszs-3′, 5′-sszszzzzzssssszsszs-3′, 5′-sszzszzzzssssszsszs-3′, 5′-sszzzszzzssssszsszs-3′, 5′-szzsszzzzssssszsszs-3′, 5′-szzzsszzzssssszsszs-3′, 5′-szzszszzzssssszsszs-3′, 5′-ssszzzzzzssssszszss-3′ 5′-sszszzzzzssssszszss-3′, 5′-sszzszzzzssssszszss-3′, 5′-sszzzszzzssssszszss-3′, 5′-szszzszzzssssszszss-3′, 5′-szzsszzzzssssszszss-3′, 5′-szzzsszzzssssszszss-3′, 5′-szzszszzzssssszszss-3′, 5′-sszszszzzssssszsszz-3′, 5′-szsszszzzssssszsszz-3′, 5′-szzssszzzssssszsszz-3′, 5′-szszsszzzssssszszsz-3′ 5′-ssszzszzzssssszsszz-3′, 5′-ssszzzsszzssssssssz-3′, 5′-ssszzzssszzsssssssz-3′, 5′-ssszzzssssszzsssssz-3′, 5′-ssszzzzzzsssssszssz-3′, 5′-ssszzzzzzzssssssssz-3′, 5′-ssszzzssssszzzssssz-3′, 5′-ssszzzszzssssszsssz-3′, 5′-ssszzzzzzsssssssssz-3′, 5′-szzszszzssssssssssz-3′, 5′-szzszszzzsssssszssz-3′, 5′-szzszszzzzssssssssz-3′, 5′-szzszsszzssssszsssz-3′, or 5′-ssszzzzzssssssssssz-3′, wherein each “s” represents a phosphorothioate internucleoside linkage, each “o” represents a phosphodiester internucleoside linkage, each “z” represents a mesyl phosphoramidate internucleoside linkage, and each “[pn]” represents a cyclic guanidine phosphoramidate internucleoside linkage as shown below:

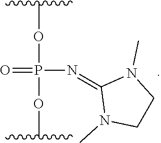 [SMILES: *OP(=O)(N=C1N(C)CCN1C)O*]

In certain embodiments, modified oligonucleotides have an internucleoside linkage motif of 5′-soossssssssssooss-3′, 5′-sosssszzzssssszoss-3′, 5′-ssoooszzsssssszsooss-3′, 5′-ssoooszzzssssszsooss-3′, 5′-ssoosszzzssssszsooss-3′, 5′-zsoooszzsssssszsoosz-3′, 5′-zsossszzzssssszsoosz-3′, 5′-soooosszzsssssszoss-3′, 5′-sooosssssssssssooss-3′, 5′-sooossszzsssssszoss-3′, 5′-sooossszzsssssszsss-3′, 5′-soooszzzsssssssssss-3′, 5′-soooszzzsssszssooss-3′, 5′-soooszzzsssszzsooss-3′, 5′-soosssszzzssssszsss-3′, 5′-soossszzsssssssssss-3′, 5′-soossszzzssssszssss-3′, 5′-soosszzzssssszsooss-3′, 5′-soosszzzssssszsssss-3′, 5′-sosssszzzssssszssss-3′, 5′-sossszzzzssssszssss-3′, 5′-soszsszzzssssszssss-3′, 5′-ssoooszzssssssssoos-3′, 5′-ssssoszzzssssszssss-3′, 5′-sssssozzzssssszssss-3′, 5′-ssszzszzzssssszsszz-3′ 5′-ssszzzssssszzsssssz-3′, 5′-ssszzzssssszzzssssz-3′, 5′-ssszzzssszzsssssssz-3′, 5′-ssszzzsszzssssssssz-3′ 5′-ssszzzszzssssszsssz-3′, 5′-ssszzzzzssssssssssz-3′, 5′-ssszzzzzzsssssssssz-3′, 5′-ssszzzzzzsssssszssz-3′, 5′-ssszzzzzzssssszsssz-3′, 5′-ssszzzzzzssssszsszs-3′, 5′-ssszzzzzzssssszszss-3′, 5′-ssszzzzzzzssssssssz-3′, 5′-sszszszzzssssszsszz-3′, 5′-sszszzzzzssssszsssz-3′, 5′-sszszzzzzssssszsszs-3′ 5′-sszszzzzzssssszszss-3′, 5′-sszzszzzzssssszsssz-3′, 5′-sszzszzzzssssszsszs-3′, 5′-sszzszzzzssssszszss-3′, 5′-sszzzszzzssssszsssz-3′, 5′-sszzzszzzssssszsszs-3′, 5′-sszzzszzzssssszszss-3′, 5′-szsszszzzssssszsszz-3′, 5′-szszsszzzssssszszsz-3′, 5′-szszszzzzssssszsssz-3′, 5′-szszszzzzssssszsszs-3′ 5′-szszszzzzssssszszss-3′, 5′-szszzszzzssssszsssz-3′, 5′-szszzszzzssssszsszs-3′, 5′-szszzszzzssssszszss-3′, 5′-szzssszzzssssszsszz-3′, 5′-szzsszzzzssssszsssz-3′, 5′-szzsszzzzssssszsszs-3′, 5′-szzsszzzzssssszszss-3′, 5′-szzszsszzssssszsssz-3′, 5′-szzszszzssssssssssz-3′, 5′-szzszszzzsssssszssz-3′ 5′-szzszszzzssssszsssz-3′, 5′-szzszszzzssssszsszs-3′, 5′-szzszszzzssssszszss-3′, 5′-szzszszzzzssssssssz-3′, 5′-szzzsszzzssssszsssz-3′, 5′-szzzsszzzssssszsszs-3′, 5′-szzzsszzzssssszszss-3′, 5′-zoosszzzssssszsssss-3′, 5′-zossszzzssssszsssss-3′, or 5′-zossszzzssssszssssz-3′, wherein each “s” represents a phosphorothioate internucleoside linkage, each “o” represents a phosphodiester internucleoside linkage, and each “z” represents a mesyl phosphoramidate internucleoside linkage. In certain embodiments, modified oligonucleotides have an internucleoside linkage motif of 5′-soosssszzzssssszsss-3′, wherein each “s” represents a phosphorothioate internucleoside linkage, each “o” represents a phosphodiester internucleoside linkage, and each “z” represents a mesyl phosphoramidate internucleoside linkage. In certain embodiments, modified oligonucleotides have an internucleoside linkage motif of 5′-zoosszzzssssszsssss-3′, wherein each “s” represents a phosphorothioate internucleoside linkage, each “o” represents a phosphodiester internucleoside linkage, and each “z” represents a mesyl phosphoramidate internucleoside linkage.

In certain embodiments, modified oligonucleotides have an internucleoside linkage motif of 5′-soooosszzsssssszoss-3′, 5′-sooosssssssssssooss-3′, 5′-sooossszzsssssszoss-3′, 5′-sooossszzsssssszsss-3′ 5′-soooszzzsssssssssss-3′, 5′-soooszzzsssszssooss-3′, 5′-soooszzzsssszzsooss-3′, 5′-soosssszzzssssszsss-3′, 5′-soossszzsssssssssss-3′, 5′-soossszzzssssszssss-3′, 5′-soosszzzssssszsooss-3′, 5′-soosszzzssssszsssss-3′, 5′-sosssszzzssssszssss-3′, 5′-sossszzzzssssszssss-3′, 5′-soszsszzzssssszssss-3′ 5′-ssoooszzssssssssoos-3′, 5′-ssssoszzzssssszssss-3′, 5′-sssssozzzssssszssss-3′, 5′-ssszzszzzssssszsszz-3′, 5′-ssszzzssssszzsssssz-3′, 5′-ssszzzssssszzzssssz-3′, 5′-ssszzzssszzsssssssz-3′, 5′-ssszzzsszzssssssssz-3′, 5′-ssszzzszzssssszsssz-3′, 5′-ssszzzzzssssssssssz-3′, 5′-ssszzzzzzsssssssssz-3′, 5′-ssszzzzzzsssssszssz-3′, 5′-ssszzzzzzssssszsssz-3′, 5′-ssszzzzzzssssszsszs-3′, 5′-ssszzzzzzssssszszss-3′ 5′-ssszzzzzzzssssssssz-3′, 5′-sszszszzzssssszsszz-3′, 5′-sszszzzzzssssszsssz-3′, 5′-sszszzzzzssssszsszs-3′, 5′-sszszzzzzssssszszss-3′, 5′-sszzszzzzssssszsssz-3′, 5′-sszzszzzzssssszsszs-3′, 5′-sszzszzzzssssszszss-3′, 5′-sszzzszzzssssszsssz-3′, 5′-sszzzszzzssssszsszs-3′, 5′-sszzzszzzssssszszss-3′ 5′-szsszszzzssssszsszz-3′, 5′-szszsszzzssssszszsz-3′, 5′-szszszzzzssssszsssz-3′, 5′-szszszzzzssssszsszs-3′, 5′-szszszzzzssssszszss-3′, 5′-szszzszzzssssszsssz-3′, 5′-szszzszzzssssszsszs-3′, 5′-szszzszzzssssszszss-3′, 5′-szzssszzzssssszsszz-3′, 5′-szzsszzzzssssszsssz-3′, 5′-szzsszzzzssssszsszs-3′ 5′-szzsszzzzssssszszss-3′, 5′-szzszsszzssssszsssz-3′, 5′-szzszszzssssssssssz-3′, 5′-szzszszzzsssssszssz-3′, 5′-szzszszzzssssszsssz-3′, 5′-szzszszzzssssszsszs-3′, 5′-szzszszzzssssszszss-3′, 5′-szzszszzzzssssssssz-3′, 5′-szzzsszzzssssszsssz-3′, 5′-szzzsszzzssssszsszs-3′, 5′-szzzsszzzssssszszss-3′ 5′-zoosszzzssssszsssss-3′, 5′-zossszzzssssszsssss-3′, or 5′-zossszzzssssszssssz-3′, wherein each “s” represents a phosphorothioate internucleoside linkage, each “o” represents a phosphodiester internucleoside linkage, and each “z” represents a mesyl phosphoramidate internucleoside linkage. In certain embodiments, modified oligonucleotides have an internucleoside linkage motif of 5′-ssszzzzzzsssssssssz-3′, wherein each “s” represents a phosphorothioate internucleoside linkage and each “z” represents a mesyl phosphoramidate internucleoside linkage.

### C. Lengths

It is possible to increase or decrease the length of an oligonucleotide without eliminating activity. For example, in Woolf et al. Proc. Natl. Acad. Sci. USA 1992, 89:7305-7309, a series of oligonucleotides 13-25 nucleobases in length were tested for their ability to induce cleavage of a target RNA in an oocyte injection model. Oligonucleotides 25 nucleobases in length with 8 or 11 mismatch bases near the ends of the oligonucleotides were able to direct specific cleavage of the target RNA, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 27, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

In certain embodiments, oligonucleotides (including modified oligonucleotides) consist of 16 linked nucleosides. In certain embodiments, oligonucleotides (including modified oligonucleotides) consist of 17 linked nucleosides. In certain embodiments, oligonucleotides (including modified oligonucleotides) consist of 18 linked nucleosides. In certain embodiments, oligonucleotides (including modified oligonucleotides) consist of 19 linked nucleosides. In certain embodiments, oligonucleotides (including modified oligonucleotides) consist of 20 linked nucleosides. In certain embodiments, oligonucleotides (including modified oligonucleotides) consist of 21 linked nucleosides.

### D. Oligomeric Agent Modifications

Provided oligomeric agents comprise one or more modifications (e.g., a modified sugar moiety, a modified nucleobase, a modified internucleoside linkage, and/or combinations thereof), incorporated into a modified oligonucleotide. In certain embodiments, a modified oligonucleotide is characterized by modification motif(s) and overall length. In certain embodiments, such parameters are each independent of one another. Thus, unless otherwise indicated, each internucleoside linkage of a modified oligonucleotide having one or more modified sugar moiety and/or sugar motif, independently, is modified or unmodified and may or may not follow the modification pattern of the sugar modifications or sugar motif. For example, internucleoside linkages within a region of a modified oligonucleotide comprising certain sugar modifications may be the same or different from one another and may be the same or different from the internucleoside linkages of the region of the modified oligonucleotide comprising different sugar modifications. Likewise, such modified oligonucleotides may comprise one or more modified nucleobase independent of the pattern of the sugar modifications or sugar motif and independent of the internucleoside linkages or internucleoside linkage motif Unless specifically indicated, all modifications are independent of nucleobase sequence.

### E. Nucleobase Sequence

In certain embodiments, oligonucleotides (unmodified or modified oligonucleotides) are further described by their nucleobase sequence. In certain embodiments, oligonucleotides have a nucleobase sequence that is complementary to a nucleobase sequence of a second strand of linked nucleosides (e.g., another oligonucleotide or an identified reference nucleic acid, such as a target nucleic acid) or a region thereof. In certain embodiments, a region of an oligonucleotide has a nucleobase sequence that is complementary to a nucleobase sequence of a second strand of linked nucleosides or a region thereof. In certain embodiments, the nucleobase sequence of a region or entire length of an oligonucleotide is at least 70%, at least 80%, at least 85%, at least 90%, at least 95%, or 100% complementary to the nucleobase sequence of a second strand of linked nucleosides or region thereof.

### F. Oligomeric Duplexes

In certain embodiments, an oligomeric agent provided herein comprises a modified oligonucleotide having a targeting region having a nucleobase sequence complementary to a sequence in an APOE nucleic acid paired with a second oligonucleotide to form an oligomeric duplex. In some embodiments, an oligomeric duplex comprises a modified oligonucleotide having a targeting region complementary to a target region of an APOE nucleic acid and a second oligonucleotide having a duplexing region complementary to the modified oligonucleotide or a region thereof. In certain embodiments, the second oligonucleotide is a modified oligonucleotide.

In some embodiments, an oligomeric duplex comprises a modified oligonucleotide having a targeting region complementary to a target region of an APOE nucleic acid and a second modified oligonucleotide having a duplexing region complementary to the modified oligonucleotide or a region thereof. In certain embodiments, the oligomeric duplex is part of an oligomeric agent, wherein the oligomeric agent comprises or consists of: (1) a first modified oligonucleotide, (2) a second oligonucleotide, and (3) optionally a terminal group and/or a conjugate group. Either or both modified oligonucleotides of an oligomeric duplex may be linked to a conjugate group. Either or both modified oligonucleotides of an oligomeric duplex may comprise a terminal group. Each modified oligonucleotide of an oligomeric duplex may include non-complementary or unpaired overhanging nucleosides. In certain embodiments, the nucleobase of the non-complementary or unpaired overhanging nucleosides is adenine or thymidine. In certain embodiments, the two modified oligonucleotides have at least one mismatch relative to one another.

In certain embodiments, an oligomeric agent comprises an oligomeric duplex described herein, which is an antisense agent. In certain embodiments, an oligomeric duplex described herein is an RNAi agent capable of reducing the amount of APOE RNA through the activation of RISC/Ago2.

In certain embodiments, an oligomeric agent comprises at least two oligomeric duplexes linked together. In certain embodiments, an oligomeric agent comprises two oligomeric duplexes wherein at least one oligomeric duplex comprises an antisense oligonucleotide that is complementary to a nucleobase sequence of APOE RNA as described herein. In certain embodiments, an oligomeric agent comprises two or more of the same oligomeric duplex, which is any of the oligomeric duplexes described herein. In certain embodiments, the two or more oligomeric duplexes are covalently linked together. In certain embodiments, the second modified oligonucleotides of the two or more oligomeric duplexes are covalently linked together. In certain embodiments, the second modified oligonucleotides of two or more oligomeric duplexes are covalently linked together at their 3′ ends. In certain embodiments, the second modified oligonucleotides of two or more oligomeric duplexes are covalently linked together at the 3′ end of one to the 5′ end of the other. In certain embodiments, the two or more oligomeric duplexes are covalently linked together by a glycol linker, such as a tetraethylene glycol linker. A structure of oligomeric duplexes covalently linked by a glycol linker is described in, e.g., Alterman, J. F., et al. Nature Biotech. 2019, 37, 844-894. In certain embodiments, an oligomeric agent comprises two oligomeric duplexes formed from one antisense oligonucleotide comprising two targeting regions, wherein each targeting region is paired with a sense oligonucleotide. In some embodiments, an oligomeric agent comprises two or more oligomeric duplexes linked, e.g., covalently linked, together in a branched structure, e.g., a di-branched, tri-branched, or tetra-branched structure (see, e.g., WO 2022/256565). In some such embodiments, the structure contains a linker (e.g., one or more subunits of an ethylene glycol, alkyl, carbohydrate, block copolymer, peptide, ester, amide, carbamate, triazole) and optionally one or more branch point moieties (e.g., phosphoramidite, tosylated solketal, 1,3-diaminopropanol, pentaerythritol).

### G. Conjugates

In certain embodiments, provided herein are oligomeric agents comprising one or more modified oligonucleotides, and one or more conjugate groups and/or one or more terminal groups. In certain embodiments, an oligomeric agent comprises one or more modified oligonucleotides and one or more conjugate groups. In certain embodiments, an oligomeric agent comprises one or more modified oligonucleotides and one or more terminal groups. Conjugate groups comprise or consist of a conjugate moiety and a conjugate linker. A conjugate group may be attached at the 5′ end of an oligonucleotide and/or at the 3′ end of an oligonucleotide and/or at any internal position of an oligonucleotide. In certain embodiments, conjugate groups are attached through a modified sugar moiety or a modified internucleoside linkage. In certain embodiments, oligomeric agents comprise a modified oligonucleotide, a cell-targeting moiety, and a conjugate linker.

### 1. Conjugate Groups

A conjugate group comprises or consists of a conjugate moiety and a conjugate linker. In certain embodiments, a conjugate moiety attached to an oligonucleotide modifies one or more properties of the attached oligonucleotide compared to the same oligonucleotide lacking the conjugate moiety, including but not limited to pharmacodynamics, pharmacokinetics, stability, binding, absorption, tissue distribution, cellular distribution, cellular uptake, charge, and clearance. In certain embodiments, a conjugate moiety imparts a new property on the attached oligonucleotide.

In some embodiments, the conjugate group comprises a small molecule drug (e.g., an active pharmaceutical ingredient), an aliphatic chain, a lipid, a peptide, a protein, a hydrocarbon, a polyamine, a polyamide, a polyether, a thioether, an aptamer, an antibody, an antibody fragment, a VHH camelid antibody fragment, a VNAR shark antibody fragment, a vitamin, a fatty acid, a carbohydrate, an intercalator, a reporter molecule, a small molecule, or an alkyl moiety, e.g., a C22 alkyl, C20 alkyl, C17 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, or C5 alkyl, wherein the alkyl chain optionally has one or more unsaturated bonds. In some embodiments, the conjugate group comprises a 6-palmitamidohexyl moiety or a 2-(hydroxymethyl)-6-palmitamidohexyl moiety. In certain embodiments, the conjugate group comprises a cell-targeting moiety.

Methods of preparing conjugated oligonucleotides are known in the art and/or described herein. For example, in one non-limiting solid phase method for large-scale synthesis of conjugated oligonucleotides, monomethoxytrityl (MMT)-protected 5′ or (3′)-amino-modified oligonucleotide intermediates are generated using the phosphoramidate monomer coupling method and detritylated as described in U.S. Pat. No. 10,450,342. The 5′ (or 3′) MMT-protected amino group may be linked to the oligonucleotide through a linker group such as an alkyl phosphate group, and the MMT group may be removed from the oligonucleotide via solution-phase detritylation conducted at certain temperatures and pH. In certain embodiments, the detritylated oligonucleotide is then reacted with a conjugate group (e.g., a GalNAc₃) to generate a conjugated oligonucleotide.

### 2. Conjugate Moieties

Conjugate moieties include, without limitation, intercalators, reporter molecules, polyamines, polyamides, peptides, carbohydrates (e.g., GalNAc), antibodies, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indomethacin, a barbiturate, a cephalosporin, a sulfa drug, an antidiabetic, an antibacterial or an antibiotic.

In certain embodiments, conjugate moieties are selected from any of C22 alkyl, C20 alkyl, C16 alkyl, C10 alkyl, C21 alkyl, C19 alkyl, C18 alkyl, C17 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C17 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl.

In certain embodiments, conjugate moieties are selected from any of C22 alkyl, C20 alkyl, C16 alkyl, C10 alkyl, C21 alkyl, C19 alkyl, C18 alkyl, C17 alkyl, C15 alkyl, C14 alkyl, C13 alkyl, C12 alkyl, C11 alkyl, C9 alkyl, C8 alkyl, C7 alkyl, C6 alkyl, or C5 alkyl, where the alkyl chain has one or more unsaturated bonds.

### 3. Cell-Targeting Moieties

In certain embodiments, a conjugate group comprises a cell-targeting moiety. In certain embodiments, the cell-targeting moiety targets neurons. In certain embodiments, the cell-targeting moiety targets a neurotransmitter receptor. In certain embodiments, the cell targeting moiety targets a neurotransmitter transporter. In certain embodiments, the cell targeting moiety targets a GABA transporter. See e.g., WO 2011/131693, WO 2014/064257.

In certain embodiments, conjugate groups comprise cell-targeting moieties that have affinities for transferrin receptor (TfR) (also referred to herein as TfR1 and CD71). In certain embodiments, a conjugate group described herein comprises an anti-TfR1 antibody or fragment thereof. In certain embodiments, the conjugate group comprises a protein or peptide capable of binding TfR1. In certain embodiments, the conjugate group comprises an aptamer capable of binding TfR1. In certain embodiments, the anti-TfR1 antibody or fragment thereof can be any known in the art including but not limited to those described in WO1991/004753; WO2013/103800; WO2014/144060; WO2016/081643; WO2016/179257; WO2016/207240; WO2017/221883; WO2018/129384; WO2018/124121; WO2019/151539; WO2020/132584; WO2020/028864; U.S. Pat. Nos. 7,208,174; 9,034,329; 10,550,188; and 11,512,136. In certain embodiments, a fragment of an anti-TfR1 antibody is F(ab′)₂, Fab, Fab′, Fv, scFv, VHH, or VNAR. In certain embodiments, an antibody binds to TfR1 through an engineered Fc domain rather than through the antigen-binding portion, as described in, e.g., US 2020/0223935.

In certain embodiments, the conjugate group comprises a protein or peptide capable of binding TfR1. In certain embodiments, the protein or peptide capable of binding TfR1 can be any known in the art including but not limited to those described in WO2019/140050; WO2020/037150; WO2020/124032; WO 2022/026555; WO 2023/027125; WO 2023/022234; and U.S. Pat. No. 10,138,483.

In certain embodiments, the conjugate group comprises an aptamer capable of binding TfR1. In certain embodiments, the aptamer capable of binding TfR1 can be any known in the art including but not limited to those described in WO2013/163303; WO2019/033051; and WO2020/245198.

### 4. Conjugate Linkers

In certain embodiments, oligomeric agents comprise an oligonucleotide and a conjugate group, wherein the conjugate group consists of a conjugate moiety and a conjugate linker. The conjugate linker links the conjugate moiety to the oligonucleotide. In certain embodiments, the conjugate linker is a single chemical bond (i.e., the conjugate moiety is attached directly to an oligonucleotide through a single bond). In certain embodiments, the conjugate linker comprises one or more atoms. In certain embodiments, the conjugate linker comprises a chain structure, such as a hydrocarbyl chain, or an oligomer of repeating units such as ethylene glycol, nucleosides, or amino acid units. In certain embodiments, the oligonucleotide is a modified oligonucleotide.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to compounds, such as the oligonucleotides provided herein. In general, a bifunctional linking moiety comprises at least two functional groups. One of the functional groups is selected to bind to a particular site on a compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, it is desirable for a conjugate group to be cleaved from the oligonucleotide. For example, in certain circumstances oligomeric agents comprising a particular conjugate moiety are better taken up by a particular cell type, but once the oligomeric agent has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety may be part of the oligonucleotide and comprises or consists of one or more linked nucleosides. In certain such embodiments, the one or more linked nucleosides are linked to one another and/or to the remainder of the oligonucleotide through cleavable bonds. In certain embodiments, such cleavable bonds are unmodified phosphodiester bonds. In certain embodiments, a cleavable moiety is 2′-deoxynucleoside that is either the 3′ or 5′-terminal nucleoside of an oligonucleotide linked by a phosphodiester internucleoside linkage to an adjacent nucleoside of the oligonucleotide and covalently attached to the conjugate linker or conjugate moiety by a phosphodiester or phosphorothioate linkage. In certain such embodiments, the cleavable moiety comprises 2′-deoxyadenosine.

In certain embodiments, oligomeric agents described herein comprise an oligonucleotide linked to a conjugate moiety by a conjugate linker, wherein the oligonucleotide is attached to the conjugate moiety using Click chemistry known in the art. Compounds have been prepared using Click chemistry wherein alkynyl phosphonate internucleoside linkages on an oligonucleotide attached to a solid support are converted into the 1,2,3-triazolylphosphonate internucleoside linkages and then cleaved from the solid support (Krishna, H. et al. J. Am. Chem. Soc. 2012, 134(28), 11618-11631). Additional conjugate linkers suitable for oligonucleotide conjugates are prepared by Click chemistry described in “Click Chemistry for Biotechnology and Materials, Science” Ed. Joerg Lahann, Wiley 2009. Further examples of linking chemistry include an inverse electron demand Diels-Alder reaction, e.g., as described in Argamunt et al., J. Org. Chem. 2020, 85, 10, 6593-6604, Sarrett et al., Nat. Protocols 2021, 16, 3348-3381; Handula et al., Molecules, 2021, 26 (15), 4640, Wiessler et al., Int. J. Med. Sci. 2010, 7 (1), 19-28; copper-catalyzed azide-alkyne cycloaddition (CuAAC) see, e.g., S. I. Presolski, et al., J. Am. Chem. Soc. 2010, 132, 14570-14576; D. Soriano Del Amo, et al., J. Am. Chem. Soc., 2010, 132, 16893-16899; Staudinger reaction, see, e.g., Saxon and C. R. Bertozzi, Science, 2000, 287, 2007-2010; B. L. Nilsson, et al., Org. Lett., 2000, 2, 1939-1941, E. Saxon, et al., Org. Lett., 2000, 2, 2141-2143; formation of hydrazones and oximes, see, e.g., J. Y. Axup, et al., Proc. Natl. Acad. Sci. U.S.A, 2012, 109, 16101-16106; photoclick reactions, see, e.g., W. Song, et al., Angew. Chem., Int. Ed., 2008, 47, 2832-2835, A. Hemer and Q. Lin, Top. Curr. Chem., 2016, 374, 1; strain-promoted alkyne-nitrone cycloaddition (SPANC) reactions, see, e.g., D. A. MacKenzie, et al., Curr. Opin. Chem. Biol., 2014, 21, 81-88; transition metal catalyzed cross coupling, see, e.g., M. Chalker, et al., J. Am. Chem. Soc., 2009, 131, 16346-16347; nucleophilic additions, in particular, of a thiol to a maleimide, see, e.g., Kang et al., Chem. Sci., 2021, 12, 13613-13647, Bernardim et al., Nat. Comm. 2016, 7, 13128, Jain et al., Pharm. Res. 2015, 32 (11), 3526-3540.

### H. Terminal Groups

In certain embodiments, provided herein are oligomeric agents comprising one or more modified oligonucleotides and one or more terminal groups. As used herein, “terminal group” means a group of atoms that is covalently linked to a terminus of an oligonucleotide. Examples of a terminal group include, but are not limited to, a capping group, a phosphate moiety, a stabilized phosphate group, and a protecting group. In certain embodiments, one or more terminal groups is attached to either or both ends of an oligonucleotide. In certain embodiments, one or more terminal groups is attached at the 3′-end and/or at the 5′-end of the oligonucleotide. In certain embodiments, one or more terminal groups is attached at the 3′-end of the oligonucleotide. In certain embodiments, one or more terminal groups is attached at the 5′-end of the oligonucleotide. In certain embodiments, one or more terminal groups is attached at the 3′-end of the oligonucleotide and one or more terminal groups is attached at the 5′-end of the oligonucleotide. In certain embodiments, a terminal group is attached at the 3′-end of the oligonucleotide and/or at the 5′-end of the oligonucleotide. In certain embodiments, a terminal group is attached at the 3′-end of the oligonucleotide. In certain embodiments, a terminal group is attached at the 5′-end of the oligonucleotide. In certain embodiments, a terminal group is attached at the 3′-end of the oligonucleotide and a terminal group is attached at the 5′-end of the oligonucleotide.

In certain embodiments, an oligonucleotide is linked to a terminal group comprising a stabilized 5′-phosphate. Stabilized 5′-phosphates include, but are not limited to 5′-phosphonates, including, but not limited to 5′-vinylphosphonate, 5′-methylphosphonate, and 5′-cyclopropyl phosphonate.

In certain embodiments, terminal groups comprise one or more abasic sugar moieties and/or inverted nucleosides. In certain embodiments, a terminal group comprises an inverted abasic sugar moiety. In certain embodiments, the inverted abasic sugar moiety may be further attached to a conjugate group. In certain embodiments, terminal groups comprise one or more 2′-linked nucleosides or sugar moieties. In certain such embodiments, the 2′-linked group is an abasic sugar moiety. Such terminal abasic sugar moieties can be attached to either or both ends of an oligonucleotide.

### II. Target Nucleic Acids

### A. APOE

In certain embodiments, oligomeric agents comprise or consist of a modified oligonucleotide comprising a targeting region that is complementary to an equal-length target region of a target nucleic acid, wherein the target nucleic acid is an APOE nucleic acid. In certain embodiments, the APOE nucleic acid has the nucleobase sequence set forth in SEQ ID NO: 1 (GENBANK Accession No. NC_000019.10, truncated from nucleotides 44903001 to 44912000), in SEQ ID NO: 2 (GENBANK Accession No. NM_001302688.1), in SEQ ID NO: 3 (GENBANK Accession No. AU126799.1), in SEQ ID NO: 4 (GENBANK Accession No. B1602495.1), in SEQ ID NO: 5 (the complement of GENBANK Accession No. CA306379.1), or in SEQ ID NO: 6 (GENBANK Accession No. NM_000041.2 with T→C at position 471 to result in APOE4 mutant mRNA). In certain embodiments, contacting a cell with an oligomeric agent comprising a modified oligonucleotide comprising a target region that is complementary to an equal-length target region of any of SEQ ID NOs: 1-6 reduces the amount of APOE RNA, and in certain embodiments reduces the amount of ApoE protein in the cell. In certain embodiments, the oligomeric agent consists of a modified oligonucleotide. In certain embodiments, the oligomeric agent consists of a modified oligonucleotide and a conjugate group. In certain embodiments, the modified oligonucleotide of the oligomeric agent is paired with an additional modified oligonucleotide to form an oligomeric duplex. In certain embodiments, the oligomeric duplex comprises a conjugate group. In certain embodiments, the modified oligonucleotide is an antisense oligonucleotide. In certain embodiments, the oligomeric agent consists of an antisense oligonucleotide. In certain embodiments, the oligomeric agent consists of an antisense oligonucleotide and a conjugate group. In certain embodiments, the oligomeric agent consists of an antisense oligonucleotide and one or more terminal group(s). In certain embodiments, the oligomeric agent consists of an antisense oligonucleotide, a conjugate group, and one or more terminal group(s). In certain embodiments, the oligomeric agent reduces the amount of APOE RNA and/or the amount of ApoE protein in vitro by at least 25%, at least 30%, at least 40%, at least 50%, at least 60%, at least 70%, at least 80%, or at least 90% when administered according to the standard in vitro assay. In certain embodiments, the oligomeric agent reduces the amount of APOE RNA and/or the amount of ApoE protein in vivo by at least 25%, at least 30%, at least 40%, at least 50%, at least 60%, at least 70%, at least 80%, or at least 90% when administered according to the standard in vivo assay. In certain embodiments, the oligomeric agent reduces the amount of APOE RNA and/or the amount of ApoE protein in the cell of a subject by at least 25%, at least 30%, at least 40%, at least 50%, at least 60%, at least 70%, at least 80%, or at least 90%.

In certain embodiments, oligomeric agents comprise an antisense oligonucleotide comprising a targeting region that is complementary to a target region of an APOE nucleic acid. In certain embodiments, oligomeric agents comprise an antisense oligonucleotide comprising a targeting region that is complementary to a target region of an APOE nucleic acid, and a sense oligonucleotide comprising a duplexing region that is complementary to the antisense oligonucleotide, or a region thereof.

In certain embodiments, the target nucleic acid is an endogenous APOE RNA molecule. In certain embodiments, the APOE nucleic acid encodes an ApoE protein. In certain embodiments, the APOE nucleic acid is a precursor to a nucleic acid that encodes an ApoE protein. In certain such embodiments, the APOE nucleic acid is selected from: a mature mRNA and a pre-mRNA, including intronic, exonic, and untranslated regions. In certain embodiments, the APOE RNA is a mature mRNA. In certain embodiments, the APOE nucleic acid is a pre-mRNA.

In certain embodiments, a targeting region is from 6 to 20, 10 to 18, 14 to 18, 16 to 20, or 18 to 20 nucleobases in length. In certain embodiments, the targeting region comprises or consists of at least 8, at least 9, at least 10, at least 11, at least 12, at least 13, at least 14, at least 15, at least 16, at least 17, at least 18, at least 19, at least 20, at least 21, at least 22, or at least 23 contiguous nucleobases. In certain embodiments, the targeting region comprises or consists of at least 8, at least 9, at least 10, at least 11, at least 12, at least 13, at least 14, at least 15, at least 16, at least 17, at least 18, at least 19, at least 20, at least 21, at least 22, or 23 contiguous nucleobases. In certain embodiments, the targeting region constitutes at least 75%, at least 80%, at least 85%, at least 90%, or at least 95% of the nucleosides of a modified oligonucleotide (e.g., an antisense oligonucleotide). In certain embodiments, the targeting region constitutes all of the nucleosides of the modified oligonucleotide (e.g., the antisense oligonucleotide). In certain embodiments, the targeting region of the modified oligonucleotide (e.g., the antisense oligonucleotide) is at least 99%, at least 95%, at least 90%, at least 85%, or at least 80% complementary to a target region of the APOE nucleic acid. In certain embodiments, the targeting region of the modified oligonucleotide (e.g., the antisense oligonucleotide) is 100% complementary to a target region of the APOE nucleic acid. In certain embodiments, the targeting region is entirely within an intron. In certain embodiments, the targeting region spans an intron/exon junction.

In certain embodiments, modified oligonucleotides (e.g., antisense oligonucleotides) comprise one or more mismatches relative to the target region of the APOE nucleic acid. In certain embodiments, the modified oligonucleotide is an antisense oligonucleotide. In certain embodiments, antisense activity against the target is reduced by such a mismatch, and activity against a non-target is reduced. In certain embodiments, activity against the non-target is reduced by a greater amount than activity against the target. Thus, in certain embodiments selectivity of the antisense oligonucleotides is improved. In certain embodiments, antisense oligonucleotides are at least 80% complementary to the target region of the APOE nucleic acid over the entire length of the antisense oligonucleotide and comprise no more than one to three mismatches with the APOE nucleic acid. In certain embodiments, antisense oligonucleotides comprise a targeting region that is at least 80% complementary to a target region of the APOE nucleic acid over the entire length of the targeting region, and the targeting region comprises no more than one to three mismatches with the target region. In certain embodiments, antisense oligonucleotides comprise a targeting region that is at least 80%, at least 85%, at least 90%, at least 95%, or 100% complementary to a target region of the APOE nucleic acid over the entire length of the targeting region. In certain embodiments, a mismatch is specifically positioned within an antisense oligonucleotide. In certain embodiments, a mismatch is at position 3, 4, 5, 6, 7, 8, 9, 10, 11, or 12 from the 5′-end of the antisense oligonucleotide. In certain embodiments, a mismatch is at position 11, 10, 9, 8, 7, 6, 5, 4, 3, or 2 from the 3′-end of the antisense oligonucleotide. In certain embodiments, 1-2 additional mismatches may be present at a terminus or at both termini of the antisense oligonucleotide, outside of the targeting region. In certain embodiments, a mismatch is at position 1, 2, 3, or 4 from the 5′-end of the antisense oligonucleotide. In certain embodiments, a mismatch is at position 4, 3, 2, or 1 from the 3′-end of the antisense oligonucleotide.

### B. Target Nucleic Acids in Certain Tissues

In certain embodiments, oligomeric agents comprise or consist of a modified oligonucleotide comprising a targeting region that is complementary to a target region in an APOE nucleic acid, wherein the APOE nucleic acid is expressed in a pharmacologically relevant tissue. In certain embodiments, the pharmacologically relevant tissues are the cells and tissues that comprise the central nervous system (CNS). Such tissues include the brain and spinal cord. In certain embodiments, the pharmacological relevant tissues include the cortex, the hippocampus, amygdala, basal ganglia, thalamus, midbrain, cerebellum, choroid plexus, spinal cord, caudate, putamen, and brain stem. In certain embodiments, the cells are brain cells. In certain embodiments, the cells include glial cells. In certain embodiments, glial cells include astrocytes, oligodendrocytes, and microglial cells. In certain embodiments, the cells include astrocytes and microglial cells. In certain embodiments, the cells include neurons. In certain embodiments, the cells include endothelial cells in the brain, e.g., brain microvascular endothelial cells (BMEC). In certain embodiments, the cells include pericytes in the brain (or brain pericytes).

### C. Oligonucleotide Sequences

Provided herein are oligomeric agents comprising modified oligonucleotides complementary to a target region in an APOE nucleic acid, such as, for example, a human APOE nucleic acid, such as SEQ ID NO: 1 (GENBANK Accession No. NC_000019.10, truncated from nucleotides 44903001 to 44912000), SEQ ID NO: 2 (GENBANK Accession No. NM_001302688.1), SEQ ID NO: 3 (GENBANK Accession No. AU126799.1), SEQ ID NO: 4 (GENBANK Accession No. B1602495.1), SEQ ID NO: 5 (the complement of GENBANK Accession No. CA306379.1), or SEQ ID NO: 6 (GENBANK Accession No. NM_000041.2 with T→C at position 471 to result in APOE4 mutant mRNA), and compositions comprising such oligomeric agents. In certain embodiments, a modified oligonucleotide has a nucleobase sequence comprising or consisting of a targeting region that is at least 80%, at least 85%, at least 90%, at least 95%, or at least 99% complementary to a region of any of SEQ ID NOs: 1-6. In certain embodiments, a modified oligonucleotide has a complementary region that is at least 80%, at least 85%, at least 90%, at least 95%, or at least 99% complementary to a targeting region that is at least 80%, at least 85%, at least 90%, at least 95%, or at least 99% complementary to a target region of any of SEQ ID NOs: 1-6. In certain embodiments, a modified oligonucleotide has a targeting region that is 100% complementary to a target region of any of SEQ ID NOs: 1-6. In certain embodiments, a modified oligonucleotide has a nucleobase sequence comprising or consisting of a complementary region that is at least 80%, at least 85%, at least 90%, at least 95%, or at least 99% complementary to a targeting region that is 100% complementary to a target region of any of SEQ ID NOs: 1-6. In certain embodiments, a modified oligonucleotide has a nucleobase sequence comprising or consisting of any of SEQ ID NOs: 22-40.

### III. Methods and Uses

### A. Antisense Activity

In certain embodiments, oligomeric agents provided herein comprise an antisense oligonucleotide that is capable of hybridizing to a target nucleic acid, resulting in at least one antisense activity; such oligomeric agents are antisense agents.

In certain antisense activities, hybridization of an antisense oligonucleotide to a target nucleic acid results in recruitment of a protein, e.g., RNase H or Argonaute, that cleaves the target nucleic acid. Certain antisense agents result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, oligomeric agents are antisense agents that are sufficiently “DNA-like” to elicit RNase H activity. In certain embodiments, one or more non-DNA-like nucleosides in the antisense agent are tolerated and RNase H activity is retained. In certain embodiments, such antisense agents reduce expression of or reduce the amount or activity of a target nucleic acid by 25% or more in the standard in vitro assay.

In certain antisense activities, an antisense oligonucleotide is loaded into an RNA-induced silencing complex (RISC), ultimately resulting in cleavage of the target nucleic acid. For example, certain antisense oligonucleotides result in cleavage of the target nucleic acid by Argonaute. Antisense agents that comprise an antisense oligonucleotide that is loaded into RISC are RNAi agents. RNAi agents may be double-stranded (siRNA or dsRNAi) or single-stranded (ssRNA). In certain embodiments, RNAi agents are capable of RISC-mediated modulation of a target nucleic acid in a cell. In certain embodiments, such RNAi agents reduce the expression of or reduce the amount or activity of a target nucleic acid by 25% or more in the standard in vitro assay. In certain embodiments, RNAi agents selectively affect one or more target nucleic acid. Such RNAi agents comprise a modified oligonucleotide having a nucleobase sequence that hybridizes to one or more target nucleic acid, resulting in one or more desired antisense activity. In certain embodiments, an RNAi agent comprises a modified oligonucleotide that does not hybridize to one or more non-target nucleic acid or does not hybridize to one or more non-target nucleic acid in such a way that results in significant undesired antisense activity.

In certain embodiments, hybridization of an antisense oligonucleotide to a target nucleic acid does not result in recruitment of a protein that cleaves that target nucleic acid. In certain embodiments, hybridization of the antisense oligonucleotide to the target nucleic acid results in alteration of splicing of the target nucleic acid. In certain embodiments, hybridization of an antisense oligonucleotide to a target nucleic acid results in exon inclusion or exon exclusion. In certain embodiments, hybridization of an antisense oligonucleotide to a target nucleic acid results in retained intron exclusion.

In certain embodiments, hybridization of an antisense oligonucleotide to a target nucleic acid results in inhibition of a binding interaction between the target nucleic acid and a protein or other nucleic acid (e.g., miRNA, lncRNA, sncRNA). In certain embodiments, hybridization of an antisense oligonucleotide to a target nucleic acid results in modulation of translation of the target nucleic acid. In certain embodiments, hybridization of an oligomeric agent to a target nucleic acid results in an increase in the amount or activity of a target nucleic acid. In certain embodiments, hybridization of an antisense oligonucleotide to a target nucleic acid results in increased translation of the target nucleic acid. In certain embodiments, hybridization of an antisense oligonucleotide to a target nucleic acid results in reduced translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein and/or a phenotypic change in a cell or animal.

### B. Treatment

In certain embodiments, provided herein are methods of reducing or inhibiting APOE expression or activity, which can be useful for treating or ameliorating a neurodegenerative disease associated with APOE in a subject. In certain embodiments, the neurodegenerative disease associated with APOE is Alzheimer's disease.

In certain embodiments, a method comprises administering to a subject an oligomeric agent, a modified oligonucleotide, an oligomeric duplex, or an antisense agent, any of which having a nucleobase sequence complementary to an APOE nucleic acid. In certain embodiments, the subject has or is at risk for developing a neurodegenerative disease associated with APOE. In certain embodiments, the subject has or is at risk for developing Alzheimer's disease. In certain embodiments, the subject has Alzheimer's disease.

In certain embodiments, a method for treating a neurodegenerative disease associated with APOE comprises administering to a subject an oligomeric agent, a modified oligonucleotide, an oligomeric duplex, or an antisense agent, any of which having a nucleobase sequence complementary to an APOE nucleic acid. In certain embodiments, the subject has or is at risk for developing a neurodegenerative disease. In certain embodiments, the subject has or is at risk for developing Alzheimer's disease. In certain embodiments, the subject has Alzheimer's disease. In certain embodiments, at least one symptom of the neurodegenerative disease associated with APOE is ameliorated. In certain embodiments, the symptom is cognitive impairment, progressive memory loss, a decline in language skills, behavioral abnormality, dementia, difficulty performing daily activities, aphasia, agnosia, apraxia, loss of motor function, amyloid plaque, neurofibrillary tangle, and/or neuroinflammation. In certain embodiments, administration of the oligomeric agent, modified oligonucleotide, oligomeric duplex, or antisense agent reduces the rate of cognitive impairment or progressive memory loss, reduces the rate of decline in language skills, reduces the rate of progression of behavioral abnormality, reduces the rate of progression of dementia, improves the performance in daily activities, reduces the rate of progression of aphasia, agnosia, and/or apraxia, and/or decreases the rate of decline in motor function in the subject. In certain embodiments, administration of the oligomeric agent, modified oligonucleotide, oligomeric duplex, or antisense agent reduces amyloid plaques, neurofibrillary tangles, and/or neuroinflammation in the brain of the subject.

In certain embodiments, a method of reducing expression of APOE, for example RNA, or reducing the expression of ApoE protein in a cell comprises contacting the cell with an oligomeric agent, a modified oligonucleotide, an oligomeric duplex, or an antisense agent, any of which having a nucleobase sequence complementary to an APOE nucleic acid. In certain embodiments, the subject has or is at risk for developing a neurodegenerative disease. In certain embodiments, the subject has or is at risk for developing Alzheimer's disease. In certain embodiments, the subject has Alzheimer's disease. In certain embodiments, the cell is a brain cell. In certain embodiments, the cell is a glial cell. In certain embodiments, the cell is an astrocyte, an oligodendrocyte, or a microglial cell. In certain embodiments, the cell is an astrocyte or a microglial cell. In certain embodiments, the cell is a neuron. In certain embodiments, the cell is an endothelial cell in the brain, e.g., a brain microvascular endothelial cell (BMEC). In certain embodiments, the cell is a pericyte in the brain (or brain pericyte). In certain embodiments, the cell is a human cell.

Certain embodiments are drawn to an oligomeric agent, a modified oligonucleotide, an oligomeric duplex, or an antisense agent, any of which having a nucleobase sequence complementary to an APOE nucleic acid, for use in treating a neurodegenerative disease associated with APOE or for use in the manufacturing of a medicament for treating a neurodegenerative disease associated with APOE. In certain embodiments, the neurodegenerative disease associated with APOE is Alzheimer's disease.

In any of the methods or uses described herein, the oligomeric agent, the modified oligonucleotide, the oligomeric duplex, or the antisense agent can be any described herein.

### IV. Pharmaceutical Compositions

In certain embodiments, described herein are pharmaceutical compositions comprising one or more oligomeric agent described herein, wherein each oligomeric agent comprises or consists of a modified oligonucleotide. In certain embodiments, the one or more oligomeric agent consists of or comprises an antisense oligonucleotide. In certain embodiments, a pharmaceutical composition comprises a pharmaceutically acceptable diluent or carrier. In certain embodiments, a pharmaceutical composition comprises or consists of a sterile saline solution and one or more oligomeric agent. In certain embodiments, the sterile saline is pharmaceutical grade saline. In certain embodiments, a pharmaceutical composition comprises or consists of one or more oligomeric agent (e.g., a modified oligonucleotide or oligomeric duplex) provided herein and sterile water. In certain embodiments, the sterile water is pharmaceutical grade water. In certain embodiments, a pharmaceutical composition comprises or consists of one or more oligomeric agent (e.g., a modified oligonucleotide or oligomeric duplex) provided herein and phosphate-buffered saline (PBS). In certain embodiments, sterile PBS is pharmaceutical grade PBS. In certain embodiments, a pharmaceutical composition comprises or consists of one or more oligomeric agent (e.g., a modified oligonucleotide or oligomeric duplex) provided herein and artificial cerebrospinal fluid (“artificial CSF” or “aCSF”). In certain embodiments, the artificial cerebrospinal fluid is pharmaceutical grade artificial cerebrospinal fluid.

In certain embodiments, a pharmaceutical composition comprises an oligomeric agent comprising or consisting of a modified oligonucleotide and sterile saline. In certain such embodiments, a pharmaceutical composition consists of such oligomeric agent and sterile saline. In certain embodiments, a pharmaceutical composition consists essentially of such oligomeric agent and sterile saline. In certain embodiments, the sterile saline is sterile PBS. In certain embodiments, the sterile saline is pharmaceutical grade.

In certain embodiments, a pharmaceutical composition comprises an oligomeric agent and artificial cerebrospinal fluid (aCSF). In certain embodiments, a pharmaceutical composition consists of an oligomeric agent and aCSF. In certain embodiments, a pharmaceutical composition consists essentially of an oligomeric agent and aCSF. In certain embodiments, the artificial cerebrospinal fluid is pharmaceutical grade. In certain embodiments, aCSF comprises sodium chloride, potassium chloride, sodium dihydrogen phosphate dihydrate, sodium phosphate dibasic anhydrous, calcium chloride dihydrate, and magnesium chloride hexahydrate. In certain embodiments, the pH of an aCSF solution is modulated with a suitable pH-adjusting agent, for example, with acids such as hydrochloric acid and alkalis such as sodium hydroxide, to a range of from about 7.1-7.3, or to about 7.2.

In certain embodiments, pharmaceutical compositions comprise one or more oligomeric agent and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, an oligomeric agent may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric agent encompass any pharmaceutically acceptable salts of the oligomeric agent, esters of the oligomeric agent, or salts of such esters. As used herein “pharmaceutically acceptable salts” means physiologically and pharmaceutically acceptable salts of compounds. Pharmaceutically acceptable salts retain the desired biological activity of the parent compound and do not impart undesired toxicological effects thereto. In certain embodiments, pharmaceutical compositions comprising an oligomeric agent comprising or consisting of one or more modified oligonucleotide, upon administration to a subject, including a human, are capable of providing (directly or indirectly) the biologically active metabolite or residue thereof. Accordingly, for example, the disclosure is also drawn to pharmaceutically acceptable salts of oligomeric agents provided herein, prodrugs, pharmaceutically acceptable salts of such prodrugs, and other bioequivalents. In certain embodiments, pharmaceutically acceptable salts comprise inorganic salts, such as monovalent or divalent inorganic salts. Suitable pharmaceutically acceptable salts include, but are not limited to, sodium, potassium, calcium, and magnesium salts. In certain embodiments, prodrugs comprise one or more conjugate group attached to an oligonucleotide, wherein the conjugate group is cleaved, for example by endogenous nucleases, within the body.

In certain embodiments, oligomeric agents are lyophilized and isolated, e.g., as sodium salts. In certain embodiments, a sodium salt of an oligomeric agent is mixed with a pharmaceutically acceptable diluent. In certain embodiments, the pharmaceutically acceptable diluent comprises sterile saline, sterile water, PBS. In certain embodiments, a sodium salt of an oligomeric agent is mixed with PBS.

Lipid moieties have been used in nucleic acid therapies in a variety of methods. In certain methods, a nucleic acid, such as an oligomeric agent comprising a modified oligonucleotide, is introduced into preformed liposomes or lipoplexes made of mixtures of cationic lipids and neutral lipids. In certain methods, nucleic acid complexes with mono- or poly-cationic lipids are formed without the presence of a neutral lipid. In certain embodiments, a lipid moiety is selected to increase distribution of an oligomeric agent to a particular cell or tissue. In certain embodiments, a lipid moiety is selected to increase distribution of an oligomeric agent to fat tissue. In certain embodiments, a lipid moiety is selected to increase distribution of an oligomeric agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more oligomeric agents to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV), etc.). In certain of such embodiments, a pharmaceutical composition comprises a carrier or diluent and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, diluents, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Under certain conditions, certain compounds disclosed herein act as acids. Although such compounds may be drawn or described in protonated (free acid) form or ionized and in association with a cation (salt) form, aqueous solutions of such compounds exist in equilibrium among such forms. For example, a phosphodiester linkage of an oligonucleotide in aqueous solution exists in equilibrium among free acid, anion and salt forms. Unless otherwise indicated, compounds described herein are intended to include all such forms. Moreover, certain oligonucleotides have several such linkages, each of which is in equilibrium. Thus, oligonucleotides in solution exist in an ensemble of forms at multiple positions all at equilibrium. The term “oligonucleotide” herein is intended to include all such forms. Drawn structures necessarily depict a single form. Nevertheless, unless otherwise indicated, such drawings are likewise intended to include corresponding forms. Herein, a structure depicting the free acid of a compound followed by the term “or a pharmaceutically acceptable salt thereof” expressly includes all such forms that may be fully or partially protonated/de-protonated/in association with a cation or a combination of cations. In certain embodiments, one or more specific cation is identified. The cations include, but are not limited to, sodium, potassium, calcium, and magnesium. In certain embodiments, a structure depicting the free acid of a compound followed by the term “or a pharmaceutically acceptable salt thereof” expressly includes all such forms that may be fully or partially protonated/de-protonated/in association with one or more cations selected from sodium, potassium, calcium, and magnesium.

In certain embodiments, oligomeric agents provided herein are in aqueous solution with sodium. In certain embodiments, oligomeric agents are in aqueous solution with potassium. In certain embodiments, oligomeric agents are in PBS. In certain embodiments, oligomeric agents are in water. In certain such embodiments, the pH of a solution is adjusted with NaOH and/or HCl to achieve a desired pH.

Herein, certain specific doses are described. A dose may be in the form of a dosage unit. For clarity, a dose (or dosage unit) of an oligomeric agent (e.g., modified oligonucleotide, oligomeric duplex, antisense agent) in milligrams indicates the mass of the free acid form of the modified oligonucleotide or oligomeric duplex. As described herein, in aqueous solution, the free acid is in equilibrium with anionic and salt forms. However, for the purpose of calculating dose, it is assumed that the oligomeric agent (e.g., modified oligonucleotide, oligomeric duplex) exists as a solvent-free, sodium-acetate free, anhydrous, free acid. In certain embodiments, where an oligomeric agent (e.g., modified oligonucleotide, oligomeric duplex) is in solution comprising sodium (e.g., saline), the oligomeric agent may be partially or fully de-protonated and in association with sodium ions. However, the mass of the protons is nevertheless counted toward the weight of the dose, and the mass of the sodium ions is not counted toward the weight of the dose. When an oligomeric agent comprises a conjugate group, the mass of the conjugate group is included in calculating the dose of such oligomeric agent. If the conjugate group also has an acid, the conjugate group is likewise assumed to be fully protonated for the purpose of calculating dose.

In certain embodiments, where a modified oligonucleotide or oligomeric agent is in a solution, such as aCSF, comprising sodium, potassium, calcium, and magnesium, the modified oligonucleotide or oligomeric agent may be partially or fully de-protonated and in association with sodium, potassium, calcium, and/or magnesium. However, the mass of the protons is nevertheless counted toward the weight of the dose, and the mass of the sodium, potassium, calcium, and magnesium ions is not counted toward the weight of the dose.

### V. Certain Oligomeric Agents

In certain embodiments, an oligomeric agent disclosed herein comprises a modified oligonucleotide consisting of 12 to 50 linked nucleosides, wherein the nucleobase sequence of the modified oligonucleotide comprises at least 12, at least 13, at least 14, at least 15, at least 16, at least 17, at least 18, at least 19, at least 20, or 21 contiguous nucleobases of any of the nucleobase sequences of SEQ ID NOs: 22-40. In certain such embodiments, the modified oligonucleotide comprises at least one modification selected from a modified sugar moiety and a modified internucleoside linkage. In certain embodiments, a T nucleobase in the modified oligonucleotide is substituted with a U nucleobase. In certain embodiments, two or more T nucleobases, three or more T nucleobases, or all of the T nucleobases are substituted with U nucleobases in the modified oligonucleotide. In certain embodiments, the oligomeric agent comprises a conjugate group. In certain embodiments, the oligomeric agent does not comprise a conjugate group. In certain embodiments, the oligomeric agent comprises a terminal group. In certain embodiments, the oligomeric agent does not comprise a terminal group. In certain embodiments, the oligomeric agent is single-stranded. In certain embodiments, the oligomeric agent is an antisense agent. In certain embodiments, the oligomeric agent is a single-stranded antisense agent.

In certain embodiments, an oligomeric agent disclosed herein comprises a modified oligonucleotide consisting of 12 to 50 linked nucleosides, wherein the nucleobase sequence of the modified oligonucleotide comprises at least 12, at least 13, at least 14, at least 15, at least 16, at least 17, at least 18, at least 19, or 20 contiguous nucleobases of 5′-ACTTGGTGAATCTTTATTAA-3′ (SEQ ID NO: 22). In certain embodiments, the modified oligonucleotide comprises at least one modification selected from a modified sugar moiety and a modified internucleoside linkage. In certain embodiments, the modified sugar moiety is selected from a 2′-MOE sugar moiety, a cEt sugar moiety, a 2′-OMe sugar moiety, and a 2′-β-D-deoxyxylosyl sugar moiety. In certain embodiments, the modified internucleoside linkage is selected from a phosphorothioate internucleoside linkage, a phosphodiester internucleoside linkage, and a mesyl phosphoramidate internucleoside linkage. In certain embodiments, each nucleobase of the modified oligonucleotide is an unmodified nucleobase. In certain embodiments, at least one nucleobase of the modified oligonucleotide is a modified nucleobase. In certain embodiments, a T nucleobase in the modified oligonucleotide is substituted with a U nucleobase. In certain embodiments, two or more T nucleobases, three or more T nucleobases, four or more T nucleobases, five or more T nucleobases, or all of the T nucleobases are substituted with U nucleobases in the modified oligonucleotide. In certain embodiments, the oligomeric agent comprises a conjugate group. In certain embodiments, the oligomeric agent does not comprise a conjugate group. In certain embodiments, the oligomeric agent comprises a terminal group. In certain embodiments, the oligomeric agent does not comprise a terminal group. In certain embodiments, the oligomeric agent is single-stranded. In certain embodiments, the oligomeric agent is an antisense agent. In certain embodiments, the oligomeric agent is a single-stranded antisense agent.

In certain embodiments, the modified oligonucleotide has a nucleobase sequence of SEQ ID NO: 22. In certain embodiments, the modified oligonucleotide has a modified sugar motif of 5′-eeeeekddddddddddkkee-3′, wherein each “e” is a 2′-MOE sugar moiety, each “k” is a cEt sugar moiety, and each “d” is a 2′-β-D-deoxyribosyl sugar moiety. In certain embodiments, the modified oligonucleotide comprises a modified internucleoside linkage selected from a phosphorothioate internucleoside linkage, a phosphodiester internucleoside linkage, and a mesyl phosphoramidate internucleoside linkage. In certain embodiments, each nucleobase of the modified oligonucleotide is an unmodified nucleobase. In certain embodiments, at least one nucleobase of the modified oligonucleotide is a modified nucleobase. In certain embodiments, at least one cytosine of the modified oligonucleotide is a modified cytosine. In certain embodiments, each cytosine of the modified oligonucleotide is a 5-methylcytosine.

In certain embodiments, the modified oligonucleotide has a nucleobase sequence of SEQ ID NO: 22, and the modified oligonucleotide has a modified sugar motif of 5′-eeeeekddddddddddkkee-3′, wherein each “e” is a 2′-MOE sugar moiety, each “k” is a cEt sugar moiety, and each “d” is a 2′-β-D-deoxyribosyl sugar moiety. In certain embodiments, the modified oligonucleotide has a modified internucleoside linkage motif of 5′-soosssszzzssssszsss-3′, wherein each “s” is a phosphorothioate internucleoside linkage, each “o” is a phosphodiester internucleoside linkage, and each “z” is a mesyl phosphoramidate internucleoside linkage. In certain embodiments, each nucleobase of the modified oligonucleotide is an unmodified nucleobase. In certain embodiments, at least one nucleobase of the modified oligonucleotide is a modified nucleobase. In certain embodiments, at least one cytosine of the modified oligonucleotide is a modified cytosine. In certain embodiments, each cytosine of the modified oligonucleotide is a 5-methylcytosine.

In certain embodiments, the modified oligonucleotide has a nucleobase sequence of SEQ ID NO: 22, and the modified oligonucleotide has a modified sugar motif of 5′-keeeekddddddddddkkee-3′, wherein each “e” is a 2′-MOE sugar moiety, each “k” is a cEt sugar moiety, and each “d” is a 2′-β-D-deoxyribosyl sugar moiety. In certain embodiments, the modified oligonucleotide comprises a modified internucleoside linkage selected from a phosphorothioate internucleoside linkage, a phosphodiester internucleoside linkage, and a mesyl phosphoramidate internucleoside linkage. In certain embodiments, each nucleobase of the modified oligonucleotide is an unmodified nucleobase. In certain embodiments, at least one nucleobase of the modified oligonucleotide is a modified nucleobase. In certain embodiments, at least one cytosine of the modified oligonucleotide is a modified cytosine. In certain embodiments, each cytosine of the modified oligonucleotide is a 5-methylcytosine.

In certain embodiments, the modified oligonucleotide has a nucleobase sequence of SEQ ID NO: 22, and the modified oligonucleotide has a modified sugar motif of 5′-keeeekddddddddddkkee-3′, wherein each “e” is a 2′-MOE sugar moiety, each “k” is a cEt sugar moiety, and each “d” is a 2′-β-D-deoxyribosyl sugar moiety. In certain embodiments, the modified oligonucleotide has a modified internucleoside linkage motif of 5′-soosssszzzssssszsss-3′, wherein each “s” is a phosphorothioate internucleoside linkage, each “o” is a phosphodiester internucleoside linkage, and each “z” is a mesyl phosphoramidate internucleoside linkage. In certain embodiments, each nucleobase of the modified oligonucleotide is an unmodified nucleobase. In certain embodiments, at least one nucleobase of the modified oligonucleotide is a modified nucleobase. In certain embodiments, at least one cytosine of the modified oligonucleotide is a modified cytosine. In certain embodiments, each cytosine of the modified oligonucleotide is a 5-methylcytosine.

In certain embodiments, an oligomeric agent disclosed herein comprises a modified oligonucleotide consisting of 12 to 50 linked nucleosides, wherein the nucleobase sequence of the modified oligonucleotide comprises at least 12, at least 13, at least 14, at least 15, at least 16, at least 17, at least 18, at least 19, or 20 contiguous nucleobases of 5′-CTTGGTGAATCTTTATTAAA-3′ (SEQ ID NO: 23). In certain embodiments, the modified oligonucleotide comprises at least one modification selected from a modified sugar moiety and a modified internucleoside linkage. In certain embodiments, the modified sugar moiety is selected from a 2′-MOE sugar moiety, a cEt sugar moiety, a 2′-OMe sugar moiety, and a 2′-β-D-deoxyxylosyl sugar moiety. In certain embodiments, the modified internucleoside linkage is selected from a phosphorothioate internucleoside linkage, a phosphodiester internucleoside linkage, and a mesyl phosphoramidate internucleoside linkage. In certain embodiments, each nucleobase of the modified oligonucleotide is an unmodified nucleobase. In certain embodiments, at least one nucleobase of the modified oligonucleotide is a modified nucleobase. In certain embodiments, a T nucleobase in the modified oligonucleotide is substituted with a U nucleobase. In certain embodiments, two or more T nucleobases, three or more T nucleobases, four or more T nucleobases, five or more T nucleobases, or all of the T nucleobases are substituted with U nucleobases in the modified oligonucleotide. In certain embodiments, the oligomeric agent comprises a conjugate group. In certain embodiments, the oligomeric agent does not comprise a conjugate group. In certain embodiments, the oligomeric agent comprises a terminal group. In certain embodiments, the oligomeric agent does not comprise a terminal group. In certain embodiments, the oligomeric agent is single-stranded. In certain embodiments, the oligomeric agent is an antisense agent. In certain embodiments, the oligomeric agent is a single-stranded antisense agent.

In certain embodiments, the modified oligonucleotide has a nucleobase sequence of SEQ ID NO: 23. In certain embodiments, the modified oligonucleotide has a modified sugar motif of 5′-kkkdddddddddddddkkdk-3′, wherein each “k” is a cEt sugar moiety, and each “d” is a 2′-β-D-deoxyribosyl sugar moiety. In certain embodiments, the modified oligonucleotide comprises a modified internucleoside linkage selected from a phosphorothioate internucleoside linkage, a phosphodiester internucleoside linkage, and a mesyl phosphoramidate internucleoside linkage. In certain embodiments, each nucleobase of the modified oligonucleotide is an unmodified nucleobase. In certain embodiments, at least one nucleobase of the modified oligonucleotide is a modified nucleobase. In certain embodiments, at least one cytosine of the modified oligonucleotide is a modified cytosine. In certain embodiments, each cytosine of the modified oligonucleotide is a 5-methylcytosine.

In certain embodiments, the modified oligonucleotide has a nucleobase sequence of SEQ ID NO: 23, and the modified oligonucleotide has a modified sugar motif of 5′-kkkdddddddddddddkkdk-3′, wherein each “k” is a cEt sugar moiety, and each “d” is a 2′-β-D-deoxyribosyl sugar moiety. In certain embodiments, the modified oligonucleotide has a modified internucleoside linkage motif of 5′-ssszzzzzzsssssssssz-3′, wherein each “s” is a phosphorothioate internucleoside linkage, and each “z” is a mesyl phosphoramidate internucleoside linkage. In certain embodiments, each nucleobase of the modified oligonucleotide is an unmodified nucleobase. In certain embodiments, at least one nucleobase of the modified oligonucleotide is a modified nucleobase. In certain embodiments, at least one cytosine of the modified oligonucleotide is a modified cytosine. In certain embodiments, each cytosine of the modified oligonucleotide is a 5-methylcytosine.

In certain embodiments, an oligomeric agent disclosed herein comprises a modified oligonucleotide consisting of 12 to 50 linked nucleosides, wherein the nucleobase sequence of the modified oligonucleotide comprises at least 12, at least 13, at least 14, at least 15, at least 16, at least 17, at least 18, at least 19, or 20 contiguous nucleobases of 5′-TTGGTGAATCTTTATTAAAC-3′ (SEQ ID NO: 27). In certain embodiments, the modified oligonucleotide comprises at least one modification selected from a modified sugar moiety and a modified internucleoside linkage. In certain embodiments, the modified sugar moiety is selected from a 2′-MOE sugar moiety, a cEt sugar moiety, a 2′-OMe sugar moiety, and a 2′-β-D-deoxyxylosyl sugar moiety. In certain embodiments, the modified internucleoside linkage is selected from a phosphorothioate internucleoside linkage, a phosphodiester internucleoside linkage, and a mesyl phosphoramidate internucleoside linkage. In certain embodiments, each nucleobase of the modified oligonucleotide is an unmodified nucleobase. In certain embodiments, at least one nucleobase of the modified oligonucleotide is a modified nucleobase. In certain embodiments, a T nucleobase in the modified oligonucleotide is substituted with a U nucleobase. In certain embodiments, two or more T nucleobases, three or more T nucleobases, four or more T nucleobases, five or more T nucleobases, or all of the T nucleobases are substituted with U nucleobases in the modified oligonucleotide. In certain embodiments, the oligomeric agent comprises a conjugate group. In certain embodiments, the oligomeric agent does not comprise a conjugate group. In certain embodiments, the oligomeric agent comprises a terminal group. In certain embodiments, the oligomeric agent does not comprise a terminal group. In certain embodiments, the oligomeric agent is single-stranded. In certain embodiments, the oligomeric agent is an antisense agent. In certain embodiments, the oligomeric agent is a single-stranded antisense agent.

In certain embodiments, the modified oligonucleotide has a nucleobase sequence of SEQ ID NO: 27. In certain embodiments, the modified oligonucleotide has a modified sugar motif of 5′-eeekddddddddddkkeeee-3′, wherein each “e” is a 2′-MOE sugar moiety, each “k” is a cEt sugar moiety, and each “d” is a 2′-β-D-deoxyribosyl sugar moiety. In certain embodiments, the modified oligonucleotide comprises a modified internucleoside linkage selected from a phosphorothioate internucleoside linkage, a phosphodiester internucleoside linkage, and a mesyl phosphoramidate internucleoside linkage. In certain embodiments, each nucleobase of the modified oligonucleotide is an unmodified nucleobase. In certain embodiments, at least one nucleobase of the modified oligonucleotide is a modified nucleobase. In certain embodiments, at least one cytosine of the modified oligonucleotide is a modified cytosine. In certain embodiments, each cytosine of the modified oligonucleotide is a 5-methylcytosine.

In certain embodiments, the modified oligonucleotide has a nucleobase sequence of SEQ ID NO: 27. In certain embodiments, the modified oligonucleotide has a modified sugar motif of 5′-eeekddddddddddkkeeee-3′, wherein each “e” is a 2′-MOE sugar moiety, each “k” is a cEt sugar moiety, and each “d” is a 2′-β-D-deoxyribosyl sugar moiety. In certain embodiments, the modified oligonucleotide has a modified internucleoside linkage motif of 5′-zoosszzzssssszsssss-3′, wherein each “s” is a phosphorothioate internucleoside linkage, each “o” is a phosphodiester internucleoside linkage, and each “z” is a mesyl phosphoramidate internucleoside linkage. In certain embodiments, each nucleobase of the modified oligonucleotide is an unmodified nucleobase. In certain embodiments, at least one nucleobase of the modified oligonucleotide is a modified nucleobase. In certain embodiments, at least one cytosine of the modified oligonucleotide is a modified cytosine. In certain embodiments, each cytosine of the modified oligonucleotide is a 5-methylcytosine.

In certain embodiments, disclosed herein is an oligomeric agent according to the following chemical notation: N¹ₑₛ^{m}CₑₒTₑₒTₑₛGₑₛGₖₛT_{ds}G_{dz}A_{dz}A_{dz}T_{ds}^{m}C_{ds}T_{ds}T_{ds}T_{ds}A_{dz}TₖₛTₖₛN³ₑₛN²ₑ (SEQ ID NO: 49), wherein:
A=an adenine nucleobase,
^{m}C=a 5-methylcytosine nucleobase,
G=a guanine nucleobase,
T=a thymine nucleobase,
N¹=an adenine nucleobase, a modified adenine, a hypoxanthine, an abasic sugar moiety, a terminal group, or is absent, wherein when N¹ is absent its sugar and internucleoside linkage are also absent,
N²=an adenine nucleobase, a modified adenine, a hypoxanthine, an abasic sugar moiety, a terminal group, or is absent, wherein when N² is absent its sugar and internucleoside linkage are also absent,
N³=an adenine nucleobase, a modified adenine, a hypoxanthine, an abasic sugar moiety, a terminal group, or is absent, wherein when N³ is absent its sugar and internucleoside linkage are also absent,
e=a 2′-MOE sugar moiety,
k=a cEt sugar moiety,
d=a 2′-β-D-deoxyribosyl sugar moiety,
s=a phosphorothioate internucleoside linkage,
o=a phosphodiester internucleoside linkage, and
z=a mesyl phosphoramidate internucleoside linkage; and
   wherein the oligomeric agent optionally comprises a conjugate group. In certain embodiments, N¹ is an adenine nucleobase. In certain embodiments, N¹ is an unmodified adenine. In certain embodiments, N¹ is a modified adenine. In certain embodiments, N¹ is a hypoxanthine. In certain embodiments, N¹ is an abasic sugar moiety. In certain embodiments, N¹ is a terminal group. In certain embodiments, N¹ is absent. In certain embodiments, N² is an adenine nucleobase. In certain embodiments, N² is an unmodified adenine. In certain embodiments, N² is a modified adenine. In certain embodiments, N² is a hypoxanthine. In certain embodiments, N² is an abasic sugar moiety. In certain embodiments, N² is a terminal group. In certain embodiments, N² is absent. In certain embodiments, N³ is an adenine nucleobase. In certain embodiments, N³ is an unmodified adenine. In certain embodiments, N³ is a modified adenine. In certain embodiments, N³ is a hypoxanthine. In certain embodiments, N³ is an abasic sugar moiety. In certain embodiments, N³ is a terminal group. In certain embodiments, N³ is absent. In certain embodiments, N¹ is an adenine nucleobase, an unmodified adenine, a modified adenine, a hypoxanthine, an abasic sugar moiety, a terminal group, or is absent, and N² is an adenine nucleobase, an unmodified adenine, a modified adenine, a hypoxanthine, an abasic sugar moiety, a terminal group, or is absent. In certain embodiments, N¹ is an adenine nucleobase and N² is an adenine nucleobase, an unmodified adenine, a modified adenine, a hypoxanthine, an abasic sugar moiety, a terminal group, or is absent. In certain embodiments, N¹ is an adenine nucleobase and N² is an unmodified adenine. In certain embodiments, N¹ is an adenine nucleobase and N² is a modified adenine. In certain embodiments, N¹ is an adenine nucleobase and N² is a hypoxanthine. In certain embodiments, N¹ is an adenine nucleobase and N² is an abasic sugar moiety. In certain embodiments, N¹ is an adenine nucleobase and N² is a terminal group. In certain embodiments, N¹ is an adenine nucleobase and N² is absent. In certain embodiments, N¹ is a modified adenine and N² is an unmodified adenine. In certain embodiments, N¹ is a modified adenine and N² is a modified adenine. In certain embodiments, N¹ is a modified adenine and N² is a hypoxanthine. In certain embodiments, N¹ is a modified adenine and N² is an abasic sugar moiety. In certain embodiments, N¹ is a modified adenine and N² is a terminal group. In certain embodiments, N¹ is a modified adenine and N² is absent. In certain embodiments, N¹ is a hypoxanthine and N² is an unmodified adenine. In certain embodiments, N¹ is a hypoxanthine and N² is a modified adenine. In certain embodiments, N¹ is a hypoxanthine and N² is a hypoxanthine. In certain embodiments, N¹ is a hypoxanthine and N² is an abasic sugar moiety. In certain embodiments, N¹ is a hypoxanthine and N² is a terminal group. In certain embodiments, N¹ is a hypoxanthine and N² is absent. In certain embodiments, N¹ is an abasic sugar moiety, a terminal group, or is absent, and N² is an unmodified adenine. In certain embodiments, N¹ is an abasic sugar moiety, a terminal group, or is absent, and N² is a modified adenine. In certain embodiments, N¹ is an abasic sugar moiety, a terminal group, or is absent, and N² is a hypoxanthine. In certain embodiments, N¹ is an abasic sugar moiety, a terminal group, or is absent, and N² is an abasic sugar moiety. In certain embodiments, N¹ is an abasic sugar moiety, a terminal group, or is absent, and N² is a terminal group. In certain embodiments, N¹ is an abasic sugar moiety, a terminal group, or is absent, and N² is absent. In certain embodiments, N¹ is an adenine nucleobase, an unmodified adenine, a modified adenine, a hypoxanthine, an abasic sugar moiety, a terminal group, or is absent; N² is absent; and N³ is an adenine nucleobase, an unmodified adenine, a modified adenine, a hypoxanthine, an abasic sugar moiety, a terminal group, or is absent. In certain embodiments, N¹ is an adenine nucleobase, N² is absent, and N³ is an unmodified adenine. In certain embodiments, N¹ is an adenine nucleobase, N² is absent, and N³ is a modified adenine. In certain embodiments, N¹ is an adenine nucleobase, N² is absent, and N³ is a hypoxanthine. In certain embodiments, N¹ is an adenine nucleobase, N² is absent, and N³ is an abasic sugar moiety. In certain embodiments, N¹ is an adenine nucleobase, N² is absent, and N³ is a terminal group. In certain embodiments, N¹ is an adenine nucleobase, N² is absent, and N³ is absent. In certain embodiments, N¹ is a modified adenine, N² is absent, and N³ is an unmodified adenine. In certain embodiments, N¹ is a modified adenine, N² is absent, and N³ is a modified adenine. In certain embodiments, N¹ is a modified adenine, N² is absent, and N³ is a hypoxanthine. In certain embodiments, N¹ is a modified adenine, N² is absent, and N³ is an abasic sugar moiety. In certain embodiments, N¹ is a modified adenine, N² is absent, and N³ is a terminal group. In certain embodiments, N¹ is a modified adenine, N² is absent, and N³ is absent. In certain embodiments, N¹ is a hypoxanthine, N² is absent, and N³ is an unmodified adenine. In certain embodiments, N¹ is a hypoxanthine, N² is absent, and N³ is a modified adenine. In certain embodiments, N¹ is a hypoxanthine, N² is absent, and N³ is a hypoxanthine. In certain embodiments, N¹ is a hypoxanthine, N² is absent, and N³ is an abasic sugar moiety. In certain embodiments, N¹ is a hypoxanthine, N² is absent, and N³ is a terminal group. In certain embodiments, N¹ is a hypoxanthine, N² is absent, and N³ is absent. In certain embodiments, N¹ is an abasic sugar moiety, a terminal group, or is absent, N² is absent, and N³ is an unmodified adenine. In certain embodiments, N¹ is an abasic sugar moiety, a terminal group, or is absent, N² is absent, and N³ is a modified adenine. In certain embodiments, N¹ is an abasic sugar moiety, a terminal group, or is absent, N² is absent, and N³ is a hypoxanthine. In certain embodiments, N¹ is an abasic sugar moiety, a terminal group, or is absent, N² is absent, and N³ is an abasic sugar moiety. In certain embodiments, N¹ is an abasic sugar moiety, a terminal group, or is absent, N² is absent, and N³ is a terminal group. In certain embodiments, N¹ is an abasic sugar moiety, a terminal group, or is absent, N² is absent, and N³ is absent. In certain embodiments, N¹ is a terminal group, N² is absent, and N³ is an unmodified adenine. In certain embodiments, N¹ is a terminal group, N² is absent, and N³ is a modified adenine. In certain embodiments, N¹ is a terminal group, N² is absent, and N³ is a hypoxanthine. In certain embodiments, N¹ is a terminal group, N² is absent, and N³ is an abasic sugar moiety. In certain embodiments, N¹ is a terminal group, N² is absent, and N³ is a terminal group. In certain embodiments, N¹ is a terminal group, N² is absent, and N³ is absent. In certain embodiments, N¹ is absent, N² is absent, and N³ is an unmodified adenine. In certain embodiments, N¹ is absent, N² is absent, and N³ is a modified adenine. In certain embodiments, N¹ is absent, N² is absent, and N³ is a hypoxanthine. In certain embodiments, N¹ is absent, N² is absent, and N³ is an abasic sugar moiety. In certain embodiments, N¹ is absent, N² is absent, and N³ is a terminal group. In certain embodiments, N¹ is absent, N² is absent, and N³ is absent. In certain embodiments, the oligomeric agent is single-stranded. In certain embodiments, the oligomeric agent is an antisense agent. In certain embodiments, the oligomeric agent is a single-stranded antisense agent.

In certain embodiments, disclosed herein is an oligomeric agent according to the following chemical notation: N⁵ₖₛTₖₛTₖₛG_{dz}G_{dz}T_{dz}G_{dz}A_{dz}A_{dz}T_{ds}^{m}C_{ds}T_{ds}T_{ds}T_{ds}A_{ds}T_{ds}TₖₛN⁴ₖₛN³_{dz}N²ₖ (SEQ ID NO: 50), wherein:
A=an adenine nucleobase,
^{m}C=a 5-methylcytosine nucleobase,
G=a guanine nucleobase,
T=a thymine nucleobase,
N²=an adenine nucleobase, a modified adenine, a hypoxanthine, an abasic sugar moiety, a terminal group, or is absent, wherein when N² is absent its sugar and internucleoside linkage are also absent,
N³=an adenine nucleobase, a modified adenine, a hypoxanthine, an abasic sugar moiety, a terminal group, or is absent, wherein when N³ is absent its sugar and internucleoside linkage are also absent,
N⁴=an adenine nucleobase, a modified adenine, a hypoxanthine, an abasic sugar moiety, a terminal group, or is absent, wherein when N⁴ is absent its sugar and internucleoside linkage are also absent,
N⁵=a cytosine nucleobase, a modified cytosine, an abasic sugar moiety, a terminal group, or is absent, wherein when N⁵ is absent its sugar is also absent,
k=a cEt sugar moiety,
d=a 2′-β-D-deoxyribosyl sugar moiety,
s=a phosphorothioate internucleoside linkage, and
z=a mesyl phosphoramidate internucleoside linkage; and
   wherein the oligomeric agent optionally comprises a conjugate group. In certain embodiments, N² is an adenine nucleobase. In certain embodiments, N² is an unmodified adenine. In certain embodiments, N² is a modified adenine. In certain embodiments, N² is a hypoxanthine. In certain embodiments, N² is an abasic sugar moiety. In certain embodiments, N² is a terminal group. In certain embodiments, N² is absent. In certain embodiments, N³ is an adenine nucleobase. In certain embodiments, N³ is an unmodified adenine. In certain embodiments, N³ is a modified adenine. In certain embodiments, N³ is a hypoxanthine. In certain embodiments, N³ is an abasic sugar moiety. In certain embodiments, N³ is a terminal group. In certain embodiments, N³ is absent. In certain embodiments, N⁴ is an adenine nucleobase. In certain embodiments, N⁴ is an unmodified adenine. In certain embodiments, N⁴ is a modified adenine. In certain embodiments, N⁴ is a hypoxanthine. In certain embodiments, N⁴ is an abasic sugar moiety. In certain embodiments, N⁴ is a terminal group. In certain embodiments, N⁴ is absent. In certain embodiments, N⁵ is a modified cytosine. In certain embodiments, N⁵ is 5-methylcytosine. In certain embodiments, N⁵ is an unmodified cytosine. In certain embodiments, N⁵ is an abasic sugar moiety. In certain embodiments, N⁵ is a terminal group. In certain embodiments, N⁵ is absent. In certain embodiments, N⁵ is a cytosine nucleobase, a modified cytosine, an abasic sugar moiety, a terminal group, or is absent, and N² is an adenine nucleobase, an unmodified adenine, a modified adenine, a hypoxanthine, an abasic sugar moiety, a terminal group, or is absent. In certain embodiments, N⁵ is a cytosine nucleobase and N² is an unmodified adenine. In certain embodiments, N⁵ is a cytosine nucleobase and N² is a modified adenine. In certain embodiments, N⁵ is a cytosine nucleobase and N² is a hypoxanthine. In certain embodiments, N⁵ is a cytosine nucleobase and N² is an abasic sugar moiety. In certain embodiments, N⁵ is a cytosine nucleobase and N² is a terminal group. In certain embodiments, N⁵ is a cytosine nucleobase and N² is absent. In certain embodiments, N⁵ is a modified cytosine and N² is an unmodified adenine. In certain embodiments, N⁵ is a modified cytosine and N² is a modified adenine. In certain embodiments, N⁵ is a modified cytosine and N² is a hypoxanthine. In certain embodiments, N⁵ is a modified cytosine and N² is an abasic sugar moiety. In certain embodiments, N⁵ is a modified cytosine and N² is a terminal group. In certain embodiments, N⁵ is a modified cytosine and N² is absent. In certain embodiments, N⁵ is an abasic sugar moiety, a terminal group, or is absent, and N² is an unmodified adenine. In certain embodiments, N⁵ is an abasic sugar moiety, a terminal group, or is absent, and N² is a modified adenine. In certain embodiments, N⁵ is an abasic sugar moiety, a terminal group, or is absent, and N² is a hypoxanthine. In certain embodiments, N⁵ is an abasic sugar moiety, a terminal group, or is absent, and N² is an abasic sugar moiety. In certain embodiments, N⁵ is an abasic sugar moiety, a terminal group, or is absent, and N² is a terminal group. In certain embodiments, N⁵ is an abasic sugar moiety, a terminal group, or is absent, and N² is absent. In certain embodiments, N⁵ is a cytosine nucleobase, a modified cytosine, an abasic sugar moiety, a terminal group, or is absent; N² is absent; and N³ is an adenine nucleobase, an unmodified adenine, a modified adenine, a hypoxanthine, an abasic sugar moiety, a terminal group, or is absent. In certain embodiments, N⁵ is a cytosine nucleobase, N² is absent, and N³ is an unmodified adenine. In certain embodiments, N⁵ is a cytosine nucleobase, N² is absent, and N³ is a modified adenine. In certain embodiments, N⁵ is a cytosine nucleobase, N² is absent, and N³ is a hypoxanthine. In certain embodiments, N⁵ is a cytosine nucleobase, N² is absent, and N³ is an abasic sugar moiety. In certain embodiments, N⁵ is a cytosine nucleobase, N² is absent, and N³ is a terminal group. In certain embodiments, N⁵ is a cytosine nucleobase, N² is absent, and N³ is absent. In certain embodiments, N⁵ is a modified cytosine, N² is absent, and N³ is an unmodified adenine. In certain embodiments, N⁵ is a modified cytosine, N² is absent, and N³ is a modified adenine. In certain embodiments, N⁵ is a modified cytosine, N² is absent, and N³ is a hypoxanthine. In certain embodiments, N⁵ is a modified cytosine, N² is absent, and N³ is an abasic sugar moiety. In certain embodiments, N⁵ is a modified cytosine, N² is absent, and N³ is a terminal group. In certain embodiments, N⁵ is a modified cytosine, N² is absent, and N³ is absent. In certain embodiments, N⁵ is an abasic sugar moiety, a terminal group, or is absent, N² is absent, and N³ is an unmodified adenine. In certain embodiments, N⁵ is an abasic sugar moiety, a terminal group, or is absent, N² is absent, and N³ is a modified adenine. In certain embodiments, N⁵ is an abasic sugar moiety, a terminal group, or is absent, N² is absent, and N³ is a hypoxanthine. In certain embodiments, N⁵ is an abasic sugar moiety, a terminal group, or is absent, N² is absent, and N³ is an abasic sugar moiety. In certain embodiments, N⁵ is an abasic sugar moiety, a terminal group, or is absent, N² is absent, and N³ is a terminal group. In certain embodiments, N⁵ is an abasic sugar moiety, a terminal group, or is absent, N² is absent, and N³ is absent. In certain embodiments, N⁵ is absent, N² is absent, and N³ is an unmodified adenine. In certain embodiments, N⁵ is absent, N² is absent, and N³ is a modified adenine. In certain embodiments, N⁵ is absent, N² is absent, and N³ is a hypoxanthine. In certain embodiments, N⁵ is absent, N² is absent, and N³ is an abasic sugar moiety. In certain embodiments, N⁵ is absent, N² is absent, and N³ is a terminal group. In certain embodiments, N⁵ is absent, N² is absent, and N³ is absent. In certain embodiments, N⁵ is a cytosine nucleobase, a modified cytosine, an abasic sugar moiety, a terminal group, or is absent; N² is absent; N³ is absent; and N⁴ is an adenine nucleobase, an unmodified adenine, a modified adenine, a hypoxanthine, an abasic sugar moiety, a terminal group, or is absent. In certain embodiments, N⁵ is a cytosine nucleobase, N² is absent, N³ is absent, and N⁴ is an unmodified adenine. In certain embodiments, N⁵ is a cytosine nucleobase, N² is absent, N³ is absent, and N⁴ is a modified adenine. In certain embodiments, N⁵ is a cytosine nucleobase, N² is absent, N³ is absent, and N⁴ is a hypoxanthine. In certain embodiments, N⁵ is a cytosine nucleobase, N² is absent, N³ is absent, and N⁴ is an abasic sugar moiety. In certain embodiments, N⁵ is a cytosine nucleobase, N² is absent, N³ is absent, and N⁴ is a terminal group. In certain embodiments, N⁵ is a cytosine nucleobase, N² is absent, N³ is absent, and N⁴ is absent. In certain embodiments, N⁵ is a modified cytosine, N² is absent, N³ is absent, and N⁴ is an unmodified adenine. In certain embodiments, N⁵ is a modified cytosine, N² is absent, N³ is absent, and N⁴ is a modified adenine. In certain embodiments, N⁵ is a modified cytosine, N² is absent, N³ is absent, and N⁴ is a hypoxanthine. In certain embodiments, N⁵ is a modified cytosine, N² is absent, N³ is absent, and N⁴ is an abasic sugar moiety. In certain embodiments, N⁵ is a modified cytosine, N² is absent, N³ is absent, and N⁴ is a terminal group. In certain embodiments, N⁵ is a modified cytosine, N² is absent, N³ is absent, and N⁴ is absent. In certain embodiments, N⁵ is an abasic sugar moiety, a terminal group, or is absent, N² is absent, N³ is absent, and N⁴ is an unmodified adenine. In certain embodiments, N⁵ is an abasic sugar moiety, a terminal group, or is absent, N² is absent, N³ is absent, and N⁴ is a modified adenine. In certain embodiments, N⁵ is an abasic sugar moiety, a terminal group, or is absent, N² is absent, N³ is absent, and N⁴ is a hypoxanthine. In certain embodiments, N⁵ is an abasic sugar moiety, a terminal group, or is absent, N² is absent, N³ is absent, and N⁴ is an abasic sugar moiety. In certain embodiments, N⁵ is an abasic sugar moiety, a terminal group, or is absent, N² is absent, N³ is absent, and N⁴ is a terminal group. In certain embodiments, N⁵ is an abasic sugar moiety, a terminal group, or is absent, N² is absent, N³ is absent, and N⁴ is absent. In certain embodiments, N⁵ is absent, N² is absent, N³ is absent, and N⁴ is an unmodified adenine. In certain embodiments, N⁵ is absent, N² is absent, N³ is absent, and N⁴ is a modified adenine. In certain embodiments, N⁵ is absent, N² is absent, N³ is absent, and N⁴ is a hypoxanthine. In certain embodiments, N⁵ is absent, N² is absent, N³ is absent, and N⁴ is an abasic sugar moiety. In certain embodiments, N⁵ is absent, N² is absent, N³ is absent, and N⁴ is a terminal group. In certain embodiments, N⁵ is absent, N² is absent, N³ is absent, and N⁴ is absent. In certain embodiments, the oligomeric agent is single-stranded. In certain embodiments, the oligomeric agent is an antisense agent. In certain embodiments, the oligomeric agent is a single-stranded antisense agent.

In certain embodiments, disclosed herein is an oligomeric agent according to the following chemical notation: N¹ₖₛ^{m}CₑₒTₑₒTₑₛGₑₛGₖₛT_{ds}G_{dz}A_{dz}A_{dz}T_{ds}^{m}C_{ds}T_{ds}T_{ds}T_{ds}A_{dz}TₖₛTₖₛN³ₑₛN²ₑ (SEQ ID NO: 51), wherein:
A=an adenine nucleobase,
^{m}C=a 5-methylcytosine nucleobase,
G=a guanine nucleobase,
T=a thymine nucleobase,
N¹=an adenine nucleobase, a modified adenine, a hypoxanthine, an abasic sugar moiety, a terminal group, or is absent, wherein when N¹ is absent its sugar and internucleoside linkage are also absent,
N²=an adenine nucleobase, a modified adenine, a hypoxanthine, an abasic sugar moiety, a terminal group, or is absent, wherein when N² is absent its sugar and internucleoside linkage are also absent,
N³=an adenine nucleobase, a modified adenine, a hypoxanthine, an abasic sugar moiety, a terminal group, or is absent, wherein when N³ is absent its sugar and internucleoside linkage are also absent,
e=a 2′-MOE sugar moiety,
k=a cEt sugar moiety,
d=a 2′-β-D-deoxyribosyl sugar moiety,
s=a phosphorothioate internucleoside linkage,
o=a phosphodiester internucleoside linkage, and
z=a mesyl phosphoramidate internucleoside linkage; and
   wherein the oligomeric agent optionally comprises a conjugate group. In certain embodiments, N¹ is an adenine nucleobase. In certain embodiments, N¹ is an unmodified adenine. In certain embodiments, N¹ is a modified adenine. In certain embodiments, N¹ is a hypoxanthine. In certain embodiments, N¹ is an abasic sugar moiety. In certain embodiments, N¹ is a terminal group. In certain embodiments, N¹ is absent. In certain embodiments, N² is an adenine nucleobase. In certain embodiments, N² is an unmodified adenine. In certain embodiments, N² is a modified adenine. In certain embodiments, N² is a hypoxanthine. In certain embodiments, N² is an abasic sugar moiety. In certain embodiments, N² is a terminal group. In certain embodiments, N² is absent. In certain embodiments, N³ is an adenine nucleobase. In certain embodiments, N³ is an unmodified adenine. In certain embodiments, N³ is a modified adenine. In certain embodiments, N³ is a hypoxanthine. In certain embodiments, N³ is an abasic sugar moiety. In certain embodiments, N³ is a terminal group. In certain embodiments, N³ is absent. In certain embodiments, N¹ is an adenine nucleobase, an unmodified adenine, a modified adenine, a hypoxanthine, an abasic sugar moiety, a terminal group, or is absent, and N² is an adenine nucleobase, an unmodified adenine, a modified adenine, a hypoxanthine, an abasic sugar moiety, a terminal group, or is absent. In certain embodiments, N¹ is an adenine nucleobase and N² is an adenine nucleobase, an unmodified adenine, a modified adenine, a hypoxanthine, an abasic sugar moiety, a terminal group, or is absent. In certain embodiments, N¹ is an adenine nucleobase and N² is an unmodified adenine. In certain embodiments, N¹ is an adenine nucleobase and N² is a modified adenine. In certain embodiments, N¹ is an adenine nucleobase and N² is a hypoxanthine. In certain embodiments, N¹ is an adenine nucleobase and N² is an abasic sugar moiety. In certain embodiments, N¹ is an adenine nucleobase and N² is a terminal group. In certain embodiments, N¹ is an adenine nucleobase and N² is absent. In certain embodiments, N¹ is a modified adenine and N² is an unmodified adenine. In certain embodiments, N¹ is a modified adenine and N² is a modified adenine. In certain embodiments, N¹ is a modified adenine and N² is a hypoxanthine. In certain embodiments, N¹ is a modified adenine and N² is an abasic sugar moiety. In certain embodiments, N¹ is a modified adenine and N² is a terminal group. In certain embodiments, N¹ is a modified adenine and N² is absent. In certain embodiments, N¹ is a hypoxanthine and N² is an unmodified adenine. In certain embodiments, N¹ is a hypoxanthine and N² is a modified adenine. In certain embodiments, N¹ is a hypoxanthine and N² is a hypoxanthine. In certain embodiments, N¹ is a hypoxanthine and N² is an abasic sugar moiety. In certain embodiments, N¹ is a hypoxanthine and N² is a terminal group. In certain embodiments, N¹ is a hypoxanthine and N² is absent. In certain embodiments, N¹ is an abasic sugar moiety, a terminal group, or is absent, and N² is an unmodified adenine. In certain embodiments, N¹ is an abasic sugar moiety, a terminal group, or is absent, and N² is a modified adenine. In certain embodiments, N¹ is an abasic sugar moiety, a terminal group, or is absent, and N² is a hypoxanthine. In certain embodiments, N¹ is an abasic sugar moiety, a terminal group, or is absent, and N² is an abasic sugar moiety. In certain embodiments, N¹ is an abasic sugar moiety, a terminal group, or is absent, and N² is a terminal group. In certain embodiments, N¹ is an abasic sugar moiety, a terminal group, or is absent, and N² is absent. In certain embodiments, N¹ is an adenine nucleobase, an unmodified adenine, a modified adenine, a hypoxanthine, an abasic sugar moiety, a terminal group, or is absent; N² is absent; and N³ is an adenine nucleobase, an unmodified adenine, a modified adenine, a hypoxanthine, an abasic sugar moiety, a terminal group, or is absent. In certain embodiments, N¹ is an adenine nucleobase, N² is absent, and N³ is an unmodified adenine. In certain embodiments, N¹ is an adenine nucleobase, N² is absent, and N³ is a modified adenine. In certain embodiments, N¹ is an adenine nucleobase, N² is absent, and N³ is a hypoxanthine. In certain embodiments, N¹ is an adenine nucleobase, N² is absent, and N³ is an abasic sugar moiety. In certain embodiments, N¹ is an adenine nucleobase, N² is absent, and N³ is a terminal group. In certain embodiments, N¹ is an adenine nucleobase, N² is absent, and N³ is absent. In certain embodiments, N¹ is a modified adenine, N² is absent, and N³ is an unmodified adenine. In certain embodiments, N¹ is a modified adenine, N² is absent, and N³ is a modified adenine. In certain embodiments, N¹ is a modified adenine, N² is absent, and N³ is a hypoxanthine. In certain embodiments, N¹ is a modified adenine, N² is absent, and N³ is an abasic sugar moiety. In certain embodiments, N¹ is a modified adenine, N² is absent, and N³ is a terminal group. In certain embodiments, N¹ is a modified adenine, N² is absent, and N³ is absent. In certain embodiments, N¹ is a hypoxanthine, N² is absent, and N³ is an unmodified adenine. In certain embodiments, N¹ is a hypoxanthine, N² is absent, and N³ is a modified adenine. In certain embodiments, N¹ is a hypoxanthine, N² is absent, and N³ is a hypoxanthine. In certain embodiments, N¹ is a hypoxanthine, N² is absent, and N³ is an abasic sugar moiety. In certain embodiments, N¹ is a hypoxanthine, N² is absent, and N³ is a terminal group. In certain embodiments, N¹ is a hypoxanthine, N² is absent, and N³ is absent. In certain embodiments, N¹ is an abasic sugar moiety, a terminal group, or is absent, N² is absent, and N³ is an unmodified adenine. In certain embodiments, N¹ is an abasic sugar moiety, a terminal group, or is absent, N² is absent, and N³ is a modified adenine. In certain embodiments, N¹ is an abasic sugar moiety, a terminal group, or is absent, N² is absent, and N³ is a hypoxanthine. In certain embodiments, N¹ is an abasic sugar moiety, a terminal group, or is absent, N² is absent, and N³ is an abasic sugar moiety. In certain embodiments, N¹ is an abasic sugar moiety, a terminal group, or is absent, N² is absent, and N³ is a terminal group. In certain embodiments, N¹ is an abasic sugar moiety, a terminal group, or is absent, N² is absent, and N³ is absent. In certain embodiments, N¹ is a terminal group, N² is absent, and N³ is an unmodified adenine. In certain embodiments, N¹ is a terminal group, N² is absent, and N³ is a modified adenine. In certain embodiments, N¹ is a terminal group, N² is absent, and N³ is a hypoxanthine. In certain embodiments, N¹ is a terminal group, N² is absent, and N³ is an abasic sugar moiety. In certain embodiments, N¹ is a terminal group, N² is absent, and N³ is a terminal group. In certain embodiments, N¹ is a terminal group, N² is absent, and N³ is absent. In certain embodiments, N¹ is absent, N² is absent, and N³ is an unmodified adenine. In certain embodiments, N¹ is absent, N² is absent, and N³ is a modified adenine. In certain embodiments, N¹ is absent, N² is absent, and N³ is a hypoxanthine. In certain embodiments, N¹ is absent, N² is absent, and N³ is an abasic sugar moiety. In certain embodiments, N¹ is absent, N² is absent, and N³ is a terminal group. In certain embodiments, N¹ is absent, N² is absent, and N³ is absent. In certain embodiments, the oligomeric agent is single-stranded. In certain embodiments, the oligomeric agent is an antisense agent. In certain embodiments, the oligomeric agent is a single-stranded antisense agent.

In certain embodiments, disclosed herein is an oligomeric agent comprising a modified oligonucleotide according to the following chemical structure:

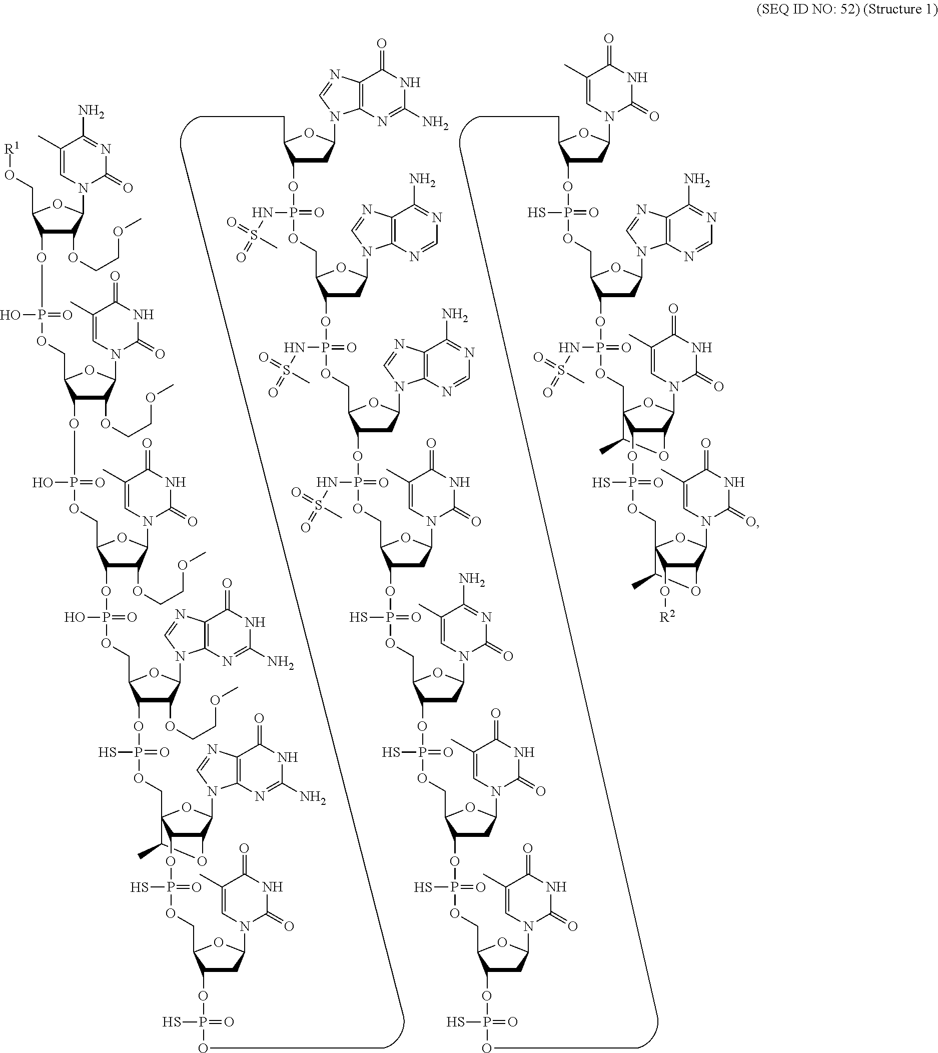

or a pharmaceutically acceptable salt thereof; wherein R¹ and R² are each independently 1, 2, or 3 linked nucleosides, an abasic nucleoside, a terminal group, or an H; and wherein the oligomeric agent optionally comprises a conjugate group. In certain embodiments, R¹ is one nucleoside. In certain embodiments, R¹ is 2 linked nucleosides. In certain embodiments, R¹ is 3 linked nucleosides. In certain embodiments, R² is one nucleoside. In certain embodiments, R² is 2 linked nucleosides. In certain embodiments, R² is 3 linked nucleosides. In certain embodiments, R¹ is one nucleoside and R² is 2 linked nucleosides. In certain embodiments, R¹ is one nucleoside and R² is 3 linked nucleosides. In certain embodiments, R¹ is an adenosine, a modified adenosine, an unmodified adenosine, or an inosine; and R² is 2 or 3 linked nucleosides. In certain embodiments, R¹ is an adenosine and R² comprises an adenosine, a modified adenosine, an unmodified adenosine, or an inosine. In certain embodiments, R¹ is an adenosine and R² comprises two or more adenosines, two or more modified adenosines, two or more unmodified adenosines, or two or more inosines. In certain embodiments, R¹ is an adenosine and R² is an abasic nucleoside, a terminal group, or an H. In certain embodiments, R¹ is a modified adenosine and R² comprises an adenosine, a modified adenosine, an unmodified adenosine, or an inosine. In certain embodiments, R¹ is a modified adenosine and R² comprises two or more adenosines, two or more modified adenosines, two or more unmodified adenosines, or two or more inosines. In certain embodiments, R¹ is a modified adenosine and R² is an abasic nucleoside, a terminal group, or an H. In certain embodiments, R¹ is an unmodified adenosine and R² comprises an adenosine, a modified adenosine, an unmodified adenosine, or an inosine. In certain embodiments, R¹ is an unmodified adenosine and R² comprises two or more adenosines, two or more modified adenosines, two or more unmodified adenosines, or two or more inosines. In certain embodiments, R¹ is an unmodified adenosine and R² is an abasic nucleoside, a terminal group, or an H. In certain embodiments, R¹ is an inosine and R² comprises an adenosine, a modified adenosine, an unmodified adenosine, or an inosine. In certain embodiments, R¹ is an inosine and R² comprises two or more adenosines, two or more modified adenosines, two or more unmodified adenosines, or two or more inosines. In certain embodiments, R¹ is an inosine and R² is an abasic nucleoside, a terminal group, or an H. In certain embodiments, R¹ is an abasic nucleoside, a terminal group, or an H; and R² comprises an adenosine, a modified adenosine, an unmodified adenosine, or an inosine. In certain embodiments, R¹ is an abasic nucleoside, a terminal group, or an H; and R² comprises two or more adenosines, two or more modified adenosines, two or more unmodified adenosines, or two or more inosines. In certain embodiments, R¹ is an abasic nucleoside, a terminal group, or an H; and R² is an abasic nucleoside, a terminal group, or an H. In certain embodiments, R¹ is an abasic nucleoside; and R² comprises an adenosine, a modified adenosine, an unmodified adenosine, or an inosine. In certain embodiments, R¹ is an abasic nucleoside; and R² comprises two or more adenosines, two or more modified adenosines, two or more unmodified adenosines, or two or more inosines. In certain embodiments, R¹ is an abasic nucleoside; and R² is an abasic nucleoside, a terminal group, or an H. In certain embodiments, R¹ is an H; and R² comprises an adenosine, a modified adenosine, an unmodified adenosine, or an inosine. In certain embodiments, R¹ is an H; and R² comprises two or more adenosines, two or more modified adenosines, two or more unmodified adenosines, or two or more inosines. In certain embodiments, R¹ is an H; and R² is an abasic nucleoside, a terminal group, or an H. In certain embodiments, R¹ is an H and R² is an H. In certain embodiments, R¹ is 2 or 3 linked nucleosides, optionally comprising an adenosine, a modified adenosine, an unmodified adenosine, or an inosine; and R² comprises an adenosine, a modified adenosine, an unmodified adenosine, or an inosine. In certain embodiments, R¹ is 2 or 3 linked nucleosides, optionally comprising an adenosine, a modified adenosine, an unmodified adenosine, or an inosine; and R² is an abasic nucleoside, a terminal group, or an H. In certain embodiments, R¹ is 2 or 3 linked nucleosides, optionally comprising an adenosine, a modified adenosine, an unmodified adenosine, or an inosine; and R² comprises an adenosine. In certain embodiments, R¹ is 2 or 3 linked nucleosides, optionally comprising an adenosine, a modified adenosine, an unmodified adenosine, or an inosine; and R² comprises a modified adenosine. In certain embodiments, R¹ is 2 or 3 linked nucleosides, optionally comprising an adenosine, a modified adenosine, an unmodified adenosine, or an inosine; and R² comprises an unmodified adenosine. In certain embodiments, R¹ is 2 or 3 linked nucleosides, optionally comprising an adenosine, a modified adenosine, an unmodified adenosine, or an inosine; and R² comprises an inosine. In certain embodiments, R¹ is 2 or 3 linked nucleosides, optionally comprising an adenosine, a modified adenosine, an unmodified adenosine, or an inosine; and R² an abasic nucleoside. In certain embodiments, R¹ is 2 or 3 linked nucleosides, optionally comprising an adenosine, a modified adenosine, an unmodified adenosine, or an inosine; and R² an H. In certain embodiments, if R¹ and R² are each independently 1, 2, or 3 linked nucleosides or are each independently an abasic nucleoside, R¹ and R² also include an internucleoside linkage selected from a phosphodiester internucleoside linkage, a phosphorothioate internucleoside linkage, and a mesyl phosphoramidate internucleoside linkage. In certain embodiments, the modified oligonucleotide is a pharmaceutically acceptable salt comprising one or more cations selected from sodium, potassium, calcium, and magnesium. In certain embodiments, the oligomeric agent is single-stranded. In certain embodiments, the oligomeric agent is an antisense agent. In certain embodiments, the oligomeric agent is a single-stranded antisense agent.

In certain embodiments, disclosed herein is an oligomeric agent comprising a modified oligonucleotide according to the following chemical structure:

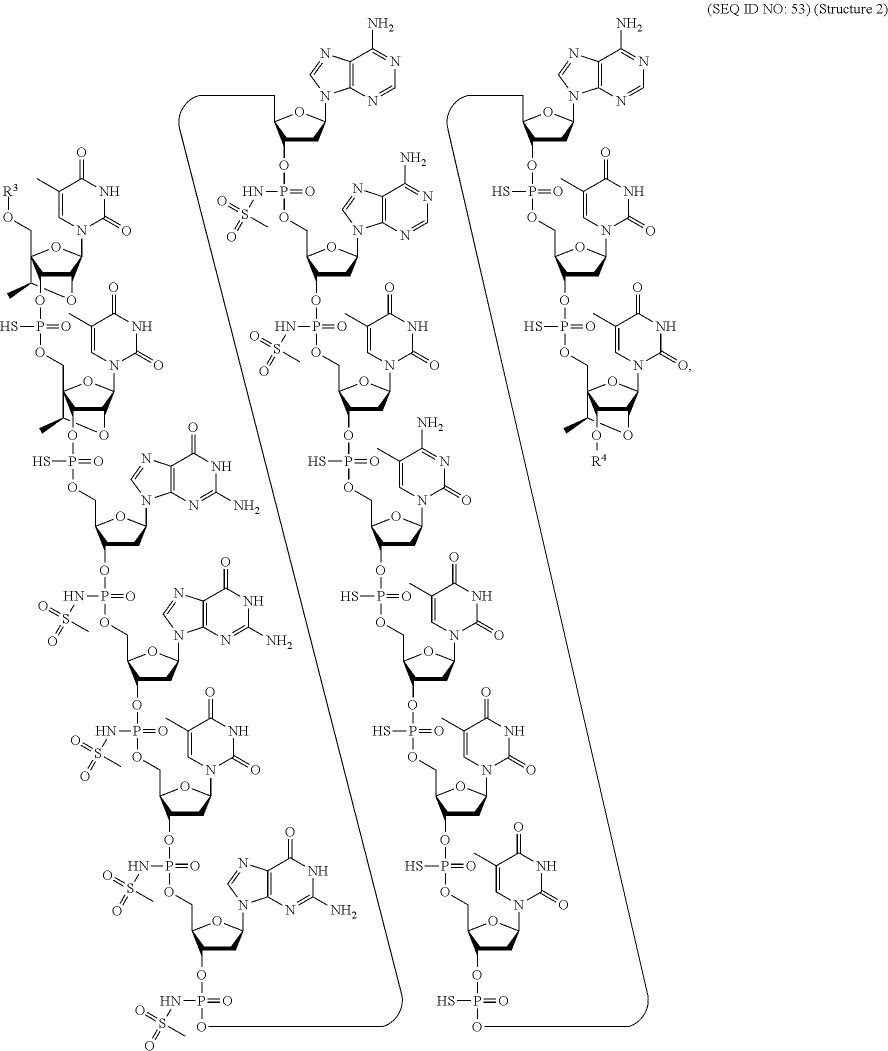

or a pharmaceutically acceptable salt thereof; wherein R³ and R⁴ are each independently 1, 2, or 3 linked nucleosides, an abasic nucleoside, a terminal group, or an H; and wherein the oligomeric agent optionally comprises a conjugate group. In certain embodiments, R³ is one nucleoside.

In certain embodiments, R³ is 2 linked nucleosides. In certain embodiments, R³ is 3 linked nucleosides. In certain embodiments, R⁴ is one nucleoside. In certain embodiments, R⁴ is 2 linked nucleosides. In certain embodiments, R⁴ is 3 linked nucleosides. In certain embodiments, R³ is one nucleoside and R⁴ is 2 or 3 linked nucleosides. In certain embodiments, R³ is a cytidine, a modified cytidine, or an unmodified cytidine, and R⁴ comprises an adenosine, a modified adenosine, an unmodified adenosine, or an inosine. In certain embodiments, R³ is a cytidine and R⁴ comprises an adenosine, a modified adenosine, an unmodified adenosine, or an inosine. In certain embodiments, R³ is a cytidine and R⁴ comprises two or more adenosines, two or more modified adenosines, two or more unmodified adenosines, or two or more inosines. In certain embodiments, R³ is a cytidine and R⁴ is an abasic nucleoside, a terminal group, or an H. In certain embodiments, R³ is a modified cytidine and R⁴ comprises an adenosine, a modified adenosine, an unmodified adenosine, or an inosine. In certain embodiments, R³ is a modified cytidine and R⁴ comprises two or more adenosines, two or more modified adenosines, two or more unmodified adenosines, or two or more inosines. In certain embodiments, R³ is a modified cytidine and R⁴ is an abasic nucleoside, a terminal group, or an H. In certain embodiments, R³ is a 5-methylcytidine and R⁴ comprises an adenosine, a modified adenosine, an unmodified adenosine, or an inosine. In certain embodiments, R³ is a 5-methylcytidine and R⁴ comprises two or more adenosines, two or more modified adenosines, two or more unmodified adenosines, or two or more inosines. In certain embodiments, R³ is a 5-methylcytidine and R⁴ is an abasic nucleoside, a terminal group, or an H. In certain embodiments, R³ is an unmodified cytidine and R⁴ comprises an adenosine, a modified adenosine, an unmodified adenosine, or an inosine. In certain embodiments, R³ is an unmodified cytidine and R⁴ comprises two or more adenosines, two or more modified adenosines, two or more unmodified adenosines, or two or more inosines. In certain embodiments, R³ is an unmodified cytidine and R⁴ is an abasic nucleoside, a terminal group, or an H. In certain embodiments, R³ is an abasic site and R⁴ is an abasic nucleoside, a terminal group, or an H. In certain embodiments, R³ is a terminal group and R⁴ is an abasic nucleoside, a terminal group, or an H. In certain embodiments, R³ is an H and R⁴ is an abasic nucleoside, a terminal group, or an H. In certain embodiments, R³ is an H and R⁴ is an H. In certain embodiments, R³ is 2 or 3 linked nucleosides, optionally comprises a cytidine, a modified cytidine, or an unmodified cytidine, and R⁴ comprises an adenosine, a modified adenosine, an unmodified adenosine, or an inosine. In certain embodiments, R³ is 2 or 3 linked nucleosides, optionally comprises a cytidine, a modified cytidine, or an unmodified cytidine, and R⁴ is an abasic nucleoside, a terminal group, or an H. In certain embodiments, R³ is 2 or 3 linked nucleosides, optionally comprises a cytidine, a modified cytidine, or an unmodified cytidine, and R⁴ comprises an adenosine. In certain embodiments, R³ is 2 or 3 linked nucleosides, optionally comprises a cytidine, a modified cytidine, or an unmodified cytidine, and R⁴ comprises a modified adenosine. In certain embodiments, R³ is 2 or 3 linked nucleosides, optionally comprises a cytidine, a modified cytidine, or an unmodified cytidine, and R⁴ comprises an unmodified adenosine. In certain embodiments, R³ is 2 or 3 linked nucleosides, optionally comprises a cytidine, a modified cytidine, or an unmodified cytidine, and R⁴ comprises an inosine. In certain embodiments, R³ is 2 or 3 linked nucleosides, optionally comprises a cytidine, a modified cytidine, or an unmodified cytidine, and R⁴ is an abasic nucleoside. In certain embodiments, R³ is 2 or 3 linked nucleosides, optionally comprises a cytidine, a modified cytidine, or an unmodified cytidine, and R⁴ a terminal group. In certain embodiments, R³ is 2 or 3 linked nucleosides, optionally comprises a cytidine, a modified cytidine, or an unmodified cytidine, and R⁴ is an H. In certain embodiments, if R³ and R⁴ are each independently 1, 2, or 3 linked nucleosides or are each independently an abasic nucleoside, R³ and R⁴ also include an internucleoside linkage selected from a phosphodiester internucleoside linkage, a phosphorothioate internucleoside linkage, and a mesyl phosphoramidate internucleoside linkage. In certain embodiments, the modified oligonucleotide is a pharmaceutically acceptable salt comprising one or more cations selected from sodium, potassium, calcium, and magnesium. In certain embodiments, the oligomeric agent is single-stranded. In certain embodiments, the oligomeric agent is an antisense agent. In certain embodiments, the oligomeric agent is a single-stranded antisense agent.

### VI. Certain Compounds

### 1. Compound No. 1757576

Compound No. 1757576 is characterized as a 6-10-4 gapmer of linked nucleosides having a nucleobase sequence (from 5′ to 3′) of ACTTGGTGAATCTTTATTAA (SEQ ID NO: 22), wherein each of nucleosides 1-5 and 19-20 (from 5′ to 3′) are 2′-MOE nucleosides, each of nucleosides 6, 17, and 18 are cEt nucleosides, and each of nucleosides 7-16 are 2′-β-D-deoxynucleosides, wherein the internucleoside linkages between nucleosides 1 to 2, 4 to 5, 5 to 6, 6 to 7, 7 to 8, 11 to 12, 12 to 13, 13 to 14, 14 to 15, 15 to 16, 17 to 18, 18 to 19, and 19 to 20 are phosphorothioate internucleoside linkages, wherein the internucleoside linkages between nucleosides 2 to 3 and 3 to 4 are phosphodiester internucleoside linkages, wherein the internucleoside linkages between nucleosides 8 to 9, 9 to 10, 10 to 11, and 16 to 17 are mesyl phosphoramidate internucleoside linkages, and wherein each cytosine is a 5-methylcytosine.

Compound No. 1757576 is represented by the following chemical notation: Aₑₛ^{m}CₑₒTₑₒTₑₛGₑₛGₖₛT_{ds}G_{dz}A_{dz}A_{dz}T_{ds}^{m}C_{ds}T_{ds}T_{ds}T_{ds}A_{dz}TₖₛTₖₛAₑₛAₑ (SEQ ID NO: 45), wherein:
A=an adenine nucleobase,
^{m}C=a 5-methylcytosine nucleobase,
G=a guanine nucleobase,
T=a thymine nucleobase,
e=a 2′-MOE sugar moiety,
k=a cEt sugar moiety,
d=a 2′-β-D-deoxyribosyl sugar moiety,
s=a phosphorothioate internucleoside linkage,
o=a phosphodiester internucleoside linkage, and
z=a mesyl phosphoramidate internucleoside linkage; and the oligomeric agent does not include a conjugate group or a terminal group.

Compound No. 1757576 is represented by the following chemical structure:

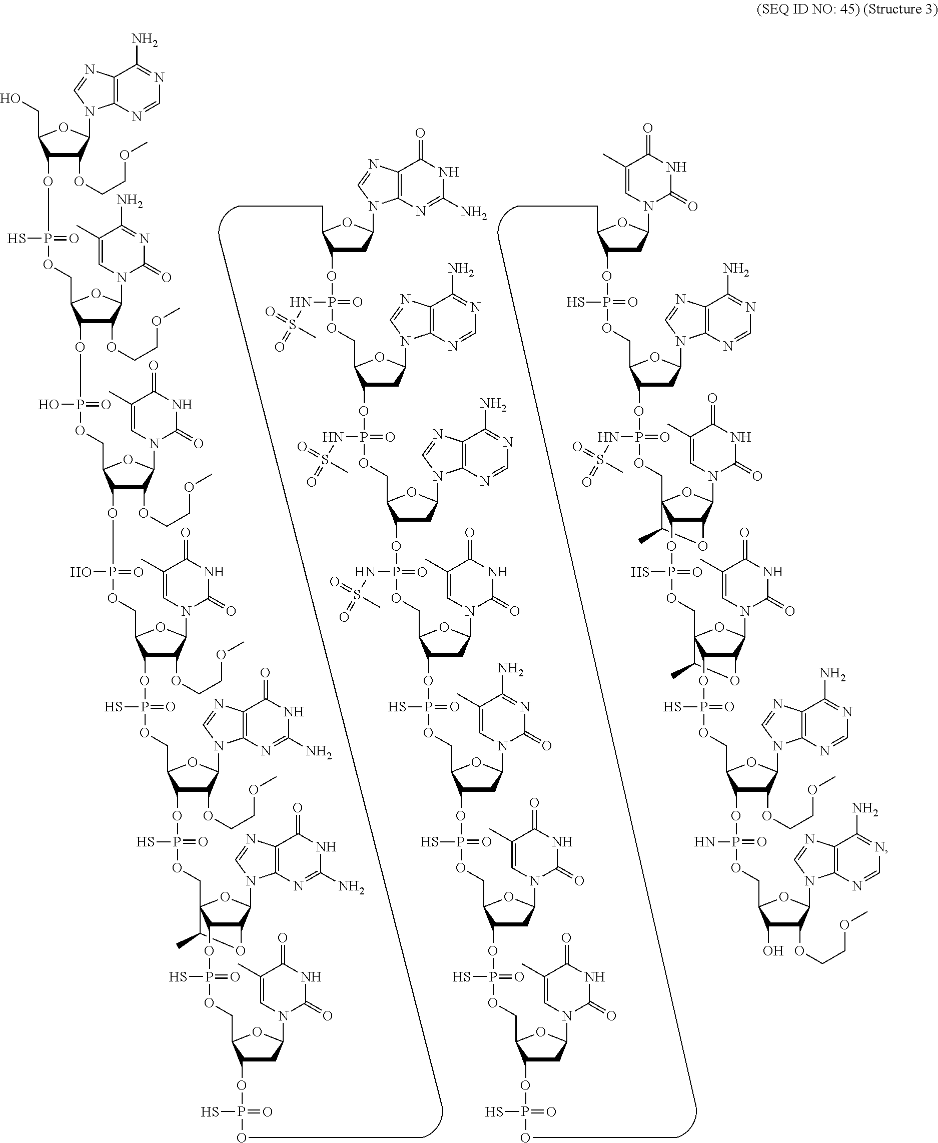

or a pharmaceutically acceptable salt thereof. The pharmaceutically acceptable salt of Compound No. 1757576 comprises one or more cations selected from sodium, potassium, calcium, and magnesium.

The sodium salt of Compound No. 1757576 is represented by the following chemical structure:

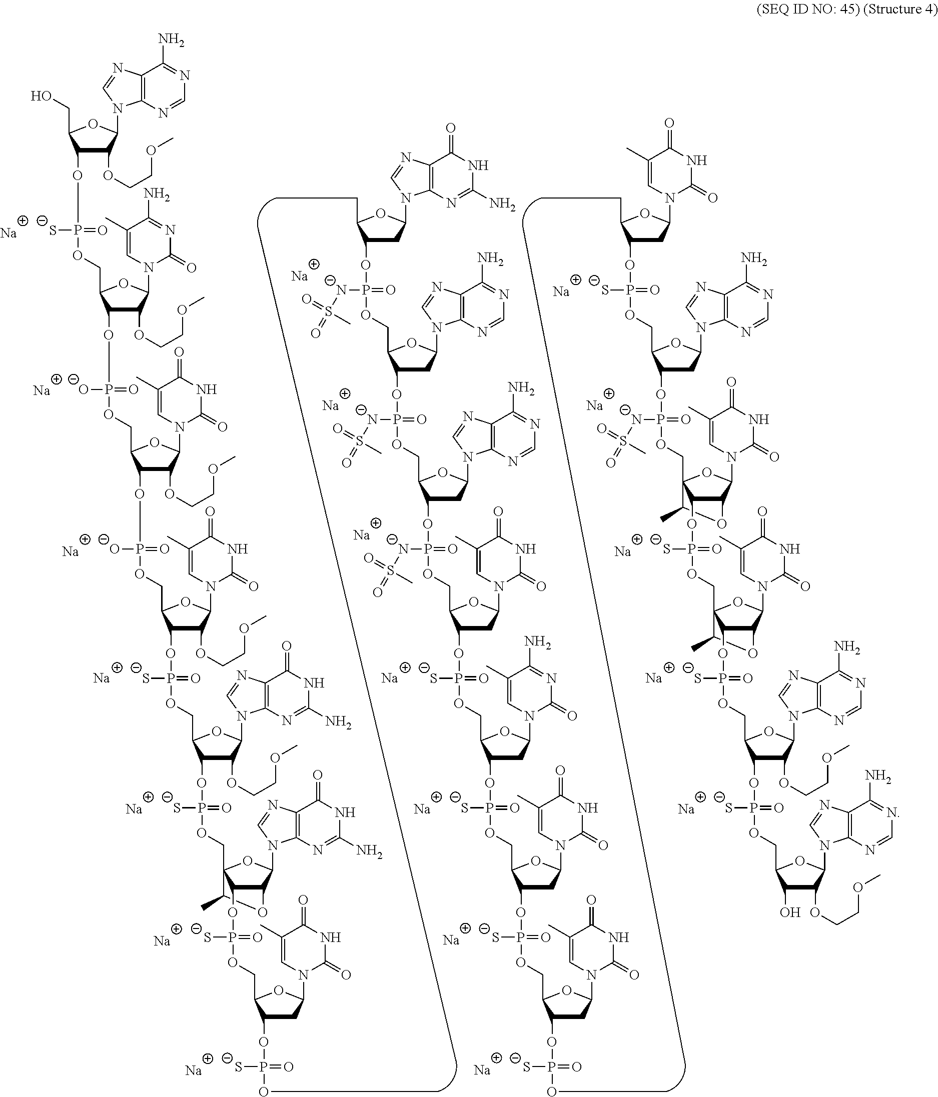

### 2. Compound No. 1785602

Compound No. 1785602 is characterized as a 3-13-4 gapmer of linked nucleosides having a nucleobase sequence (from 5′ to 3′) of CTTGGTGAATCTTTATTAAA (SEQ ID NO: 23), wherein each of nucleosides 1-3, 17, 18, and 20 (from 5′ to 3′) are cEt nucleosides and each of nucleosides 4-16 and 19 are 2′-β-D-deoxynucleosides, wherein the internucleoside linkages between nucleosides 1 to 2, 2 to 3, 3 to 4, 10 to 11, 11 to 12, 12 to 13, 13 to 14, 14 to 15, 15 to 16, 16 to 17, 17 to 18, and 18 to 19 are phosphorothioate internucleoside linkages, wherein the internucleoside linkages between nucleosides 4 to 5, 5 to 6, 6 to 7, 7 to 8, 8 to 9, 9 to 10, and 19 to 20 are mesyl phosphoramidate internucleoside linkages, and wherein each cytosine is a 5-methylcytosine.

Compound No. 1785602 is represented by the following chemical notation: ^{m}CₖₛTₖₛTₖₛG_{dz}G_{dz}T_{dz}G_{dz}A_{dz}A_{dz}T_{ds}^{m}C_{ds}T_{ds}T_{ds}T_{ds}A_{ds}T_{ds}TₖₛAₖₛA_{dz}Aₖ (SEQ ID NO: 46), wherein:
A=an adenine nucleobase,
^{m}C=a 5-methylcytosine nucleobase,
G=a guanine nucleobase,
T=a thymine nucleobase,
k=a cEt sugar moiety,
d=a 2′-β-D-deoxyribosyl sugar moiety,
s=a phosphorothioate internucleoside linkage, and
z=a mesyl phosphoramidate internucleoside linkage; and the oligomeric agent does not include a conjugate group or a terminal group.

Compound No. 1785602 is represented by the following chemical structure:

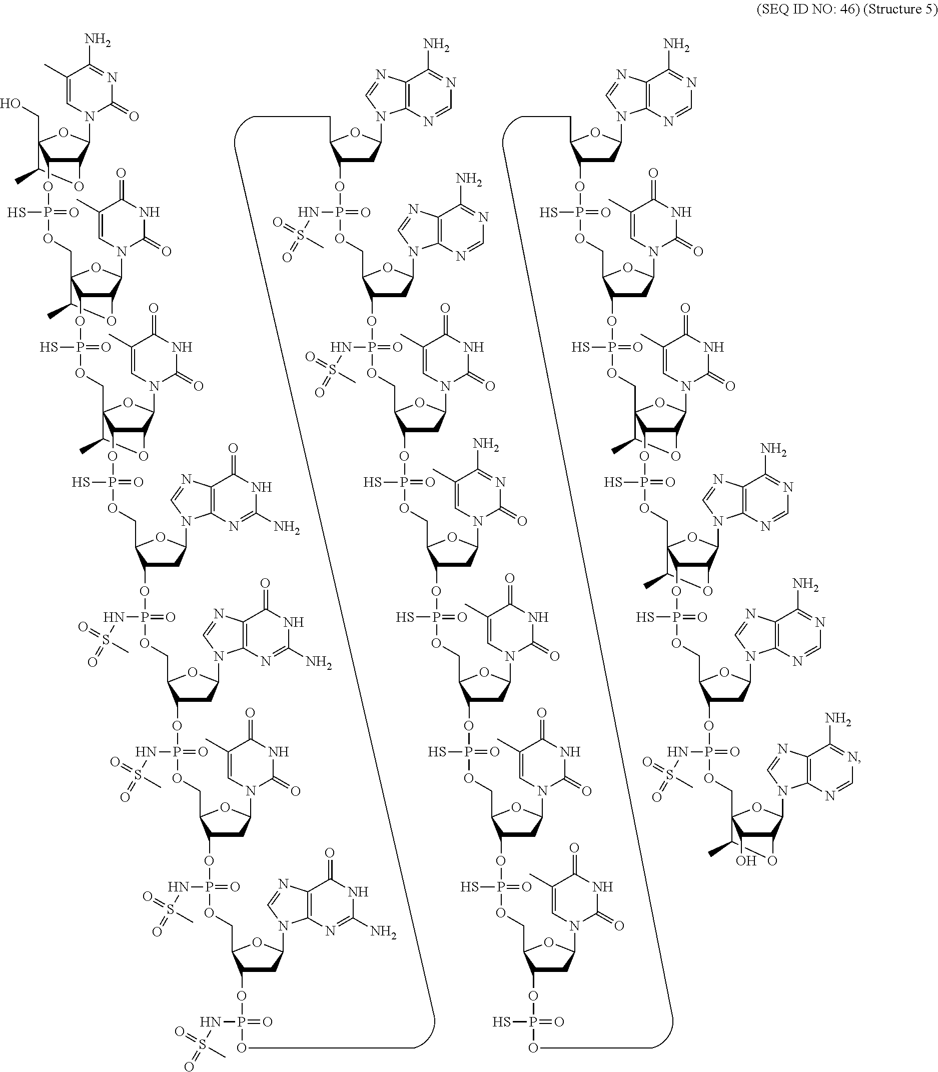

or a pharmaceutically acceptable salt thereof. The pharmaceutically acceptable salt of Compound No. 1785602 comprises one or more cations selected from sodium, potassium, calcium, and magnesium.

The sodium salt of Compound No. 1785602 is represented by the following chemical structure:

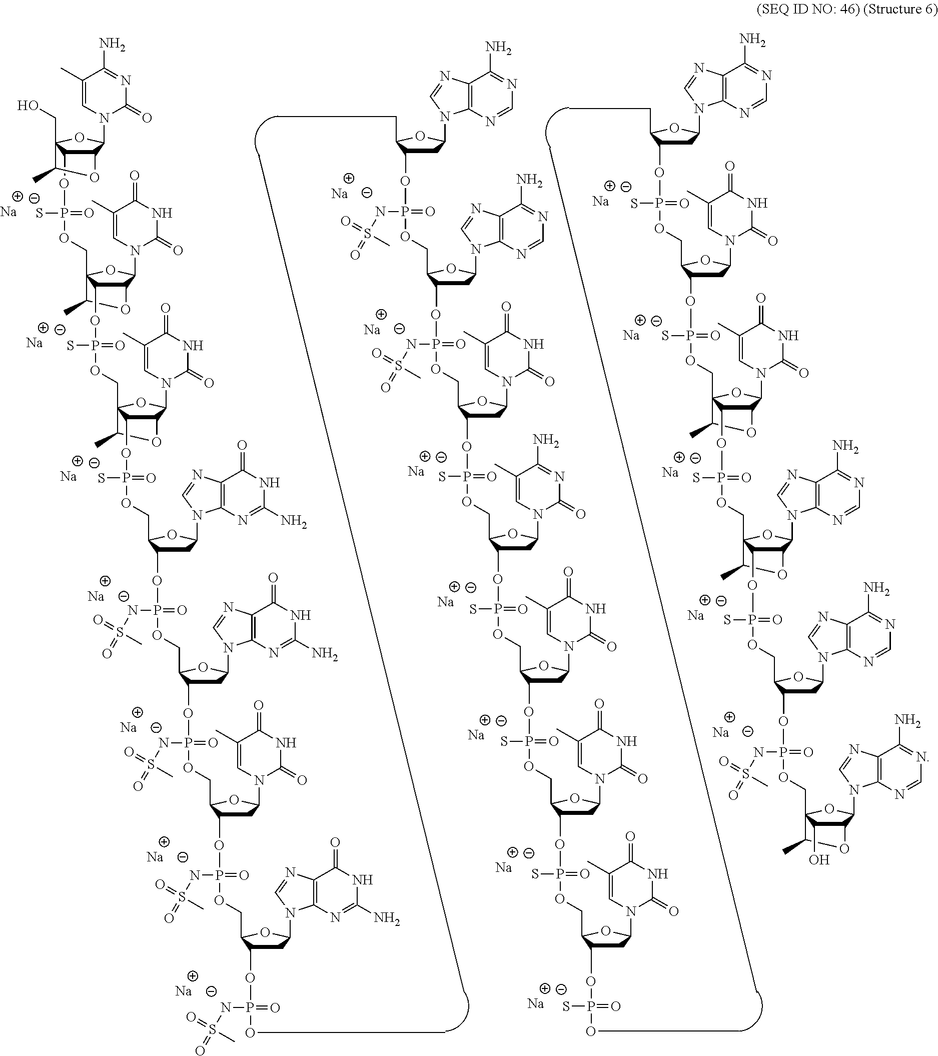

### 3. Compound No. 1809635

Compound No. 1809635 is characterized as a 6-10-4 gapmer of linked nucleosides having a nucleobase sequence (from 5′ to 3′) of ACTTGGTGAATCTTTATTAA (SEQ ID NO: 22), wherein each of nucleosides 2-5 and 19-20 (from 5′ to 3′) are 2′-MOE nucleosides, each of nucleosides 1, 6, 17, and 18 are cEt nucleosides, and each of nucleosides 7-16 are 2′-β-D-deoxynucleosides, wherein the internucleoside linkages between nucleosides 1 to 2, 4 to 5, 5 to 6, 6 to 7, 7 to 8, 11 to 12, 12 to 13, 13 to 14, 14 to 15, 15 to 16, 17 to 18, 18 to 19, and 19 to 20 are phosphorothioate internucleoside linkages, wherein the internucleoside linkages between nucleosides 2 to 3 and 3 to 4 are phosphodiester internucleoside linkages, wherein the internucleoside linkages between nucleosides 8 to 9, 9 to 10, 10 to 11, and 16 to 17 are mesyl phosphoramidate internucleoside linkages, and wherein each cytosine is a 5-methylcytosine.

Compound No. 1809635 is represented by the following chemical notation: Aₖₛ^{m}CₑₒTₑₒTₑₛGₑₛGₖₛT_{ds}G_{dz}A_{dz}A_{dz}T_{ds}^{m}C_{ds}T_{ds}T_{ds}T_{ds}A_{dz}TₖₛTₖₛAₑₛAₑ (SEQ ID NO: 47), wherein:
A=an adenine nucleobase,
^{m}C=a 5-methylcytosine nucleobase,
G=a guanine nucleobase,
T=a thymine nucleobase,
e=a 2′-MOE sugar moiety,
k=a cEt sugar moiety,
d=a 2′-β-D-deoxyribosyl sugar moiety,
s=a phosphorothioate internucleoside linkage,
o=a phosphodiester internucleoside linkage, and
z=a mesyl phosphoramidate internucleoside linkage; and the oligomeric agent does not include a conjugate group or a terminal group.

Compound No. 1809635 is represented by the following chemical structure:

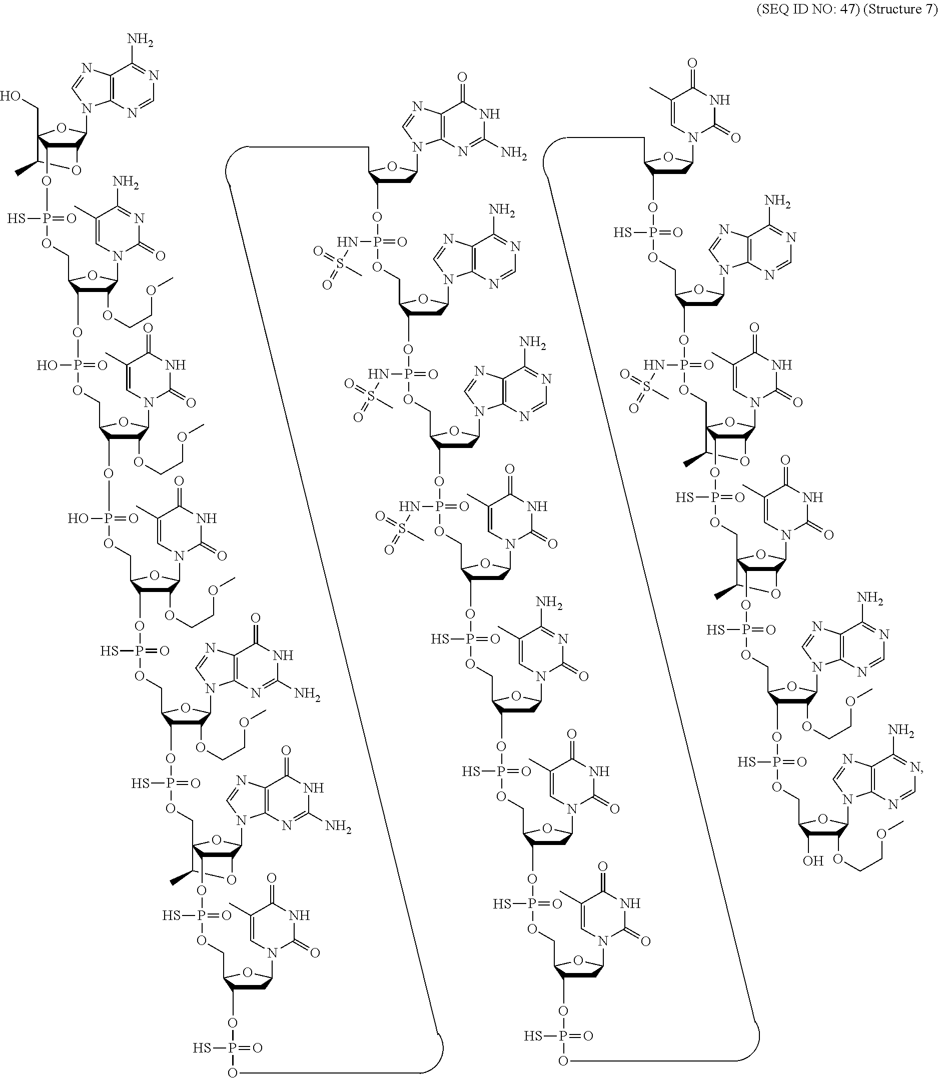

or a pharmaceutically acceptable salt thereof. The pharmaceutically acceptable salt of Compound No. 1809635 comprises one or more cations selected from sodium, potassium, calcium, and magnesium.

The sodium salt of Compound No. 1809635 is represented by the following chemical structure:

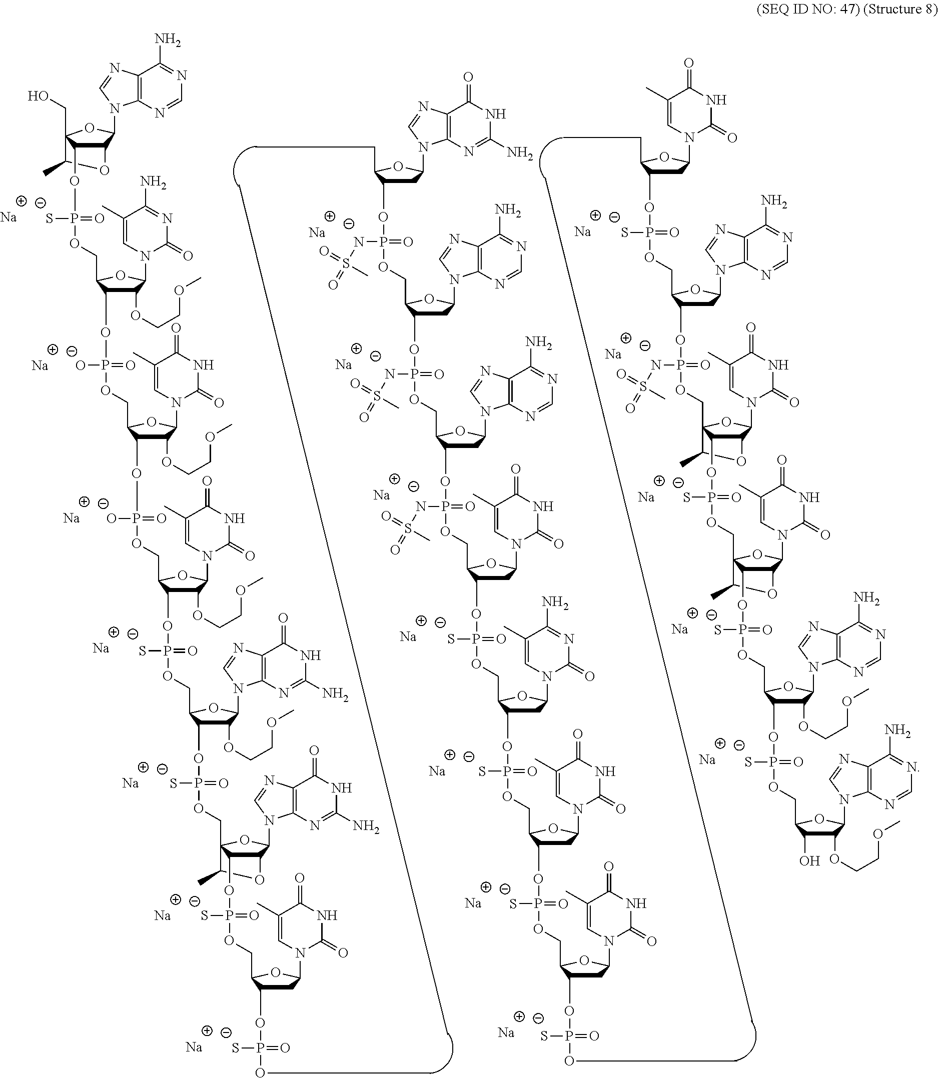

### 4. Compound No. 1809814

Compound No. 1809814 is characterized as a 4-10-6 gapmer of linked nucleosides having a nucleobase sequence (from 5′ to 3′) of TTGGTGAATCTTTATTAAAC (SEQ ID NO: 27), wherein each of nucleosides 1-3 and 17-20 (from 5′ to 3′) are 2′-MOE nucleosides, each of nucleosides 4, 15, and 16 are cEt nucleosides, and each of nucleosides 5-14 are 2′-β-D-deoxynucleosides, wherein the internucleoside linkages between nucleosides 1 to 2, 6 to 7, 7 to 8, 8 to 9, and 14 to 15 are mesyl phosphoramidate internucleoside linkages, wherein the internucleoside linkages between nucleosides 2 to 3 and 3 to 4 are phosphodiester internucleoside linkages, wherein the internucleoside linkages between nucleosides 4 to 5, 5 to 6, 9 to 10, 10 to 11, 11 to 12, 12 to 13, 13 to 14, 15 to 16, 16 to 17, 17 to 18, 18 to 19, and 19 to 20 are phosphorothioate internucleoside linkages, and wherein each cytosine is a 5-methylcytosine.

Compound No. 1809814 is represented by the following chemical notation: T_{ez}TₑₒGₑₒGₖₛT_{ds}G_{dz}A_{dz}A_{dz}T_{ds}^{m}C_{ds}T_{ds}T_{ds}T_{ds}A_{dz}TₖₛTₖₛAₑₛAₑₛAₑₛ^{m}Cₑ (SEQ ID NO: 48), wherein:
A=an adenine nucleobase,
^{m}C=a 5-methylcytosine nucleobase,
G=a guanine nucleobase,
T=a thymine nucleobase,
e=a 2′-MOE sugar moiety,
k=a cEt sugar moiety,
d=a 2′-β-D-deoxyribosyl sugar moiety,
s=a phosphorothioate internucleoside linkage,
o=a phosphodiester internucleoside linkage, and
z=a mesyl phosphoramidate internucleoside linkage; and the oligomeric agent does not include a conjugate group or a terminal group.

Compound No. 1809814 is represented by the following chemical structure:

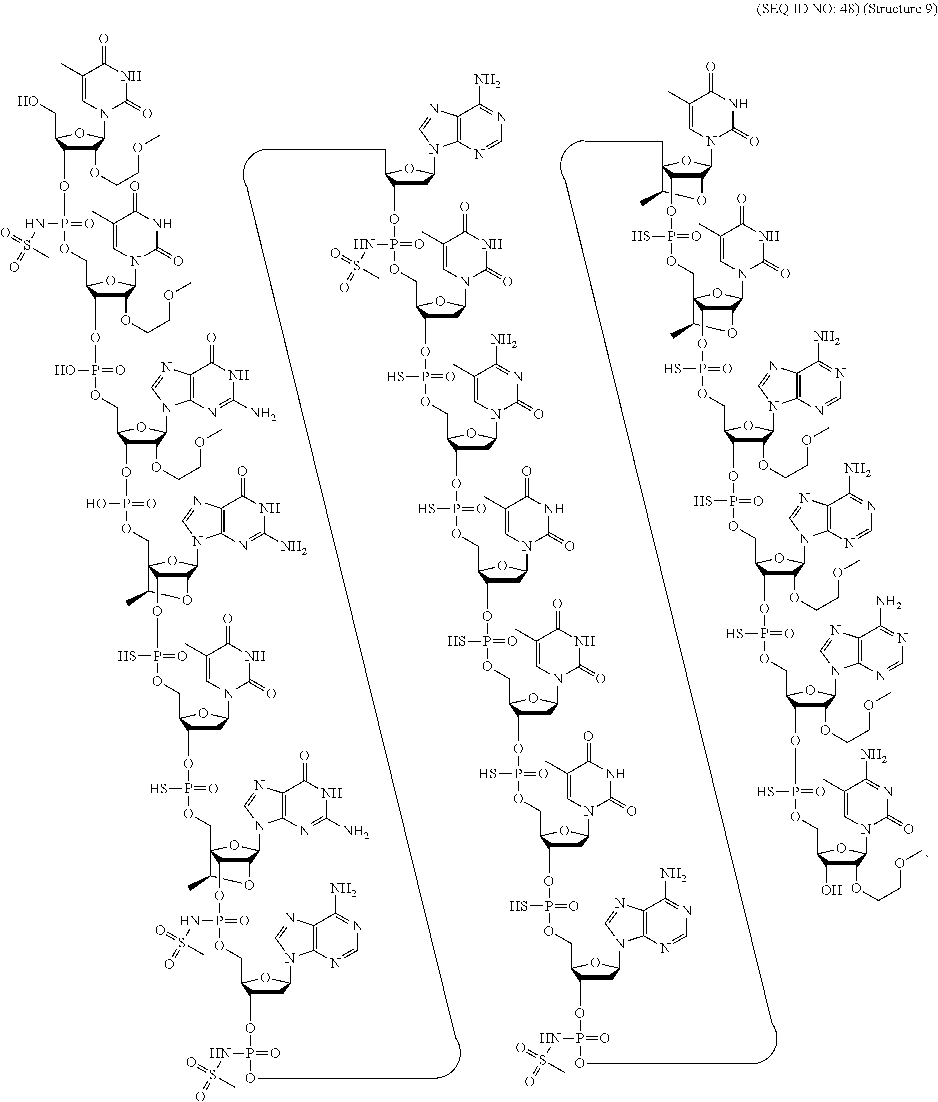

or a pharmaceutically acceptable salt thereof. The pharmaceutically acceptable salt of Compound No. 1809814 comprises one or more cations selected from sodium, potassium, calcium, and magnesium.

The sodium salt of Compound No. 1809814 is represented by the following chemical structure:

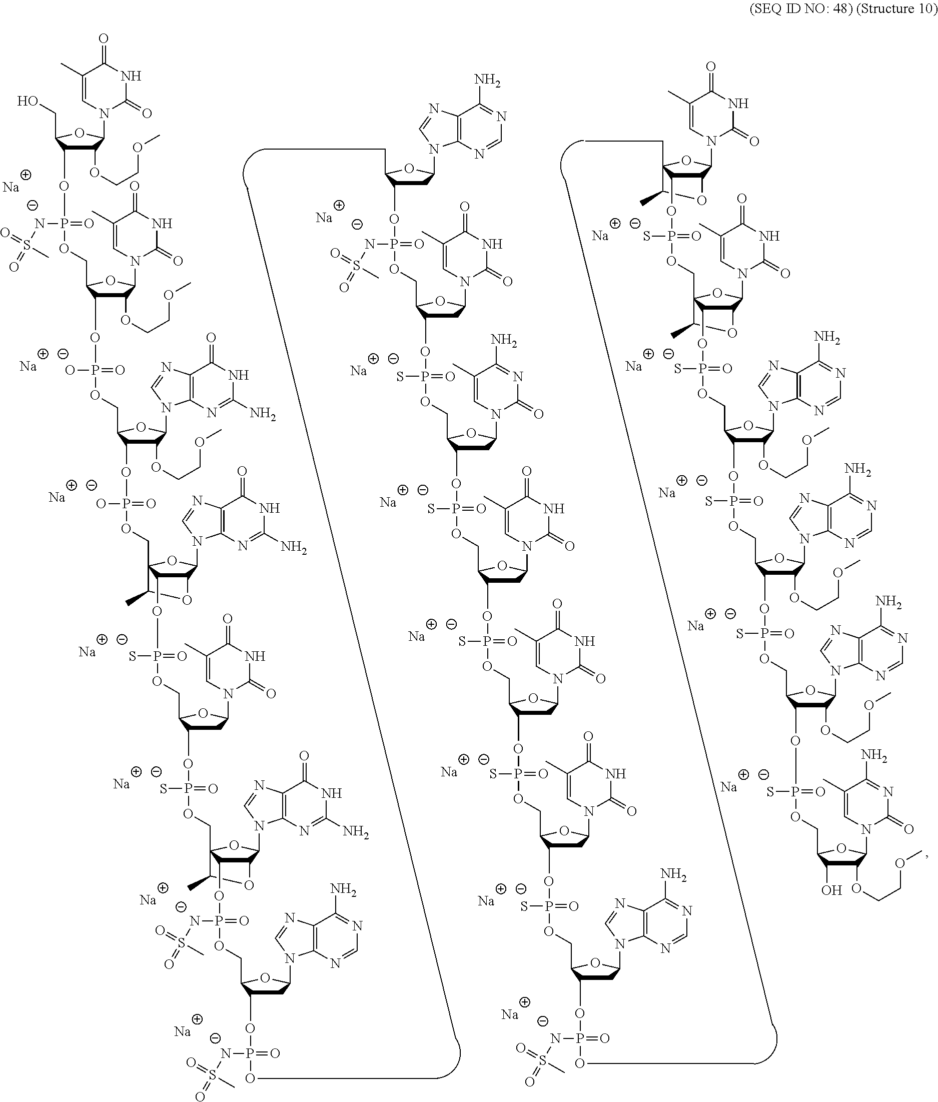

### VII. Comparator Compounds

In certain embodiments, Compound No. 1517891 is a comparator compound and is previously described in International Patent No. WO 2022/066956. Compound No. 1517891 consists of the nucleobase sequence (from 5′ to 3′): ACTTGGTGAATCTTTATTAA (SEQ ID NO: 22). The sugar motif for Compound No. 1517891 is (from 5′ to 3′): eeeeeddddddddddeeeee; wherein each “d” represents a 2′-β-D-deoxyribosyl sugar moiety, and each “e” represents a 2′-MOE sugar moiety. The internucleoside linkage motif for Compound No. 1517891 is (from 5′ to 3′): sooosssssssssssooss; wherein each “s” represents a phosphorothioate internucleoside linkage, and each “o” represents a phosphodiester internucleoside linkage. Each cytosine nucleobase in Compound No. 1517891 is a 5-methyleytosine

In certain embodiments, Compound No. 1517578 is a comparator compound and is previously described in International Patent No. WO 2022/066956. Compound No. 1517578 consists of the nucleobase sequence (from 5′ to 3′): CTTGGTGAATCTTTATTAAA (SEQ ID NO: 23). The sugar motif for Compound No. 1517578 is (from 5′ to 3′): eeeeeddddddddddeeeee; wherein each “d” represents a 2′-β-D-deoxyribosyl sugar moiety, and each “e” represents a 2′-MOE sugar moiety. The internucleoside linkage motif for Compound No. 1517578 is (from 5′ to 3′): sooosssssssssssooss; wherein each “s” represents a phosphorothioate internucleoside linkage, and each “o” represents a phosphodiester internucleoside linkage. Each cytosine nucleobase in Compound No. 1517578 is a 5-methylcytosine.

In certain embodiments, Compound No. 1517853 is a comparator compound and is previously described in International Patent No. WO 2022/066956. Compound No. 1517853 consists of the nucleobase sequence (from 5′ to 3′): TTGGTGAATCTTTATTAAAC (SEQ ID NO: 27). The sugar motif for Compound No. 1517853 is (from 5′ to 3′): eeeeeddddddddddeeeee; wherein each “d” represents a 2′-β-D-deoxyribosyl sugar moiety, and each “e” represents a 2′-MOE sugar moiety. The internucleoside linkage motif for Compound No. 1517853 is (from 5′ to 3′): sooosssssssssssooss; wherein each “s” represents a phosphorothioate internucleoside linkage, and each “o” represents a phosphodiester internucleoside linkage. Each cytosine nucleobase in Compound No. 1517853 is a 5-methylcytosine.

In certain embodiments, compounds described herein are superior relative to compounds described in WO 2022/066956, because they demonstrate one or more improved properties, such as knockdown activity and proinflammatory effects.

For example, Compound No. 1757576, Compound No. 1785602, Compound No. 1809635, and Compound No. 1809814 each demonstrated improved 8-week knockdown activity in vivo compared to Compound No. 1517891, Compound No. 1517578, and Compound No. 1517853 in the assay shown in Example 11. In particular, Compound No. 1757576, Compound No. 1785602, Compound No. 1809635, and Compound No. 1809814 achieved a 79%, 73%, 74%, and 64% reduction of human APOE RNA in the cortex of an APOE knock-in mouse model, respectively, at 8 weeks post administration. In comparison, Compound No. 1517891, Compound No. 1517578, and Compound No. 1517853 achieved a 44%, 34%, and 33% reduction of human APOE RNA in the cortex of an APOE knock-in mouse model, respectively, at 8 weeks post administration. Therefore, each of Compound No. 1757576, Compound No. 1785602, Compound No. 1809635, and Compound No. 1809814 exhibited a better activity toward APOE suppression than Compound No. 1517891, Compound No. 1517578, and Compound No. 1517853 in this assay.

For example, Compound No. 1757576, Compound No. 1785602, Compound No. 1809635, and Compound No. 1809814 each demonstrated lower proinflammatory effect in vitro compared to Compound No. 1517891, Compound No. 1517578, and Compound No. 1517853 in the assay shown in Example 17. In particular, Compound No. 1757576, Compound No. 1785602, Compound No. 1809635, and Compound No. 1809814 did not induce any inflammatory response in human BJAB cells after a 24-hour culturing period. In comparison, Compound No. 1517891 induced a mildly inflammatory response and Compound No. 1517578 and Compound No. 1517853 induced an inflammatory and a highly inflammatory response, respectively, in human BJAB cells post 24-hour culture. Therefore, each of Compound No. 1757576, Compound No. 1785602, Compound No. 1809635, and Compound No. 1809814 demonstrated a lower proinflammatory effect compared to Compound No. 1517891, Compound No. 1517578, and Compound No. 1517853 in this assay.

### Nonlimiting Disclosure and Incorporation by Reference

Each of the literature and patent publications listed herein is incorporated by reference in its entirety.

While certain compounds, compositions, and methods have been described herein with specificity in accordance with certain embodiments, the following examples serve only to illustrate the compounds described herein and are not intended to limit the same. Each of the references, GenBank accession numbers, ENSEMBL identifiers, and the like recited in the present application, is incorporated herein by reference in its entirety.

The sequence listing accompanying this filing identifies each nucleic acid sequence as either “RNA” or “DNA” as required; however, one of skill in the art will readily appreciate that designation of “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (i.e., 2′-OH in place of one 2′-H of DNA) or as an RNA having a modified base (i.e., thymine (5-methyl uracil) in place of an uracil of RNA); and certain nucleic acid compounds described herein comprise one or more nucleosides comprising modified sugar moieties having 2′-substituent(s) that are neither OH nor H. One of skill in the art will readily appreciate that labeling such nucleic acid compounds “RNA” or “DNA” does not alter or limit the description of such nucleic acid compounds.

Herein, the description of compounds as having “the nucleobase sequence of” a SEQ ID NO. describes only the nucleobase sequence. Accordingly, absent additional description, such description of compounds by reference to a nucleobase sequence of a SEQ ID NO. does not limit sugar or internucleoside linkage modifications or presence or absence of additional substituents such as a conjugate group. Further, absent additional description, the nucleobases of a compound “having the nucleobase sequence of” a SEQ ID NO. include such compounds having modified forms of the identified nucleobases as described herein.

Herein, the description of compounds by chemical notation (subscripts and/or superscripts to indicate chemical modifications) without reference to a specific Compound No. include only each noted modification, but may include additional substituents, such as a conjugate group, unless otherwise indicated. For example, the chemical notation of “AₑₛTₖₒ^{m}C_{ez}GₐₛC_{d}” indicates a compound wherein the first nucleoside comprises a 2′-MOE sugar moiety (indicated by the “e” subscript) and an unmodified adenine nucleobase linked to the second nucleoside via a phosphorothioate linkage (indicated by the “s” subscript); the second nucleoside comprises a cEt sugar moiety (indicated by the “k” subscript) and an unmodified thymine nucleobase linked to the third nucleoside via a phosphodiester linkage (indicated by the “o” subscript); the third nucleoside comprises a 2′-MOE sugar moiety and a 5-methyl modified cytosine nucleobase (indicated by the “m” superscript) linked to the fourth nucleoside via a mesyl phosphoramidate linkage (indicated by the “z” subscript); the fourth nucleoside comprises a 2′-β-D-deoxyribosyl sugar moiety (indicated by the “d” subscript) and an unmodified guanine nucleobase linked to the fifth nucleoside with a phosphorothioate linkage; and the fifth nucleoside comprises a 2′-β-D-deoxyribosyl sugar moiety and an unmodified cytosine nucleobase; and the compound may include additional substituents, such as a conjugate group.

Herein, where a specific compound (e.g., with reference to a Compound No.) is described (as in the examples) by chemical notation, each nucleobase, sugar, and internucleoside linkage of such specific compound is modified only as indicated. Accordingly, in the context of a description of a specific compound having a particular Compound No., “AₑₛTₖₒ^{m}C_{ez}G_{ds}C_{d}” indicates a compound wherein the first nucleoside comprises a 2′-MOE sugar moiety (indicated by the “e” subscript) and an unmodified adenine nucleobase linked to the second nucleoside via a phosphorothioate linkage (indicated by the “s” subscript); the second nucleoside comprises a cEt sugar moiety (indicated by the “k” subscript) and an unmodified thymine nucleobase linked to the third nucleoside via a phosphodiester linkage (indicated by the “o” subscript); the third nucleoside comprises a 2′-MOE sugar moiety and a 5-methyl modified cytosine nucleobase (indicated by the “m” superscript) linked to the fourth nucleoside via a mesyl phosphoramidate linkage (indicated by the “z” subscript); the fourth nucleoside comprises a 2′-β-D-deoxyribosyl sugar moiety (indicated by the “d” subscript) and an unmodified guanine nucleobase linked to the fifth nucleoside with a phosphorothioate linkage; and the fifth nucleoside comprises a 2′-β-D-deoxyribosyl sugar moiety and an unmodified cytosine nucleobase; and the compound does not include additional substituents.

Herein, sugar, internucleoside linkage, and nucleobase modifications may be indicated within a nucleotide or nucleobase sequence (e.g., by superscript or subscript, as shown above) or may be indicated in text accompanying a sequence (e.g., in separate text that appears within or above or below a table of compounds).

Where a specific compound is described herein by way of a drawn chemical structure, each nucleobase, sugar, and internucleoside linkage of such a specific compound includes only the modifications indicated in the drawn chemical structure. One of skill will appreciate, however, that drawn compounds may exist in equilibrium between tautomeric forms and/or as salts in equilibrium with protonated or ionic forms. Drawn structures are intended to capture all such forms of such compounds.

While effort has been made to accurately describe compounds in the accompanying sequence listing, should there be any discrepancies between a description in this specification and in the accompanying sequence listing, the description in the specification and not in the sequence listing is the accurate description.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric agent that are beneficial for use as a therapeutic or research tool. In certain embodiments, radioactive isotopic substitutions may make the compound suitable for research or diagnostic purposes such as imaging.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif And, for example, where a particular high-affinity modification appears at a particular position, other high-affinity modifications at the same position are considered suitable, unless otherwise indicated.

### Example 1: Design of MOE Gapmers Complementary to Human APOE RNA

Modified oligonucleotides complementary to a human APOE RNA were designed and synthesized following standard techniques.

Each modified oligonucleotide listed in the tables below are 100% complementary to SEQ ID NO: 1 (GENBANK Accession No. NC_000019.10 truncated from nucleosides 44903001 to 44912000), to SEQ ID NO: 2 (GENBANK Accession No. NM_001302688.1), or to both. “N/A” indicates that the modified oligonucleotide is not 100% complementary to that particular target nucleic acid sequence. “Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence.

The modified oligonucleotides in the table below are 6-10-4 MOE modified oligonucleotides with mixed internucleoside linkages. Each cytosine residue in the modified oligonucleotides below is a 5-methylcytosine. The modified oligonucleotides in the table below are 20 nucleosides in length, and the sugar motif of each modified oligonucleotide is (from 5′ to 3′): eeeeeeddddddddddeeee, wherein each “e” represents a 2′-MOE sugar moiety, and each “d” represents a 2′-β-D-deoxyribosyl sugar moiety. The internucleoside linkage motif for each modified oligonucleotides is presented in the column labeled “Internucleoside Linkage (5′ to 3′)”, wherein each “s” represents a phosphorothioate internucleoside linkage, each “o” represents a phosphodiester internucleoside linkage, each “z” represents a mesyl phosphoramidate internucleoside linkage, and each “[pn]” represents a cyclic guanidine phosphoramidate internucleoside linkage as shown below:

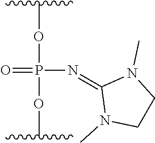 [SMILES: *OP(=O)(N=C1N(C)CCN1C)O*]

TABLE 1
6-10-4 MOE gapmers with mixed internucleoside linkages complementary to human APOE

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | Internucleoside | ID |
| Number | Site | Site | Site | Site | Sequence (5′ to 3′) | Linkage (5′ to 3′) | NO |
| 1693144 | 6366 | 6385 | 1268 | 1287 | ACTTGGTGAATCTTTATTAA | soosssszzzssssszsss | 22 |
| 1736520 | 6365 | 6384 | 1267 | 1286 | CTTGGTGAATCTTTATTAAA | sossoszzzssssssssss | 23 |
| 1736522 | 6365 | 6384 | 1267 | 1286 | CTTGGTGAATCTTTATTAAA | sooosszzssssssssoss | 23 |
| 1736523 | 6365 | 6384 | 1267 | 1286 | CTTGGTGAATCTTTATTAAA | ssoooszzssssssssoss | 23 |
| 1736524 | 6365 | 6384 | 1267 | 1286 | CTTGGTGAATCTTTATTAAA | sssooozzssssssssoss | 23 |
| 1736525 | 6365 | 6384 | 1267 | 1286 | CTTGGTGAATCTTTATTAAA | sooosszzsssssssssss | 23 |
| 1736526 | 6365 | 6384 | 1267 | 1286 | CTTGGTGAATCTTTATTAAA | ssoooszzsssssssssss | 23 |
| 1736527 | 6365 | 6384 | 1267 | 1286 | CTTGGTGAATCTTTATTAAA | sssooozzsssssssssss | 23 |
| 1652902 | 6365 | 6384 | 1267 | 1286 | CTTGGTGAATCTTTATTAAA | sooooossssssssssoss | 23 |
| 1693101 | 6365 | 6384 | 1267 | 1286 | CTTGGTGAATCTTTATTAAA | sssssozzzssssszssss | 23 |
| 1693102 | 6365 | 6384 | 1267 | 1286 | CTTGGTGAATCTTTATTAAA | ssssoszzzssssszssss | 23 |
| 1693105 | 6365 | 6384 | 1267 | 1286 | CTTGGTGAATCTTTATTAAA | sosssszzzssssszssss | 23 |
| 1736518 | 6365 | 6384 | 1267 | 1286 | CTTGGTGAATCTTTATTAAA | soossszzzssssssssss | 23 |
| 1736519 | 6365 | 6384 | 1267 | 1286 | CTTGGTGAATCTTTATTAAA | sososszzzssssssssss | 23 |
| 1736521 | 6365 | 6384 | 1267 | 1286 | CTTGGTGAATCTTTATTAAA | sosssszzzsssssssoss | 23 |
| 1652898 | 6362 | 6381 | 1264 | 1283 | GGTGAATCTTTATTAAACTA | sooooossssssssssoss | 24 |
| 1652899 | 6363 | 6382 | 1265 | 1284 | TGGTGAATCTTTATTAAACT | sooooossssssssssoss | 25 |
| 1652900 | 6361 | 6380 | 1263 | 1282 | GTGAATCTTTATTAAACTAG | sooooossssssssssoss | 26 |
| 1652901 | 6364 | 6383 | 1266 | 1285 | TTGGTGAATCTTTATTAAAC | sooooossssssssssoss | 27 |
| 1652903 | 6366 | 6385 | 1268 | 1287 | ACTTGGTGAATCTTTATTAA | sooooossssssssssoss | 22 |
| 1652905 | 6362 | 6381 | 1264 | 1283 | GGTGAATCTTTATTAAACTA | sooossssssssssssoss | 24 |
| 1652917 | 6362 | 6381 | 1264 | 1283 | GGTGAATCTTTATTAAACTA | sooooossssszsszzoss | 24 |
| 1652918 | 6362 | 6381 | 1264 | 1283 | GGTGAATCTTTATTAAACTA | sooooozzzsssssssoss | 24 |
| 1652919 | 6362 | 6381 | 1264 | 1283 | GGTGAATCTTTATTAAACTA | soooooszzzssssssoss | 24 |
| 1693077 | 6364 | 6383 | 1266 | 1285 | TTGGTGAATCTTTATTAAAC | sosssszzszssszssoss | 27 |
| 1693078 | 6364 | 6383 | 1266 | 1285 | TTGGTGAATCTTTATTAAAC | ssossszzszssszssoss | 27 |
| 1693079 | 6364 | 6383 | 1266 | 1285 | TTGGTGAATCTTTATTAAAC | sssosszzszssszssoss | 27 |
| 1693080 | 6364 | 6383 | 1266 | 1285 | TTGGTGAATCTTTATTAAAC | ssssoszzszssszssoss | 27 |
| 1693081 | 6364 | 6383 | 1266 | 1285 | TTGGTGAATCTTTATTAAAC | sssssozzszssszssoss | 27 |
| 1693082 | 6364 | 6383 | 1266 | 1285 | TTGGTGAATCTTTATTAAAC | ssssoozzszssszsssss | 27 |
| 1693084 | 6364 | 6383 | 1266 | 1285 | TTGGTGAATCTTTATTAAAC | ssssszzzszsszzsssss | 27 |
| 1693085 | 6364 | 6383 | 1266 | 1285 | TTGGTGAATCTTTATTAAAC | ssssszzzsssszzsssss | 27 |
| 1693100 | 6365 | 6384 | 1267 | 1286 | CTTGGTGAATCTTTATTAAA | sssssszzzssssszsoss | 23 |
| 1693104 | 6365 | 6384 | 1267 | 1286 | CTTGGTGAATCTTTATTAAA | ssossszzzssssszssss | 23 |
| 1693110 | 6365 | 6384 | 1267 | 1286 | CTTGGTGAATCTTTATTAAA | sssssszzzsssszzssss | 23 |
| 1693138 | 6365 | 6384 | 1267 | 1286 | CTTGGTGAATCTTTATTAAA | sssssozzzsssszzsoss | 23 |
| 1693139 | 6366 | 6385 | 1268 | 1287 | ACTTGGTGAATCTTTATTAA | ssssssszzzssssszsss | 22 |
| 1693140 | 6366 | 6385 | 1268 | 1287 | ACTTGGTGAATCTTTATTAA | sssssoszzzssssszoss | 22 |
| 1693141 | 6366 | 6385 | 1268 | 1287 | ACTTGGTGAATCTTTATTAA | ssssooszzzssssszsss | 22 |
| 1693142 | 6366 | 6385 | 1268 | 1287 | ACTTGGTGAATCTTTATTAA | sssoosszzzssssszsss | 22 |
| 1693143 | 6366 | 6385 | 1268 | 1287 | ACTTGGTGAATCTTTATTAA | ssossszzzssssszsss | 22 |
| 1721303 | 6364 | 6383 | 1266 | 1285 | TTGGTGAATCTTTATTAAAC | s[pn][pn][pn]oossss | 27 |
| | | | | | | ssssssoss | |
| 1721304 | 6364 | 6383 | 1266 | 1285 | TTGGTGAATCTTTATTAAAC | s[pn][pn][pn]oossss | 27 |
| | | | | | | sszzssoss | |
| 1721305 | 6364 | 6383 | 1266 | 1285 | TTGGTGAATCTTTATTAAAC | s[pn][pn][pn]oossss | 27 |
| | | | | | | ssssss[pn]ss | |
| 1721306 | 6364 | 6383 | 1266 | 1285 | TTGGTGAATCTTTATTAAAC | s[pn][pn][pn]oossss | 27 |
| | | | | | | sszzss[n]ss | |
| 1721307 | 6365 | 6384 | 1267 | 1286 | CTTGGTGAATCTTTATTAAA | s[pn][pn][pn]oossss | 23 |
| | | | | | | ssssssoss | |
| 1721308 | 6365 | 6384 | 1267 | 1286 | CTTGGTGAATCTTTATTAAA | s[pn][pn][pn]oossss | 23 |
| | | | | | | ssszzsoss | |
| 1721309 | 6365 | 6384 | 1267 | 1286 | CTTGGTGAATCTTTATTAAA | s[pn][pn][pn]oossss | 23 |
| | | | | | | ssssss[pn]ss | |
| 1721310 | 6365 | 6384 | 1267 | 1286 | CTTGGTGAATCTTTATTAAA | s[pn][pn][pn]oossss | 23 |
| | | | | | | ssszzs[pn]ss | |
| 1601942 | 3229 | 3248 | N/A | N/A | CCGTGTTCCATTTATGAGCT | sooooossssssssssoss | 28 |
| 1601943 | 3203 | 3222 | N/A | N/A | GTCCTCATTTTAAAGTTCTC | sooooossssssssssoss | 29 |
| 1601944 | 3406 | 3425 | N/A | N/A | CTGGTCTTCTCTTATCTCCC | sooooossssssssssoss | 30 |
| 1601948 | 3339 | 3358 | N/A | N/A | GTTCAAATTCCATCCCCCCA | sooooossssssssssoss | 31 |
| 1601949 | 4192 | 4211 | N/A | N/A | CTGCAATGCATTAGAAACCT | sooooossssssssssoss | 32 |
| 1601955 | 3212 | 3231 | N/A | N/A | GCTAATTCAGTCCTCATTTT | sooooossssssssssoss | 33 |
| 1601956 | 3191 | 3210 | N/A | N/A | AAGTTCTCCAATCGACGGCT | sooooossssssssssoss | 34 |
| 1601957 | 3202 | 3221 | N/A | N/A | TCCTCATTTTAAAGTTCTCC | sooooossssssssssoss | 35 |
| 1601958 | 3264 | 3283 | N/A | N/A | CTTCACATTCTAAGCTCCAA | sooooossssssssssoss | 36 |
| 1601959 | 3166 | 3185 | N/A | N/A | CCGTGTCGCTGCCCCTGGCT | sooooossssssssssoss | 37 |
| 1693103 | 6365 | 6384 | 1267 | 1286 | CTTGGTGAATCTTTATTAAA | sssosszzzssssszssss | 23 |
| 1693106 | 6365 | 6384 | 1267 | 1286 | CTTGGTGAATCTTTATTAAA | sssssszzzssssszssss | 23 |
| 1778107 | 6366 | 6385 | 1268 | 1287 | ACTTGGTGAATCTTTATTAA | sooossszzsssssszsss | 22 |
| 1778109 | 6366 | 6385 | 1268 | 1287 | ACTTGGTGAATCTTTATTAA | sooossszzsssssszoss | 22 |
| 1778110 | 6366 | 6385 | 1268 | 1287 | ACTTGGTGAATCTTTATTAA | soooosszzsssssszoss | 22 |

The modified oligonucleotides in the table below are 5-10-5 MOE modified oligonucleotides with mixed PS/PO/mesyl internucleoside linkages. The modified oligonucleotides in the table below are 20 nucleosides in length, and the sugar motif of each modified oligonucleotide is (from 5′ to 3′): eeeeeddddddddddeeeee, wherein each “e” represents a 2′-MOE sugar moiety, and each “d” represents a 2′-β-D-deoxyribosyl sugar moiety. The internucleoside linkage motif for each modified oligonucleotides is presented in the column labeled “Internucleoside Linkage (5′ to 3′)”, wherein each s represents a phosphorothioate internucleoside linkage, each “o” represents a phosphodiester internucleoside linkage, and each “z” represents a mesyl phosphoramidate internucleoside linkage. Each cytosine residue is a 5-methylcytosine.

TABLE 2
5-10-5 MOE gapmers with mixed internucleoside linkages complementary to human APOE

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | Internucleoside | ID |
| Number | Site | Site | Site | Site | Sequence (5′ to 3′) | Linkage (5′ to 3′) | NO |
| 1652907 | 6362 | 6381 | 1264 | 1283 | GGTGAATCTTTATTAAACTA | soooszzssssssssooss | 24 |
| 1652908 | 6362 | 6381 | 1264 | 1283 | GGTGAATCTTTATTAAACTA | soooszzzsssssssooss | 24 |
| 1652909 | 6362 | 6381 | 1264 | 1283 | GGTGAATCTTTATTAAACTA | soooszzzzssssssooss | 24 |
| 1652910 | 6362 | 6381 | 1264 | 1283 | GGTGAATCTTTATTAAACTA | sooosszzsssssssooss | 24 |
| 1652911 | 6362 | 6381 | 1264 | 1283 | GGTGAATCTTTATTAAACTA | sooosszzzssssssooss | 24 |
| 1652912 | 6362 | 6381 | 1264 | 1283 | GGTGAATCTTTATTAAACTA | sooossssssszsszooss | 24 |
| 1652913 | 6362 | 6381 | 1264 | 1283 | GGTGAATCTTTATTAAACTA | soooszssssszsszooss | 24 |
| 1652914 | 6362 | 6381 | 1264 | 1283 | GGTGAATCTTTATTAAACTA | soooszzssssssszooss | 24 |
| 1652916 | 6362 | 6381 | 1264 | 1283 | GGTGAATCTTTATTAAACTA | soooszzzsssssszooss | 24 |
| 1681003 | 6362 | 6381 | 1264 | 1283 | GGTGAATCTTTATTAAACTA | soooszsssszzszsooss | 24 |
| 1681004 | 6362 | 6381 | 1264 | 1283 | GGTGAATCTTTATTAAACTA | ssooszsssszzszsooss | 24 |
| 1681005 | 6362 | 6381 | 1264 | 1283 | GGTGAATCTTTATTAAACTA | ssssszsssszzszsssss | 24 |
| 1681006 | 6362 | 6381 | 1264 | 1283 | GGTGAATCTTTATTAAACTA | ssssszsssszzszzssss | 24 |
| 1681007 | 6362 | 6381 | 1264 | 1283 | GGTGAATCTTTATTAAACTA | zsssszsssszzszssszz | 24 |
| 1681020 | 6362 | 6381 | 1264 | 1283 | GGTGAATCTTTATTAAACTA | zsssszsssszzszssssz | 24 |
| 1681021 | 6362 | 6381 | 1264 | 1283 | GGTGAATCTTTATTAAACTA | zsssszsssszzssssssz | 24 |
| 1693039 | 6365 | 6384 | 1267 | 1286 | CTTGGTGAATCTTTATTAAA | sssosszzzssssssooss | 23 |
| 1693040 | 6365 | 6384 | 1267 | 1286 | CTTGGTGAATCTTTATTAAA | sssosszzzssssssosss | 23 |
| 1693041 | 6365 | 6384 | 1267 | 1286 | CTTGGTGAATCTTTATTAAA | ssossszzzssssssosss | 23 |
| 1693042 | 6365 | 6384 | 1267 | 1286 | CTTGGTGAATCTTTATTAAA | sssosszzzssssssssss | 23 |
| 1693043 | 6365 | 6384 | 1267 | 1286 | CTTGGTGAATCTTTATTAAA | sssoszzzzssssssosss | 23 |
| 1693044 | 6365 | 6384 | 1267 | 1286 | CTTGGTGAATCTTTATTAAA | sssoszzzzssssssssss | 23 |
| 1693045 | 6365 | 6384 | 1267 | 1286 | CTTGGTGAATCTTTATTAAA | ssssozzzzssssssssss | 23 |
| 1693046 | 6365 | 6384 | 1267 | 1286 | CTTGGTGAATCTTTATTAAA | ssssszzzzzsssssssss | 23 |
| 1693047 | 6365 | 6384 | 1267 | 1286 | CTTGGTGAATCTTTATTAAA | sssoszzzzssssszosss | 23 |
| 1693048 | 6365 | 6384 | 1267 | 1286 | CTTGGTGAATCTTTATTAAA | ssssszzzzssssszosss | 23 |
| 1693049 | 6365 | 6384 | 1267 | 1286 | CTTGGTGAATCTTTATTAAA | ssssszzzzssssszssss | 23 |
| 1693050 | 6364 | 6383 | 1266 | 1285 | TTGGTGAATCTTTATTAAAC | soosszzzsssssssosss | 27 |
| 1693051 | 6364 | 6383 | 1266 | 1285 | TTGGTGAATCTTTATTAAAC | ssosszzzsssssssosss | 27 |
| 1693052 | 6364 | 6383 | 1266 | 1285 | TTGGTGAATCTTTATTAAAC | sossszzzsssssssosss | 27 |
| 1693053 | 6364 | 6383 | 1266 | 1285 | TTGGTGAATCTTTATTAAAC | ssssszzzsssssssosss | 27 |
| 1693055 | 6364 | 6383 | 1266 | 1285 | TTGGTGAATCTTTATTAAAC | sososzzzsssssssosss | 27 |
| 1693056 | 6364 | 6383 | 1266 | 1285 | TTGGTGAATCTTTATTAAAC | ssosszzzssssszsosss | 27 |
| 1693057 | 6364 | 6383 | 1266 | 1285 | TTGGTGAATCTTTATTAAAC | ssssszzzssssszsosss | 27 |
| 1693058 | 6364 | 6383 | 1266 | 1285 | TTGGTGAATCTTTATTAAAC | sssoszzzssssszsosss | 27 |
| 1693059 | 6364 | 6383 | 1266 | 1285 | TTGGTGAATCTTTATTAAAC | ssssszzzssssszsssss | 27 |
| 1693060 | 6364 | 6383 | 1266 | 1285 | TTGGTGAATCTTTATTAAAC | ssssszzzsssszzsosss | 27 |
| 1693061 | 6364 | 6383 | 1266 | 1285 | TTGGTGAATCTTTATTAAAC | sssoszzzsssszzsssss | 27 |
| 1693062 | 6364 | 6383 | 1266 | 1285 | TTGGTGAATCTTTATTAAAC | ssssozzzsssszzsssss | 27 |
| 1693063 | 6364 | 6383 | 1266 | 1285 | TTGGTGAATCTTTATTAAAC | ssssszzzsssszzsssss | 27 |
| 1693064 | 6366 | 6385 | 1268 | 1287 | ACTTGGTGAATCTTTATTAA | soossssszzsssssosss | 22 |
| 1693065 | 6366 | 6385 | 1268 | 1287 | ACTTGGTGAATCTTTATTAA | soossssszzsssssssss | 22 |
| 1693066 | 6366 | 6385 | 1268 | 1287 | ACTTGGTGAATCTTTATTAA | sssssssszzsssssooss | 22 |
| 1693067 | 6366 | 6385 | 1268 | 1287 | ACTTGGTGAATCTTTATTAA | ssosssszzzszsssssss | 22 |
| 1693068 | 6366 | 6385 | 1268 | 1287 | ACTTGGTGAATCTTTATTAA | sssossszzzszsssssss | 22 |
| 1693069 | 6366 | 6385 | 1268 | 1287 | ACTTGGTGAATCTTTATTAA | ssssosszzzszsssssss | 22 |
| 1693070 | 6366 | 6385 | 1268 | 1287 | ACTTGGTGAATCTTTATTAA | ssssssszzzszsssosss | 22 |
| 1693071 | 6366 | 6385 | 1268 | 1287 | ACTTGGTGAATCTTTATTAA | ssssssszzzzzsssosss | 22 |
| 1693072 | 6366 | 6385 | 1268 | 1287 | ACTTGGTGAATCTTTATTAA | ssssosszzzzzsssssss | 22 |
| 1693073 | 6366 | 6385 | 1268 | 1287 | ACTTGGTGAATCTTTATTAA | sssossszzzzzsssssss | 22 |
| 1693074 | 6366 | 6385 | 1268 | 1287 | ACTTGGTGAATCTTTATTAA | ssosssszzzzzsssssss | 22 |
| 1693075 | 6366 | 6385 | 1268 | 1287 | ACTTGGTGAATCTTTATTAA | ssssssszzzzzsssssss | 22 |

### Example 2: Design of Mixed MOE/cEt Gapmers Complementary to Human APOE RNA

Modified oligonucleotides complementary to a human APOE RNA were designed and synthesized following standard techniques.

The modified oligonucleotides in the tables below are mixed MOE/cEt modified oligonucleotides with mixed PS/PO/mesyl internucleoside linkages. The modified oligonucleotides in the tables below are 18-21 nucleosides in length, as indicated by each table title. The sugar motifs for the modified oligonucleotides are presented in the column labeled “Sugar Motif (5′ to 3′)”, wherein each “e” represents a 2′-MOE sugar moiety, each “k” represents a cEt sugar moiety, and each “d” represents a 2′-β-D-deoxyribosyl sugar moiety. The internucleoside linkage motifs for the modified oligonucleotides are presented in the column labeled “Internucleoside Linkage (5′ to 3′)”, wherein each “s” represents a phosphorothioate internucleoside linkage, each “o” represents a phosphodiester internucleoside linkage, and each “z” represents a mesyl phosphoramidate internucleoside linkage. Each cytosine residue is a 5-methylcytosine.

Each modified oligonucleotide listed in the table below is 10000 complementary to SEQ ID NO: 1 (described herein above) and to SEQ ID NO: 2 (described herein above). “Start site” indicates the 5′-most nucleoside to which the modified oligonucleotide is complementary in the target nucleic acid sequence. “Stop site” indicates the 3-most nucleoside to which the modified oligonucleotide is complementary in the target nucleic acid sequence.

TABLE 3
Mixed MOE/cEt gapmers with mixed internucleoside linkages complementary
to human APOE, 18 nucleosides

| | SEQ | SEQ | SEQ | SEQ | | | | |
|---|---|---|---|---|---|---|---|---|
| | ID | ID | ID | ID | | | | |
| | NO: 1 | NO: 1 | NO: 2 | NO: 2 | | | | SEQ |
| Compound | Start | Stop | Start | Stop | | Sugar Motif | Internucleoside | ID |
| Number | Site | Site | Site | Site | Sequence (5′ to 3′) | (5′ to 3′) | Linkage (5′ to 3′) | NO |
| 1757557 | 6366 | 6383 | 1268 | 1285 | TTGGTGAATCTTTATTAA | eeeeedddddddd | soossssssssssooss | 38 |
| | | | | | | eekke | | |
| 1757556 | 6366 | 6383 | 1268 | 1285 | TTGGTGAATCTTTATTAA | eeeeedddddddd | soossssssssssooss | 38 |
| | | | | | | ekkee | | |
| 1757541 | 6366 | 6383 | 1268 | 1285 | TTGGTGAATCTTTATTAA | eeeekdddddddd | soossssssssssooss | 38 |
| | | | | | | eeeee | | |
| 1757559 | 6366 | 6383 | 1268 | 1285 | TTGGTGAATCTTTATTAA | eeeekdddddddd | soossssssssssooss | 38 |
| | | | | | | keeee | | |
| 1757564 | 6366 | 6383 | 1268 | 1285 | TTGGTGAATCTTTATTAA | eeeekdddddddd | soossssssssssooss | 38 |
| | | | | | | keeke | | |
| 1757563 | 6366 | 6383 | 1268 | 1285 | TTGGTGAATCTTTATTAA | eeeekdddddddd | soossssssssssooss | 38 |
| | | | | | | kekee | | |
| 1757562 | 6366 | 6383 | 1268 | 1285 | TTGGTGAATCTTTATTAA | eeeekdddddddd | soossssssssssooss | 38 |
| | | | | | | kkeee | | |
| 1757551 | 6366 | 6383 | 1268 | 1285 | TTGGTGAATCTTTATTAA | eeekkdddddddd | soossssssssssooss | 38 |
| | | | | | | eeeee | | |
| 1757561 | 6366 | 6383 | 1268 | 1285 | TTGGTGAATCTTTATTAA | eeekkdddddddd | soossssssssssooss | 38 |
| | | | | | | keeee | | |
| 1757544 | 6366 | 6383 | 1268 | 1285 | TTGGTGAATCTTTATTAA | ekeeedddddddd | soossssssssssooss | 38 |
| | | | | | | eeeee | | |
| 1757554 | 6366 | 6383 | 1268 | 1285 | TTGGTGAATCTTTATTAA | kkeeedddddddd | soossssssssssooss | 38 |
| | | | | | | eeeee | | |

TABLE 4
Mixed MOE/cEt gapmers with mixed internucleoside linkages complementary
to human APOE, 19 nucleosides

| | SEQ | SEQ | SEQ | SEQ | | | | |
|---|---|---|---|---|---|---|---|---|
| | ID | ID | ID | ID | | | | |
| | NO: 1 | NO: 1 | NO: 2 | NO: 2 | | | | SEQ |
| Compound | Start | Stop | Start | Stop | | Sugar Motif | Internucleoside | ID |
| Number | Site | Site | Site | Site | Sequence (5′ to 3′) | (5′ to 3′) | Linkage (5′ to 3′) | NO |
| 1809691 | 6366 | 6384 | 1268 | 1286 | CTTGGTGAATCTTTATTAA | eeeekdddddddd | sosssszzzssssszoss | 39 |
| | | | | | | ddkeek | | |
| 1809688 | 6366 | 6384 | 1268 | 1286 | CTTGGTGAATCTTTATTAA | eeeekdddddddd | sosssszzzssssszoss | 39 |
| | | | | | | ddkkee | | |
| 1809690 | 6366 | 6384 | 1268 | 1286 | CTTGGTGAATCTTTATTAA | eeeekdddddddd | sosssszzzssssszoss | 39 |
| | | | | | | ddkkek | | |

TABLE 5
Mixed MOE/cEt gapmers with mixed internucleoside linkages complementary
to human APOE, 20 nucleosides

| | SEQ | SEQ | SEQ | SEQ | | | | |
|---|---|---|---|---|---|---|---|---|
| | ID | ID | ID | ID | | | | |
| | NO: 1 | NO: 1 | NO: 2 | NO: 2 | | | | SEQ |
| Compound | Start | Stop | Start | Stop | | Sugar Motif | Internucleoside | ID |
| Number | Site | Site | Site | Site | Sequence (5′ to 3′) | (5′ to 3′) | Linkage (5′ to 3′) | NO |
| 1757567 | 6366 | 6385 | 1268 | 1287 | ACTTGGTGAATCTTTAT | eeeeeddddddd | sooosssssssssssooss | 22 |
| | | | | | TAA | dddkeeee | | |
| 1757583 | 6364 | 6383 | 1266 | 1285 | TTGGTGAATCTTTATTA | eeeeeddddddd | sooosssssssssssooss | 27 |
| | | | | | AAC | dddkeeee | | |
| 1757568 | 6366 | 6385 | 1268 | 1287 | ACTTGGTGAATCTTTAT | eeeeeddddddd | sooosssssssssssooss | 22 |
| | | | | | TAA | dddkkeee | | |
| 1757584 | 6364 | 6383 | 1266 | 1285 | TTGGTGAATCTTTATTA | eeeeeddddddd | sooosssssssssssooss | 27 |
| | | | | | AAC | dddkkeee | | |
| 1766537 | 6364 | 6383 | 1266 | 1285 | TTGGTGAATCTTTATTA | eeeeeddddddd | soooszzzsssszssooss | 27 |
| | | | | | AAC | dddkkeee | | |
| 1766559 | 6364 | 6383 | 1266 | 1285 | TTGGTGAATCTTTATTA | eeeeeddddddd | soooszzzsssszzsooss | 27 |
| | | | | | AAC | dddkkeee | | |
| 1757573 | 6366 | 6385 | 1268 | 1287 | ACTTGGTGAATCTTTAT | eeeeeedddddd | soosssszzzssssszsss | 22 |
| | | | | | TAA | ddddkeee | | |
| 1778117 | 6366 | 6385 | 1268 | 1287 | ACTTGGTGAATCTTTAT | eeeeeedddddd | sooossszzsssssszsss | 22 |
| | | | | | TAA | ddddkeee | | |
| 1778119 | 6366 | 6385 | 1268 | 1287 | ACTTGGTGAATCTTTAT | eeeeeedddddd | sooossszzsssssszoss | 22 |
| | | | | | TAA | ddddkeee | | |
| 1778120 | 6366 | 6385 | 1268 | 1287 | ACTTGGTGAATCTTTAT | eeeeeedddddd | soooosszzsssssszoss | 22 |
| | | | | | TAA | ddddkeee | | |
| 1757574 | 6366 | 6385 | 1268 | 1287 | ACTTGGTGAATCTTTAT | eeeeeedddddd | soosssszzzssssszsss | 22 |
| | | | | | TAA | ddddkkee | | |
| 1778123 | 6366 | 6385 | 1268 | 1287 | ACTTGGTGAATCTTTAT | eeeeeedddddd | sooossszzsssssszoss | 22 |
| | | | | | TAA | ddddkkee | | |
| 1778125 | 6366 | 6385 | 1268 | 1287 | ACTTGGTGAATCTTTAT | eeeeeedddddd | soooosszzsssssszoss | 22 |
| | | | | | TAA | ddddkkee | | |
| 1778154 | 6365 | 6384 | 1267 | 1286 | CTTGGTGAATCTTTATT | eeeeeedddddd | ssoooszzssssssssoos | 23 |
| | | | | | AAA | ddddkkee | | |
| 1757571 | 6366 | 6385 | 1268 | 1287 | ACTTGGTGAATCTTTAT | eeeeekdddddd | soosssszzzssssszsss | 22 |
| | | | | | TAA | ddddeeee | | |
| 1778111 | 6366 | 6385 | 1268 | 1287 | ACTTGGTGAATCTTTAT | eeeeekdddddd | sooossszzsssssszoss | 22 |
| | | | | | TAA | ddddeeee | | |
| 1778112 | 6366 | 6385 | 1268 | 1287 | ACTTGGTGAATCTTTAT | eeeeekdddddd | soooosszzsssssszoss | 22 |
| | | | | | TAA | ddddeeee | | |
| 1757576 | 6366 | 6385 | 1268 | 1287 | ACTTGGTGAATCTTTAT | eeeeekdddddd | soosssszzzssssszsss | 22 |
| | | | | | TAA | ddddkkee | | |
| 1778129 | 6366 | 6385 | 1268 | 1287 | ACTTGGTGAATCTTTAT | eeeeekdddddd | sooossszzsssssszsss | 22 |
| | | | | | TAA | ddddkkee | | |
| 1778130 | 6366 | 6385 | 1268 | 1287 | ACTTGGTGAATCTTTAT | eeeeekdddddd | sooossszzsssssszoss | 22 |
| | | | | | TAA | ddddkkee | | |
| 1778131 | 6366 | 6385 | 1268 | 1287 | ACTTGGTGAATCTTTAT | eeeeekdddddd | soooosszzsssssszoss | 22 |
| | | | | | TAA | ddddkkee | | |
| 1757565 | 6366 | 6385 | 1268 | 1287 | ACTTGGTGAATCTTTAT | eeeekddddddd | sooosssssssssssooss | 22 |
| | | | | | TAA | dddeeeee | | |
| 1757581 | 6364 | 6383 | 1266 | 1285 | TTGGTGAATCTTTATTA | eeeekddddddd | sooosssssssssssooss | 27 |
| | | | | | AAC | dddeeeee | | |
| 1757570 | 6366 | 6385 | 1268 | 1287 | ACTTGGTGAATCTTTAT | eeeekddddddd | sooosssssssssssooss | 22 |
| | | | | | TAA | dddkkeee | | |
| 1757586 | 6364 | 6383 | 1266 | 1285 | TTGGTGAATCTTTATTA | eeeekddddddd | sooosssssssssssooss | 27 |
| | | | | | AAC | dddkkeee | | |
| 1757572 | 6366 | 6385 | 1268 | 1287 | ACTTGGTGAATCTTTAT | eeeekkdddddd | soosssszzzssssszsss | 22 |
| | | | | | TAA | ddddeeee | | |
| 1778113 | 6366 | 6385 | 1268 | 1287 | ACTTGGTGAATCTTTAT | eeeekkdddddd | sooossszzsssssszsss | 22 |
| | | | | | TAA | ddddeeee | | |
| 1778114 | 6366 | 6385 | 1268 | 1287 | ACTTGGTGAATCTTTAT | eeeekkdddddd | sooossszzsssssszoss | 22 |
| | | | | | TAA | ddddeeee | | |
| 1778115 | 6366 | 6385 | 1268 | 1287 | ACTTGGTGAATCTTTAT | eeeekkdddddd | soooosszzsssssszoss | 22 |
| | | | | | TAA | ddddeeee | | |
| 1757575 | 6366 | 6385 | 1268 | 1287 | ACTTGGTGAATCTTTAT | eeeekkdddddd | soosssszzzssssszsss | 22 |
| | | | | | TAA | ddddkeee | | |
| 1778126 | 6366 | 6385 | 1268 | 1287 | ACTTGGTGAATCTTTAT | eeeekkdddddd | sooossszzsssssszsss | 22 |
| | | | | | TAA | ddddkeee | | |
| 1778127 | 6366 | 6385 | 1268 | 1287 | ACTTGGTGAATCTTTAT | eeeekkdddddd | soooosszzsssssszozz | 22 |
| | | | | | TAA | ddddkeee | | |
| 1778128 | 6366 | 6385 | 1268 | 1287 | ACTTGGTGAATCTTTAT | eeeekkdddddd | soooosszzsssssszoss | 22 |
| | | | | | TAA | ddddkeee | | |
| 1809813 | 6364 | 6383 | 1266 | 1285 | TTGGTGAATCTTTATTA | eeekdddddddd | soosszzzssssszsssss | 27 |
| | | | | | AAC | ddkkeeee | | |
| 1809814 | 6364 | 6383 | 1266 | 1285 | TTGGTGAATCTTTATTA | eeekdddddddd | zoosszzzssssszsssss | 27 |
| | | | | | AAC | ddkkeeee | | |
| 1809818 | 6364 | 6383 | 1266 | 1285 | TTGGTGAATCTTTATTA | eeekdddddddd | soosszzzssssszsooss | 27 |
| | | | | | AAC | ddkkeeee | | |
| 1757566 | 6366 | 6385 | 1268 | 1287 | ACTTGGTGAATCTTTAT | eeekkddddddd | sooosssssssssssooss | 22 |
| | | | | | TAA | dddeeeee | | |
| 1757582 | 6364 | 6383 | 1266 | 1285 | TTGGTGAATCTTTATTA | eeekkddddddd | sooosssssssssssooss | 27 |
| | | | | | AAC | dddeeeee | | |
| 1757569 | 6366 | 6385 | 1268 | 1287 | ACTTGGTGAATCTTTAT | eeekkddddddd | sooosssssssssssooss | 22 |
| | | | | | TAA | dddkeeee | | |
| 1757585 | 6364 | 6383 | 1266 | 1285 | TTGGTGAATCTTTATTA | eeekkddddddd | sooosssssssssssooss | 27 |
| | | | | | AAC | dddkeeee | | |
| 1809635 | 6366 | 6385 | 1268 | 1287 | ACTTGGTGAATCTTTAT | keeeekdddddd | soosssszzzssssszsss | 22 |
| | | | | | TAA | ddddkkee | | |
| 1809636 | 6366 | 6385 | 1268 | 1287 | ACTTGGTGAATCTTTAT | keeeekdddddd | soosssszzzssssszsss | 22 |
| | | | | | TAA | ddddkkek | | |
| 1809637 | 6366 | 6385 | 1268 | 1287 | ACTTGGTGAATCTTTAT | keeeekdddddd | soosssszzzssssszsss | 22 |
| | | | | | TAA | ddddkkkk | | |
| 1736492 | 6365 | 6384 | 1267 | 1286 | CTTGGTGAATCTTTATT | eeeeeedddddd | sosssszzzssssszssss | 23 |
| | | | | | AAA | ddddkeee | | |
| 1736528 | 6365 | 6384 | 1267 | 1286 | CTTGGTGAATCTTTATT | eeeeeedddddd | sssssozzzssssszssss | 23 |
| | | | | | AAA | ddddkeee | | |
| 1736533 | 6365 | 6384 | 1267 | 1286 | CTTGGTGAATCTTTATT | eeeeeedddddd | ssssoszzzssssszssss | 23 |
| | | | | | AAA | ddddkeee | | |
| 1736493 | 6365 | 6384 | 1267 | 1286 | CTTGGTGAATCTTTATT | eeeeeedddddd | sosssszzzssssszssss | 23 |
| | | | | | AAA | ddddkkee | | |
| 1736529 | 6365 | 6384 | 1267 | 1286 | CTTGGTGAATCTTTATT | eeeeeedddddd | sssssozzzssssszssss | 23 |
| | | | | | AAA | ddddkkee | | |
| 1736534 | 6365 | 6384 | 1267 | 1286 | CTTGGTGAATCTTTATT | eeeeeedddddd | ssssoszzzssssszssss | 23 |
| | | | | | AAA | ddddkkee | | |
| 1736513 | 6365 | 6384 | 1267 | 1286 | CTTGGTGAATCTTTATT | eeeeekdddddd | sosssszzzssssszssss | 23 |
| | | | | | AAA | ddddeeee | | |
| 1736516 | 6365 | 6384 | 1267 | 1286 | CTTGGTGAATCTTTATT | eeeeekdddddd | soossszzzssssszssss | 23 |
| | | | | | AAA | ddddeeee | | |
| 1736531 | 6365 | 6384 | 1267 | 1286 | CTTGGTGAATCTTTATT | eeeeekdddddd | sssssozzzssssszssss | 23 |
| | | | | | AAA | ddddeeee | | |
| 1736535 | 6365 | 6384 | 1267 | 1286 | CTTGGTGAATCTTTATT | eeeeekdddddd | ssssoszzzssssszssss | 23 |
| | | | | | AAA | ddddeeee | | |
| 1736514 | 6365 | 6384 | 1267 | 1286 | CTTGGTGAATCTTTATT | eeeekkdddddd | sosssszzzssssszssss | 23 |
| | | | | | AAA | ddddeeee | | |
| 1736517 | 6365 | 6384 | 1267 | 1286 | CTTGGTGAATCTTTATT | eeeekkdddddd | soossszzzssssszssss | 23 |
| | | | | | AAA | ddddeeee | | |
| 1736532 | 6365 | 6384 | 1267 | 1286 | CTTGGTGAATCTTTATT | eeeekkdddddd | sssssozzzssssszssss | 23 |
| | | | | | AAA | ddddeeee | | |
| 1736536 | 6365 | 6384 | 1267 | 1286 | CTTGGTGAATCTTTATT | eeeekkdddddd | ssssoszzzssssszssss | 23 |
| | | | | | AAA | ddddeeee | | |
| 1779699 | 6365 | 6384 | 1267 | 1286 | CTTGGTGAATCTTTATT | eeekkedddddd | sosssszzzssssszssss | 23 |
| | | | | | AAA | ddddeeee | | |
| 1779701 | 6365 | 6384 | 1267 | 1286 | CTTGGTGAATCTTTATT | eeekkddddddd | sossszzzzssssszssss | 23 |
| | | | | | AAA | ddddeeee | | |
| 1779706 | 6365 | 6384 | 1267 | 1286 | CTTGGTGAATCTTTATT | eeekkddddddd | soooszzzsssssssssss | 23 |
| | | | | | AAA | ddddeeee | | |
| 1779961 | 6365 | 6384 | 1267 | 1286 | CTTGGTGAATCTTTATT | eeekkedddddd | soszsszzzssssszssss | 23 |
| | | | | | AAA | ddddeeee | | |
| 1779970 | 6365 | 6384 | 1267 | 1286 | CTTGGTGAATCTTTATT | eeekkedddddd | soossszzsssssssssss | 23 |
| | | | | | AAA | ddddeeee | | |
| 1778108 | 6366 | 6385 | 1268 | 1287 | ACTTGGTGAATCTTTAT | eeeeekdddddd | sooossszzsssssszsss | 22 |
| | | | | | TAA | ddddeeee | | |
| 1778122 | 6366 | 6385 | 1268 | 1287 | ACTTGGTGAATCTTTAT | eeeeeedddddd | sooossszzsssssszsss | 22 |
| | | | | | TAA | ddddkkee | | |
| 1809815 | 6364 | 6383 | 1266 | 1285 | TTGGTGAATCTTTATTA | eeekdddddddd | zossszzzssssszsssss | 27 |
| | | | | | AAC | ddkkeeee | | |
| 1809816 | 6364 | 6383 | 1266 | 1285 | TTGGTGAATCTTTATTA | eeekdddddddd | zossszzzssssszssssz | 27 |
| | | | | | AAC | ddkkeeee | | |

TABLE 6
Design of mixed MOE/cEt gapmers with mixed internucleoside linkages complementary
to human APOE, 21 nucleosides

| | SEQ | SEQ | SEQ | SEQ | | | | |
|---|---|---|---|---|---|---|---|---|
| | ID | ID | ID | ID | | | | |
| | NO: 1 | NO: 1 | NO: 2 | NO: 2 | | | | SEQ |
| Compound | Start | Stop | Start | Stop | | Sugar Motif | Internucleoside | ID |
| Number | Site | Site | Site | Site | Sequence (5′ to 3′) | (5′ to 3′) | Linkage (5′ to 3′) | NO |
| 1810063 | 6364 | 6384 | 1266 | 1286 | CTTGGTGAATCTTTATT | eeeeeeddddd | ssoosszzzssssszsooss | 40 |
| | | | | | AAAC | dddddkkeee | | |
| 1809741 | 6364 | 6384 | 1266 | 1286 | CTTGGTGAATCTTTATT | eeeeekddddd | ssoooszzsssssszsooss | 40 |
| | | | | | AAAC | dddddeeeee | | |
| 1809745 | 6364 | 6384 | 1266 | 1286 | CTTGGTGAATCTTTATT | eeeeekddddd | zsoooszzsssssszsoosz | 40 |
| | | | | | AAAC | dddddeeeee | | |
| 1810064 | 6364 | 6384 | 1266 | 1286 | CTTGGTGAATCTTTATT | eeeeekddddd | ssoooszzzssssszsooss | 40 |
| | | | | | AAAC | dddddeeeee | | |
| 1810065 | 6364 | 6384 | 1266 | 1286 | CTTGGTGAATCTTTATT | eeeeekddddd | ssoosszzzssssszsooss | 40 |
| | | | | | AAAC | dddddeeeee | | |
| 1810069 | 6364 | 6384 | 1266 | 1286 | CTTGGTGAATCTTTATT | eeeeekddddd | zsossszzzssssszsoosz | 40 |
| | | | | | AAAC | dddddeeeee | | |
| 1809744 | 6364 | 6384 | 1266 | 1286 | CTTGGTGAATCTTTATT | eeeekkddddd | zsoooszzsssssszsoosz | 40 |

| | |
|---|---|
| AAAC | dddddeeeee |

### Example 3: Design of cEt Gapmers Complementary to Human APOE RNA

Modified oligonucleotides complementary to a human APOE RNA were designed and synthesized following standard techniques.

The modified oligonucleotides in the table below are mixed cEt gapmers with mixed PS/mesyl internucleoside linkages. The modified oligonucleotides in the table below are 20 nucleosides in length. The sugar motifs for the modified oligonucleotides are presented in the column labeled “Sugar Motif (5′ to 3′)”, wherein each “k” represents a cEt sugar moiety, and each “d” represents a 2′-β-D-deoxyribosyl sugar moiety. The internucleoside linkage motifs for the modified oligonucleotides are presented in the column labeled “Internucleoside Linkage (5′ to 3′)”, wherein each “s” represents a phosphorothioate internucleoside linkage, and each “z” represents a mesyl phosphoramidate internucleoside linkage. Each cytosine residue is a 5-methylcytosine.

Each modified oligonucleotide listed in the table below is 10000 complementary to SEQ ID NO: 1 (described herein above) and to SEQ ID NO: 2 (described herein above). “Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence.

TABLE 7
Design of cEt gapmers with mixed PS/mesyl internucleoside linkages
complementary to human APOE

| | SEQ | SEQ | SEQ | SEQ | | | | |
|---|---|---|---|---|---|---|---|---|
| | ID | ID | ID | ID | | | | |
| | NO: 1 | NO: 1 | NO: 2 | NO: 2 | | | | SEQ |
| Compound | Start | Stop | Start | Stop | | Sugar Motif | Internucleoside | ID |
| Number | Site | Site | Site | Site | Sequence (5′ to 3′) | (5′ to 3′) | Linkage (5′ to 3′) | NO |
| 1746784 | 6365 | 6384 | 1267 | 1286 | CTTGGTGAATCTTTATTA | kkkdddddddddd | ssszzzzzzssssszss | 23 |
| | | | | | AA | dddkkdk | sz | |
| 1746799 | 6365 | 6384 | 1267 | 1286 | CTTGGTGAATCTTTATTA | kdkdkdddddddd | szszszzzzssssszss | 23 |
| | | | | | AA | dddkdkk | zs | |
| 1746800 | 6365 | 6384 | 1267 | 1286 | CTTGGTGAATCTTTATTA | kdkddkddddddd | szszzszzzssssszss | 23 |
| | | | | | AA | dddkdkk | zs | |
| 1746809 | 6365 | 6384 | 1267 | 1286 | CTTGGTGAATCTTTATTA | kdkdkdddddddd | szszszzzzssssszsz | 23 |
| | | | | | AA | ddddkkk | ss | |
| 1746785 | 6365 | 6384 | 1267 | 1286 | CTTGGTGAATCTTTATTA | kkdkddddddddd | sszszzzzzssssszss | 23 |
| | | | | | AA | dddkkdk | sz | |
| 1746786 | 6365 | 6384 | 1267 | 1286 | CTTGGTGAATCTTTATTA | kkddkdddddddd | sszzszzzzssssszss | 23 |
| | | | | | AA | dddkkdk | sz | |
| 1746787 | 6365 | 6384 | 1267 | 1286 | CTTGGTGAATCTTTATTA | kkdddkddddddd | sszzzszzzssssszss | 23 |
| | | | | | AA | dddkkdk | sz | |
| 1746789 | 6365 | 6384 | 1267 | 1286 | CTTGGTGAATCTTTATTA | kdkdkdddddddd | szszszzzzssssszss | 23 |
| | | | | | AA | dddkkdk | sz | |
| 1746790 | 6365 | 6384 | 1267 | 1286 | CTTGGTGAATCTTTATTA | kdkddkddddddd | szszzszzzssssszss | 23 |
| | | | | | AA | dddkkdk | sz | |
| 1746791 | 6365 | 6384 | 1267 | 1286 | CTTGGTGAATCTTTATTA | kddkkdddddddd | szzsszzzzssssszss | 23 |
| | | | | | AA | dddkkdk | sz | |
| 1746792 | 6365 | 6384 | 1267 | 1286 | CTTGGTGAATCTTTATTA | kddkdkddddddd | szzszszzzssssszss | 23 |
| | | | | | AA | dddkkdk | sz | |
| 1746793 | 6365 | 6384 | 1267 | 1286 | CTTGGTGAATCTTTATTA | kdddkkddddddd | szzzsszzzssssszss | 23 |
| | | | | | AA | dddkkdk | sz | |
| 1746794 | 6365 | 6384 | 1267 | 1286 | CTTGGTGAATCTTTATTA | kkkdddddddddd | ssszzzzzzssssszss | 23 |
| | | | | | AA | dddkdkk | zs | |
| 1746795 | 6365 | 6384 | 1267 | 1286 | CTTGGTGAATCTTTATTA | kkdkddddddddd | sszszzzzzssssszss | 23 |
| | | | | | AA | dddkdkk | zs | |
| 1746796 | 6365 | 6384 | 1267 | 1286 | CTTGGTGAATCTTTATTA | kkddkdddddddd | sszzszzzzssssszss | 23 |
| | | | | | AA | dddkdkk | zs | |
| 1746797 | 6365 | 6384 | 1267 | 1286 | CTTGGTGAATCTTTATTA | kkdddkddddddd | sszzzszzzssssszss | 23 |
| | | | | | AA | dddkdkk | zs | |
| 1746801 | 6365 | 6384 | 1267 | 1286 | CTTGGTGAATCTTTATTA | kddkkdddddddd | szzsszzzzssssszss | 23 |
| | | | | | AA | dddkdkk | zs | |
| 1746802 | 6365 | 6384 | 1267 | 1286 | CTTGGTGAATCTTTATTA | kdddkkddddddd | szzzsszzzssssszss | 23 |
| | | | | | AA | dddkdkk | zs | |
| 1746803 | 6365 | 6384 | 1267 | 1286 | CTTGGTGAATCTTTATTA | kddkdkddddddd | szzszszzzssssszss | 23 |
| | | | | | AA | dddkdkk | zs | |
| 1746804 | 6365 | 6384 | 1267 | 1286 | CTTGGTGAATCTTTATTA | kkkdddddddddd | ssszzzzzzssssszsz | 23 |
| | | | | | AA | ddddkkk | ss | |
| 1746805 | 6365 | 6384 | 1267 | 1286 | CTTGGTGAATCTTTATTA | kkdkddddddddd | sszszzzzzssssszsz | 23 |
| | | | | | AA | ddddkkk | ss | |
| 1746806 | 6365 | 6384 | 1267 | 1286 | CTTGGTGAATCTTTATTA | kkddkdddddddd | sszzszzzzssssszsz | 23 |
| | | | | | AA | ddddkkk | ss | |
| 1746807 | 6365 | 6384 | 1267 | 1286 | CTTGGTGAATCTTTATTA | kkdddkddddddd | sszzzszzzssssszsz | 23 |
| | | | | | AA | ddddkkk | ss | |
| 1746810 | 6365 | 6384 | 1267 | 1286 | CTTGGTGAATCTTTATTA | kdkddkddddddd | szszzszzzssssszsz | 23 |
| | | | | | AA | ddddkkk | ss | |
| 1746811 | 6365 | 6384 | 1267 | 1286 | CTTGGTGAATCTTTATTA | kddkkdddddddd | szzsszzzzssssszsz | 23 |
| | | | | | AA | ddddkkk | ss | |
| 1746812 | 6365 | 6384 | 1267 | 1286 | CTTGGTGAATCTTTATTA | kdddkkddddddd | szzzsszzzssssszsz | 23 |
| | | | | | AA | ddddkkk | ss | |
| 1746813 | 6365 | 6384 | 1267 | 1286 | CTTGGTGAATCTTTATTA | kddkdkddddddd | szzszszzzssssszsz | 23 |
| | | | | | AA | ddddkkk | ss | |
| 1747051 | 6365 | 6384 | 1267 | 1286 | CTTGGTGAATCTTTATTA | kkdkdkddddddd | sszszszzzssssszss | 23 |
| | | | | | AA | dddkddk | zz | |
| 1747052 | 6365 | 6384 | 1267 | 1286 | CTTGGTGAATCTTTATTA | kdkkdkddddddd | szsszszzzssssszss | 23 |
| | | | | | AA | dddkddk | zz | |
| 1747053 | 6365 | 6384 | 1267 | 1286 | CTTGGTGAATCTTTATTA | kddkkkddddddd | szzssszzzssssszss | 23 |
| | | | | | AA | dddkddk | zz | |
| 1753989 | 6365 | 6384 | 1267 | 1286 | CTTGGTGAATCTTTATTA | kdkdkdddddddd | szszsszzzssssszsz | 23 |
| | | | | | AA | ddkdkdk | sz | |
| 1747050 | 6365 | 6384 | 1267 | 1286 | CTTGGTGAATCTTTATTA | kkkddkddddddd | ssszzszzzssssszss | 23 |
| | | | | | AA | dddkddk | zz | |
| 1785588 | 6365 | 6384 | 1267 | 1286 | CTTGGTGAATCTTTATTA | kkkdddddddddd | ssszzzsszzsssssss | 23 |
| | | | | | AA | dddkkdk | sz | |
| 1785589 | 6365 | 6384 | 1267 | 1286 | CTTGGTGAATCTTTATTA | kkkdddddddddd | ssszzzssszzssssss | 23 |
| | | | | | AA | dddkkdk | sz | |
| 1785591 | 6365 | 6384 | 1267 | 1286 | CTTGGTGAATCTTTATTA | kkkdddddddddd | ssszzzssssszzssss | 23 |
| | | | | | AA | dddkkdk | sz | |
| 1785598 | 6365 | 6384 | 1267 | 1286 | CTTGGTGAATCTTTATTA | kkkdddddddddd | ssszzzzzzsssssszs | 23 |
| | | | | | AA | dddkkdk | sz | |
| 1785599 | 6365 | 6384 | 1267 | 1286 | CTTGGTGAATCTTTATTA | kkkdddddddddd | ssszzzzzzzsssssss | 23 |
| | | | | | AA | dddkkdk | sz | |
| 1785600 | 6365 | 6384 | 1267 | 1286 | CTTGGTGAATCTTTATTA | kkkdddddddddd | ssszzzssssszzzsss | 23 |
| | | | | | AA | dddkkdk | sz | |
| 1785601 | 6365 | 6384 | 1267 | 1286 | CTTGGTGAATCTTTATTA | kkkdddddddddd | ssszzzszzssssszss | 23 |
| | | | | | AA | dddkkdk | sz | |
| 1785602 | 6365 | 6384 | 1267 | 1286 | CTTGGTGAATCTTTATTA | kkkdddddddddd | ssszzzzzzssssssss | 23 |
| | | | | | AA | dddkkdk | sz | |
| 1785604 | 6365 | 6384 | 1267 | 1286 | CTTGGTGAATCTTTATTA | kddkdkddddddd | szzszszzsssssssss | 23 |
| | | | | | AA | dddkkdk | sz | |
| 1785616 | 6365 | 6384 | 1267 | 1286 | CTTGGTGAATCTTTATTA | kddkdkddddddd | szzszszzzsssssszs | 23 |
| | | | | | AA | dddkkdk | sz | |
| 1785617 | 6365 | 6384 | 1267 | 1286 | CTTGGTGAATCTTTATTA | kddkdkddddddd | szzszszzzzsssssss | 23 |
| | | | | | AA | dddkkdk | sz | |
| 1785619 | 6365 | 6384 | 1267 | 1286 | CTTGGTGAATCTTTATTA | kddkdkddddddd | szzszsszzssssszss | 23 |
| | | | | | AA | dddkkdk | sz | |
| 1785586 | 6365 | 6384 | 1267 | 1286 | CTTGGTGAATCTTTATTA | kkkdddddddddd | ssszzzzzsssssssss | 23 |
| | | | | | AA | dddkkdk | sz | |

### Example 4: Dose-Dependent Inhibition of Human APOE RNA in HepG2 Cells by Modified Oligonucleotides

Modified oligonucleotides selected from the examples above were tested at various doses in HepG2 cells. The modified oligonucleotides were tested in a series of experiments that had the same culture conditions. Each experiment is identified in separate tables below.

HepG2 cells plated at a density of 50,000 cells per well were treated by electroporation with various concentrations of modified oligonucleotide as specified in the tables below. After a treatment period of approximately 16-24 hours as indicated in the table titles below, total RNA was isolated from the cells, and APOE RNA levels were measured by quantitative real-time RT-PCR. APOE RNA levels were measured using the human primer probe set RTS3073 (forward sequence TGGGTCGCTTTTGGGATTAC, designated herein as SEQ ID NO: 7; reverse sequence CCATCAGCGCCCTCAGTT, designated herein as SEQ ID NO: 8; probe sequence CTGCTCAGCTCCCAGGTCACCCA, designated herein as SEQ ID NO:9). APOE RNA levels were normalized to human GAPDH. Human GAPDH was amplified using human primer probe set RTS 104 (forward sequence GAAGGTGAAGGTCGGAGTC, designated herein as SEQ ID NO: 10; reverse sequence GAAGATGGTGATGGGATTTC, designated herein as SEQ ID NO: 11; probe sequence CAAGCTTCCCGTTCTCAGCC, designated herein as SEQ ID NO: 12). Results are presented as percent APOE RNA, relative to the amount of APOE RNA in untreated control cells (UTC). The halfmaximal inhibitory concentration (IC₅₀) of each modified oligonucleotide was calculated in GraphPad Prism 10 (GraphPad Software, San Diego, CA) and is also presented in the tables below. ‘N.D.’ indicates that the % UTC value is not defined in that instance.

Compound No. 1517891 is a comparator compound and was previously described in International Patent No. WO 2022/066956. Compound No. 1517891 has the sequence (from 5′ to 3′): ACTTGGTGAATCTTTATTAA (SEQ ID NO: 22), wherein each cytosine is a 5-methylcytosine. The sugar motif for Compound No. 1517891 is (from 5′ to 3′): eeeeeddddddddddeeeee; wherein each “d” represents a 2′-β-D-deoxyribosyl sugar moiety, and each “e” represents a 2′-MOE sugar moiety. The internucleoside linkage motif for Compound No. 1517891 is (from 5′ to 3′): sooosssssssssssooss; wherein each “s” represents a phosphorothioate internucleoside linkage, and each “o” represents a phosphodiester internucleoside linkage.

TABLE 8
Dose-dependent reduction of human APOE RNA in HepG2
cells by modified oligonucleotides, 24-hour treatment

| | APOE RNA (% UTC) | | | | | | | | | | |
|---|---|---|---|---|---|---|---|---|---|---|---|
| Compound | 20,000 | 8,000 | 3,200 | 1,280 | 512 | 205 | 82 | 33 | 13 | 5 | IC₅₀ |
| No. | nM | nM | nM | nM | nM | nM | nM | nM | nM | nM | (μM) |
| 1757576 | 2 | 15 | 11 | 24 | 23 | 46 | 67 | 63 | 81 | 86 | 0.14 |
| 1778129 | 2 | 14 | 9 | 29 | 44 | 60 | 77 | 110 | 105 | 124 | 0.41 |
| 1785604 | 7 | 13 | 47 | 44 | 47 | 69 | 86 | 89 | 108 | 92 | 0.87 |
| 1778111 | 9 | 12 | 17 | 27 | 44 | 53 | 61 | 73 | 96 | 75 | 0.24 |
| 1778130 | 6 | 10 | 19 | 26 | 44 | 61 | 75 | 93 | 106 | 100 | 0.40 |
| 1785617 | 11 | 27 | 18 | 47 | 50 | 93 | 83 | 93 | 104 | 91 | 0.95 |
| 1778114 | 7 | 11 | 20 | 42 | 45 | 67 | 78 | 78 | 82 | 78 | 0.43 |
| 1778131 | 5 | 7 | 28 | 35 | 50 | 71 | 78 | 101 | 117 | 104 | 0.63 |
| 1778115 | 6 | 32 | 29 | 38 | 43 | 72 | 85 | 73 | 75 | 76 | 0.53 |
| 1778154 | 8 | 35 | 22 | 47 | 58 | 76 | 98 | 91 | 97 | 101 | 1.08 |
| 1778123 | 5 | 6 | 11 | 25 | 51 | 55 | 76 | 84 | 91 | 79 | 0.32 |
| 1785598 | 9 | 8 | 21 | 33 | 73 | 70 | 95 | 110 | 109 | 112 | 0.84 |
| 1778125 | 3 | 5 | 17 | 22 | 39 | 63 | 74 | 77 | 100 | 108 | 0.32 |
| 1785599 | 2 | 9 | 21 | 43 | 48 | 65 | 87 | 100 | 116 | 116 | 0.64 |
| 1778127 | 4 | 14 | 16 | 30 | 31 | 55 | 82 | 85 | 88 | 98 | 0.31 |
| 1785601 | 10 | 16 | 15 | 25 | 36 | 63 | 75 | 101 | 115 | 111 | 0.38 |
| 1778128 | 5 | 8 | 9 | 22 | 28 | 39 | 53 | 66 | 83 | 81 | 0.10 |
| 1785602 | 4 | 8 | 11 | 18 | 27 | 55 | 64 | 83 | 109 | 96 | 0.22 |

TABLE 9
Dose-dependent reduction of human APOE RNA in HepG2
cells by modified oligonucleotides, 16-hour treatment

| | APOE RNA (% UTC) | | | | | | | | | | |
|---|---|---|---|---|---|---|---|---|---|---|---|
| Compound | 20,000 | 8,000 | 3,200 | 1,280 | 512 | 205 | 82 | 33 | 13 | 5 | IC₅₀ |
| No. | nM | nM | nM | nM | nM | nM | nM | nM | nM | nM | (μM) |
| 1757569 | 18 | 18 | 24 | 24 | 38 | 60 | 81 | 82 | 91 | 101 | 0.39 |
| 1757559 | 25 | 39 | 42 | 60 | 89 | 81 | 87 | 89 | 80 | 90 | 3.11 |
| 1757585 | 1 | 12 | 32 | 19 | 53 | 33 | 38 | 44 | 49 | 45 | 0.01 |
| 1757563 | 8 | 17 | 41 | 54 | 67 | 76 | 94 | 84 | 103 | 95 | 1.38 |
| 1757576 | 7 | 22 | 32 | 45 | 61 | 74 | 84 | 80 | 74 | 86 | 0.82 |
| 1517891 | N.D. | 17 | 36 | 36 | 48 | 65 | 83 | 81 | 75 | 82 | 0.55 |
| 1757561 | 12 | 35 | 38 | 45 | 71 | 130 | 104 | 240 | 191 | 96 | 2.09 |
| 1757564 | 33 | 22 | 39 | 62 | 72 | 82 | 93 | 92 | 86 | 91 | 2.21 |
| 1757575 | 15 | 25 | 37 | 44 | 51 | 81 | 78 | 87 | 80 | 84 | 0.94 |
| 1757556 | 13 | 35 | 60 | 71 | 65 | 101 | 103 | 98 | 93 | 92 | 3.53 |
| 1757570 | 18 | 18 | 22 | 42 | 26 | 62 | 151 | N.D. | N.D. | 154 | 0.58 |
| 1757541 | 8 | 22 | 38 | 60 | 66 | 91 | 99 | 100 | 103 | 104 | 1.79 |
| 1757566 | 21 | 17 | 22 | 31 | 49 | 77 | 73 | 85 | 87 | 82 | 0.55 |
| 1757581 | 8 | 15 | 25 | 33 | 58 | 76 | 78 | 100 | 85 | 83 | 0.68 |
| 1757586 | N.D. | 19 | 20 | 27 | 34 | 129 | 157 | 52 | 246 | 202 | 0.50 |
| 1757554 | 13 | 28 | 27 | 52 | 71 | 89 | 106 | 101 | 113 | 91 | 1.57 |
| 1757565 | 22 | 55 | 30 | 49 | 64 | 89 | 84 | 76 | 108 | 95 | 2.13 |
| 1757567 | 23 | 19 | 37 | 59 | 64 | 95 | 91 | 97 | 108 | 103 | 1.82 |
| 1757562 | 16 | 28 | 29 | 40 | 45 | 52 | 87 | 56 | 86 | 160 | 0.57 |
| 1757544 | 12 | 26 | 42 | 59 | 75 | 90 | 111 | 100 | 86 | 104 | 2.18 |
| 1757582 | 9 | 37 | 12 | 29 | 64 | 70 | 92 | 72 | 107 | 87 | 0.71 |
| 1757557 | 7 | 36 | 59 | 94 | 89 | 86 | 88 | 89 | 88 | 79 | 4.68 |
| 1757551 | 9 | 14 | 29 | 35 | 41 | 52 | 55 | 88 | 248 | 91 | 0.43 |
| 1766537 | 5 | 21 | 36 | 62 | 85 | 90 | 89 | 108 | 111 | 95 | 2.01 |
| 1757568 | 19 | 21 | 31 | 47 | 63 | 93 | 96 | 104 | 87 | 86 | 1.39 |
| 1757572 | 10 | 26 | 51 | 66 | 67 | 83 | 96 | 107 | 95 | 114 | 2.35 |
| 1766559 | 36 | 35 | 21 | 30 | 41 | 46 | 47 | 65 | N.D. | 166 | 0.34 |
| 1757574 | 20 | 19 | 28 | 47 | 66 | 93 | 78 | 85 | 80 | 90 | 1.17 |
| 1757571 | 11 | 14 | 24 | 26 | 38 | 103 | 62 | 98 | 86 | 68 | 0.55 |

TABLE 10
Dose-dependent reduction of human APOE RNA in HepG2
cells by modified oligonucleotides, 24-hour treatment

| | APOE RNA (% UTC) | | | | | | | | | | |
|---|---|---|---|---|---|---|---|---|---|---|---|
| Compound | 20,000 | 8,000 | 3,200 | 1,280 | 512 | 205 | 82 | 33 | 13 | | IC₅₀ |
| No. | nM | nM | nM | nM | nM | nM | nM | nM | nM | 5 nM | (μM) |
| 1746784 | 7 | 7 | 10 | 29 | 48 | 61 | 80 | N.D. | 74 | 78 | 0.31 |
| 1746793 | 2 | 1 | 31 | 34 | 54 | 53 | 96 | 96 | N.D. | 102 | 0.56 |
| 1746802 | 3 | 39 | 47 | 36 | 57 | 58 | 72 | 74 | 108 | 76 | 0.78 |
| 1746811 | 5 | 12 | 23 | 47 | 54 | 70 | 95 | 83 | 86 | 99 | 0.74 |
| 1746785 | 35 | 8 | 29 | 48 | 53 | 83 | 84 | 97 | 93 | 90 | 1.04 |
| 1746794 | 3 | 9 | 16 | 37 | 72 | 67 | 91 | 94 | 107 | 119 | 0.80 |
| 1746803 | 39 | 22 | 29 | 31 | 61 | 68 | 114 | 84 | 83 | 93 | 1.10 |
| 1746812 | 11 | 19 | 31 | 51 | 76 | 86 | 102 | 90 | 109 | 105 | 1.50 |
| 1746786 | 14 | 18 | 34 | 39 | 63 | 87 | 75 | 90 | 93 | 85 | 1.01 |
| 1746795 | 6 | 12 | 28 | 57 | 53 | 99 | 108 | 110 | 115 | 119 | 1.29 |
| 1746804 | 2 | 4 | 40 | 45 | 57 | 66 | 81 | 80 | 88 | 89 | 0.68 |
| 1746813 | 21 | 19 | 41 | 57 | 83 | 100 | 101 | 119 | 117 | 137 | 2.25 |
| 1746787 | 14 | 11 | 37 | 56 | 68 | 87 | 97 | 92 | 108 | 148 | 1.52 |
| 1746796 | 20 | 28 | 40 | 71 | 89 | 119 | 137 | 128 | 126 | 110 | 3.01 |
| 1746805 | 11 | 29 | 39 | 41 | 75 | 65 | 94 | 93 | 109 | 95 | 1.36 |
| 1747050 | 10 | 14 | 31 | 49 | 76 | 110 | 111 | 115 | 135 | 182 | 1.54 |
| 1746789 | 4 | 10 | 19 | 37 | 64 | 76 | 102 | 84 | 106 | 98 | 0.83 |
| 1746797 | 18 | 42 | 37 | 71 | 113 | 129 | 143 | 150 | 128 | 139 | 3.77 |
| 1746806 | 37 | 25 | 39 | 57 | 80 | 83 | 91 | 107 | 101 | 122 | 2.58 |
| 1747051 | 9 | 31 | 42 | 76 | 67 | 111 | 128 | 152 | 117 | 111 | 2.90 |
| 1746790 | 8 | 15 | 24 | 20 | 63 | 89 | 112 | 94 | 133 | 104 | 0.75 |
| 1746799 | 33 | 24 | 23 | 52 | 84 | 106 | 125 | 153 | 130 | 115 | 1.74 |
| 1746807 | 30 | 16 | 29 | 52 | 62 | 88 | 86 | 111 | 127 | 119 | 1.43 |
| 1747052 | 19 | 31 | 46 | 53 | 79 | 108 | 127 | 148 | 113 | 150 | 2.71 |
| 1746791 | 21 | 16 | 21 | 35 | 62 | 80 | 88 | 93 | 104 | 88 | 0.85 |
| 1746800 | 8 | 35 | 39 | 87 | 77 | 97 | 115 | 137 | 111 | 108 | 3.27 |
| 1746809 | 14 | 23 | 33 | 35 | 54 | 81 | 74 | 98 | N.D. | 91 | 0.84 |
| 1747053 | 32 | 38 | 39 | 57 | 49 | 70 | 107 | 124 | 131 | 115 | 2.17 |
| 1746792 | 6 | 8 | 12 | 17 | 44 | 68 | 87 | 90 | 102 | 80 | 0.40 |
| 1746801 | 2 | 14 | 11 | 33 | 56 | 76 | 99 | 114 | 98 | 93 | 0.67 |
| 1746810 | 5 | 8 | 13 | 36 | 46 | 68 | 77 | 95 | 106 | 109 | 0.49 |
| 1753989 | 16 | 25 | 33 | 49 | 76 | 94 | 79 | 102 | 108 | 113 | 1.67 |

HepG2 cells plated at a density of 20,000 cells per well were treated by electroporation with various concentrations of modified oligonucleotide as specified in the tables below. After a treatment period of approximately 24 hours as indicated in the table titles below, total RNA was isolated from the cells, and APOE RNA levels were measured by quantitative real-time RT-PCR. APOE RNA levels were measured using the human primer probe set RTS3073 (described herein above). APOE RNA levels were normalized to human PPIA. Human PPIA was amplified using human primer probe set mk_cycloA_2nd (forward sequence TGCTGGACCCAACACAAATG, designated herein as SEQ ID NO: 13; reverse sequence TGCCATCCAACCACTCAGTC, designated herein as SEQ ID NO: 14; probe sequence TTCCCAGTTTTTCATCTGCACTGCCA, designated herein as SEQ ID NO: 15). Normalized results are presented in the table below as percent APOE RNA, relative to the amount of APOE RNA in untreated control cells (% UTC). The half maximal inhibitory concentration (IC₅₀) of each modified oligonucleotide was calculated in GraphPad Prism 10 (GraphPad Software, San Diego, CA) and is also presented in the table below.

TABLE 11
Dose-dependent reduction of human APOE RNA
in HepG2 cells by modified oligonucleotides

| | APOE RNA (% UTC) | | | | | | | | | | |
|---|---|---|---|---|---|---|---|---|---|---|---|
| Compound | 20,000 | 8,000 | 3,200 | 1,280 | 512 | 205 | 82 | 33 | 13 | | IC₅₀ |
| No. | nM | nM | nM | nM | nM | nM | nM | nM | nM | 5 nM | (μM) |
| 1757576 | 7 | 3 | 6 | 12 | 25 | 41 | 56 | 70 | 74 | 78 | 0.09 |
| 1785602 | 2 | 8 | 10 | 16 | 31 | 44 | 62 | 75 | 85 | 75 | 0.14 |
| 1809635 | 2 | 4 | 16 | 18 | 31 | 55 | 66 | 78 | 87 | 105 | 0.21 |
| 1809814 | 13 | 8 | 31 | 19 | 49 | 71 | 88 | 95 | 95 | 102 | 0.54 |

HepG2 cells plated at a density of 20,000 cells per well were treated by electroporation with various concentrations of modified oligonucleotide as specified in the tables below. After a treatment period of approximately 24 hours as indicated in the table titles below, total RNA was isolated from the cells, and APOE RNA levels were measured by quantitative real-time RT-PCR. APOE RNA levels were measured using the human primer probe set RTS3073 (described herein above). APOE RNA levels were normalized to human GAPDH, and human PPIA. Human GAPDH was amplified using human primer probe set RTS104 (described herein above). Human PPIA was amplified using human primer probe set mk_cycloA_2nd (described herein above) The average of the GAPDH-normalized results and the PPIA-normalized results are presented in the table below as percent APOE RNA, relative to the amount of APOE RNA in untreated control cells (% UTC). The half maximal inhibitory concentration (IC₅₀) of each modified oligonucleotide was calculated in GraphPad Prism 10 (GraphPad Software, San Diego, CA) and is also presented in the table below.

TABLE 12
Dose-dependent reduction of human APOE RNA
in HepG2 cells by modified oligonucleotides

| | APOE RNA (% UTC) | | | | | | | | | | |
|---|---|---|---|---|---|---|---|---|---|---|---|
| Compound | 20,000 | 8,000 | 3,200 | 1,280 | 512 | 205 | 82 | 33 | 13 | 5 | IC₅₀ |
| No. | nM | nM | nM | nM | nM | nM | nM | nM | nM | nM | (μM) |
| 1746809 | 10 | 15 | 11 | 28 | 34 | 59 | 72 | 86 | 94 | 97 | 0.31 |
| 1757575 | 5 | 10 | 27 | 34 | 47 | 72 | 81 | 93 | 96 | 92 | 0.57 |
| 1757576 | 6 | 11 | 22 | 29 | 40 | 61 | 77 | 83 | 88 | 94 | 0.37 |
| 1778111 | 8 | 14 | 21 | 32 | 43 | 62 | 77 | 91 | 88 | 106 | 0.44 |
| 1778128 | 14 | 22 | 25 | 24 | 37 | 60 | 67 | 83 | 95 | 96 | 0.35 |
| 1778130 | 9 | 18 | 12 | 20 | 45 | 52 | 72 | 85 | 100 | 99 | 0.31 |
| 1778131 | 6 | 5 | 25 | 25 | 36 | 56 | 70 | 85 | 86 | 102 | 0.29 |
| 1785598 | 11 | 11 | 9 | 23 | 36 | 56 | 75 | 88 | 97 | 103 | 0.29 |
| 1785599 | 15 | 10 | 18 | 32 | 43 | 71 | 81 | 93 | 106 | 100 | 0.51 |
| 1785602 | 3 | 21 | 8 | 33 | 36 | 56 | 70 | 85 | 91 | 97 | 0.30 |
| 1785617 | 6 | 20 | 16 | 23 | 32 | 55 | 68 | 82 | 93 | 95 | 0.26 |

### Example 5: Dose-Dependent Inhibition of Human APOE RNA in Differentiated SH-SY5Y Cells by Modified Oligonucleotides

Modified oligonucleotides selected from the examples above were tested at various doses in differentiated SH-SY5Y cells. The modified oligonucleotides were tested in a series of experiments that had the same culture conditions.

Differentiated SH-SY5Y cells plated at a density of 10,000 cells per well were treated by free uptake with various concentrations of modified oligonucleotide as specified in the tables below. After a treatment period of approximately 5 days, total RNA was isolated from the cells, and APOE RNA levels were measured by quantitative real-time RT-PCR. APOE RNA levels were measured using the human primer probe set RTS3073 (described herein above). APOE RNA levels were normalized to human PPIA. Human PPIA was amplified using human primer probe set mk_cycloA_2nd (described herein above). Results are presented as percent APOE RNA, relative to the amount of APOE RNA in untreated control cells (% UTC). The half maximal inhibitory concentration (IC₅₀) of each modified oligonucleotide was calculated in GraphPad Prism 10 (GraphPad Software, San Diego, CA) and is also presented in the tables below.

TABLE 13
Dose-dependent reduction of human APOE RNA in differentiated
SH-SY5Y cells by modified oligonucleotides

| | APOE RNA (% UTC) | | | | | | | | | | |
|---|---|---|---|---|---|---|---|---|---|---|---|
| Compound | 20,000 | 8,000 | 3,200 | 1,280 | 512 | 205 | 82 | 33 | 13 | 5 | IC₅₀ |
| No. | nM | nM | nM | nM | nM | nM | nM | nM | nM | nM | (uM) |
| 1757576 | 18 | 12 | 27 | 29 | 31 | 43 | 42 | 45 | 71 | 75 | 0.07 |
| 1785602 | 6 | 15 | 32 | 36 | 30 | 40 | 49 | 64 | 60 | 74 | 0.09 |
| 1809635 | 20 | 20 | 28 | 40 | 27 | 33 | 43 | 52 | 62 | 67 | 0.05 |
| 1809814 | 33 | 41 | 39 | 39 | 53 | 47 | 63 | 61 | 66 | 91 | 0.55 |

TABLE 14
Dose-dependent reduction of human APOE RNA in differentiated
SH-SY5Y cells by modified oligonucleotides

| | APOE RNA (% UTC) | | | | | | | | | | |
|---|---|---|---|---|---|---|---|---|---|---|---|
| Compound | 20,000 | 8,000 | 3,200 | 1,280 | 512 | 205 | 82 | 33 | 13 | | IC₅₀ |
| No. | nM | nM | nM | nM | nM | nM | nM | nM | nM | 5 nM | (μM) |
| 1757575 | 11 | 12 | 23 | 26 | 30 | 34 | 50 | 39 | 56 | 44 | 0.01 |
| 1757576 | 6 | 17 | 23 | 27 | 32 | 32 | 35 | 44 | 51 | 68 | 0.02 |
| 1778111 | 17 | 25 | 43 | 59 | 62 | 32 | 41 | 41 | 68 | 67 | 0.11 |
| 1778128 | 23 | 33 | 40 | 30 | 32 | 74 | 63 | 99 | 51 | 91 | 0.53 |
| 1778130 | 12 | 24 | 31 | 51 | 56 | 54 | 51 | 75 | 75 | 78 | 0.37 |
| 1778131 | 12 | 25 | 55 | 45 | 31 | 64 | 69 | 89 | 103 | 69 | 0.69 |
| 1746809 | 8 | 12 | 34 | 31 | 57 | 77 | 68 | 80 | 118 | 93 | 0.66 |
| 1785598 | 11 | 9 | 23 | 23 | 39 | 59 | 57 | 90 | 59 | 72 | 0.18 |
| 1785599 | 6 | 12 | 17 | 21 | 20 | 46 | 60 | 72 | 61 | 77 | 0.10 |
| 1785602 | 8 | 10 | 19 | 26 | 32 | 42 | 69 | 83 | 106 | 84 | 0.23 |
| 1785617 | 10 | 22 | 21 | 29 | 56 | 59 | 71 | 84 | 116 | 118 | 0.52 |
| 1746784 | 6 | 13 | 24 | 36 | 62 | 49 | 61 | 83 | 118 | 82 | 0.44 |
| 1746804 | 18 | 32 | 48 | 61 | 82 | 84 | 97 | 120 | 117 | 114 | 2.83 |

### Example 6: Dose-Dependent Inhibition of Human APOE RNA in HepG2 Cells by Modified Oligonucleotides, Electroporation

Modified oligonucleotides selected from the examples above were tested at various doses in HepG2.

HepG2 cells plated at a density of 50,000 cells per well were treated by electroporation with various concentrations of modified oligonucleotide as specified in the tables below. After a treatment period of approximately 24 hours, total RNA was isolated from the cells, and APOE RNA levels were measured by quantitative real-time RT-PCR. APOE RNA levels were measured using the human primer probe set RTS3073 (described herein above). APOE RNA levels were normalized to human GAPDH. Human GAPDH was amplified using human primer probe set RTS 104 (described herein above). Results are presented as percent APOE RNA, relative to the amount of APOE RNA in untreated control cells (% UTC). The half maximal inhibitory concentration (IC₅₀) of each modified oligonucleotide was calculated in GraphPad Prism 10 (GraphPad Software, San Diego, CA) and is also presented in the tables below.

TABLE 15
Dose-dependent reduction of human APOE RNA
in HepG2 cells by modified oligonucleotides

| | APOE RNA (% UTC) | | | | | | | | | | |
|---|---|---|---|---|---|---|---|---|---|---|---|
| Compound | 20,000 | 8,000 | 3,200 | 1,280 | 512 | 205 | 82 | 33 | 13 | 5 | IC₅₀ |
| No. | nM | nM | nM | nM | nM | nM | nM | nM | nM | nM | (μM) |
| 1652902 | 2 | 3 | 4 | 10 | 19 | 26 | 44 | 54 | 94 | 88 | 0.07 |
| 1693101 | 20 | 49 | 58 | 53 | 75 | 95 | 111 | 120 | 91 | 70 | 4.03 |
| 1693102 | 10 | 22 | 32 | 48 | 64 | 80 | 92 | 87 | 95 | 89 | 1.17 |
| 1693105 | 6 | 21 | 23 | 43 | 59 | 101 | 91 | 118 | 104 | 117 | 1.1 |
| 1736492 | 20 | 15 | 41 | 52 | 52 | 71 | 82 | 76 | 87 | 95 | 0.94 |
| 1736493 | 8 | 6 | 8 | 25 | 35 | 50 | 58 | 67 | 85 | 73 | 0.14 |
| 1736513 | 4 | 9 | 33 | 37 | 47 | 72 | 89 | 114 | 110 | 106 | 0.72 |
| 1736514 | 6 | 23 | 25 | 33 | 46 | 80 | 77 | 96 | 123 | 121 | 0.67 |
| 1736516 | 22 | 32 | 29 | 39 | 59 | 58 | 117 | 121 | 110 | 106 | 1.14 |
| 1736517 | 10 | 13 | 22 | 35 | 42 | 57 | 84 | 94 | 92 | 106 | 0.47 |
| 1736518 | 13 | 30 | 31 | 37 | 44 | 64 | 83 | 99 | 97 | 93 | 0.72 |
| 1736519 | 5 | 16 | 15 | 25 | 42 | 53 | 75 | 91 | 104 | 107 | 0.34 |
| 1736520 | 83 | 11 | 11 | 28 | 34 | 62 | 72 | 92 | 87 | 109 | 0.57 |
| 1736521 | 3 | 10 | 18 | 34 | 40 | 65 | 69 | 84 | 109 | 99 | 0.4 |
| 1736522 | 43 | 18 | 41 | 45 | 59 | 67 | 82 | 90 | 88 | 110 | 1.38 |
| 1736523 | 4 | 5 | 39 | 29 | 47 | 78 | 79 | 108 | 114 | 97 | 0.67 |
| 1736524 | 17 | 28 | 34 | 39 | 66 | 66 | 91 | 89 | 101 | 97 | 1.09 |
| 1736525 | 7 | 22 | 42 | 37 | 69 | 70 | 113 | 106 | 100 | 101 | 1.28 |
| 1736526 | 18 | 25 | 28 | 58 | 51 | 71 | 85 | 100 | 114 | 89 | 1.15 |
| 1736527 | 23 | 34 | 37 | 37 | 63 | 92 | 97 | 96 | 108 | 104 | 1.58 |
| 1736528 | 16 | 23 | 39 | 50 | 62 | 84 | 116 | 107 | 112 | 100 | 1.63 |
| 1736529 | 30 | 13 | 29 | 45 | 56 | 75 | 115 | 92 | 102 | 92 | 1.12 |
| 1736531 | 20 | 12 | 33 | 49 | 53 | 77 | 86 | 89 | 91 | 97 | 0.94 |
| 1736532 | 5 | 23 | 17 | 32 | 41 | 54 | 71 | 74 | 85 | 81 | 0.28 |
| 1736533 | 5 | 13 | 28 | 38 | 50 | 55 | 74 | 78 | 83 | 83 | 0.39 |
| 1736534 | 25 | 19 | 24 | 45 | 50 | 65 | 103 | 93 | 110 | 99 | 0.89 |
| 1736535 | 13 | 51 | 24 | 53 | 69 | 67 | 93 | 99 | 96 | 90 | 1.66 |
| 1736536 | 6 | 9 | 25 | 43 | 58 | 63 | 79 | 79 | 102 | 97 | 0.62 |

### Example 7: Activity of Modified Oligonucleotides Complementary to Human APOE in APOE Knock-In Mice, 2-Weeks

Modified oligonucleotides described above were tested in APOE knock-in mice.

APOE knock-in mice (model 41549) were obtained from Taconic Biosciences. APOE knock-in mice were divided into groups of 2 mice each. Each mouse received a single intracerebroventricular (ICV) bolus of 300 μg of modified oligonucleotide. A group of 4 mice received a single ICV bolus with PBS as a negative control.

Two weeks post treatment, mice were sacrificed and RNA was extracted from cortical brain tissue and spinal cord for quantitative real-time RT-PCR analysis of RNA expression of APOE using primer probe set RTS3073 (described herein above). APOE RNA levels were normalized to mouse Ppia. Mouse Ppia was amplified using primer probe set m_cyclo24 (forward sequence TCGCCGCTTGCTGCA, designated herein as SEQ ID NO: 16; reverse sequence ATCGGCCGTGATGTCGA, designated herein as SEQ ID NO: 17; probe sequence CCATGGTCAACCCCACCGTGTTC, designated herein as SEQ ID NO: 18). Results are presented as percent human APOE RNA relative to the amount of human APOE RNA in PBS treated animals, (% control). Each experiment is identified in separate tables below.

Compound No. 1517853 is a comparator compound and was previously described in International Patent No. WO 2022/066956. Compound No. 1517853 has the sequence (from 5′ to 3′): TTGGTGAATCTTTATTAAAC (SEQ ID NO: 27). The sugar motif for Compound No. 1517853 is (from 5′ to 3′): eeeeeddddddddddeeeee; wherein each “d” represents a 2′-β-D-deoxyribosyl sugar moiety, and each “e” represents a 2′-MOE sugar moiety. The internucleoside linkage motif for Compound No. 1517853 is (from 5′ to 3′): sooosssssssssssooss; wherein each “s” represents a phosphorothioate internucleoside linkage, and each “o” represents a phosphodiester internucleoside linkage. Each cytosine nucleobase in Compound No. 1517853 is a 5-methylcytosine.

Compound No. 1517891 is a comparator compound and is described herein above.

TABLE 16
Reduction of human APOE RNA in APOE 1549 knock-in mice

| | |
|---|---|
| Compound | APOE RNA (% control) |

| No. | Cortex | Spinal Cord |
|---|---|---|
| 1601942 | 84 | 91 |
| 1601943 | 115 | 113 |
| 1601944 | 109 | 95 |
| 1601948 | 112 | 105 |
| 1601949 | 77 | 92 |
| 1601955 | 98 | 111 |
| 1601956 | 104 | 107 |
| 1601957 | 101 | 90 |
| 1601958 | 109 | 102 |
| 1601959 | 107 | 85 |
| 1517853 | 53 | 65 |
| 1517891 | 49 | 65 |

TABLE 17
Reduction of human APOE RNA in APOE 1549 knock-in mice

| | |
|---|---|
| Compound | APOE RNA (% control) |

| No. | Cortex | Spinal Cord |
|---|---|---|
| PBS | 100 | 100 |
| 1652902 | 31 | 171 |
| 1693101 | 79 | 57 |
| 1693102 | 60 | 45 |
| 1693105 | 51 | 38 |

### Example 8: Activity of Modified Oligonucleotides Complementary to Human APOE in APOE Knock-In Mice, 4-Weeks

Modified oligonucleotides described above were tested in the 1549 APOE knock-in mouse model described above.

APOE knock-in mice were divided into groups of either 2 or 6 mice each as described in the tables below. Each mouse received a single intracerebroventricular (ICV) bolus of 300 μg of modified oligonucleotide. A group of either 3 or 6 mice received a single ICV bolus with PBS as a negative control.

Four weeks post treatment, mice were sacrificed and RNA was extracted from cortical brain tissue and spinal cord for quantitative real-time RT-PCR analysis of RNA expression of APOE using primer probe set RTS3073 (described herein above). APOE RNA levels were normalized to mouse Ppia. Mouse Ppia was amplified using primer probe set m_cyclo24 (described herein above). Results are presented as percent human APOE RNA relative to the amount of human APOE RNA in PBS treated animals, (% control). Each experiment is identified in separate tables below.

TABLE 18
Reduction of human APOE RNA in
APOE 1549 knock-in mice, n = 2

| | |
|---|---|
| Compound | APOE RNA (% control) |

| No. | Cortex | Spinal Cord |
|---|---|---|
| PBS | 100 | 100 |
| 1736520 | 41 | 32 |
| 1736521 | 52 | 50 |
| 1736522 | 41 | 39 |
| 1736523 | 42 | 34 |
| 1736524 | 36 | 31 |
| 1736525 | 35 | 41 |
| 1736526 | 35 | 42 |
| 1736527 | 37 | 54 |

TABLE 19
Reduction of human APOE RNA in
APOE 1549 knock-in mice, n = 6

| | |
|---|---|
| Compound | APOE RNA (% control) |

| No. | Cortex | Spinal Cord |
|---|---|---|
| PBS | 100 | 100 |
| 1757576 | 26 | 17 |

### Example 9: Activity of Modified Oligonucleotides Complementary to Human APOE in APOE Knock-In Mice, 2-Weeks

Modified oligonucleotides described above were tested in the APOE knock-in mouse model C57BL/6NTac-Apoeem7250_B-A02(APOE)Tac, a heterozygous APOE mouse model. APOE knock-in mice used in this study express the full-length human APOE gene knocked into the mouse locus. Humanization of APOE gene was done via CRISPR/Cas-9-mediated gene editing, allowing for generation of a model with constitutive expression of human APOE gene. Targeting strategy was based on NCBI transcripts NM_009696.4 (mouse) and NM_000041.4 (human). Mouse genomic sequence from exon 1 to exon 4 (including the 5′ and 3′ UTRs) was replaced with the human counterpart from 141 bp upstream of exon 1 to 28 bp downstream of exon 4. A plasmid allowing expression of Cas9 mRNA, specific gRNA, and the puromycin resistance cassette, and a plasmid containing the homology regions of the mouse APOE gene, and the replaced human region were co-transfected into the Taconic Biosciences C57BL/6N Tac ES cell line. Homologous recombination clones were isolated using positive puromycin selection, and humanized allele was obtained after Cas9-mediated gene editing. C57BL/6NTac-Apoeem7250_B-A02(APOE)Tac mice were used in the in vivo experiments described below.

APOE C57BL/6NTac-Apoeem7250 knock-in mice were divided into groups of 3 mice each. Each mouse received a single ICV bolus of 300 μg of modified oligonucleotide. A group of 3 mice received a single ICV bolus with PBS as a negative control.

Two weeks post treatment, mice were sacrificed and RNA was extracted from cortical brain tissue and spinal cord for quantitative real-time RT-PCR analysis of RNA expression of APOE using human primer probe set RTS3073 (described herein above). APOE RNA levels were normalized to mouse Ppia. Mouse Ppia was amplified using primer probe set m_cyclo24 (described herein above). Results are presented as percent human APOE RNA relative to the amount of human APOE RNA in PBS treated animals, (% control).

TABLE 20
Reduction of human APOE RNA in APOE
C57BL/6NTac-Apoeem7250 knock-in mice

| | |
|---|---|
| Compound | APOE RNA (% control) |

| No. | Cortex | Spinal Cord |
|---|---|---|
| PBS | 100 | 100 |
| 1693101 | 62 | 55 |
| 1693102 | 51 | 51 |
| 1693105 | 65 | 54 |

### Example 10: Activity of Modified Oligonucleotides Complementary to Human APOE in APOE Knock-In Mice, 4-Weeks

Modified oligonucleotides described above were tested in the APOE C57BL/6NTac-Apoeem7250 knock-in mouse model described above.

APOE C57BL/6NTac-Apoeem7250 knock-in mice were divided into groups of 2-4 mice each. Each mouse received a single ICV bolus of 300 μg of modified oligonucleotide. A group of 3-4 mice received a single ICV bolus with PBS as a negative control.

Four weeks post treatment, mice were sacrificed and RNA was extracted from cortical brain tissue and spinal cord for quantitative real-time RT-PCR analysis of RNA expression of APOE using human primer probe set RTS3073 (described herein above). APOE RNA levels were normalized to mouse Ppia. Mouse Ppia was amplified using primer probe set m_cyclo24 (described herein above). Results are presented as percent human APOE RNA relative to the amount of human APOE RNA in PBS treated animals, (% control). Each experiment is identified in separate tables below.

TABLE 21
Reduction of human APOE RNA in APOE
C57BL/6NTac-Apoeem7250 knock-in mice

| | |
|---|---|
| Compound | APOE RNA (% control) |

| No. | Cortex | Spinal Cord |
|---|---|---|
| PBS | 100 | 100 |
| 1693101 | 71 | 50 |
| 1693102 | 61 | 48 |
| 1693105 | 54 | 51 |

TABLE 22
Reduction of human APOE RNA in APOE
C57BL/6NTac-Apoeem7250 knock-in mice

| | |
|---|---|
| Compound | APOE RNA (% control) |

| No. | Cortex | Spinal Cord |
|---|---|---|
| PBS | 100 | 100 |
| 1693101 | 57 | 77 |
| 1693102 | 55 | 72 |
| 1693105 | 65 | 87 |
| 1736492 | 45 | 46 |
| 1736493 | 47 | 51 |
| 1736513 | 42 | 52 |
| 1736514 | 28 | 54 |
| 1736528 | 57 | 56 |
| 1736529 | 51 | 46 |
| 1736531 | 42 | 52 |
| 1736532 | 30 | 38 |
| 1736533 | 34 | 54 |
| 1736534 | 31 | 55 |
| 1736535 | 39 | 63 |
| 1736536 | 31 | 53 |

TABLE 23
Reduction of human APOE RNA in APOE
C57BL/6NTac-Apoeem7250 knock-in mice

| | |
|---|---|
| Compound | APOE RNA (% control) |

| No. | Cortex | Spinal Cord |
|---|---|---|
| PBS | 100 | 100 |
| 1693101 | 57 | 56 |
| 1746784 | 49 | 26 |
| 1746786 | 79 | 52 |
| 1746787 | 76 | 71 |
| 1746789 | 60 | 41 |
| 1746790 | 53 | 61 |
| 1746792 | 71 | 49 |
| 1746793 | 86 | 47 |
| 1746794 | 76 | 41 |
| 1746795 | 75 | 75 |
| 1746796 | 81 | 68 |
| 1746797 | 72 | 68 |
| 1746799 | 47 | 42 |
| 1746800 | 41 | 40 |
| 1746801 | 58 | 44 |
| 1746802 | 72 | 52 |
| 1746803 | 51 | 39 |
| 1746804 | 51 | 52 |
| 1746806 | 92 | 75 |
| 1746807 | 90 | 119 |
| 1746809 | 36 | 59 |
| 1746810 | 56 | 80 |
| 1746811 | 61 | 62 |
| 1746812 | 68 | 79 |
| 1747050 | 29 | 45 |
| 1753989 | 54 | 49 |

TABLE 24
Reduction of human APOE RNA in APOE
C57BL/6NTac-Apoeem7250 knock-in mice

| | |
|---|---|
| Compound | APOE RNA (% control) |

| No. | Cortex | Spinal Cord |
|---|---|---|
| PBS | 100 | 100 |
| 1766559 | 79 | 81 |
| 1778107 | 70 | 70 |
| 1778108 | 60 | 41 |
| 1778109 | 59 | 61 |
| 1778110 | 51 | 51 |
| 1778111 | 34 | 53 |
| 1778112 | 46 | 49 |
| 1778113 | 48 | 88 |
| 1778114 | 37 | 57 |
| 1778115 | 37 | 66 |
| 1778117 | 46 | 114 |
| 1778119 | 45 | 56 |
| 1778120 | 47 | 58 |
| 1778122 | 53 | 62 |
| 1778123 | 37 | 59 |
| 1778125 | 40 | 52 |
| 1778126 | 41 | 72 |
| 1778127 | 32 | 53 |
| 1778128 | 31 | 40 |
| 1778129 | 25 | 47 |
| 1778130 | 31 | 34 |
| 1778131 | 29 | 49 |
| 1778154 | 30 | 44 |
| 1779699 | 44 | 54 |
| 1779706 | 35 | 53 |
| 1779961 | 39 | 53 |

TABLE 25
Reduction of human APOE RNA in APOE
C57BL/6NTac-Apoeem7250 knock-in mice

| | |
|---|---|
| Compound | APOE RNA (% control) |

| No. | Cortex | Spinal Cord |
|---|---|---|
| PBS | 100 | 100 |
| 1757571 | 48 | 35 |
| 1757572 | 50 | 38 |
| 1757573 | 53 | 38 |
| 1757574 | 43 | 32 |
| 1757575 | 38 | 27 |
| 1757576 | 29 | 23 |
| 1785588 | 30 | 29 |
| 1785589 | 32 | 40 |
| 1785591 | 37 | 39 |
| 1785598 | 29 | 40 |
| 1785599 | 32 | 38 |
| 1785600 | 46 | 51 |
| 1785601 | 33 | 40 |
| 1785602 | 34 | 33 |
| 1785604 | 39 | 55 |
| 1785616 | 47 | 66 |
| 1785617 | 39 | 38 |
| 1785619 | 41 | 41 |

TABLE 26
Reduction of human APOE RNA in APOE
C57BL/6NTac-Apoeem7250 knock-in mice

| | |
|---|---|
| Compound | APOE RNA (% control) |

| No. | Cortex | Spinal Cord |
|---|---|---|
| PBS | 100 | 100 |
| 1746784 | 39 | 26 |
| 1746804 | 46 | 40 |

### Example 11: Activity of Modified Oligonucleotides Complementary to Human APOE in APOE Knock-In Mice, 8-Weeks

Modified oligonucleotides described above were tested in the APOE C57BL/6NTac-Apoeem7250 knock-in mouse model described above.

APOE C57BL/6NTac-Apoeem7250 knock-in mice were divided into groups of 3-4 mice or otherwise indicated in the tables below. Each mouse received a single ICV bolus of 300 μg of modified oligonucleotide. A group of 3-4 mice received a single ICV bolus with PBS as a negative control.

Eight weeks post treatment, mice were sacrificed and RNA was extracted from cortical brain tissue and spinal cord for quantitative real-time RT-PCR analysis of RNA expression of APOE using human primer probe set RTS3073 (described herein above). APOE RNA levels were normalized to mouse Ppia. Mouse Ppia was amplified using primer probe set m_cyclo24 (described herein above). Results are presented as percent human APOE RNA relative to the amount of human APOE RNA in PBS treated animals, (% control). Each experiment is identified in separate tables below.

Compound No. 1517578 is a comparator compound and was previously described in International Patent No. WO 2022/066956. Compound No. 1517578 has the sequence (from 5′ to 3′): CTTGGTGAATCTTTATTAAA (SEQ ID NO: 23). The sugar motif for Compound No. 1517578 is (from 5′ to 3′): eeeeeddddddddddeeeee; wherein each “d” represents a 2′-β-D-deoxyribosyl sugar moiety, and each “e” represents a 2′-MOE sugar moiety. The internucleoside linkage motif for Compound No. 1517578 is (from 5′ to 3′): sooosssssssssssooss; wherein each “s” represents a phosphorothioate internucleoside linkage, and each “o” represents a phosphodiester internucleoside linkage. Each cytosine nucleobase in Compound No. 1517578 is a 5-methylcytosine.

Compound No. 1517853 is a comparator compound and is described herein above.

Compound No. 1517891 is a comparator compound and is described herein above.

TABLE 27
Reduction of human APOE RNA in APOE
C57BL/6NTac-Apoeem7250 knock-in mice

| | |
|---|---|
| Compound | APOE RNA (% control) |

| No. | Cortex | Spinal Cord |
|---|---|---|
| PBS | 100 | 100 |
| 1517578 | 66 | 47 |
| 1517853 | 67 | 50 |
| 1517891 | 56 | 29 |

TABLE 28
Reduction of human APOE RNA in APOE
C57BL/6NTac-Apoeem7250 knock-in mice

| | |
|---|---|
| Compound | APOE RNA (% control) |

| No. | Cortex | Spinal Cord |
|---|---|---|
| PBS | 100 | 100 |
| 1652898 | 56 | 20 |
| 1652899 | 57 | 16 |
| 1652900 | 83 | 47 |
| 1652901 | 47 | 18 |
| 1652902 | 44 | 12 |
| 1652903 | 49 | 19 |
| 1652905 | 44 | 15 |
| 1652907 | 54 | 16 |
| 1652908 | 54 | 18 |
| 1652909 | 55 | 23 |

TABLE 29
Reduction of human APOE RNA in APOE
C57BL/6NTac-Apoeem7250 knock-in mice

| | |
|---|---|
| Compound | APOE RNA (% control) |

| No. | Cortex | Spinal Cord |
|---|---|---|
| PBS | 100 | 100 |
| 1652910 | 82 | 39 |
| 1652911 | 130 | 57 |
| 1652912 | 95 | 41 |
| 1652913 | 75 | 33 |
| 1652914 | 108 | 43 |
| 1652916 | 128 | 49 |
| 1652917 | 107 | 41 |
| 1652918 | 91 | 47 |
| 1652919 | 90 | 54 |

TABLE 30
Reduction of human APOE RNA in APOE
C57BL/6NTac-Apoeem7250 knock-in mice

| | |
|---|---|
| Compound | APOE RNA (% control) |

| No. | Cortex | Spinal Cord |
|---|---|---|
| PBS | 100 | 100 |
| 1681003 | 90 | 48 |
| 1681004 | 120 | 89 |
| 1681005 | 101 | 70 |
| 1681006 | 85 | 71 |
| 1681007 | 71 | 47 |
| 1681020 | 63 | 35 |
| 1681021 | 55 | 68 |

TABLE 31
Reduction of human APOE RNA in APOE
C57BL/6NTac-Apoeem7250 knock-in mice

| | |
|---|---|
| Compound | APOE RNA (% control) |

| No. | Cortex | Spinal Cord |
|---|---|---|
| PBS | 100 | 100 |
| 1693039 | 90 | 71 |
| 1693040 | 80 | 77 |
| 1693041 | 75 | 74 |
| 1693042 | 77 | 81 |
| 1693043 | 89 | 83 |
| 1693044 | 86 | 95 |
| 1693045 | 88 | 86 |
| 1693047 | 78 | 77 |
| 1693048 | 97 | 87 |
| 1693049 | 101 | 96 |
| 1693082 | 113 | 54 |
| 1693084 | 72 | 115 |
| 1693085 | 86 | 79 |
| 1693050 | 77 | 63 |
| 1693051 | 114 | 76 |
| 1693052 | 65 | 79 |

TABLE 32
Reduction of human APOE RNA in APOE C57BL/6NTac-
Apoeem7250 knock-in mice; n = 1-3

| | |
|---|---|
| Compound | APOE RNA (% control) |

| No | Cortex | Spinal Cord |
|---|---|---|
| PBS | 100 | 100 |
| 1693144 | 67 | 43 |
| 1746784 | 38 | 138 |
| 1746799 | 45 | 39 |
| 1746800 | 40 | 30 |
| 1746809 | 47 | 44 |
| 1747050 | 36 | 23 |
| 1757571 | 66 | 33 |
| 1757574 | 44 | 21 |
| 1757575 | 35 | 32 |
| 1757576 | 29 | 15 |
| 1778111 | 41 | 17 |
| 1778112 | 49 | 23 |
| 1778113 | 53 | 28 |
| 1778114 | 47 | 32 |
| 1778115 | 46 | 27 |
| 1778117 | 49 | 28 |
| 1778119 | 52 | 27 |
| 1778120 | 57 | 24 |
| 1778123 | 49 | 29 |
| 1778125 | 59 | 37 |
| 1778126 | 23 | 20 |
| 1778127 | 28 | 14 |
| 1778128 | 27 | 25 |
| 1778129 | 19 | 9 |
| 1778130 | 23 | 18 |
| 1778131 | 29 | 24 |
| 1778154 | 29 | 32 |
| 1785598 | 23 | 16 |
| 1785599 | 26 | 34 |
| 1785600 | 36 | 26 |
| 1785601 | 28 | 211 |
| 1785602 | 27 | 26 |
| 1785604 | 28 | 35 |
| 1785616 | 47 | 27 |
| 1785617 | 30 | 213 |
| 1785619 | 40 | 27 |

TABLE 33
Reduction of human APOE RNA in APOE
C57BL/6NTac-Apoeem7250 knock-in mice

| | |
|---|---|
| Compound | APOE RNA (% control) |

| No. | Cortex | Spinal Cord |
|---|---|---|
| PBS | 100 | 100 |
| 1746804 | 40 | 25 |

TABLE 34
Reduction of human APOE RNA in APOE
C57BL/6NTac-Apoeem7250 knock-in mice

| | |
|---|---|
| Compound | APOE RNA (% control) |

| No. | Cortex | Spinal Cord |
|---|---|---|
| PBS | 100 | 100 |
| 1757576 | 21 | 10 |
| 1809635 | 26 | 15 |
| 1809636 | 16 | 11 |
| 1809637 | 20 | 9 |
| 1809688 | 22 | 37 |
| 1809690 | 26 | 24 |
| 1809691 | 27 | 17 |
| 1809741 | 33 | 28 |
| 1809744 | 19 | 11 |
| 1809745 | 26 | 16 |
| 1809813 | 37 | 32 |
| 1809814 | 36 | 33 |
| 1809818 | 42 | 36 |
| 1810063 | 43 | 40 |
| 1810064 | 23 | 15 |
| 1810065 | 29 | 21 |
| 1810069 | 24 | 27 |

TABLE 35
Reduction of human APOE RNA in APOE
C57BL/6NTac-Apoeem7250 knock-in mice

| | |
|---|---|
| Compound | APOE RNA (% control) |

| No. | Cortex | Spinal Cord |
|---|---|---|
| PBS | 100 | 100 |
| 1652901 | 49 | 27 |
| 1693101 | 58 | 46 |
| 1693102 | 58 | 43 |
| 1693105 | 58 | 43 |

### Example 12: Activity of Modified Oligonucleotides Complementary to Human APOE in APOE Knock-In Mice, 16-Weeks

Modified oligonucleotides described above were tested in the APOE C57BL/6NTac-Apoeem7250 knock-in mouse model described above.

APOE C57BL/6NTac-Apoeem7250 knock-in mice were divided into groups of 3-4 mice each. Each mouse received a single intracerebroventricular (ICV) bolus of 300 μg of modified oligonucleotide. A group of 4 mice received a single ICV bolus with PBS as a negative control.

Sixteen weeks post treatment, mice were sacrificed and RNA was extracted from cortical brain tissue and spinal cord for quantitative real-time RT-PCR analysis of RNA expression of APOE using primer probe set RTS3073 (described herein above). APOE RNA levels were normalized to mouse Ppia. Mouse Ppia was amplified using primer probe set m_cyclo24 (described herein above). Results are presented as percent human APOE RNA relative to the amount of human APOE RNA in PBS treated animals, (% control). Each experiment is identified in separate tables below.

TABLE 36
Reduction of human APOE RNA in APOE
C57BL/6NTac-Apoeem7250 knock-in mice

| | |
|---|---|
| Compound | APOE RNA (% control) |

| No. | Cortex | Spinal Cord |
|---|---|---|
| PBS | 100 | 100 |
| 1652901 | 104 | 61 |
| 1652902 | 105 | 60 |
| 1652903 | 87 | 39 |
| 1681003 | 106 | 61 |
| 1681004 | 127 | 61 |
| 1681005 | 116 | 68 |
| 1681006 | 120 | 80 |
| 1681007 | 96 | 67 |
| 1681020 | 92 | 59 |
| 1681021 | 91 | 40 |

TABLE 37
Reduction of human APOE RNA in APOE
C57BL/6NTac-Apoeem7250 knock-in mice

| | |
|---|---|
| Compound | APOE RNA (% control) |

| No. | Cortex | Spinal Cord |
|---|---|---|
| PBS | 100 | 100 |
| 1693039 | 127 | 99 |
| 1693040 | 92 | 71 |
| 1693041 | 84 | 85 |
| 1693044 | 98 | 63 |
| 1693045 | 93 | 60 |
| 1693046 | 106 | 80 |
| 1693047 | 97 | 71 |
| 1693048 | 105 | 71 |
| 1693049 | 93 | 81 |
| 1693042 | 90 | 65 |
| 1693043 | 80 | 73 |
| 1693050 | 100 | 63 |
| 1693051 | 97 | 79 |
| 1693052 | 91 | 85 |
| 1693053 | 99 | 99 |
| 1693055 | 95 | 67 |
| 1693056 | 105 | 118 |
| 1693057 | 125 | 101 |
| 1693058 | 113 | 80 |
| 1693059 | 89 | 98 |

TABLE 38
Reduction of human APOE RNA in APOE
C57BL/6NTac-Apoeem7250 knock-in mice

| | |
|---|---|
| Compound | APOE RNA (% control) |

| No. | Cortex | Spinal Cord |
|---|---|---|
| PBS | 100 | 100 |
| 1693060 | 93 | 93 |
| 1693061 | 91 | 99 |
| 1693062 | 98 | 110 |
| 1693063 | 97 | 87 |
| 1693064 | 83 | 48 |
| 1693065 | 82 | 46 |
| 1693066 | 88 | 73 |
| 1693067 | 97 | 162 |
| 1693068 | 86 | 72 |
| 1693069 | 78 | 66 |
| 1693082 | 81 | 72 |
| 1693084 | 89 | 86 |
| 1693085 | 93 | 71 |
| 1693100 | 87 | 51 |
| 1693101 | 71 | 56 |
| 1693102 | 69 | 41 |
| 1693103 | 84 | 56 |
| 1693104 | 129 | 52 |
| 1693105 | 65 | 44 |
| 1693106 | 78 | 71 |

TABLE 39
Reduction of human APOE RNA in APOE
C57BL/6NTac-Apoeem7250 knock-in mice

| | |
|---|---|
| Compound | APOE RNA (% control) |

| No. | Cortex | Spinal Cord |
|---|---|---|
| PBS | 100 | 100 |
| 1693070 | 86 | 76 |
| 1693071 | 68 | 66 |
| 1693072 | 82 | 61 |
| 1693073 | 107 | 52 |
| 1693074 | 84 | 71 |
| 1693075 | 87 | 64 |
| 1693077 | 98 | 76 |
| 1693078 | 98 | 99 |
| 1693079 | 93 | 106 |
| 1693080 | 99 | 88 |
| 1693081 | 91 | 100 |
| 1693110 | 113 | 92 |
| 1693138 | 90 | 85 |
| 1693139 | 92 | 97 |
| 1693140 | 109 | 68 |
| 1693141 | 82 | 66 |
| 1693142 | 98 | 92 |
| 1693143 | 76 | 64 |
| 1693144 | 113 | 114 |

TABLE 40
Reduction of human APOE RNA in APOE
C57BL/6NTac-Apoeem7250 knock-in mice

| | |
|---|---|
| Compound | APOE RNA (% control) |

| No. | Cortex | Spinal Cord |
|---|---|---|
| PBS | 100 | 100 |
| 1693144 | 61 | 54 |
| 1757571 | 47 | 26 |
| 1757574 | 55 | 22 |
| 1757575 | 33 | 21 |
| 1757576 | 45 | 14 |
| 1778111 | 49 | 27 |
| 1778112 | 63 | 25 |
| 1778113 | 46 | 27 |
| 1778114 | 52 | 27 |
| 1778115 | 44 | 28 |
| 1778117 | 65 | 36 |
| 1778119 | 62 | 38 |
| 1778120 | 75 | 46 |
| 1778123 | 54 | 31 |
| 1778125 | 75 | 42 |
| 1778126 | 37 | 22 |
| 1778127 | 48 | 25 |
| 1778128 | 34 | 22 |
| 1778129 | 34 | 14 |
| 1778130 | 34 | 15 |
| 1778131 | 34 | 17 |
| 1778154 | 50 | 29 |
| 1785598 | 36 | 24 |
| 1785599 | 35 | 54 |
| 1785600 | 39 | 30 |
| 1785601 | 39 | 21 |
| 1785602 | 38 | 20 |
| 1785604 | 41 | 27 |
| 1785616 | 47 | 34 |
| 1785617 | 37 | 28 |
| 1785619 | 40 | 41 |

TABLE 41
Reduction of human APOE RNA in APOE
C57BL/6NTac-Apoeem7250 knock-in mice

| | |
|---|---|
| Compound | APOE RNA (% control) |

| No. | Cortex | Spinal Cord |
|---|---|---|
| PBS | 100 | 100 |
| 1736492 | 53 | 52 |
| 1736493 | 57 | 51 |
| 1736513 | 53 | 30 |
| 1736514 | 39 | 16 |
| 1736529 | 60 | 28 |
| 1736531 | 54 | 74 |
| 1736532 | 34 | 45 |
| 1736533 | 57 | 49 |
| 1736534 | 46 | 34 |
| 1736535 | 47 | 26 |
| 1736536 | 36 | 31 |

### Example 13: Activity of Modified Oligonucleotides Complementary to Human APOE in APOE Knock-In Mice, 20-Weeks

Modified oligonucleotides described above were tested in the APOE C57BL/6NTac-Apoeem7250 knock-in mouse model described above.

APOE C57BL/6NTac-Apoeem7250 knock-in mice were divided into groups of 4 mice each. Each mouse received a single ICV bolus of 300 μg of modified oligonucleotide. A group of 2-4 mice received a single ICV bolus with PBS as a negative control.

Twenty weeks post treatment, mice were sacrificed and RNA was extracted from cortical brain tissue and spinal cord for quantitative real-time RT-PCR analysis of RNA expression of APOE using human primer probe set RTS3073 (described herein above). APOE RNA levels were normalized to mouse Ppia. Mouse Ppia was amplified using primer probe set m_cyclo24 (described herein above). Results are presented as percent human APOE RNA relative to the amount of human APOE RNA in PBS treated animals, (% control). Each experiment is identified in separate tables below.

Compound No. 1517853 is a comparator compound and is described herein above.

Compound No. 1517891 is a comparator compound and is described herein above.

TABLE 42
Reduction of human APOE RNA in APOE
C57BL/6NTac-Apoeem7250 knock-in mice

| | |
|---|---|
| Compound | APOE RNA (% control) |

| No. | Cortex | Spinal Cord |
|---|---|---|
| PBS | 100 | 100 |
| 1517853 | 99 | 98 |
| 1517891 | 93 | 92 |
| 1693144 | 65 | 71 |
| 1736520 | 65 | 62 |
| 1736522 | 64 | 56 |
| 1736523 | 73 | 44 |
| 1736524 | 67 | 62 |
| 1736525 | 71 | 118 |
| 1736526 | 67 | 68 |
| 1736527 | 64 | 58 |
| 1757565 | 82‡ | 73‡ |
| 1757567 | 80 | 65 |
| 1757568 | 73 | 58 |
| 1757570 | 64 | 48 |
| 1757571 | 53 | 73 |
| 1757572 | 51 | 69 |
| 1757573 | 61 | 42 |
| 1757574 | 60 | 68 |
| 1757575 | 39 | 88 |
| 1757576 | 32 | 31 |
| 1757581 | 87 | 75 |
| 1757582 | 62 | 44 |
| 1757583 | 97 | 100 |
| 1757584 | 92 | 95 |
| 1757585 | 62 | 61 |
| 1757586 | 88 | 83 |

‡indicates fewer than 4 samples available

TABLE 43
Reduction of human APOE RNA in APOE
C57BL/6NTac-Apoeem7250 knock-in mice

| | |
|---|---|
| Compound | APOE RNA (% control) |

| No. | Cortex | Spinal Cord |
|---|---|---|
| PBS | 100 | 100 |
| 1517891 | 97 | 71 |
| 1757541 | 88 | 80 |
| 1757544 | 101 | 96 |
| 1757551 | 93 | 53 |
| 1757554 | 95 | 68 |
| 1757556 | 99 | 82 |
| 1757559 | 94 | 69 |
| 1757561 | 92 | 64 |
| 1757562 | 96 | 88 |
| 1757563 | 93 | 71 |
| 1757564 | 95 | 69 |
| 1766559 | 100 | 74 |
| 1778107 | 76 | 32 |
| 1778108 | 70 | 21 |
| 1778109 | 81 | 34 |
| 1778110 | 81 | 35 |
| 1778111 | 59 | 25 |
| 1778112 | 67 | 25 |
| 1778113 | 61 | 24 |
| 1778114 | 56 | 26 |

TABLE 44
Reduction of human APOE RNA in APOE
C57BL/6NTac-Apoeem7250 knock-in mice

| | |
|---|---|
| Compound | APOE RNA (% control) |

| No. | Cortex | Spinal Cord |
|---|---|---|
| PBS | 100 | 100 |
| 1778115 | 77 | 28 |
| 1778117 | 72 | 36 |
| 1778119 | 84 | 51 |
| 1778120 | 78 | 44 |
| 1778122 | 76 | 34 |
| 1778123 | 77 | 36 |
| 1778125 | 81 | 49 |
| 1778126 | 52 | 35 |
| 1778127 | 57 | 43 |
| 1778128 | 60 | 22 |
| 1778129 | 52 | 24 |
| 1778130 | 48 | 19 |
| 1778131 | 65 | 43 |
| 1778154 | 82 | 46 |
| 1779699 | 53 | 25 |
| 1779701 | 56 | 24 |
| 1779706 | 46 | 36 |
| 1779961 | 59 | 26 |
| 1779970 | 63 | 35 |

TABLE 45
Reduction of human APOE RNA in APOE
C57BL/6NTac-Apoeem7250 knock-in mice

| | |
|---|---|
| Compound | APOE RNA (% control) |

| No. | Cortex | Spinal Cord |
|---|---|---|
| PBS | 100 | 100 |
| 1785588 | 40 | 18 |
| 1785589 | 49 | 30 |
| 1785591 | 51 | 42 |
| 1785598 | 72 | 30 |
| 1785599 | 55 | 20 |
| 1785600 | 60 | 33 |
| 1785601 | 43 | 31 |
| 1785602 | 53 | 28 |
| 1785604 | 55 | 47 |
| 1785616 | 77 | 57 |
| 1785617 | 61 | 38 |
| 1785619 | 58 | 59 |

### Example 14: Potency of Modified Oligonucleotides Complementary to Human APOE in APOE Knock-In Mice, 4-Weeks

Modified oligonucleotides described above were tested in the APOE C57BL/6NTac-Apoeem7250 knock-in mouse model described above.

APOE C57BL/6NTac-Apoeem7250 knock-in mice were divided into groups of 3-4 mice each. Each mouse received a single ICV bolus of modified oligonucleotide at various doses indicated in the tables below. A group of 3-4 mice received a single ICV bolus with PBS as a negative control.

Four weeks post treatment, mice were sacrificed, and RNA was extracted from cortical brain tissue and spinal cord for quantitative real-time RT-PCR analysis of RNA expression of SNCA using human primer probe set RTS3073 (described herein above). APOE RNA levels were normalized to mouse Ppia. Mouse Ppia was amplified using primer probe set m_cyclo24 (described herein above). Results are presented as percent human APOE RNA relative to the amount of APOE RNA in PBS treated animals, (% control). N.C. indicates values that were not calculated. Each experiment is identified in separate tables below.

The half maximal effective dose (ED₅₀) of each modified oligonucleotide was calculated using GraphPad Prism 10 software (GraphPad Software, San Diego, CA).

TABLE 46
Dose-dependent reduction of human APOE mRNA
in APOE C57BL/6NTac-Apoeem7250 knock-in mice

| | | APOE RNA (% control) | | | |
|---|---|---|---|---|---|
| Compound | Dose | | ED 50 | Spinal | ED 50 |
| No. | (μg) | Cortex | (μg) | Cord | (μg) |
| PBS | — | 100 | — | 100 | — |
| 1693105 | 10 | 100 | 573.3 | 71 | 892.8 |
| | 30 | 85 | | 71 | (above |
| | 100 | 77 | | 47 | upper limit) |
| | 300 | 59 | | 49 | |
| | 700 | 48 | | 61 | |

TABLE 47
Dose-dependent reduction of human APOE mRNA
in APOE C57BL/6NTac-Apoeem7250 knock-in mice

| | | APOE RNA (% control) | | | |
|---|---|---|---|---|---|
| Compound | Dose | | ED 50 | Spinal | ED 50 |
| No. | (μg) | Cortex | (μg) | Cord | (μg) |
| PBS | — | 100 | — | 100 | — |
| 1757575 | 10 | 100 | 337.5 | 82 | 84.6 |
| | 30 | 82 | | 48 | |
| | 100 | 88 | | 40 | |
| | 300 | 51 | | 28 | |
| | 700 | 30 | | 51 | |
| 1757576 | 10 | 108 | 235.5 | 58 | 23.0 |
| | 30 | 121 | | 55 | |
| | 100 | 63 | | 23 | |
| | 300 | 37 | | 20 | |
| | 700 | 31 | | 18 | |
| 1778130 | 10 | 123 | 502.4 | 88 | 35.9 |
| | 30 | 100 | | 35 | |
| | 100 | 69 | | 27 | |
| | 300 | 74 | | 22 | |
| | 700 | 37 | | 32 | |
| 1778131 | 10 | 115 | 216.5 | 79 | 94.0 |
| | 30 | 105 | | 64 | |
| | 100 | 61 | | 36 | |
| | 300 | 37 | | 40 | |
| | 700 | 29 | | 34 | |

TABLE 48
Dose-dependent reduction of human APOE mRNA
in APOE C57BL/6NTac-Apoeem7250 knock-in mice

| | | APOE RNA (% control) | | | |
|---|---|---|---|---|---|
| Compound | Dose | | ED 50 | Spinal | ED 50 |
| No. | (μg) | Cortex | (μg) | Cord | (μg) |
| PBS | — | 100 | — | 100 | — |
| 1746809 | 10 | 98 | 446.6 | 105 | 652.7 |
| | 30 | 89 | | 92 | |
| | 100 | 79 | | 59 | |
| | 300 | 57 | | 48 | |
| | 700 | 41 | | 64 | |
| 1778154 | 10 | 89 | 279.4 | 105 | 243.1 |
| | 30 | 89 | | 137 | |
| | 100 | 70 | | 38 | |
| | 300 | 56 | | 43 | |
| | 700 | 27 | | 47 | |
| 1785598 | 10 | 104 | 175.4 | 97 | 416.7 |
| | 30 | 84 | | 70 | |
| | 100 | 60 | | 65 | |
| | 300 | 37 | | 60 | |
| | 700 | 23 | | 41 | |

### Example 15: Potency of Modified Oligonucleotides Complementary to Human APOE in APOE Knock-In Mice, 8-Weeks

Modified oligonucleotides described above were tested in the APOE C57BL/6NTac-Apoeem7250 knock-in mouse model described above.

APOE C57BL/6NTac-Apoeem7250 knock-in mice were divided into groups of 3-4 mice each. Each mouse received a single ICV bolus of modified oligonucleotide at various doses indicated in the tables below. A group of 4 mice received a single ICV bolus with PBS as a negative control.

Eight weeks post treatment, mice were sacrificed, and RNA was extracted from cortical brain tissue and spinal cord for quantitative real-time RT-PCR analysis of RNA expression of SNCA using human primer probe set RTS3073 (described herein above). APOE RNA levels were normalized to mouse Ppia. Mouse Ppia was amplified using primer probe set m_cyclo24 (described herein above). Results are presented as percent human APOE RNA relative to the amount of APOE RNA in PBS treated animals, (% control). N.C. indicates values that were not calculated.

The half maximal effective dose (ED₅₀) of each modified oligonucleotide was calculated using GraphPad Prism 10 software (GraphPad Software, San Diego, CA).

TABLE 49
Dose-dependent reduction of human APOE mRNA
in APOE C57BL/6NTac-Apoeem7250 knock-in mice

APOE RNA (% control)

| Compound | Dose | | ED₅₀ | Spinal | ED₅₀ |
|---|---|---|---|---|---|
| No. | (μg) | Cortex | (μg) | Cord | (μg) |
| PBS | — | 100 | — | 100 | — |
| 1757576 | 10 | 93 | 135.7 | 33 | N.C. |
| | 30 | 92 | | 15 | |
| | 100 | 52 | | 13 | |
| | 300 | 30 | | 9 | |
| | 700 | 18 | | 9 | |

TABLE 50
Dose-dependent reduction of human APOE mRNA
in APOE C57BL/6NTac-Apoeem7250 knock-in mice

APOE RNA (% control)

| Compound | Dose | | ED₅₀ | Spinal | ED₅₀ |
|---|---|---|---|---|---|
| No. | (μg) | Cortex | (μg) | Cord | (μg) |
| PBS | — | 100 | — | 100 | — |
| 1785602 | 10 | 95 | 153.2 | 73 | 26.9 |
| | 30 | 90 | | 40 | |
| | 100 | 57 | | 26 | |
| | 300 | 31 | | 20 | |
| | 700 | 21 | | 22 | |
| 1809635 | 10 | 95 | 146.7 | 51 | N.C. |
| | 30 | 84 | | 27 | |
| | 100 | 62 | | 13 | |
| | 300 | 30 | | 13 | |
| | 700 | 16 | | 18 | |

TABLE 51
Dose-dependent reduction of human APOE mRNA
in APOE C57BL/6NTac-Apoeem7250 knock-in mice

APOE RNA (% control)

| Compound | Dose | | ED₅₀ | Spinal | ED₅₀ |
|---|---|---|---|---|---|
| No. | (μg) | Cortex | (μg) | Cord | (μg) |
| PBS | — | 100 | — | 100 | — |
| 1785599 | 10 | 94 | 143.3 | 54 | 12.18 |
| | 30 | 72 | | 38 | |
| | 100 | 56 | | 16 | |
| | 300 | 37 | | 13 | |
| | 700 | 25 | | 12 | |
| 1809814 | 10 | 97 | 309.5 | 81 | 78.7 |
| | 30 | 85 | | 60 | |
| | 100 | 72 | | 45 | |
| | 300 | 47 | | 29 | |
| | 700 | 37 | | 28 | |

### Example 16: Duration of Action Study of Modified Oligonucleotides Complementary to Human APOE in APOE Knock-In Mice

Modified oligonucleotides described above were tested in the APOE C57BL/6NTac-Apoeem7250 knock-in mouse model described above.

APOE C57BL/6NTac-Apoeem7250 knock-in mice were divided into groups of 2-4 mice each. Each mouse received a single intracerebroventricular (ICV) bolus of 300 μg of modified oligonucleotide. A group of 2-4 mice received a single ICV bolus with PBS as a negative control.

Mice were sacrificed at various timepoints as indicated in the table below and RNA was extracted from cortical brain tissue and spinal cord for quantitative real-time RT-PCR analysis of RNA expression of APOE using primer probe set RTS3073 (described herein above). APOE RNA levels were normalized to mouse Ppia. Mouse Ppia was amplified using primer probe set m_cyclo24 (described herein above). Results are presented as percent human APOE RNA relative to the amount of human APOE RNA in PBS treated animals, (% control). Each experiment is identified in separate tables below.

TABLE 52
Reduction of human APOE RNA in APOE
C57BL/6NTac-Apoeem7250 knock-in mice

| | | |
|---|---|---|
| Compound | Timepoint | APOE RNA (% control) |

| No. | (Days post-dose) | Cortex | Spinal Cord |
|---|---|---|---|
| 1652901 | 14 | 43 | 33 |
| | 112 | 90 | 51 |
| 1721303 | 14 | 36 | 32 |
| | 112 | 75 | 40 |
| 1721304 | 14 | 46 | 45 |
| | 112 | 67 | 42 |
| 1721305 | 14 | 52 | 37 |
| | 112 | 81 | 40 |
| 1721306 | 14 | 58 | 63 |
| | 112 | 89 | 60 |
| 1652902 | 14 | 43 | 27 |
| | 112 | 75 | 32 |
| 1721307 | 14 | 38 | 31 |
| | 112 | 73 | 34 |
| 1721308 | 14 | 48 | 45 |
| | 112 | 85 | 42 |
| 1721309 | 14 | 45 | 34 |
| | 112 | 93 | 39 |
| 1721310 | 14 | 35 | 38 |
| | 112 | 88 | 65 |

TABLE 53
Reduction of human APOE RNA in APOE
C57BL/6NTac-Apoeem7250 knock-in mice

| | | |
|---|---|---|
| Compound | Timepoint | APOE RNA (% control) |

| No. | (Days post-dose) | Cortex | Spinal Cord |
|---|---|---|---|
| 1746784 | 112 | 38 | 24 |
| | 140 | 45 | 56 |
| 1746789 | 112 | 63 | 83 |
| | 140 | 75 | 80 |
| 1746790 | 112 | 75 | 62 |
| | 140 | 77 | 108 |
| 1746799 | 112 | 46 | 78 |
| | 140 | 70 | 66 |
| 1746800 | 112 | 52 | 88 |
| | 140 | 75 | 58 |
| 1746801 | 112 | 47 | 70 |
| | 140 | 67 | 138 |
| 1746803 | 112 | 54 | 74 |
| | 140 | 73 | 82 |
| 1746804 | 112 | 37 | 205 |
| | 140 | 60 | 207 |
| 1746809 | 112 | 55 | 86 |
| | 140 | 66 | 107 |
| 1746810 | 112 | 63 | 80 |
| | 140 | 82 | 106 |
| 1746811 | 112 | 71 | 95 |
| | 140 | 89 | 103 |
| 1747050 | 112 | 63 | 93 |
| | 140 | 74 | 86 |
| 1753989 | 112 | 58 | 117 |
| | 140 | 72 | 79 |

TABLE 54
Reduction of human APOE RNA in APOE
C57BL/6NTac-Apoeem 7250 knock-in mice

| | | |
|---|---|---|
| Compound | Timepoint | APOE RNA (% control) |

| No. | (Days post-dose) | Cortex | Spinal Cord |
|---|---|---|---|
| 1757576 | 7 | 45 | 42 |
| | 14 | 32 | 25 |
| 1785602 | 7 | 40 | 56 |
| | 14 | 30 | 28 |
| 1809635 | 7 | 40 | 44 |
| | 14 | 30 | 27 |
| 1809814 | 7 | 62 | 84 |
| | 14 | 57 | 53 |

### Example 17: Evaluation of Proinflammatory Effects in BJAB Assay

Human APOE modified oligonucleotides were tested for potential immunostimulatory properties in an in vitro human BJAB activation assay.

Human Burkitt lymphoma B cells, BJAB cells (DSMZ, Cat #ACC 757) were cultured in RPMI1640 medium containing 20% fetal bovine serum at 37° C. and 5% CO2. Cells were maintained at a density of 0.5-0.7×10⁶ cells per milliliter. Cells were transferred to 50 mL conical Falcon tubes and centrifuged at 330 RCF for 5 minutes before being resuspended at a concentration of 7.5×10⁴ cells per milliliter in RPMI culture medium containing penicillin and streptomycin. 50 μL of the cell suspension was added to a v-bottom tissue culture treated 96-well microplate. 11 μl of 10× concentrated modified oligonucleotides was then added to the plate for a final concentration of 1.6 μM and incubated for 24 hours at 37° C. and 5% CO₂.

Compound No. 353512 (3-14-3 MOE gapmer, TCCCATTTCAGGAGACCTGG, designated herein as SEQ ID NO.: 41) is an internal standard known to be a high responder (inflammatory) for CCL22 release in the assay. Compound No. 104838 (5-10-5 MOE gapmer, GCTGATTAGAGAGAGGTCCC, designated herein as SEQ ID NO.: 42) is an internal standard known to be a non-responder (non-inflammatory) in the assay (a negative control). Compound No. 735746 (3-10-3 cEt gapmer, TTCAAGGTGAACTGTT, designated herein as SEQ ID NO.: 43) is an internal standard known to be a moderate responder (mildly inflammatory) for CCL22 release in the assay. Compound No. 785674 (kk-9-eeekk cEt gapmer with MOE modifications, TATTTCTGCTCCAGGT, designated herein as SEQ ID NO.: 44) is an internal standard known to be a high responder (inflammatory) for CCL22 release in the assay.

After a treatment period of 24 hours, total RNA was isolated from the cells and CCL22 RNA levels were measured by quantitative real-time RT-PCR. CCL22 RNA levels were measured by human primer-probe set RTS49260 (forward sequence CGCGTGGTGAAACACTTCTA, designated herein as SEQ ID NO: 19; reverse sequence GATCGGCACAGATCTCCTTATC, designated herein as SEQ ID NO: 20; probe sequence TGGCGTGGTGTTGCTAACCTTCA, designated herein as SEQ ID NO: 21). CCL22 RNA levels were normalized to total RNA content, as measured by GAPDH. Human GAPDH was measured using the human primer-probe set RTS104 (forward sequence GAAGGTGAAGGTCGGAGTC, designated herein as SEQ ID NO: 10; reverse sequence GAAGATGGTGATGGGATTTC, designated herein as SEQ ID NO: 11; probe sequence CAAGCTTCCCGTTCTCAGCC, designated herein as SEQ ID NO: 12). Levels of CCL22 RNA is presented log fold increase of CCL22, relative to untreated control cells (UTC). The assignment of the immunostimulatory properties of the test compounds is based on the immunostimulatory properties of the internal control compounds in each assay.

TABLE 55
Levels of CCL22 in BJAB cells treated with modified
oligonucleotides targeting human APOE

| Compound | LOG10 CCL22 | |
|---|---|---|
| No. | relative to UTC | Immunostimulatory Properties |
| 104838 | 0.09 | Non-Inflammatory-Control |
| 353512 | 0.57 | Inflammatory-Control |
| 735746 | 0.22 | Mildly inflammatory- control |
| 785674 | 0.39 | Inflammatory-Control |
| 1517853 | 1.71 | Highly Inflammatory |
| 1517891 | 0.33 | Mildly Inflammatory |
| 1757576 | 0.01 | Non-Inflammatory |

TABLE 56
Levels of CCL22 in BJAB cells treated with modified
oligonucleotides targeting human APOE

| Compound | LOG10 CCL22 | |
|---|---|---|
| No. | relative to UTC | Immunostimulatory Properties |
| 104838 | 0.04 | Non-Inflammatory-Control |
| 353512 | 0.90 | Inflammatory-Control |
| 735746 | 0.17 | Mildly inflammatory- control |
| 785674 | 0.47 | Inflammatory-Control |
| 1809635 | −0.10 | Non-Inflammatory |

TABLE 57
Levels of CCL22 in BJAB cells treated with modified
oligonucleotides targeting human APOE

| Compound | LOG10 CCL22 | |
|---|---|---|
| No. | relative to UTC | Immunostimulatory Properties |
| 104838 | 0.21 | Non-Inflammatory-Control |
| 353512 | 1.14 | Inflammatory-Control |
| 735746 | 0.50 | Mildly inflammatory- control |
| 785674 | 0.88 | Inflammatory-Control |
| 1785586 | 0.64 | Mildly inflammatory |
| 1785602 | 0.34 | Non-Inflammatory |

TABLE 58
Levels of CCL22 in BJAB cells treated with modified
oligonucleotides targeting human APOE

| Compound | LOG10 CCL22 | |
|---|---|---|
| No. | relative to UTC | Immunostimulatory Properties |
| 104838 | 0.20 | Non-Inflammatory-Control |
| 353512 | 0.81 | Inflammatory-Control |
| 735746 | 0.28 | Mildly inflammatory- control |
| 785674 | 0.54 | Inflammatory-Control |
| 1809814 | 0.07 | Non-Inflammatory |
| 1809815 | 0.81 | Highly inflammatory |
| 1809816 | 0.82 | Highly inflammatory |

TABLE 59
Levels of CCL22 in BJAB cells treated with modified
oligonucleotides targeting human APOE

| Compound | LOG10 CCL22 | |
|---|---|---|
| No. | relative to UTC | Immunostimulatory Properties |
| 104838 | 0.44 | Non-Inflammatory-Control |
| 353512 | 1.04 | Inflammatory-Control |
| 735746 | 0.39 | Mildly inflammatory- control |
| 785674 | 0.77 | Inflammatory-Control |
| 1517578 | 0.73 | Inflammatory |

### Example 18: Duration of Action Study of Modified Oligonucleotides Complementary to Human APOE in APOE Knock-In Mice

Modified oligonucleotides described above were tested in the APOE C57BL/6NTac-Apoeem7250 knock-in model (described above).

APOE knock-in mice were divided into groups of 3-6 mice each as indicated in the tables below. Each mouse received a single intracerebroventricular (ICV) bolus of 300 μg of modified oligonucleotide. A group of 4-6 mice received a single ICV bolus with PBS as a negative control.

Mice were sacrificed at various timepoints as indicated in the table below and RNA was extracted from cortical brain tissue and spinal cord for quantitative real-time RT-PCR analysis of RNA expression of APOE using primer probe set RTS3073 (described herein above). APOE RNA levels were normalized to mouse Ppia. Mouse Ppia was amplified using primer probe set m_cyclo24 (described herein above). Results are presented as percent human APOE RNA relative to the amount of human APOE RNA in PBS treated animals, (% control). Each experiment is identified in separate tables below.

TABLE 60
Reduction of human APOE RNA in APOE C57BL/6NTac-
Apoeem7250 knock-in mice; n = 3-4

| | | |
|---|---|---|
| Compound | Timepoint | APOE RNA (% control) |

| No. | (Days post-dose) | Cortex | Spinal Cord |
|---|---|---|---|
| 1809635 | 28 | 29 | 11 |
| | 112 | 24 | 13 |
| | 140 | 52‡ | 27‡ |
| 1809814 | 28 | 55 | 52 |
| | 112 | 57 | 39 |
| | 140 | 71 | 43 |
| 1810064 | 28 | 40 | 22 |
| | 112 | 62 | 31 |
| | 140 | 63 | 48 |

‡indicates fewer than 4 samples available

TABLE 61
Reduction of human APOE RNA in APOE C57BL/6NTac-
Apoeem7250 knock-in mice; n = 6

| | | |
|---|---|---|
| Compound | Timepoint | APOE RNA (% control) |

| No. | (Days post-dose) | Cortex | Spinal Cord |
|---|---|---|---|
| 1809635 | 28 | 25 | 18 |
| | 56 | 19 | 11 |
| | 112 | 35 | 14 |
| | 168 | 35 | 26 |
| | 342 | 68 | 36 |

TABLE 62
Reduction of human APOE RNA in APOE C57BL/6NTac-
Apoeem7250 knock-in mice; n = 6

| | | |
|---|---|---|
| Compound | Timepoint | APOE RNA (% control) |

| No. | (Days post-dose) | Cortex | Spinal Cord |
|---|---|---|---|
| 1809636 | 56 | 32 | 14 |
| | 112 | 33 | 13 |
| | 224 | 65‡ | 41‡ |
| 1809637 | 56 | 26‡ | 19 |
| | 112 | 42 | 17 |
| | 224 | 58‡ | 36‡ |

‡indicates fewer than 6 samples available

TABLE 63
Reduction of human APOE RNA in APOE C57BL/6NTac-
Apoeem7250 knock-in mice; n = 4-6

| | | |
|---|---|---|
| Compound | Timepoint | APOE RNA (% control) |

| No. | (Days post-dose) | Cortex | Spinal Cord |
|---|---|---|---|
| 1757576 | 196 | 71 | 28 |
| | 224 | 92‡ | 33‡ |
| | 285 | 87 | 86 |
| 1785602 | 196 | 56 | 34 |
| | 224 | 79 | 56 |
| | 285 | 84† | 72† |
| 1809635 | 196 | 50 | 33 |
| | 224 | 55 | 26 |
| | 285 | 67 | 41 |
| 1809814 | 196 | 68 | 57 |
| | 224 | 90 | 54 |
| | 285 | 95 | 84 |

‡indicates fewer than 6 samples
†indicates fewer than 4 samples

What is claimed:

1. An oligomeric agent according to the following chemical notation: N¹ₖₛ^{m}CₑₒTₑₒTₑₛGₑₛGₖₛT_{ds}G_{dz}A_{dz}A_{dz}T_{ds}^{m}C_{ds}T_{ds}T_{ds}T_{ds}A_{dz}TₖₛTₖₛN³ₑₛN²ₑ (SEQ ID NO: 51), wherein:
A=an adenine nucleobase,
^{m}C=a 5-methylcytosine nucleobase,
G=a guanine nucleobase,
T=a thymine nucleobase,
N¹=an adenine nucleobase, a modified adenine, a hypoxanthine, an abasic sugar moiety, a terminal group, or is absent, wherein when N¹ is absent its sugar and internucleoside linkage are also absent,
N²=an adenine nucleobase, a modified adenine, a hypoxanthine, an abasic sugar moiety, a terminal group, or is absent, wherein when N² is absent its sugar and internucleoside linkage are also absent,
N³=an adenine nucleobase, a modified adenine, a hypoxanthine, an abasic sugar moiety, a terminal group, or is absent, wherein when N³ is absent its sugar and internucleoside linkage are also absent,
e=a 2′-MOE sugar moiety,
k=a cEt sugar moiety,
d=a 2′-β-D-deoxyribosyl sugar moiety,
s=a phosphorothioate internucleoside linkage,
o=a phosphodiester internucleoside linkage, and
z=a mesyl phosphoramidate internucleoside linkage; and
wherein the oligomeric agent optionally comprises a conjugate group.

2. The oligomeric agent of claim 1, wherein N¹, N², and N³ is each independently selected from an adenine nucleobase, a modified adenine, and a hypoxanthine or is absent.

3. A modified oligonucleotide according to the following chemical structure: 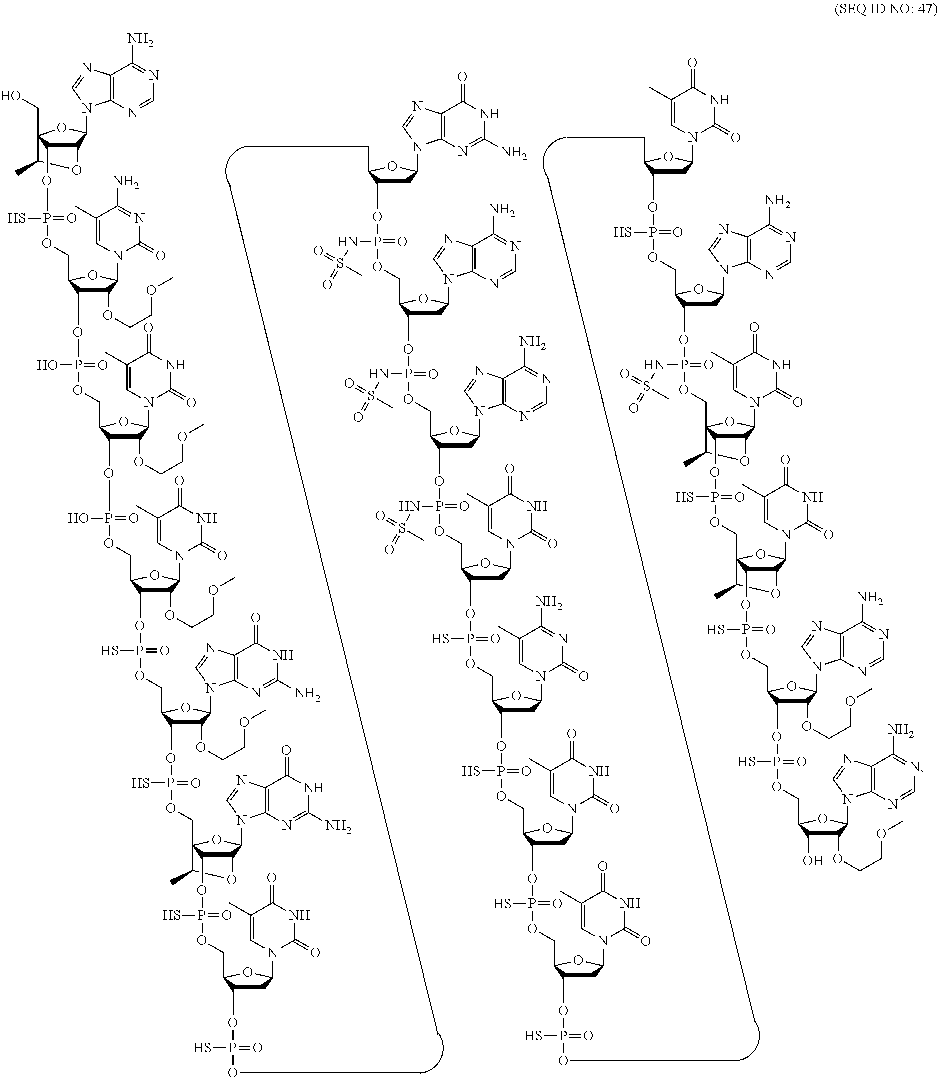
or a pharmaceutically acceptable salt thereof.

4. The modified oligonucleotide of claim 3, wherein the modified oligonucleotide is a pharmaceutically acceptable salt comprising one or more cations selected from sodium, potassium, calcium, and magnesium.

5. A modified oligonucleotide according to the following chemical structure:

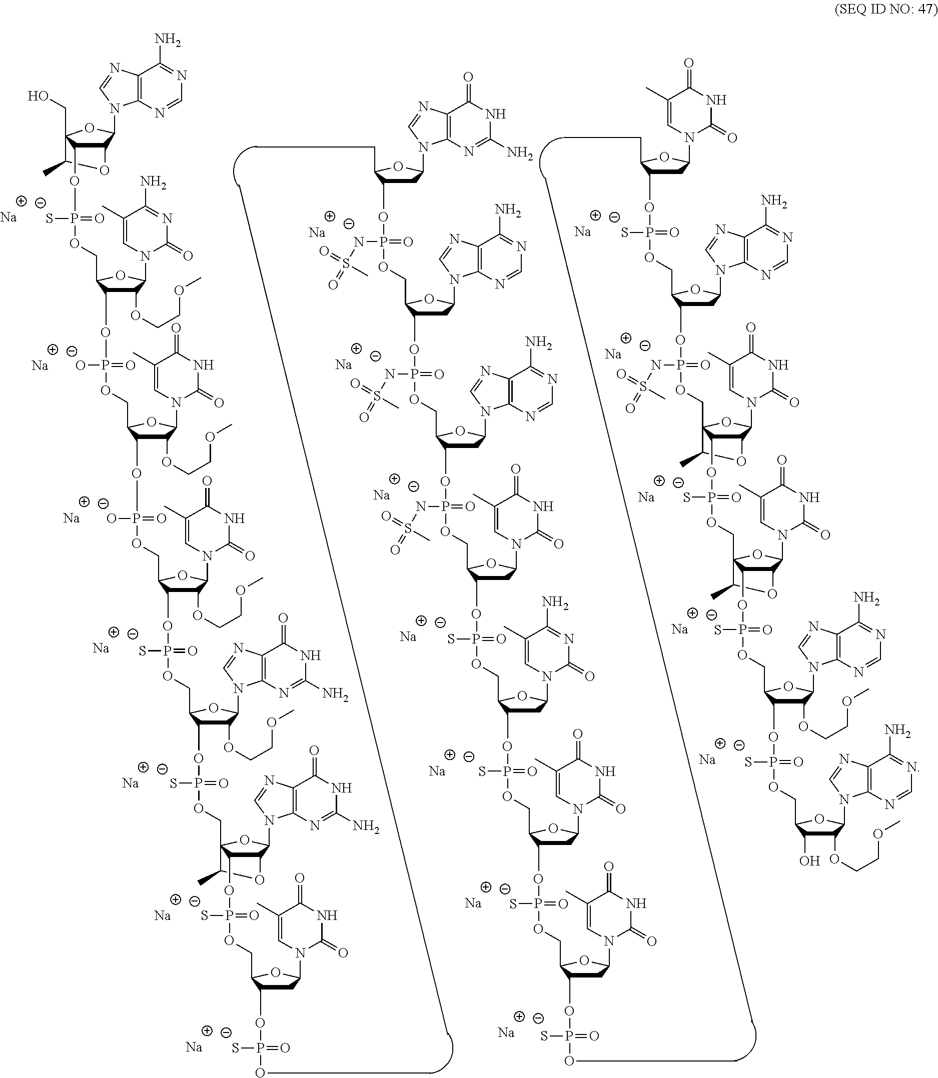

6. An oligomeric agent comprising a modified oligonucleotide according to the following chemical notation: Aₖₛ^{m}CₑₒTₑₒTₑₛGₑₛGₖₛT_{ds}G_{dz}A_{dz}A_{dz}T_{ds}^{m}C_{ds}T_{ds}T_{ds}T_{ds}A_{dz}TₖₛTₖₛAₑₛAₑ (SEQ ID NO: 47), wherein:
A=an adenine nucleobase,
^{m}C=a 5-methylcytosine nucleobase,
G=a guanine nucleobase,
T=a thymine nucleobase,
e=a 2′-MOE sugar moiety,
k=a cEt sugar moiety,
d=a 2′-β-D-deoxyribosyl sugar moiety,
s=a phosphorothioate internucleoside linkage,
o=a phosphodiester internucleoside linkage, and
z=a mesyl phosphoramidate internucleoside linkage; and the oligomeric agent does not include a conjugate group or a terminal group.

7. The oligomeric agent of claim 6, wherein the modified oligonucleotide is a pharmaceutically acceptable salt.

8. The oligomeric agent of claim 7, wherein the pharmaceutically acceptable salt comprises one or more cations selected from sodium, potassium, calcium, and magnesium.

9. A population of modified oligonucleotides of claim 3, wherein all of the phosphorothioate internucleoside linkages of the modified oligonucleotide are stereorandom, all of the mesyl phosphoramidate internucleoside linkages of the modified oligonucleotide are stereorandom, or a combination thereof.

10. A population of modified oligonucleotides of claim 4, wherein all of the phosphorothioate internucleoside linkages of the modified oligonucleotide are stereorandom, all of the mesyl phosphoramidate internucleoside linkages of the modified oligonucleotide are stereorandom, or a combination thereof.

11. A population of modified oligonucleotides of claim 5, wherein all of the phosphorothioate internucleoside linkages of the modified oligonucleotide are stereorandom, all of the mesyl phosphoramidate internucleoside linkages of the modified oligonucleotide are stereorandom, or a combination thereof.

12. A population of oligomeric agents of claim 6, wherein all of the phosphorothioate internucleoside linkages of the modified oligonucleotide are stereorandom, all of the mesyl phosphoramidate internucleoside linkages of the modified oligonucleotide are stereorandom, or a combination thereof.

13. A pharmaceutical composition comprising the modified oligonucleotide of claim 3, and a pharmaceutically acceptable diluent.

14. The pharmaceutical composition of claim 13, wherein the pharmaceutically acceptable diluent is artificial cerebrospinal fluid (aCSF) or phosphate-buffered saline (PBS).

15. The pharmaceutical composition of claim 14, wherein the pharmaceutical composition consists essentially of the modified oligonucleotide and aCSF.

16. The pharmaceutical composition of claim 14, wherein the pharmaceutical composition consists essentially of the modified oligonucleotide and PBS.

17. A pharmaceutical composition comprising the modified oligonucleotide of claim 4, and a pharmaceutically acceptable diluent.

18. The pharmaceutical composition of claim 17, wherein the pharmaceutically acceptable diluent is artificial cerebrospinal fluid (aCSF) or phosphate-buffered saline (PBS).

19. The pharmaceutical composition of claim 18, wherein the pharmaceutical composition consists essentially of the modified oligonucleotide and aCSF.

20. The pharmaceutical composition of claim 18, wherein the pharmaceutical composition consists essentially of the modified oligonucleotide and PBS.

21. A pharmaceutical composition comprising the modified oligonucleotide of claim 5, and a pharmaceutically acceptable diluent.

22. The pharmaceutical composition of claim 21, wherein the pharmaceutically acceptable diluent is artificial cerebrospinal fluid (aCSF) or phosphate-buffered saline (PBS).

23. The pharmaceutical composition of claim 22, wherein the pharmaceutical composition consists essentially of the modified oligonucleotide and aCSF.

24. The pharmaceutical composition of claim 22, wherein the pharmaceutical composition consists essentially of the modified oligonucleotide and PBS.

25. A pharmaceutical composition comprising the oligomeric agent of claim 6, and a pharmaceutically acceptable diluent.

26. The pharmaceutical composition of claim 25, wherein the pharmaceutically acceptable diluent is artificial cerebrospinal fluid (aCSF) or phosphate-buffered saline (PBS).

27. The pharmaceutical composition of claim 26, wherein the pharmaceutical composition consists essentially of the modified oligonucleotide and aCSF.

28. The pharmaceutical composition of claim 26, wherein the pharmaceutical composition consists essentially of the modified oligonucleotide and PBS.

29. A pharmaceutical composition comprising the population of modified oligonucleotides of claim 9, and a pharmaceutically acceptable diluent.

30. The pharmaceutical composition of claim 29, wherein the pharmaceutically acceptable diluent is artificial cerebrospinal fluid (aCSF) or phosphate-buffered saline (PBS).

31. A pharmaceutical composition comprising the population of modified oligonucleotides of claim 10, and a pharmaceutically acceptable diluent.

32. The pharmaceutical composition of claim 31, wherein the pharmaceutically acceptable diluent is artificial cerebrospinal fluid (aCSF) or phosphate-buffered saline (PBS).

33. A pharmaceutical composition comprising the population of modified oligonucleotides of claim 11, and a pharmaceutically acceptable diluent.

34. The pharmaceutical composition of claim 33, wherein the pharmaceutically acceptable diluent is artificial cerebrospinal fluid (aCSF) or phosphate-buffered saline (PBS).

35. A pharmaceutical composition comprising the population of oligomeric agents of claim 12, and a pharmaceutically acceptable diluent.

36. The pharmaceutical composition of claim 35, wherein the pharmaceutically acceptable diluent is artificial cerebrospinal fluid (aCSF) or phosphate-buffered saline (PBS).
